(19) United States
(12) Patent Application Publication
(10) Pub. No.: US 2023/0226119 A1
(43) Pub. Date: Jul. 20, 2023

(54) UMBILICAL CORD DERIVED REGENERATIVE AND IMMUNE MODULATORY STEM CELL POPULATIONS

(71) Applicant: Therapeutic Solutions International, Inc., Oceanside, CA (US)

(72) Inventors: Thomas E. Ichim, Oceanside, CA (US); Timothy G. Dixon, Oceanside, CA (US)

(73) Assignee: Therapeutic Solutions International, Inc., Oceanside, CA (US)

(21) Appl. No.: 18/074,405

(22) Filed: Dec. 2, 2022

Related U.S. Application Data
(60) Provisional application No. 63/254,469, filed on Oct. 11, 2021.

(51) Int. Cl. A61K 35/51; C12N 5/073; A61P 1/00; A61P 37/06

(52) U.S. Cl. CPC A61K 35/51; A61P 1/00; A61P 37/06; C12N 5/0605; C12N 2500/02; C12N 2501/02; C12N 2501/15; C12N 2501/165; C12N 2501/2333; C12N 2509/00

(57) ABSTRACT

The present invention provides universal donor cellular populations derived from umbilical cords possessing ability to elicit immune modulation and evoke regeneration when administered into a mammalian host. Generation of cellular products for clinical use are provided including methodologies of expansion, characterization, and means of therapeutic implementation.

### CROSS-REFERENCE TO RELATED APPLICATIONS

This application is a non-provisional of, and claims priority to U.S. Provisional Application No. 63/254,469, titled “Umbilical Cord Derived Regenerative and Immune Modulatory Stem Cell Populations”, filed on Oct. 11, 2021, which is hereby incorporated by reference in its entirety.

### FIELD OF THE INVENTION

The teachings herein are directed to Wharton Jelly derived cellular compositions possessing immune modulatory, neurogenic, anti-inflammatory, angiogenic and regenerative activity and methods of generating the same.

### BACKGROUND OF THE INVENTION

In science and medicine, the definition of the words “stem cells” is a cell that can make copies of itself, and when needed, make other types of cells, the words “stem cells” evokes a variety of different reactions from different people. In some situations, “stem cells” is associated with futuristic research that one day will help patients suffering from previously incurable diseases. In other situations, “stem cells” represents controversial research into areas using tissue such as of embryonic or fetal origin. For us, “stem cells” represents “repair cells” of the body.

To those of skill in the art, it has become known that there exist numerous types of stem cells, there are hematopoietic stem cells, which are responsible for production of blood. There are embryonic stem cells, which are capable of generating any tissue in the body, however, suffer from the predisposition to generate tumors termed “teratomas”. There are tissue-specific stem cells, such as brain stem cells in the dentate gyrus and subventricular zone, whose proliferation is associated with higher mental activity and resistance to depression. While numerous types of stem cells have been discovered, and most likely, many will be discovered, we decided to focus on one particular type of stem cell: the mesenchymal stem cell (MSC).

The reason use of MSC as compared to other cell types for development of therapeutics is exciting, in our opinion, because of the following: a) MSC are true “repair cells”. In many conditions, healing is associated with MSC producing growth factors, which coordinate numerous cell types to initiate and maintain recovery of bodily function after injury; b) MSC do not require matching between donor and recipient. This is very important because it allows for the use of the stem cell as a “drug”. This means that large quantity of cells can be grown, standardized, characterized and subsequently used to treat a variety of different patients; c) MSC act as “biological anti-inflammatories”. Specifically, not only do MSC promote healing of injured tissues, but they also reduce inflammation. One very important aspect of MSC reducing inflammation is that they only reduce inflammation in the presence of tissue injury. This means that if MSC are injected into a patient with no inflammation, then the MSC do not produce anti-inflammatory products. In other cases, when the patient suffers from an inflammatory condition, the MSC actually produce more anti-inflammatory agents in order to reduce the inflammation. Essentially, the MSC act as a “natural” anti-inflammatory cell, specifically “knowing” how many and what factors to producing; d) MSC home to tissues of injury. It is known that most injury is associated with generation of blood clotting. This process results in lack of oxygen to the damaged tissue. When tissue lacks oxygen, cells of the tissue start producing “chemokines” which act as local beacons, calling in MSC only to the area of tissue injury.

In addition to conceptual advantages of MSC, one very important aspect of this particular family of stem cells is that they have been used in numerous clinical trials with an excellent safety profile. While not all clinical trials have shown specific improvements from an efficacy perspective, adverse reactions to MSC administration, whether intravenous, intrathecal, intra-arterial, or intramuscular, have largely not been observed.

Unfortunately, there appears to be room for improvement in the area of stem cells in general, and particularly in the area of mesenchymal stem cells. We believe that types of mesenchymal stem cells are needed because of: a) need for enhanced growth factor production by MSC; b) need for augmentation of homing by MSC to tissue of injury; and c) desire for MSC to be more economical. Here we describe the previously type of MSC from umbilical cord matrix/wharton’s jelly, which have been modified to possess therapeutic activity that is superior to other stem cell types.

### SUMMARY

Preferred embodiments are directed to a Wharton Jelly derived cellular composition possessing immune modulatory, neurogenic, anti-inflammatory, angiogenic and regenerative activity.

Preferred embodiments are directed to compositions produced by the following steps: a) obtaining an isolated umbilical cord; b) dissociating Wharton’s Jelly to obtain mononuclear cells; c) optionally purifying subsets of said mononuclear cells; d) exposing said mononuclear cells to a regenerative adjuvant; e) culturing said mononuclear cells in absence of, or in the presence of said regenerative adjuvant and/or adding a secondary regenerative adjuvant; and f) selecting said cultured cells for further use, optionally carrying out another purification step.

Preferred embodiments are directed to compositions wherein said cell isolated from said Wharton’s Jelly express a marker selected from a group of markers comprising: CD144, CD105, and CD31.

Preferred embodiments are directed to compositions wherein said cells are obtained from placenta perivascular tissue as a substitute for Wharton’s Jelly.

Preferred embodiments are directed to compositions wherein said cells are s isolated from fetal vascular lobules of a hemochorial placenta.

Preferred embodiments are directed to compositions wherein said cells are isolated by: dissociating fetal vascular lobules from a hemochorial placenta; digesting the dissociated fetal vascular lobes with an enzymatic mixture or by mechanical means; applying a filtration means to said dissociated lobes in order to remove particulates; obtaining mononuclear cells; plating said mononuclear cells in a substrate allowing for growth of said mononuclear cells to confluency; detaching the confluent cells from the plate; and isolating for expression of CD144 and substantially lack of expression of CD45, optionally one or more steps are performed in the presence of hypoxia, wherein hypoxia is sufficient to induce translocation of HIF-1 alpha.

Preferred embodiments are directed to compositions wherein dissociation of fetal vascular lobes is accomplishing by incubation with a mixture of about 2% collagenase, about 0.25% trypsin and about 0.1% DNAse in tissue culture medium.

Preferred embodiments are directed to compositions wherein dissociation of fetal vascular lobes is accomplishing by incubation with a mixture of about 2% collagenase, about 0.25% trypsin and about 0.1% DNAse in tissue culture medium.

Preferred embodiments are directed to compositions wherein cells isolated are comprised of adherent cells expressing the marker CD73 but substantially lacking CD105.

Preferred embodiments are directed to compositions wherein cells isolated are comprised of adherent cells expressing the marker CD73 and CD105 but lacking in CD90.

Preferred embodiments are directed to compositions wherein said regenerative adjuvant is an anti-inflammatory cytokine.

Preferred embodiments are directed to compositions wherein said anti-inflammatory cytokine is selected from a group comprising of IL-4, IL-10, IL-13, IL-20, IL-22 and IL-35.

Preferred embodiments are directed to compositions wherein said anti-inflammatory cytokine is TGF-beta.

Preferred embodiments are directed to compositions wherein said anti-inflammatory cytokine is PGE-2.

Preferred embodiments are directed to compositions wherein said anti-inflammatory cytokine is VEGF.

Preferred embodiments are directed to compositions wherein said composition is composed of mesenchymal stem cells, and wherein said first regenerative adjuvant is hypoxia.

Preferred embodiments are directed to compositions wherein said mesenchymal stem cells possess one or more markers selected from a group comprising of: a) CD11b; b) CD11c; c) CD20; d) CD56; e) CD57 f) CD73; g) CD90; h) CD105; i) membrane bound TGF-beta; and j) neuropilin.

Preferred embodiments are directed to compositions wherein said composition is capable of inhibiting T cell mediated immune responses.

Preferred embodiments are directed to compositions wherein said T cell mediated immune responses comprise of Th1 cell production of cytokines.

Preferred embodiments are directed to compositions wherein said cytokine is selected from a group of cytokines comprising of: a) IL-2; b) IL-6; c) IL-8; d) IL-12; e) IL-15; f) IL-18; g) interferon gamma; h) TNF-alpha; and i) interleukin-33.

Preferred embodiments are directed to compositions wherein said T cell mediated immune responses is activation of gamma delta T cells.

Preferred embodiments are directed to compositions wherein said gamma delta T cell activation is production of granzyme.

Preferred embodiments are directed to compositions wherein said gamma delta T cell activation is production of perforin.

Preferred embodiments are directed to compositions wherein said gamma delta T cell activation is production of interferon gamma.

Preferred embodiments are directed to compositions wherein said T cell mediated immune response is proliferation of a T cell.

Preferred embodiments are directed to compositions wherein said T cell mediated immune response is activation of a CD8 T cell.

Preferred embodiments are directed to compositions wherein said activation of CD8 cell is proliferation of said CD8 cell.

Preferred embodiments are directed to compositions wherein said activation of CD8 cell is production of perforin.

Preferred embodiments are directed to compositions wherein said activation of CD8 cell is production of granzyme.

Preferred embodiments are directed to compositions wherein said activation of CD8 cells is induction of cytotoxicity.

Preferred embodiments are directed to compositions wherein said activation of CD8 cells is production of inflammatory cytokines.

Preferred embodiments are directed to compositions wherein said inflammatory cytokines are selected from a group comprising of: a) RANTES; b) MIP-1 alpha; c) MIP-1 beta; d) IL-2; e) IL-6; f) IL-8; g) IL-12; h) IL-15; i) IL-18; j) interferon gamma; k) TNF-alpha; and 1) interleukin-33.

Preferred embodiments are directed to compositions wherein said T cell mediated immune response is a homeostatic expansion of said T cells.

Preferred embodiments are directed to compositions wherein said homeostatic expansion comprises proliferation of a T cell in absence of need for a second signal.

Preferred embodiments are directed to compositions wherein said second signal is CD28.

Preferred embodiments are directed to compositions wherein said second signal is ICOS.

Preferred embodiments are directed to compositions wherein said second signal is CD40.

Preferred embodiments are directed to compositions wherein said homeostatic expansion is ability of said T cell to proliferate independent of T cell receptor ligation.

Preferred embodiments are directed to compositions wherein said homeostatic expansion is ability of said T cell to proliferate in response to IL-7.

Preferred embodiments are directed to compositions wherein said homeostatic expansion is ability of said T cell to proliferate in response to IL-15.

Preferred embodiments are directed to compositions wherein said inhibition of T cell mediated responses is performed by augmentation of immune regulatory cells.

Preferred embodiments are directed to compositions wherein said immune regulatory cells are cells possessing ability to suppress T cell activation, and/or proliferation, and/or cytokine secretion.

Preferred embodiments are directed to compositions wherein said immune regulatory cells are T cells.

Preferred embodiments are directed to compositions wherein said immune regulatory T cells are T regulatory cells.

Preferred embodiments are directed to compositions wherein said T regulatory cells possess ability to suppress a conventional T cell in an antigen specific manner.

Preferred embodiments are directed to compositions wherein said T regulatory cells possess ability to suppress a conventional T cell in an antigen non-specific manner.

Preferred embodiments are directed to compositions wherein said T regulatory cells possess ability to suppress a conventional T cell in a contact dependent manner.

Preferred embodiments are directed to compositions wherein said T regulatory cells possess ability to suppress a conventional T cell in a contact independent manner.

Preferred embodiments are directed to compositions wherein said T regulatory cells are CD4 positive and CD25 positive.

Preferred embodiments are directed to compositions wherein said conventional T cells are CD4 positive and CD25 negative.

Preferred embodiments are directed to compositions wherein said T regulatory cells are CD4 positive and CTLA-4 positive.

Preferred embodiments are directed to compositions wherein said conventional T cells are CD4 positive and CTLA-4 negative.

Preferred embodiments are directed to compositions wherein said T regulatory cells are CD4 positive and GITR positive.

Preferred embodiments are directed to compositions wherein said conventional T cells are CD4 positive and GITR negative.

Preferred embodiments are directed to compositions wherein said T regulatory cells are CD4 positive and CD39 and/or CD73 positive.

Preferred embodiments are directed to compositions wherein said conventional T cells are CD4 positive and CD39 and/or CD73 negative.

Preferred embodiments are directed to compositions wherein said T regulatory cells are CD4 positive and IL-7 receptor negative

Preferred embodiments are directed to compositions wherein said conventional T cells are CD4 positive and IL-7 receptor positive.

Preferred embodiments are directed to compositions wherein said T regulatory cells are capable of inhibiting maturation of dendritic cells in response to ligation of a “danger signal”.

Preferred embodiments are directed to compositions wherein said “danger signal” is activation of a toll like receptor (TLR).

Preferred embodiments are directed to compositions wherein said TLR is TRL-4.

Preferred embodiments are directed to compositions wherein said activation of dendritic cell maturation endows said dendritic cell ability to induce proliferation of naive T cells.

Preferred embodiments are directed to compositions wherein said activation of dendritic cell maturation endows said dendritic cell ability to induce cytokine secretion of naive T cells.

Preferred embodiments are directed to compositions wherein said activation of dendritic cell maturation endows said dendritic cell ability to induce cytotoxic activity to naive T cells.

Preferred embodiments are directed to compositions wherein said activation of dendritic cell maturation endows said dendritic cell ability to induce differentiation of naive CD4 T cells into a helper phenotype.

Preferred embodiments are directed to compositions wherein said helper phenotype is T helper 1, characterized by expression of interferon gamma, interleukin 2, interleukin 7, interleukin 12, interleukin 15, interleukin 18, and interleukin 23.

Preferred embodiments are directed to compositions wherein said Th1 cells is characterized by expression of STAT4.

Preferred embodiments are directed to compositions wherein said helper phenotype is T helper 2, characterized by expression of IL-4, IL-5, IL-13, and IL-10.

Preferred embodiments are directed to compositions wherein said T helper 2 cell is characterized by expression of STAT6.

Preferred embodiments are directed to compositions wherein said T helper cell is a Th9 cell.

Preferred embodiments are directed to compositions wherein said Th9 cell is characterized by expression of the FOXO1 transcription factor.

Preferred embodiments are directed to compositions wherein said Th9 cell is characterized by expression of interleukin-9

Preferred embodiments are directed to compositions wherein said T helper cell is a Th17 cell.

Preferred embodiments are directed to compositions wherein said Th17 cell produces cytokines selected from a group comprising of: a) IL-17; b) IL-17A); c) IL-17F, and d) IL-6.

Preferred embodiments are directed to compositions wherein said Th17 cell expresses BATF.

Preferred embodiments are directed to compositions wherein said Th17 cell expresses RORgamma.

Preferred embodiments are directed to embodiments wherein said composition is capable of inhibiting antigen presenting cell function.

Preferred embodiments are directed to compositions wherein said antigen presenting cell is a B cell.

Preferred embodiments are directed to compositions wherein said B cell is a CD5 positive B cell.

Preferred embodiments are directed to compositions wherein said antigen presenting cell is an endothelial cell.

Preferred embodiments are directed to compositions wherein said endothelial cell is activated with interferon gamma.

Preferred embodiments are directed to compositions wherein said antigen presenting cell is an epithelial cell.

Preferred embodiments are directed to compositions wherein said antigen presenting cell is activated with interferon gamma.

Preferred embodiments are directed to compositions wherein said antigen presenting cell is a monocyte.

Preferred embodiments are directed to compositions wherein said antigen presenting cell is a macrophage.

Preferred embodiments are directed to compositions wherein said antigen macrophage is an M1 macrophage.

Preferred embodiments are directed to compositions wherein said antigen macrophage is an M2 macrophage.

Preferred embodiments are directed to compositions wherein said M1 macrophage expresses markers selected from a group comprising of: CD80, CD86, CD64, CD16 and CD32.

Preferred embodiments are directed to compositions wherein said M2 macrophage expresses markers selected from a group comprising of: CD68. CD163, and CD206.

Preferred embodiments are directed to compositions wherein said antigen presenting cell is a dendritic cell.

Preferred embodiments are directed to compositions wherein said dendritic cell is a myeloid dendritic cell.

Preferred embodiments are directed to compositions wherein said dendritic cell is a lymphoid dendritic cell.

Preferred embodiments are directed to compositions wherein said inhibition of antigen presenting cell function is associated with suppression of MHC I expression.

Preferred embodiments are directed to compositions wherein said inhibition of antigen presenting cell function is associated with suppression of MHC II expression.

Preferred embodiments are directed to compositions wherein said inhibition of antigen presenting cell function is associated with suppression of peptide generation from proteins inside said antigen presenting cell.

Preferred embodiments are directed to compositions wherein said inhibition of antigen presenting cell function is associated with suppression of peptide loading into MHC I.

Preferred embodiments are directed to compositions wherein said inhibition of antigen presenting cell function is associated with suppression of peptide loading into MHC II.

Preferred embodiments are directed to compositions wherein said inhibition of antigen presenting cell function is associated with suppression of T cell stimulatory cytokine production by said antigen presenting cell.

Preferred embodiments are directed to compositions wherein said T cell stimulatory cytokine is interleukin-2.

Preferred embodiments are directed to compositions wherein said T cell stimulatory cytokine is interleukin-7.

Preferred embodiments are directed to compositions wherein said T cell stimulatory cytokine is interleukin-12.

Preferred embodiments are directed to compositions wherein said T cell stimulatory cytokine is interleukin-15.

Preferred embodiments are directed to compositions wherein said T cell stimulatory cytokine is interleukin-17.

Preferred embodiments are directed to compositions wherein said T cell stimulatory cytokine is interleukin-18.

Preferred embodiments are directed to compositions wherein said inhibition antigen presenting cell function is associated with suppression of costimulatory molecule expression.

Preferred embodiments are directed to compositions wherein said costimulatory molecule expression is CD40.

Preferred embodiments are directed to compositions wherein said costimulatory molecule expression is ICOS ligand.

Preferred embodiments are directed to compositions wherein said costimulatory molecule expression is CD80.

Preferred embodiments are directed to compositions wherein said costimulatory molecule expression is CD86.

Preferred embodiments are directed to compositions wherein said costimulatory molecule expression is CD137.

Preferred embodiments are directed to compositions wherein said inhibition of antigen presenting cell function is associated with suppression of ability of said antigen presenting cell to form an immunological synapse with the T cell.

Preferred embodiments are directed to compositions wherein said inhibition of antigen presenting cell function is associated with suppression of ability of said antigen presenting cell to produce exosomes.

Preferred embodiments are directed to compositions wherein said exosomes possess ability to activate a T cell.

Preferred embodiments are directed to compositions wherein said exosomes possess MHC I.

Preferred embodiments are directed to compositions wherein said exosomes possess MHC II.

Preferred embodiments are directed to compositions wherein said exosomes possess CD40.

Preferred embodiments are directed to compositions wherein said exosomes possess ICOS ligand.

Preferred embodiments are directed to compositions wherein said exosomes possess CD80.

Preferred embodiments are directed to compositions wherein said exosomes possess CD86.

Preferred embodiments are directed to compositions wherein said exosomes possess ability to induce antigen cross presentation.

Preferred embodiments are directed to embodiments wherein said cells induce anti-inflammatory cytokine levels which are sufficient to induce a regulatory T cell immunophenotype.

Preferred embodiments are directed to compositions wherein anti-inflammatory cytokine levels are sufficient to inhibit production of inflammatory cytokine by stimulated T cells by at least 20% relative to baseline.

Preferred embodiments are directed to compositions wherein said inflammatory cytokines are selected from IFN-gamma, IL-17A, IL-1-beta, IL-6, IL-33, HMGB1 and TNF-alpha.

Preferred embodiments are directed to compositions wherein said T cells are selected from CD8.sup.+ T cells, CD4.sup.+ T cells, gamma-delta T cells, and T regulatory cells.

Preferred embodiments are directed to embodiments wherein said cell is engineered to produce at least three anti-inflammatory cytokines at levels sufficient to inhibit an inflammatory response by at least 20% relative to a control.

Preferred embodiments are directed to embodiments wherein said cell is engineered to express a homing molecule.

Preferred embodiments are directed to compositions wherein said engineering is performed by alteration of culture conditions.

Preferred embodiments are directed to compositions wherein said culture conditions comprises exposure to hypoxia.

Preferred embodiments are directed to compositions wherein said hypoxia is sufficient to induce translation of hypoxia inducible factor (HIF)-1.

Preferred embodiments are directed to compositions wherein said hypoxia consists of culture cell in 1-19% oxygen.

Preferred embodiments are directed to compositions wherein said alteration in culture condition is exposure to hyperthermia.

Preferred embodiments are directed to compositions wherein said alteration in culture condition is exposure to acidic conditions.

Preferred embodiments are directed to compositions wherein said alteration in culture condition is exposure to hypotonic conditions.

Preferred embodiments are directed to compositions wherein said homing molecule is anti-integrin alpha4,beta7.

Preferred embodiments are directed to compositions wherein said homing molecule is MAdCAM.

Preferred embodiments are directed to compositions wherein said homing molecule is CCR9.

Preferred embodiments are directed to compositions wherein said homing molecule is CXCR4.

Preferred embodiments are directed to compositions wherein said homing molecule is CXCR7.

Preferred embodiments are directed to compositions wherein said homing molecule is CCR2.

Preferred embodiments are directed to compositions wherein said homing molecule is GPR15.

Preferred embodiments are directed to embodiments wherein Wharton jelly mononuclear cells re-programmed to posses a state of enhanced immaturity as compared to naturally residing Wharton jelly mononuclear cells.

Preferred embodiments are directed to compositions wherein said enhanced immaturity is increased differentiation efficacy.

Preferred embodiments are directed to compositions wherein said differentiation efficacy means ability to differentiate into other tissues and/or at a greater percentage of cells differentiating.

Preferred embodiments are directed to compositions wherein tissues in which said cells are capable of differentiating into are endodermal, ectodermal and mesodermal derived tissues.

Preferred embodiments are directed to compositions wherein said enhanced immaturity is increased ability to produce therapeutic factors.

Preferred embodiments are directed to compositions wherein said therapeutic factors are selected from: a) angiogenic factors; b) neurogenic factors; c) antiapoptotic factors and d) immune modulatory factors.

Preferred embodiments are directed to compositions wherein said angiogenic factors are selected from a group comprising of: a) VEGF; b) HGF-1; c) FGF-1; d) FGF-2; e) angiopoietin; f) interleukin-20.

Preferred embodiments are directed to compositions wherein said neurogenic factors are selected from a group comprising of: a) NGF; b) BDNF; c) CNTF; and d) neurotrophin.

Preferred embodiments are directed to compositions wherein said antiapoptotic factors are selected from a group comprising of: a) molecules capable of increasing bcl-2 expression; b) molecules capable of decreasing BAD expression; c) molecules capable of increasing expression of bcl-2Xl; d) molecules capable of decreasing bcl-2Xs; e) molecules capable of decreasing expression of members of the caspase family; f) molecules capable of increasing survivin expression; and g) molecules capable of increasing livin expression.

Preferred embodiments are directed to compositions wherein said immunomodulatory factors are factors capable of inhibiting T cell activation.

Preferred embodiments are directed to compositions wherein said immunomodulatory factors are factors capable of inhibiting NK cell activation.

Preferred embodiments are directed to compositions wherein said immunomodulatory factors are factors capable of inhibiting NKT cell activation.

Preferred embodiments are directed to compositions wherein said immunomodulatory factors are factors capable of inhibiting gamma delta cell activation.

Preferred embodiments are directed to compositions wherein said immunomodulatory factors are factors capable of inhibiting dendritic cell maturation.

Preferred embodiments are directed to compositions wherein said immunomodulatory factors are factors capable of inhibiting complement activation.

Preferred embodiments are directed to compositions wherein said immunomodulatory factors are factors capable of accelerating neutrophil apoptosis.

Preferred embodiments are directed to compositions wherein said immunomodulatory factors are factors capable of inhibiting mast cell activation.

Preferred embodiments are directed to compositions wherein said immunomodulatory factors are factors capable of inhibiting eosinophil activation.

Preferred embodiments are directed to compositions wherein said immunomodulatory factors are factors capable of inhibiting basophil activation.

Preferred embodiments are directed to compositions wherein said enhanced immaturity is obtained by culture with an agent associated with embryonic microenvironment.

Preferred embodiments are directed to compositions wherein said agent is leukemia inhibitory factor.

Preferred embodiments are directed to compositions wherein said leukemia inhibitory factor is administered to cells that have been synchronized in cell cycle.

Preferred embodiments are directed to compositions wherein said synchronization of cells in cell cycle is achieved by treatment with a mitotic inhibitor.

Preferred embodiments are directed to compositions wherein said mitotic inhibitor is mitomycin C.

Preferred embodiments are directed to compositions wherein synchronization is achieved by serum deprivation.

Preferred embodiments are directed to compositions wherein said leukemia inhibitory factor is administered at a concentration of 1 pg/ml to 100 ng/ml.

Preferred embodiments are directed to compositions wherein said leukemia inhibitory factor is administered at a concentration of 10 pg/ml to 10 ng/ml.

Preferred embodiments are directed to compositions wherein said leukemia inhibitory factor is administered at a concentration of 100 pg/ml to 5 ng/ml.

Preferred embodiments are directed to compositions wherein said leukemia inhibitory factor is administered together with a histone deacetylase inhibitor.

Preferred embodiments are directed to compositions wherein said histone deacetylase inhibitor is valproic acid.

Preferred embodiments are directed to compositions wherein said valproic acid is administered at a concentration of 1 pg/ml to 1 mg/ml.

Preferred embodiments are directed to compositions wherein said valproic acid is administered at a concentration of 100 pg/ml to 100 ng/ml.

Preferred embodiments are directed to compositions wherein said valproic acid is administered at a concentration of 1 ng/ml to 100 ng/ml.

Preferred embodiments are directed to compositions wherein said histone deacetylase inhibitor is vorinostat.

Preferred embodiments are directed to compositions wherein said histone deacetylase inhibitor is belinostat.

Preferred embodiments are directed to compositions wherein said histone deacetylase inhibitor is LAQ824.

Preferred embodiments are directed to compositions wherein said histone deacetylase inhibitor is trichostatin A.

Preferred embodiments are directed to compositions wherein said histone deacetylase inhibitor is Panobinostat.

Preferred embodiments are directed to compositions wherein said histone deacetylase inhibitor is entinostat.

Preferred embodiments are directed to compositions wherein said histone deacetylase inhibitor is CI994.

Preferred embodiments are directed to compositions wherein said histone deacetylase inhibitor is mocetinostat.

Preferred embodiments are directed to compositions wherein said histone deacetylase inhibitor is trapoxin B.

Preferred embodiments are directed to compositions wherein said histone deacetylase inhibitor is phenylbutyrate.

Preferred embodiments are directed to compositions wherein a DNA methyltransferase inhibitor is added to said combination leukemia inhibitory factor and said DNA histone deacetylase inhibitor.

Preferred embodiments are directed to compositions wherein said DNA methyltransferase inhibitory is a nucleic acid derivative.

Preferred embodiments are directed to compositions wherein said DNA methyltransferase inhibitory is decitabine.

Preferred embodiments are directed to compositions wherein said DNA methyltransferase inhibitory is 5-azacytabine.

Preferred embodiments are directed to compositions wherein said reprogramming is induced through suppression of the enzyme GSK-3.

Preferred embodiments are directed to compositions wherein said suppression of said enzyme GSK-3 is accomplished by treatment with lithium.

Preferred embodiments are directed to compositions wherein said lithium is administered at a concentration of 1 pg/ml to 1 mg/ml.

Preferred embodiments are directed to compositions wherein said lithium is administered at a concentration of 10 pg/ml to 100 ng/ml.

Preferred embodiments are directed to compositions wherein said lithium is administered at a concentration of 100 pg/ml to 10 ng/ml.

Preferred embodiments are directed to compositions wherein said reprogramming is induced through induction of expression of the gene OCT¾.

Preferred embodiments are directed to compositions wherein said reprogramming is induced through induction of expression of the gene SOX2.

Preferred embodiments are directed to compositions wherein said reprogramming is induced through induction of expression of the gene KLF4.

Preferred embodiments are directed to compositions wherein said reprogramming is induced through induction of expression of the gene L-MYC.

Preferred embodiments are directed to compositions wherein said reprogramming is induced through induction of expression of the gene LIN28.

Preferred embodiments are directed to compositions wherein said reprogramming is induced through induction of expression of the gene BCL-xL.

Preferred embodiments are directed to compositions wherein said reprogramming is induced through induction of expression of the gene BCL-xL.

Preferred embodiments are directed to compositions wherein said reprogramming is induced through inhibition of p53 expression.

Preferred embodiments are directed to compositions wherein p53 expression is suppression of p53 activity.

Preferred embodiments are directed to compositions wherein said suppression of p53 activity is mediated through administration of a small molecule inhibitor of p53.

Preferred embodiments are directed to compositions wherein said suppression of p53 activity is mediated through administration of decoy oligonucleotides.

Preferred embodiments are directed to compositions wherein said suppression of p53 activity is mediated through administration of decoy peptides.

Preferred embodiments are directed to compositions wherein said suppression of p53 expression is achieved through induction of RNA interference targeting p53.

Preferred embodiments are directed to compositions wherein said induction of RNA interference targeting p53 is induced through administration of short interfering RNA.

Preferred embodiments are directed to compositions wherein said induction of RNA interference targeting p53 is induced through administration of short hairpin RNA.

Preferred embodiments are directed to compositions wherein said suppression of p53 expression is achieved through administration of antisense oligonucleotides targeting p53.

Preferred embodiments are directed to compositions wherein said antisense oligonucleotides induce cleavage of nucleic acids through activation of RNAse H.

Preferred embodiments are directed to compositions wherein said suppression of p53 expression is achieved through administration of ribozymes targeting p53.

Preferred embodiments are directed to compositions wherein said suppression of p53 expression is achieved through gene editing.

Preferred embodiments are directed to compositions wherein said reprogramming is induced by culture of said cells in a liquid media containing ascorbic acid.

Preferred embodiments are directed to compositions wherein said reprogramming is induced by culture of said cells in a liquid media containing transferrin.

Preferred embodiments are directed to compositions wherein said reprogramming is induced by culture of said cells in a liquid media containing sodium bicarbonate.

Preferred embodiments are directed to compositions wherein said reprogramming is induced by culture of said cells in a liquid media containing insulin.

Preferred embodiments are directed to compositions wherein said reprogramming is induced by culture of said cells in a liquid media containing sodium selenite.

Preferred embodiments are directed to compositions wherein said reprogramming is induced by culture of said cells in a hypoxic environment.

Preferred embodiments are directed to compositions wherein said hypoxic environment comprises of oxygen levels low enough to induce activation of hypoxia inducible factor (HIF)-1.

Preferred embodiments are directed to compositions wherein said hypoxic environment is culture of said cells in an environment less than 21% oxygen.

Preferred embodiments are directed to compositions wherein said hypoxic environment is culture of said cells in an environment less than 15% oxygen.

Preferred embodiments are directed to compositions wherein said hypoxic environment is culture of said cells in an environment less than 10% oxygen.

Preferred embodiments are directed to compositions wherein said hypoxic environment is culture of said cells in an environment containing approximately 5% oxygen.

Preferred embodiments are directed to compositions wherein said reprogramming is induced by culture of cells in a liquid media containing a MAP Kinase inhibitor.

Preferred embodiments are directed to compositions wherein said MAP kinase inhibitor is PD0325901/

Preferred embodiments are directed to compositions wherein said reprogramming is induced by culture of cells in a liquid media containing SB431542.

Preferred embodiments are directed to compositions wherein said reprogramming is induced by culture of cells in a liquid media containing CHIR99021.

Preferred embodiments are directed to compositions wherein said reprogramming is induced by culture of cells in a liquid media containing Y-27632.

Preferred embodiments are directed to compositions wherein said reprogramming is induced by culture of cells in a liquid media containing Y-thiazovivin.

Preferred embodiments are directed to compositions wherein said reprogramming is induced by culture of cells in a liquid media containing FGF-1.

Preferred embodiments are directed to compositions wherein said reprogramming is induced by culture of cells in a liquid media containing FGF-2.

Preferred embodiments are directed to compositions wherein said reprogramming is induced by culture of cells in a liquid media containing FGF-5.

Preferred embodiments are directed to compositions wherein said reprogramming is induced by culture of cells in a liquid media containing sodium borate.

Preferred embodiments are directed to compositions wherein said reprogramming is induced by culture of cells in a liquid media containing erythropoietin (EPO).

Preferred embodiments are directed to compositions wherein said reprogramming is induced by culture of cells in a liquid media containing interleukin-3.

Preferred embodiments are directed to compositions wherein said reprogramming is induced by culture of cells in a liquid media containing interleukin-6.

Preferred embodiments are directed to compositions wherein said reprogramming is induced by culture of cells in a liquid media containing interleukin-8.

Preferred embodiments are directed to compositions wherein said reprogramming is induced by culture of cells in a liquid media containing interleukin-10.

Preferred embodiments are directed to compositions wherein said reprogramming is induced by culture of cells in a liquid media containing interleukin-18.

Preferred embodiments are directed to compositions wherein said reprogramming is induced by culture of cells in a liquid media containing interleukin-20.

Preferred embodiments are directed to compositions wherein said reprogramming is induced by culture of cells in a liquid media containing interleukin-25.

Preferred embodiments are directed to compositions wherein said reprogramming is induced by culture of cells in a liquid media containing insulin-like growth factor-1 (IGF-1).

Preferred embodiments are directed to compositions wherein said reprogramming is induced by culture of cells in a liquid media containing dexamethasone.

Preferred embodiments are directed to compositions wherein said reprogramming is induced by culture of cells in a liquid media containing holo-transferrin.

Preferred embodiments are directed to compositions wherein said reprogramming is induced by culture of cells in a liquid media containing amino acids selected from a group comprising of Glycine, L-histidine, L-isoleucine, L-methionine, L-phenylalanine, L-proline, L-hydroxyproline, L-serine, L-threonine, L-tryptophan, and L-tyrosine, L-valine.

Preferred embodiments are directed to compositions wherein said reprogramming is induced by culture of cells in a liquid media containing vitamins and/or antioxidants selected from a group comprising of thiamine, reduced glutathione, ascorbic acid and 2-PO.sub.4.

Preferred embodiments are directed to compositions wherein said reprogramming is induced by culture of cells in a liquid media containing trace elements selected from a group comprising of: Ag.sup.+, Al.sup.3+, Ba.sup.2+, Cd.sup.2+, Co.sup.2+, Cr.sup.3+, Ge.sup.4+, Se.sup.4+, Br.sup.-, I.sup.-, F.sup.-, Mn.sup.2+, Si.sup.4+, V.sup.5+, MO.sup.6+, Ni.sup.2+, Rb.sup.+, Sn.sup.2+, and Zr.sup.4+.

Preferred embodiments are directed to compositions wherein said reprogramming is induced by culture of cells in a liquid media containing cytoplasm of an undifferentiated cell.

Preferred embodiments are directed to compositions wherein said cell being reprogrammed has its membrane temporarily permeabilized.

The composition of 248, wherein said temporary permeabilization allows for entry of cytoplasm of undifferentiated cell into cytoplasm of said cell to be reprogrammed.

Preferred embodiments are directed to compositions wherein said undifferentiated cell is syngeneic with the cell whose reprogramming is desired.

Preferred embodiments are directed to compositions wherein said undifferentiated cell is allogeneic with the cell whose reprogramming is desired.

Preferred embodiments are directed to compositions wherein said undifferentiated cell is xenogeneic with the cell whose reprogramming is desired.

Preferred embodiments are directed to compositions wherein said permeabilization is mediated by electroporation.

Preferred embodiments are directed to compositions wherein said permeabilization is mediated by Streptolysin O treatment.

Preferred embodiments are directed to compositions wherein said permeabilization is mediated by transient treatment with complement membrane attack complex.

Preferred embodiments are directed to compositions wherein said permeabilization is mediated by transient treatment with perforin.

Preferred embodiments are directed to compositions wherein said permeabilization is mediated by transient treatment with granzyme.

Preferred embodiments are directed to compositions wherein said undifferentiated cell is an oocyte.

Preferred embodiments are directed to compositions wherein said oocyte is programmed to be at G0/G1 of cell cycle.

Preferred embodiments are directed to compositions wherein said programming to be at G0/G1 of cell cycle is accomplished by exposure to mitomycin C.

Preferred embodiments are directed to compositions wherein said programming to be at G0/G1 of cell cycle is accomplished by exposure to serum starvation.

Preferred embodiments are directed to compositions wherein said undifferentiated cell is an inducible pluripotent stem cell.

Preferred embodiments are directed to compositions wherein said undifferentiated cell is a parthenogenic derived stem cell.

Preferred embodiments are directed to compositions wherein said undifferentiated cell is an embryonic stem cell.

Preferred embodiments are directed to compositions wherein said undifferentiated cell is a somatic cell nuclear transfer derived stem cell.

Preferred embodiments are directed to compositions wherein said undifferentiated cell is a cytoplasmically reprogrammed stem cell.

Preferred embodiments are directed to compositions wherein said undifferentiated cell is a cell obtained by fusion of an adult cell with a pluripotent stem cell.

Preferred embodiments are directed to compositions wherein said fusion is accomplished by the use of polyethylene glycol.

Preferred embodiments are directed to compositions wherein said fusion is accomplished by the use of electrically mediated fusion.

A method for generating T regulatory cells comprising the steps of: a) obtaining a population of naive T cells; b) contacting said naive T cells with the compositions herein in a manner capable of eliciting immune modulation; and c) providing conditions so as to enable differentiation of naive T cells into T regulatory cells.

Preferred embodiments are directed to methods wherein said naive T cells are CD4 T cells.

Preferred embodiments are directed to methods wherein said naive T cells are CD8 T cells.

Preferred embodiments are directed to methods wherein said naive T cells are CD45RO T cells.

Preferred embodiments are directed to methods wherein said naive T cells are antigenically naive.

Preferred embodiments are directed to methods wherein said naive T cells express IL-2 receptor alpha chain.

Preferred embodiments are directed to methods wherein said cells are capable of immune modulation have been cultured in interferon gamma.

Preferred embodiments are directed to methods wherein said wherein said cells of composition of claim 1 are capable of immune modulation have been cultured in interferon gamma.

Preferred embodiments are directed to methods wherein said wherein said cells are capable of immune modulation have been cultured under hypoxic conditions.

Preferred embodiments are directed to methods wherein said wherein said cells are capable of immune modulation have been transfected with cytoplasm from immature dendritic cells.

Preferred embodiments are directed to methods wherein said immature dendritic cells lack substantial expression of CD40.

Preferred embodiments are directed to methods wherein said immature dendritic cells lack substantial expression of CD80.

Preferred embodiments are directed to methods wherein said immature dendritic cells lack substantial expression of CD86.

Preferred embodiments are directed to methods wherein said immature dendritic cells lack substantial expression of HLA II.

Preferred embodiments are directed to methods wherein said immature dendritic cells possess PD-1L.

Preferred embodiments are directed to methods wherein said immature dendritic cells possess ILT-3.

Preferred embodiments are directed to methods wherein said immature dendritic cells secrete IL-10.

Preferred embodiments are directed to methods wherein said immature dendritic cells are derived from a cell line.

Preferred embodiments are directed to methods wherein said immature dendritic cells are derived from primary donors.

Preferred embodiments are directed to methods wherein said wherein said cells are capable of immune modulation have been cultured in platelet rich plasma.

Preferred embodiments are directed to methods wherein said wherein said cells are capable of immune modulation have been genetically modified to express an immune suppressive protein.

Preferred embodiments are directed to methods wherein said immune suppressive protein is IL-10.

Preferred embodiments are directed to methods wherein said immune suppressive protein is TGF-beta.

Preferred embodiments are directed to methods wherein said immune suppressive protein is IL-32.

Preferred embodiments are directed to methods wherein said immune suppressive protein is IL-35.

Preferred embodiments are directed to methods wherein said immune suppressive protein is IL-12p40 homodimers.

Preferred embodiments are directed to methods wherein said immune suppressive protein is HLA-G.

Preferred embodiments are directed to methods wherein said immune suppressive protein is ILT-3.

Preferred embodiments are directed to methods wherein said immune suppressive protein is indolamide 2,3 deoxygenase.

Preferred embodiments are directed to methods wherein said immune suppressive protein is indolamide 2,3 deoxygenase.

A method of treating an inflammatory condition comprising the steps of: a) obtaining an umbilical cord derived mesenchymal stem cell population; b) culturing said umbilical cord derived mesenchymal stem cell population in conditions to allow for augmentation of an immune modulating effect; and c) administering said cell population into a patient in need of treatment.

Preferred embodiments are directed to methods wherein said inflammatory condition is an autoimmune condition.

Preferred embodiments are directed to methods wherein said autoimmune condition is a state in which immune cells of the patient recognize and attack tissue of said patient.

Preferred embodiments are directed to methods wherein said immune cells are T cells.

Preferred embodiments are directed to methods wherein said immune cells are B cells.

Preferred embodiments are directed to methods wherein said immune cells are NK cells.

Preferred embodiments are directed to methods wherein said inflammatory condition is characterized by increased production of inflammatory cytokines as compared to an age matched patient not suffering from said inflammatory condition.

Preferred embodiments are directed to methods wherein said inflammatory cytokine is TNF-alpha.

Preferred embodiments are directed to methods wherein said inflammatory cytokine is IL-1.

Preferred embodiments are directed to methods wherein said inflammatory cytokine is IL-6.

Preferred embodiments are directed to methods wherein said inflammatory cytokine is IL-11.

Preferred embodiments are directed to methods wherein said inflammatory cytokine is IL-12.

Preferred embodiments are directed to methods wherein said inflammatory cytokine is IL-17.

Preferred embodiments are directed to methods wherein said inflammatory cytokine is IL-18.

Preferred embodiments are directed to methods wherein said inflammatory cytokine is IL-21.

Preferred embodiments are directed to methods wherein said inflammatory cytokine is IL-33.

Preferred embodiments are directed to methods wherein said inflammatory condition is characterized by increased activation of the complement system as compared to an age matched patient not suffering from said inflammatory condition.

Preferred embodiments are directed to methods wherein said umbilical cord stem cell populations are cultured in interferon gamma at a concentration and duration sufficient to induce anti-inflammatory properties of said umbilical cord stem cell.

Preferred embodiments are directed to methods wherein said anti-inflammatory properties are selected from a group consisting of: a) suppression of ongoing mixed lymphocyte reaction; b) suppression of inflammatory cytokine production; and c) stimulation of T regulatory cells.

Preferred embodiments are directed to methods wherein said umbilical cord stem cell populations are cultured in the presence of interferon gamma at a concentration of 1-100 IU/ml.

Preferred embodiments are directed to methods wherein said interferon gamma is used to treat umbilical cord stem cells at a concentration of 10-75 IU/ml.

Preferred embodiments are directed to methods wherein said interferon gamma is used to treat umbilical cord stem cells at a concentration of 25-50 IU/ml.

Preferred embodiments are directed to methods wherein said interferon gamma is used to treat umbilical cord stem cells for a period of time ranging from 1 hour to 14 days.

Preferred embodiments are directed to methods wherein said interferon gamma is used to treat umbilical cord stem cells for a period of time ranging from 1 to 7 days.

Preferred embodiments are directed to methods wherein said interferon gamma is used to treat umbilical cord stem cells for a period of time ranging from 1 to 7 days.

Preferred embodiments are directed to methods wherein said biological response modifier is platelet rich plasma.

Preferred embodiments are directed to methods wherein platelet rich plasma used to treat umbilical cord stem cell at a concentration of 1-50% volume by volume in tissue culture media in which said umbilical cord stem cell are cultured.

Preferred embodiments are directed to methods wherein said platelet rich plasma used to treat umbilical cord stem cells at a concentration of 5-20% volume by volume in tissue culture media in which said umbilical cord stem cells are cultured.

Preferred embodiments are directed to methods wherein said platelet rich plasma used to treat umbilical cord stem cells at a concentration of 5-10% volume by volume in tissue culture media in which said umbilical cord stem cells are cultured.

Preferred embodiments are directed to methods wherein said platelet rich plasma is used to treat umbilical cord stem cells for a period of time ranging from 1 hour to 14 days.

Preferred embodiments are directed to methods wherein said platelet rich plasma is used to treat umbilical cord stem cells for a period of time ranging from 1 to 7 days.

Preferred embodiments are directed to methods wherein said platelet rich plasma is used to treat umbilical cord stem cells for a period of time ranging from 1 to 7 days.

Preferred embodiments are directed to methods wherein said umbilical cord stem cells are cultured in a media selected from a group comprising of: a) Roswell Park Memorial Institute (RPMI-1640); b) Dublecco’s Modified Essential Media (DMEM), c) Eagle’s Modified Essential Media (EMEM), d) Optimem, and e) Iscove’s Media.

Preferred embodiments are directed to methods wherein said umbilical cord stem cells are selected for expression of CD73.

Preferred embodiments are directed to methods wherein selection for CD73 is performed during isolation of umbilical cord mononuclear cells from said umbilical cord tissue.

Preferred embodiments are directed to methods wherein said isolation of said umbilical cord mononuclear cells is performed from said umbilical cord tissue by use of enzymatic digestion.

Preferred embodiments are directed to methods wherein said isolation of said umbilical cord mononuclear cells is performed from said umbilical cord tissue by use of mechanical dissociation.

Preferred embodiments are directed to methods wherein said isolation of said umbilical cord mononuclear cells is performed from said umbilical cord tissue by use of mechanical dissociation and enzymatic digestion.

Preferred embodiments are directed to methods wherein said CD73 expressing cells are further selected for expression of interleukin-7 receptor.

Preferred embodiments are directed to methods wherein said CD73 expressing cells are further selected for expression of interleukin-3 receptor.

Preferred embodiments are directed to methods wherein said CD73 expressing cells are further selected for expression of Receptor for Advanced Glycation End Products (RAGE).

Preferred embodiments are directed to methods wherein said CD73 expressing cells are further selected for expression of TNF-alpha receptor p55.

Preferred embodiments are directed to methods wherein said CD73 expressing cells are further selected for expression of TNF-alpha receptor p75.

Preferred embodiments are directed to methods wherein said CD73 expressing cells are further selected for expression of stem cell factor receptor.

Preferred embodiments are directed to methods wherein said CD73 expressing cells are further selected for expression of GM-CSF receptor alpha.

Preferred embodiments are directed to methods wherein said CD73 expressing cells are further selected for expression of VPR-Binding Protein.

Preferred embodiments are directed to methods wherein said CD73 expressing cells are further selected for expression of transferrin receptor.

Preferred embodiments are directed to methods wherein said CD73 expressing cells are further selected for expression of Tmc5 protein.

Preferred embodiments are directed to methods wherein said CD73 expressing cells are further selected for expression of TLR-9.

Preferred embodiments are directed to methods wherein said CD73 expressing cells are further selected for expression of zinc transporter 9.

Preferred embodiments are directed to methods wherein said CD73 expressing cells are further selected for expression of seminal vesicle antigen-like 3.

Preferred embodiments are directed to methods wherein said CD73 expressing cells are further selected for expression of sarcoma antigen NY-SAR-41.

Preferred embodiments are directed to methods wherein said CD73 expressing cells are further selected for expression of cell surface vimentin.

Preferred embodiments are directed to methods wherein said CD73 expressing cells are further selected for expression of fibrosin-1.

Preferred embodiments are directed to methods wherein said CD73 expressing cells are further selected for expression of IL-1 receptor.

Preferred embodiments are directed to methods wherein said CD73 expressing cells are further selected for expression of IL-3 receptor.

Preferred embodiments are directed to methods wherein said CD73 expressing cells are further selected for expression of IL-6 receptor.

Preferred embodiments are directed to methods wherein said CD73 expressing cells are further selected for expression of HGF receptor.

Preferred embodiments are directed to methods wherein said CD73 expressing cells are further selected for expression of thrombopoietin receptor.

Preferred embodiments are directed to methods wherein said CD73 expressing cells are further selected for expression of prolactin receptor.

Preferred embodiments are directed to methods wherein said CD73 expressing cells are further selected for expression of IGF-1 receptor.

Preferred embodiments are directed to methods wherein said CD73 expressing cells are further selected for expression of PDGF-BB receptor.

Preferred embodiments are directed to methods wherein said CD73 expressing cells are further selected for expression of angiopoietin receptor.

Preferred embodiments are directed to methods wherein said CD73 expressing cells are further selected for expression of VEGF receptor.

Preferred embodiments are directed to methods wherein said CD73 expressing cells are further selected for expression of TLR-2.

Preferred embodiments are directed to methods wherein said CD73 expressing cells are further selected for expression of TLR-3.

Preferred embodiments are directed to methods wherein said CD73 expressing cells are further selected for expression of TLR-4.

Preferred embodiments are directed to methods wherein said CD73 expressing cells are further selected for expression of TLR-5.

Preferred embodiments are directed to methods wherein said CD73 expressing cells are further selected for expression of TLR-7.

Preferred embodiments are directed to methods wherein said CD73 expressing cells are further selected for expression of TLR-8.

Preferred embodiments are directed to methods wherein said CD73 expressing cells are further selected for expression of oxysterol-binding protein 1.

Preferred embodiments are directed to methods wherein said CD73 expressing cells are further selected for expression of nesprin-2.

Preferred embodiments are directed to methods wherein said CD73 expressing cells are further selected for expression of myomesin-3.

Preferred embodiments are directed to methods wherein said CD73 expressing cells are further selected for expression of mucin-2.

Preferred embodiments are directed to methods wherein said CD73 expressing cells are further selected for expression of FRAS1-related extracellular matrix protein 3.

Preferred embodiments are directed to methods wherein said CD73 expressing cells are further selected for expression of C-C chemokine receptor type 10.

Preferred embodiments are directed to methods wherein said CD73 expressing cells are further selected for expression of CXCR4.

Preferred embodiments are directed to methods wherein said CD73 expressing cells are further selected for expression of CCR5

Preferred embodiments are directed to methods wherein said CD73 expressing cells are further selected for expression of cartilage intermediate layer protein 2.

Preferred embodiments are directed to methods wherein said CD73 expressing cells are further selected for expression of HLA-DR.

Preferred embodiments are directed to methods wherein said CD73 expressing cells are further selected for expression of oxytocin receptor.

Preferred embodiments are directed to methods wherein said CD73 expressing cells are further selected for expression of CD77.

Preferred embodiments are directed to methods wherein said CD73 expressing cells are further selected for expression of CD56.

Preferred embodiments are directed to methods wherein said CD73 expressing cells are further selected for expression of poliovirus receptor.

Preferred embodiments are directed to methods wherein said CD73 expressing cells are further selected for expression of plexin A2.

Preferred embodiments are directed to methods wherein said CD73 expressing cells are further selected for expression of plexin A4.

Preferred embodiments are directed to methods wherein said CD73 expressing cells are further selected for expression of HLA-G.

Preferred embodiments are directed to methods wherein said CD73 expressing cells are further selected for expression of membrane bound TGF-beta.

Preferred embodiments are directed to methods wherein said CD73 expressing cells are further selected for expression of membrane bound TNF-alpha.

Preferred embodiments are directed to methods wherein said CD73 expressing cells are further selected for expression of plasticity-related protein 2.

Preferred embodiments are directed to methods wherein said CD73 expressing cells are further selected for expression of BDNF receptor.

Preferred embodiments are directed to methods wherein said CD73 expressing cells are further selected for expression of occludin.

Preferred embodiments are directed to methods wherein said CD73 expressing cells are further selected for expression of neuronal pentraxin receptor.

Preferred embodiments are directed to methods wherein said CD73 expressing cells are further selected for expression of neuropilin-1.

Preferred embodiments are directed to methods wherein said CD73 expressing cells are further selected for expression of netrin 2-like.

Preferred embodiments are directed to methods wherein said CD73 expressing cells are further selected for expression of mucolipin 1.

Preferred embodiments are directed to methods wherein said CD73 expressing cells are further selected for expression of MEGF10 protein.

Preferred embodiments are directed to methods wherein said CD73 expressing cells are further selected for expression of mannose binding lectin (A).

Preferred embodiments are directed to methods wherein said CD73 expressing cells are further selected for expression of Leukemia Inhibitory Factor Receptor.

Preferred embodiments are directed to methods wherein said CD73 expressing cells are further selected for expression of lipocalin 3.

Preferred embodiments are directed to methods wherein said CD73 expressing cells are further selected for expression of lipocalin 12.

Preferred embodiments are directed to methods wherein said CD73 expressing cells are further selected for expression of lipocalin 13.

A method of treating cancer comprising: a) selecting an umbilical cord derived mononuclear cell population, ideally of mesenchymal phenotype; b) increasing ability said cells to home to tumor microenvironment; c) increasing ability of cells to inhibit cancer; and d) administering said cells to a patient in need of treatment.

Preferred embodiments are directed to methods wherein said increased ability of said cell to home to tumors comprises incubation of said cell in a hypoxic environment from 0.1% oxygen to 10% oxygen for a period of 30 minutes to 3 days.

Preferred embodiments are directed to methods wherein said cell is cultured at 3% oxygen for 24 hours.

Preferred embodiments are directed to methods wherein said increased ability of said cell to inhibit cancer is accomplished by infection of said cell with an oncolytic virus.

Preferred embodiments are directed to methods wherein said oncolytic virus is selected from a group comprising of: a) vaccinia virus; b) reovirus; c) Newcastle Disease Virus; d) herpes virus; e) parvovirus; f) measles virus; g) vesicular stomatitis virus (VSV); h) adenovirus; i) poliovirus; j) a poxvirus; k) coxsackie virus (CXV); and 1) Seneca Valley virus (SVV).

Preferred embodiments are directed to methods wherein the vaccinia virus is selected from among a Lister strain, Western Reserve (WR) strain, Copenhagen (Cop) strain, Bern strain, Paris strain, Tashkent strain, Tian Tan strain, Wyeth strain (DRYVAX), IHD-J strain, IHD-W strain, Brighton strain, Ankara strain, CVA382 strain, Dairen I strain, LC16m8 strain, LC16M0 strain, modified vaccinia Ankara (MVA) strain, ACAM strain, WR 65-16 strain, Connaught strain, New York City Board of Health (NYCBH) strain, EM-63 strain, NYVAC strain, Lister strain LIVP, JX-594 strain, GL-ONC1 strain, a vvDD TK mutant strain with deletions in VGF and TK, ACAM2000, and ACAM1000

Preferred embodiments are directed to methods wherein said increased ability of cells to inhibit cancer is endowed by transfection of said cells with a cancer-inhibitory gene.

Preferred embodiments are directed to methods wherein said cancer inhibitory gene is under control of an inducible promoter.

Preferred embodiments are directed to methods wherein said inducible promoter is a rheoswitch.

Preferred embodiments are directed to methods wherein said cancer-inhibitory gene is TNF-alpha.

Preferred embodiments are directed to methods wherein said cancer-inhibitory gene is TRAIL.

Preferred embodiments are directed to methods wherein said cancer-inhibitory gene is a suicide gene.

Preferred embodiments are directed to methods wherein said cancer-inhibitory gene is thymidylate synthesase.

Preferred embodiments are directed to methods wherein said increased ability of cells to inhibit cancer is endowed by transfection of said cells with an immune stimulatory gene.

Preferred embodiments are directed to methods wherein said immune stimulatory gene is IL-2.

Preferred embodiments are directed to methods wherein said immune stimulatory gene is IL-15.

Preferred embodiments are directed to methods wherein said immune stimulatory gene is IL-7.

Preferred embodiments are directed to methods wherein said immune stimulatory gene is IL-12.

Preferred embodiments are directed to methods wherein said immune stimulatory gene is IL-15.

Preferred embodiments are directed to methods wherein said immune stimulatory gene is IL-22.

Preferred embodiments are directed to methods wherein said immune stimulatory gene is IL-18.

Preferred embodiments are directed to methods wherein said immune stimulatory gene is IL-27.

Preferred embodiments are directed to methods wherein said immune stimulatory gene is a bispecific antibody.

Preferred embodiments are directed to methods wherein said infection with an oncolytic virus is performed in an umbilical cord derived mesenchymal stem cell that is treated with hypoxia in a manner sufficient to induce translocation of HIF-1 alpha.

Preferred embodiments are directed to methods wherein said cell is engineered to express an oncogene.

428. Preferred embodiments are directed to methods wherein said oncogene is selected from a group comprising of: ABCB1, ABCG2, ABI1, ABL1, ABL2, ACKR3, ACSL3, ACSL6, ACVR1B, ACVR2A, AFF1, AFF3, AFF4, AKAP9, AKT1, AKT2, AKT3, ALDH1A1, ALDH2, ALK, AMER1, ANGPT1, ANGPT2, ANKRD23, APC, AR, ARAF, AREG, ARFRP1, ARHGAP26, ARHGEF12, ARID1A, ARID1B, ARID2, ARNT, ASPSCR1, ASXL1, ATF1, ATIC, ATM, ATP1A1, ATP2B3, ATR, ATRX, AURKA, AURKB, AXIN1, AXL, BAP1, BARD1, BBC3, BCL10, BCL11A, BCL11B, BCL2, BCL2L1, BCL2L11, BCL2L2, BCL3, BCL6, BCL7A, BCL9, BCOR, BCORL1, BCR, BIRC3, BLM, BMPR1A, BRAF, BRCA1, BRCA2, BRD3, BRD4, BRINP3, BRIP1, BTG1, BTG2, BTK, BUB1B, C11orf30, C15orf65, C2orf44, CA6, CACNA1D, CALR, CAMTA1, CANT1, CARD11, CARS, CASC5, CASP8, CBFA2T3, CBFB, CBL, CBLB, CBLC, CCDC6, CCNB1IP1, CCND1, CCND2, CCND3, CCNE1, CD19, CD22, CD274, CD38, CD4, CD70, CD74, CD79A, CD79B, CD83, CDC73, CDH1, CDH11, CDK12, CDK4, CDK6, CDK7, CDK8, CDK9, CDKN1A, CDKN1B, CDKN2A, CDKN2B, CDKN2C, CDX2, CEBPA, CHCHD7, CHD2, CHD4, CHEK1, CHEK2, CHIC2, CHN1, CHORDC1, CIC, CIITA, CLP1, CLTC, CLTCL1, CNBP, CNOT3, CNTRL, COL1A1, COPB1, COX6C, CRBN, CREB1, CREB3L1, CREB3L2, CREBBP, CRKL, CRLF2, CRTC1, CRTC3, CSF1R, CSF3R, CTCF, CTLA4, CTNNA1, CTNNB1, CUL3, CXCR4, CYLD, CYP17A1, CYP2D6, DAXX, DDB2, DDIT3, DDR1, DDR2, DDX10, DDX3X, DDX5, DDX6, DEK, DICER1, DIS3, DLL4, DNM2, DNMT1, DNMT3A, DOT1L, DPYD, DUSP4, DUSP6, EBF1, ECT2L, EDNRB, EED, EGFR, EIF4A2, ELF4, ELK4, ELL, ELN, EML4, EP300, EPHA3, EPHA5, EPHA7, EPHA8, EPHB1, EPHB2, EPHB4, EPS15, ERBB2, ERBB3, ERBB4, ERC1, ERCC1, ERCC2, ERCC3, ERCC4, ERCC5, EREG, ERG, ERN1, ERRFI1, ESR1, ETV1, ETV4, ETV5, ETV6, EWSR1, EXT1, EXT2, EZH2, EZR, FAF1, FAIM3, FAM46C, FANCA, FANCC, FANCD2, FANCE, FANCF, FANCG, FANCL, FAS, FAT1, FBXO11, FBXW7, FCRL4, FEV, FGF10, FGF14, FGF19, FGF2, FGF23, FGF3, FGF4, FGF6, FGFR1, FGFR1OP, FGFR2, FGFR3, FGFR4, FH, FHIT, FIP1L1, FKBP1A, FLCN, FLI1, FLT1, FLT3, FLT4, FNBP1, FOXA1, FOXL2, FOXO1, FOXO3, FOXO4, FOXP1, FRS2, FSTL3, FUBP1, FUS, GABRA6, GAS7, GATA1, GATA2, GATA3, GATA4, GATA6, GID4, GLI1, GMPS, GNA11, GNA12, GNA13, GNAQ, GNAS, GNRH1, GOLGA5, GOPC, GPC3, GPHN, GPR124, GRIN2A, GRM3, GSK3B, GUCY2C, H3F3A, H3F3B, HCK, HDAC1, HERPUD1, HEY1, HGF, HIP1, HIST1H1E, HIST1H3B, HIST1H4I, HLF, HMGA1, HMGA2, HMGN2P46, HNF1A, HNMT, HNRNPA2B1, HNRNPK, HOOK3, HOXA11, HOXA13, HOXA9, HOXC11, HOXC13, HOXD11, HOXD13, HRAS, HSD3B1, HSP90AA1, HSP90AB1, IAPP, ID3, IDH1, IDH2, IGF1R, IGF2, IKBKE, IKZF1, IL2, IL21R, IL3RA, IL6, IL6ST, IL7R, INHBA, INPP4B, IRF2, IRF4, IRS2, ITGAV, ITGB1, ITK, ITPKB, JAK1, JAK2, JAK3, JAZF1, JUN, KAT6A, KAT6B, KCNJ5, KDM1A, KDM5A, KDM5C, KDM6A, KDR, KDSR, KEAP1, KEL, KIAA1549, KIF5B, KIR3DL1, KIT, KLF4, KLHL6, KLK2, KMT2A, KMT2C, KMT2D, KRAS, KTN1, LASP1, LCK, LCP1, LGALS3, LGR5, LHFP, LIFR, LMO1, LMO2, LOXL2, LPP, LRIG3, LRP1B, LUC7L2, LYL1, LYN, LZTR1, MAF, MAFB, MAGED1, MAGI2, MALT1, MAML2, MAP2K1, MAP2K2, MAP2K4, MAP3K1, MAPK1, MAPK11, MAX, MCL1, MDM2, MDM4, MDS2, MECOM, MED12, MEF2B, MEN1, MET, MITF, MKI67, MKL1, MLF1, MLH1, MLLT1, MLLT10, MLLT11, MLLT3, MLLT4, MLLT6, MMP9, MN1, MNX1, MPL, MRE11A, MS4A1, MSH2, MSH6, MSI2, MSN, MST1R, MTCP1, MTF2, MTOR, MUC1, MUC16, MUTYH, MYB, MYC, MYCL, MYCN, MYD88, MYH11, MYH9, NACA, NAE1, NBN, NCKIPSD, NCOA1, NCOA2, NCOA4, NDRG1, NF1, NF2, NFE2L2, NFIB, NFKB2, NFKBIA, NIN, NKX2-1, NONO, NOTCH1, NOTCH2, NOTCH3, NPM1, NR4A3, NRAS, NSD1, NT5C2, NTRK1, NTRK2, NTRK3, NUMA1, NUP214, NUP93, NUP98, NUTM1, NUTM2B, OLIG2, OMD, P2RY8, PAFAH1B2, PAK3, PALB2, PARK2, PARP1, PATZ1, PAX3, PAX5, PAX7, PAX8, PBRM1, PBX1, PCM1, PCSK7, PDCD1, PDCD1LG2, PDE4DIP, PDGFB, PDGFRA, PDGFRB, PDK1, PECAM1, PER1, PHF6, PHOX2B, PICALM, PIK3C2B, PIK3CA, PIK3CB, PIK3CD, PIK3CG, PIK3R1, PIK3R2, PIM1, PLAG1, PLCG2, PML, PMS1, PMS2, POLD1, POLE, POT1, POU2AF1, POU5F1, PPARG, PPP2R1A, PRCC, PRDM1, PRDM16, PREX2, PRF1, PRKAR1A, PRKCI, PRKDC, PRLR, PRPF40B, PRRT2, PRRX1, PRSS8, PSIP1, PSMD4, PTBP1, PTCH1, PTEN, PTK2, PTPN11, PTPRC, PTPRD, QKI, RABEP1, RAC1, RAD21, RAD50, RAD51, RAD51B, RAD51C, RAD51D, RAF1, RALGDS, RANBP17, RANBP2, RAP1GDS1, RARA, R131, RBM10, RBM15, RCOR1, RECQL4, REL, RELN, RET, RHOA, RHOH, RICTOR, RIPK1, RMI2, RNF213, RNF43, ROS1, RPL10, RPL22, RPL5, RPN1, RPS6KB1, RPTOR, RUNX1, RUNX1T1, S1PR2, SAMHD1, SBDS, SDC4, SDHA, SDHAF2, SDHB, SDHC, SDHD, SEPT5, SEPT6, SEPT9, SET, SETBP1, SETD2, SF1, SF3A1, SF3B1, SF3B2, SFPQ, SGK1, SH2B3, SH3GL1, SLAMF7, SLC34A2, SLC45A3, SLIT2, SMAD2, SMAD3, SMAD4, SMARCA4, SMARCB1, SMARCE1, SMC1A, SMC3, SMO, SNCAIP, SNX29, SOCS1, SOX10, SOX11, SOX2, SOX9, SPECC1, SPEN, SPOP, SPTA1, SRC, SRGAP3, SRSF2, SRSF3, SS18, SS18L1, SSX1, STAG2, STAT3, STAT4, STAT5B, STEAP1, STIL, STK11, SUFU, SUZ12, SYK, TAF1, TAF15, TAL1, TAL2, TBL1XR1, TBX3, TCEA1, TCF12, TCF3, TCF7L2, TCL1A, TEK, TERC, TERT, TET1, TET2, TFE3, TFEB, TFG, TFPT, TFRC, TGFB1, TGFBR2, THRAP3, TIMP1, TJP1, TLX1, TLX3, TM7SF2, TMPRSS2, TNFAIP3, TNFRSF14, TNFRSF17, TNFRSF18, TNFRSF9, TNFSF11, TOP1, TOP2A, TP53, TP63, TPBG, TPM3, TPM4, TPR, TRAF2, TRAF3, TRAF3IP3, TRAF7, TRIM26, TRIM27, TRIM33, TRIP11, TRRAP, TSC1, TSC2, TSHR, TTK, TTL, TYMS, U2AF1, U2AF2, UBA1, UBR5, USP6, VEGFA, VEGFB, VHL, VPS51, VTI1A, WAS, WEE1, WHSC1, WHSC1L1, WIF1, WISP3, WNT11, WNT2B, WNT3, WNT3A, WNT4, WNT5A, WNT6, WNT7B, WRN, WT1, WWTR1, XBP1, XPA, XPC, XPO1, YWHAE, YWHAZ, ZAK, ZBTB16, ZBTB2, ZMYM2, ZMYM3, ZNF217, ZNF331, ZNF384, ZNF521, ZNF703 and ZRSR2

429. Preferred embodiments are directed to methods wherein said cell is engineered to express a tumor suppressor gene.

430. Preferred embodiments are directed to methods wherein said tumor suppressor gene is selected from a group comprising of: P53. RB1, WT1, NF1, NF2, APC, TSC1, TSC2, DPC4, DCC, BRCA1, BRCA2, PTEN, STK11, MSH2, MLH1, CDH1, VHL, CDKN2A, PTCH, MEN1.

432. Preferred embodiments are directed to methods wherein said cell is gene edited, and/or treated with an agent or plurality of agents which induce RNA interference, and/or treated with an agent or plurality of agents which induce RNAse U in order to reduce or remove expression of a checkpoint molecule.

433. Preferred embodiments are directed to methods wherein said checkpoint molecules are selected from a group comprising of: PD1 (also called PDCD1 or CD279); PD-L1 (also called B7-H1 or CD274); PD-L2 (also called B7-DC or CD273); CTLA-4 (also called CD152); B7-H3 (also called CD276); B7-H4 (also called B7S1 or B7x); CD66a (CEACAM1); VISTA (also called B7-H5 or GI24); BTLA; CD160; LAG3 (also called CD223 or Lymphocyte activation gene 3); Indoleamine 2,3-dioxygenase (also called IDO); Galectin-9 (also called LGALS9); TIM-3 (also called HAVCR2); 2B4 (also called CD244); SIRP alpha (also called CD172a); CD39; CD47; CD48 (also called SLAMF2); A2AR; KIRs; and TIGIT (also called VSTM3).

434. Preferred embodiments are directed to methods wherein said cell is incubated with the virus for at least 16 hours or at least 20 hours or at least 24 hours.

435. Preferred embodiments are directed to methods wherein said cell is incubated with the virus for up to 48 hours.

436. Preferred embodiments are directed to methods wherein said cell is infected with virus at a MOI less than or equal to 0.8.

437. Preferred embodiments are directed to methods wherein said cell is infected with virus at a MOI less than or equal to 0.5.

438. Preferred embodiments are directed to methods wherein said cell isolated from said Wharton’s Jelly express a marker selected from a group of markers comprising: CD56, CD57, CD144, CD105, and CD31.

439. Preferred embodiments are directed to methods wherein said cells are obtained from placenta perivascular tissue as a substitute for Wharton’s Jelly.

440. Preferred embodiments are directed to methods wherein said cells are s isolated from fetal vascular lobules of a hemochorial placenta.

441. Preferred embodiments are directed to methods wherein said cells are isolated by: dissociating fetal vascular lobules from a hemochorial placenta; digesting the dissociated fetal vascular lobes with an enzymatic mixture or by mechanical means; applying a filtration means to said dissociated lobes in order to remove particulates; obtaining mononuclear cells; plating said mononuclear cells in a substrate allowing for growth of said mononuclear cells to confluency; detaching the confluent cells from the plate; and isolating for expression of CD144 and substantially lack of expression of CD45, optionally one or more steps are performed in the presence of hypoxia, wherein hypoxia is sufficient to induce translocation of HIF-1 alpha.

442. Preferred embodiments are directed to methods wherein dissociation of fetal vascular lobes is accomplishing by incubation with a mixture of about 2% collagenase, about 0.25% trypsin and about 0.1% DNAse in tissue culture medium.

443. Preferred embodiments are directed to methods wherein dissociation of fetal vascular lobes is accomplishing by incubation with a mixture of about 2% collagenase, about 0.25% trypsin and about 0.1% DNAse in tissue culture medium.

444. Preferred embodiments are directed to methods wherein cells isolated are comprised of adherent cells expressing the marker CD73 but substantially lacking CD105.

445. Preferred embodiments are directed to methods wherein cells isolated are comprised of adherent cells expressing the marker CD73 and CD105 but lacking in CD90.

446. Preferred embodiments are directed to methods wherein said regenerative adjuvant is an anti-inflammatory cytokine.

447. Preferred embodiments are directed to methods wherein said anti-inflammatory cytokine is selected from a group comprising of IL-4, IL-10, IL-13, IL-20, IL-22 and IL-35.

448. Preferred embodiments are directed to methods wherein said anti-inflammatory cytokine is TGF-beta.

449. Preferred embodiments are directed to methods wherein said anti-inflammatory cytokine is PGE-2.

450. Preferred embodiments are directed to methods wherein said anti-inflammatory cytokine is VEGF.

451. Preferred embodiments are directed to methods wherein said composition is composed of mesenchymal stem cells, and wherein said first regenerative adjuvant is hypoxia.

452. Preferred embodiments are directed to methods wherein said mesenchymal stem cells possess one or more markers selected from a group comprising of: a) CD11b; b) CD11c; c) CD20; d) CD56; e) CD57 f) CD73; g) CD90; h) CD105; i) membrane bound TGF-beta; and j) neuropilin.

453. Preferred embodiments are directed to methods wherein said composition is capable of inhibiting T cell mediated immune responses.

454. Preferred embodiments are directed to methods wherein said T cell mediated immune responses comprise of Th1 cell production of cytokines.

455. Preferred embodiments are directed to methods wherein said cancers are selected from a group of malignancies comprising of: acute lymphoblastic leukemia; acute myeloid leukemia; adrenocortical carcinoma; AIDS-related cancer; AIDS-related lymphoma; anal cancer; appendix cancer; astrocytomas; atypical teratoid/rhabdoid tumor; basal cell carcinoma; bladder cancer; brain stem glioma; brain tumor, brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma; breast cancer; bronchial tumors; Burkitt lymphoma; cancer of unknown primary site (CUP); carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sezary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenstrom macroglobulinemia; or Wilm’s tumor.

456. A method of inducing immunological tolerance, and/or predisposing to immunological tolerance, comprising the steps of: a) obtaining a population of umbilical cord mononuclear cells; b) exposing said cells to conditions capable of triggering apoptosis; c) extracting said apoptotic bodies; and d) administering said apoptotic bodies to a patient in need of treatment at a concentration and frequency sufficient to induce and/or predispose to immune tolerance.

457. Preferred embodiments are directed to methods wherein said tolerance is a state of non-responsiveness of an immune cell to a condition or plurality of conditions normally results in responsiveness of said immune cell.

458. Preferred embodiments are directed to methods wherein said immune cell is an adaptive or innate immune cell.

459. Preferred embodiments are directed to methods wherein said adaptive immune cell is a T cell.

460. Preferred embodiments are directed to methods wherein said adaptive immune cell is a B cell.

461. Preferred embodiments are directed to methods wherein said innate immune cell is a monocyte.

462. Preferred embodiments are directed to methods wherein said innate immune cell is a natural killer cell.

463. Preferred embodiments are directed to methods wherein said innate immune cell is a gamma delta T cell.

464. Preferred embodiments are directed to methods wherein said innate immune cell is a natural killer T cell.

465. Preferred embodiments are directed to methods wherein said innate immune cell is a neutrophil.

466. Preferred embodiments are directed to methods wherein said innate immune cell is a dendritic cell.

467. Preferred embodiments are directed to methods wherein immunological tolerance is associated with remission in an autoimmune condition.

468. Preferred embodiments are directed to methods wherein said cells are adherent cells.

469. Preferred embodiments are directed to methods wherein said cells are obtained by positive selection of umbilical cord blood mononuclear cells for CD56.

470. Preferred embodiments are directed to methods wherein said cells express CD90.

471. Preferred embodiments are directed to methods wherein said cells express vimentin.

472. Preferred embodiments are directed to methods wherein said cells express CD57.

473. Preferred embodiments are directed to methods wherein said non-responsiveness of said T cell comprises reduction of interleukin-2 production in response to stimulation via T cell receptor.

474. Preferred embodiments are directed to methods wherein said non-responsiveness of said T cell comprises reduction of interferon gamma production in response to stimulation via T cell receptor.

475. Preferred embodiments are directed to methods wherein said non-responsiveness of said T cell comprises reduction of proliferation in response to stimulation via T cell receptor.

476. Preferred embodiments are directed to methods wherein said non-responsiveness of said T cell comprises reduction of interleukin-4 production in response to stimulation via T cell receptor.

477. Preferred embodiments are directed to methods wherein said non-responsiveness of said T cell comprises enhanced need for costimulation in order to activate said T cell subsequent to stimulation via T cell receptor.

478. Preferred embodiments are directed to methods wherein said non-responsiveness of said T cell comprises enhanced need for costimulation in order to activate said T cell subsequent to stimulation via T cell receptor.

479. Preferred embodiments are directed to methods wherein said non-responsiveness comprises reduction in cytotoxicity subsequent to stimulation via T cell receptor.

480. Preferred embodiments are directed to methods wherein said non-responsiveness comprises reduction in antibody production subsequent to stimulation via B cell receptor.

481. Preferred embodiments are directed to methods wherein said non-responsiveness comprises reduction in monocyte phagocytic activity.

482. Preferred embodiments are directed to methods wherein said non-responsiveness comprises reduction in monocyte cytokine production activity.

483. Preferred embodiments are directed to methods wherein said cytokines are selected from a group comprising of: a) TNF-alpha; b) IL-1 beta; c) IL-6; d) IL-8; e) IL-12; f) IL-18; g) IL-17; h) IL-21; i) IL-23; j) IL-27; k) IL-33; and 1) RANTES.

484. Preferred embodiments are directed to methods wherein said non-responsiveness comprises reduction of M1 polarization.

485. Preferred embodiments are directed to methods wherein said non-responsiveness comprises enhancement M2 polarization.

486. Preferred embodiments are directed to methods wherein said non-responsiveness comprises inhibition of maturation to macrophages.

487. Preferred embodiments are directed to methods wherein said non-responsiveness comprises inhibition of maturation to dendritic cells.

488. Preferred embodiments are directed to methods wherein said non-responsiveness comprises inhibition of antigen presentation.

489. Preferred embodiments are directed to methods wherein said non-responsiveness comprises inhibition of NK cytotoxicity.

490. Preferred embodiments are directed to methods wherein said non-responsiveness comprises inhibition of NK ability to induce dendritic cell maturation.

491. Preferred embodiments are directed to methods wherein dendritic cell maturation comprises augmented expression of costimulatory molecules.

492. Preferred embodiments are directed to methods wherein said costimulatory molecule is CD40.

493. Preferred embodiments are directed to methods wherein said costimulatory molecule is CD80.

494. Preferred embodiments are directed to methods wherein said costimulatory molecule is CD86.

495. Preferred embodiments are directed to methods wherein said costimulatory molecule is IL-12.

496. Preferred embodiments are directed to methods wherein said non-responsiveness comprises inhibition of IL-12 secretion by gamma delta T cells.

497. Preferred embodiments are directed to methods wherein said non-responsiveness comprises inhibition of interferon gamma secretion by gamma delta T cells.

498. Preferred embodiments are directed to methods wherein said non-responsiveness comprises inhibition of cytotoxicity by gamma delta T cells.

499. Preferred embodiments are directed to methods wherein said non-responsiveness comprises inhibition of dendritic cell maturation by gamma delta T cells.

500. Preferred embodiments are directed to methods wherein said non-responsiveness comprises inhibition of natural killer T cell production of IL-18.

501. Preferred embodiments are directed to methods wherein said non-responsiveness comprises inhibition of neutrophil production of reactive oxidative intermediaries.

502. Preferred embodiments are directed to methods wherein said non-responsiveness comprises inhibition of neutrophil production of DNA extracellular traps.

503. Preferred embodiments are directed to methods wherein said non-responsiveness comprises induction of neutrophil apoptosis.

504. Preferred embodiments are directed to methods wherein said extraction of apoptotic bodies is performed by means of a density gradient.

505. Preferred embodiments are directed to methods wherein said extraction of apoptotic bodies is performed by an affinity means.

506. Preferred embodiments are directed to methods wherein said affinity means comprises binding to annexin V.

507. Preferred embodiments are directed to methods wherein apoptosis is induced by irradiation.

508. Preferred embodiments are directed to methods wherein said irradiation is gamma irradiation.

509. Preferred embodiments are directed to methods wherein said irradiation is X-irradiation.

510. Preferred embodiments are directed to methods wherein said irradiation is bombardment with helium nuclei.

511. Preferred embodiments are directed to methods wherein said irradiation is beta irradiation.

512. Preferred embodiments are directed to methods wherein said irradiation is muon irradiation.

513. Preferred embodiments are directed to methods wherein said irradiation is exposure to ultraviolet irradiation.

514. Preferred embodiments are directed to methods wherein a photosensitizer is added.

515. Preferred embodiments are directed to methods wherein said photosensitizer is psoralen.

516. Preferred embodiments are directed to methods wherein apoptosis is induced by exposure to an agent stimulating oxidative stress.

517. Preferred embodiments are directed to methods wherein said agent stimulating oxidative stress is ozone.

518. Preferred embodiments are directed to methods wherein said agent stimulating oxidative stress is hydrogen peroxide.

519. Preferred embodiments are directed to methods wherein said agent stimulating oxidative stress is hydroxyl radical.

520. Preferred embodiments are directed to methods wherein said agent stimulating oxidative stress is hyperthermia.

521. Preferred embodiments are directed to methods wherein said agent stimulating oxidative stress is non-isotonic conditions.

522. Preferred embodiments are directed to methods wherein said apoptosis is induced by exposure to TNF-alpha.

523. Preferred embodiments are directed to methods wherein said apoptosis is induced by exposure to TRAIL.

524. Preferred embodiments are directed to methods wherein said apoptosis is induced by exposure to Fas Ligand.

525. Preferred embodiments are directed to methods wherein immunological tolerance is used to promote pregnancy in a patient suffering from recurrent spontaneous abortions.

526. Preferred embodiments are directed to methods wherein said immunological tolerance is used to allow for decreased immune suppressants in a patient having received an organ, tissue, or cellular transplant.

527. Preferred embodiments are directed to methods wherein said immunological tolerance is used to allow for use of no immune suppressants in a patient having received an organ, tissue, or cellular transplant.

528. A method of stimulating angiogenesis comprising the steps of: a) obtaining one or more umbilical cord adherent cell populations; b) culturing said umbilical cord cell populations in a manner to allow for production of exosomes into culture media in which said cells are cultured in; c) extracting exosomes from said culture media; and d) administering said extracted exosomes into a patient in need of treatment.

529. Preferred embodiments are directed to methods wherein said umbilical cord cells are derived from a term pregnancy, wherein said donor providing said cells is either the patient to be treated (autologous) or said donor is different from the patient to be treated (allogeneic).

530. Preferred embodiments are directed to methods wherein said cells are cultured in a media allowing for cell proliferation.

531. Preferred embodiments are directed to methods wherein said media allowing for cell proliferation contains one or more factors known to be mitogenic for cells.

532. Preferred embodiments are directed to methods wherein said factors known to be mitogenic for umbilical cord cells include one or more factors selected from a group comprising of: a) FGF-1; b) FGF-2; c) FGF-5; d) EGF; e) CNTF; f) KGF-1; g) PDGF; h) platelet rich plasma; i) TGF-alpha; and j) HGF-1.

533. Preferred embodiments are directed to methods wherein said umbilical cord cells are cultured under hypoxia.

534. Preferred embodiments are directed to methods wherein exosomes are collected from umbilical cord cells while said umbilical cord cells are in a proliferating state.

535. Preferred embodiments are directed to methods wherein said exosomes are collected from umbilical cord cells while said umbilical cord cells are cultured in a media containing no proliferative factors or largely reduced levels of said proliferation inducing growth factors.

536. Preferred embodiments are directed to methods wherein said exosomes are collected from said umbilical cord cells that have been cultured in 2-8% oxygen for at least 1 day.

537. Preferred embodiments are directed to methods wherein the cells are cultured for 1-15 days.

538. Preferred embodiments are directed to methods wherein the cells are cultured for 5-10 days.

539. Preferred embodiments are directed to methods wherein the cells are passaged for at least 1 passage.

540. Preferred embodiments are directed to methods wherein said exosomes are in a preparation, said preparation comprises less than 5% polyethylene glycol.

541. Preferred embodiments are directed to methods wherein the exosomes are purified using polyethylene glycol.

542. Preferred embodiments are directed to methods wherein the exosomes are purified using ultrafiltration.

543. Preferred embodiments are directed to methods wherein polyethylene glycol is added to the exosomes after purification.

544. Preferred embodiments are directed to methods wherein said exosomes express markers selected from a group comprising of: a) CD63; b) CD9; c) MHC I; d) CD56

545. Preferred embodiments are directed to methods wherein said umbilical cord cells are cultured in a media selected from a group comprising of: a) Roswell Park Memorial Institute (RPMI-1640); b) Dublecco’s Modified Essential Media (DMEM), c) Eagle’s Modified Essential Media (EMEM), d) Optimem, and e) Iscove’s Media.

546. Preferred embodiments are directed to methods wherein said exosomes are administered to a mammal in need of treatment suffering from a pulmonary disorder.

547. Preferred embodiments are directed to methods wherein said pulmonary disorder is associated with pulmonary inflammation.

548. Preferred embodiments are directed to methods wherein said pulmonary inflammation is associated with increased neutrophils in the lung area.

549. Preferred embodiments are directed to methods wherein said pulmonary inflammation is associated with increased mast cells in the lung area.

550. Preferred embodiments are directed to methods wherein said pulmonary inflammation is associated with increased T cells in the lung area.

551. Preferred embodiments are directed to methods wherein said pulmonary inflammation is associated with increased B cells in the lung area.

552. Preferred embodiments are directed to methods wherein said pulmonary inflammation is associated with increased NK cells in the lung area.

553. Preferred embodiments are directed to methods wherein said pulmonary inflammation is associated with increased gamma delta T cells in the lung area.

554. Preferred embodiments are directed to methods wherein said pulmonary inflammation is associated with increased monocytes in the lung area.

555. Preferred embodiments are directed to methods wherein said pulmonary inflammation is associated with increased complement activation in the lung area.

556. Preferred embodiments are directed to methods wherein said pulmonary inflammation is associated with increased coagulopathy in the lung area.

557. Preferred embodiments are directed to methods wherein an antifibrotic drug is administered together with said exosomes.

558. Preferred embodiments are directed to methods wherein said anti-fibrotic agent is relaxin-2, a recombinant form of relaxin-2 or a functional derivative or variant or mimetic of relaxin-2.

559. Preferred embodiments are directed to methods wherein said recombinant form of relaxin-2 is serelaxin.

560. Preferred embodiments are directed to methods wherein the derivative of relaxin-2 is a single B chain derivative or an A and B chain truncate of relaxin-2.

561. Preferred embodiments are directed to methods wherein the single B chain derivative of relaxin-2 is H2-(B7-33) or the A and B chain truncate is H2-(A4-24)(B7-24).

562. Preferred embodiments are directed to methods further comprising the administration of an RXFP1 activating agent or agonist.

563. Preferred embodiments are directed to methods wherein said umbilical cord derived cells are modified to express the relaxin or its derivative or variant.

564. Preferred embodiments are directed to methods wherein said umbilical cord exosomes are modified to contain the relaxin or its recombinant or derivative or variant form.

565. Preferred embodiments are directed to methods wherein said umbilical cord cells or exosomes are co-administered simultaneously or sequentially with relaxin.

566. Preferred embodiments are directed to methods wherein said umbilical cord cells or exosomes are co-administered simultaneously or sequentially with VEGF.

567. Preferred embodiments are directed to methods wherein said umbilical cord cells or exosomes are co-administered simultaneously or sequentially with EGF-1.

568. Preferred embodiments are directed to methods wherein said umbilical cord cells or exosomes are co-administered simultaneously or sequentially with IGF-1.

569. Preferred embodiments are directed to methods wherein said umbilical cord cells or exosomes are co-administered simultaneously or sequentially with PDGF-BB.

570. Preferred embodiments are directed to methods wherein said umbilical cord cells or exosomes are co-administered simultaneously or sequentially with hCG.

571. Preferred embodiments are directed to methods wherein said umbilical cord cells or exosomes are co-administered simultaneously or sequentially with FGF-1.

572. Preferred embodiments are directed to methods wherein said umbilical cord cells or exosomes are co-administered simultaneously or sequentially with FGF-2.

573. Preferred embodiments are directed to methods wherein said umbilical cord cells or exosomes are co-administered simultaneously or sequentially with FGF-5.

574. Preferred embodiments are directed to methods wherein said umbilical cord cells or exosomes are co-administered simultaneously or sequentially with TGF-beta.

575. Preferred embodiments are directed to methods wherein said umbilical cord cells or exosomes are co-administered simultaneously or sequentially with TGF-beta.

576. Preferred embodiments are directed to methods wherein said umbilical cord cells or exosomes are co-administered simultaneously or sequentially with interleukin 1 receptor antagonist.

577. Preferred embodiments are directed to methods wherein said umbilical cord cells or exosomes are co-administered simultaneously or sequentially with interleukin 4.

578. Preferred embodiments are directed to methods wherein said umbilical cord cells or exosomes are co-administered simultaneously or sequentially with angiopoietin.

579. Preferred embodiments are directed to methods wherein said umbilical cord cells or exosomes are co-administered simultaneously or sequentially with G-CSF.

580. Preferred embodiments are directed to methods wherein said umbilical cord cells or exosomes are co-administered simultaneously or sequentially with GM-CSF.

581. Preferred embodiments are directed to methods wherein said umbilical cord cells or exosomes are co-administered simultaneously or sequentially with M-CSF.

582. Preferred embodiments are directed to methods wherein said umbilical cord cells or exosomes are co-administered simultaneously or sequentially with low dose naltrexone.

583. Preferred embodiments are directed to methods wherein said umbilical cord cells, and/or exosomes and/or additives are administered intranasally, intramuscularly, intraperitoneally, intralymphatically, intraomentally, intrarespiratorily, intranasopharyngeally or intravenously administration.

584. Preferred embodiments are directed to methods wherein the airways disease is selected from the list consisting of asthma, allergic rhinitis, severe acute respiratory syndrome, COVID-19, virally associated lung damage, radiation associated lung damage, chronic obstructive pulmonary disease, pulmonary fibrosis, upper respiratory infection and reactive airways dysfunction syndrome.

585. Preferred embodiments are directed to methods wherein said umbilical cord cells, and/or exosomes are administered together with a cellular population capable of reducing inflammation.

586. Preferred embodiments are directed to methods wherein said cellular population capable of reducing inflammation is a monocyte.

587. Preferred embodiments are directed to methods wherein said cellular population capable of reducing inflammation is a T regulatory cell.

588. Preferred embodiments are directed to methods wherein said cellular population capable of reducing inflammation is a Th2 cell.

589. Preferred embodiments are directed to methods wherein said cellular population capable of reducing inflammation is a Th9 cell.

590. Preferred embodiments are directed to methods wherein said cellular population capable of reducing inflammation is a Th3 cell.

591. Preferred embodiments are directed to methods wherein said cellular population capable of reducing inflammation is a Tr1 cell.

592. Preferred embodiments are directed to methods wherein said monocyte is a M2 monocyte.

593. Preferred embodiments are directed to methods wherein said M2 monocyte is derived from placenta, umbilical cord, bone marrow, wharton’s jelly, or adipose derived.

594. Preferred embodiments are directed to methods wherein said M2 monocyte is cultured in the presence of interleukin 4.

595. Preferred embodiments are directed to methods wherein said M2 monocyte is cultured in the presence of interleukin 13.

596. Preferred embodiments are directed to methods wherein said M2 monocyte is cultured in the presence of interleukin 10.

597. Preferred embodiments are directed to methods wherein said M2 monocyte is capable of stimulating angiogenesis.

598. Preferred embodiments are directed to methods wherein said T regulatory cell expresses FOXP3.

599. Preferred embodiments are directed to methods wherein said T regulatory cell expresses membrane bound TGF-beta.

600. Preferred embodiments are directed to methods wherein said T regulatory cell is capable of suppressing dendritic cell maturation.

601. Preferred embodiments are directed to methods wherein said T regulatory cell is capable of suppressing proliferation of T cells.

602. Preferred embodiments are directed to methods wherein said T regulatory cell is capable of suppressing cytotoxicity of T cells.

603. Preferred embodiments are directed to methods wherein said T regulatory cell is capable of suppressing type 1 cytokine production of T cells.

604. Preferred embodiments are directed to methods wherein said type 1 cytokines are selected from a group comprising of: a) IL-2; b) IL-7; c) IL-12; d) IL-15; and e) IL-18.

605. Preferred embodiments are directed to methods wherein said Th2 cell expresses STAT-6.

606. Preferred embodiments are directed to methods wherein said Th2 cell expresses IL-4 or IL-13.

607. Preferred embodiments are directed to methods wherein said Th9 cell expresses IL-9.

608. Preferred embodiments are directed to methods wherein said Th3 cell expresses TGF-beta.

609. Preferred embodiments are directed to methods wherein said Th3 cell is associated with induction of oral tolerance.

610. Preferred embodiments are directed to methods wherein said Tr1 cells are associated with production of IL-10.

611. Preferred embodiments are directed to methods wherein said exosomes are used to stimulate new blood vessel formation in a patient suffering from peripheral artery disease.

612. Preferred embodiments are directed to methods wherein said exosomes are used to stimulate new blood vessel formation in a patient suffering from critical limb ischemia.

613. Preferred embodiments are directed to methods wherein said exosomes are used to stimulate new blood vessel formation in a patient suffering from SARS-CoV-2 virus associated blood vessel dysfunction.

614. Preferred embodiments are directed to methods wherein said SARS-CoV-2 virus associated blood vessel dysfunction is associated with coagulopathy.

615. Preferred embodiments are directed to methods wherein said SARS-CoV-2 virus associated blood vessel dysfunction is associated with enhanced expression of tissue factor on endothelial cells.

616. Preferred embodiments are directed to methods wherein said SARS-CoV-2 virus associated blood vessel dysfunction is associated with enhanced endothelial cell activation.

617. Preferred embodiments are directed to methods wherein said SARS-CoV-2 virus associated blood vessel dysfunction is associated with reduced production of antithrombotic molecules on the endothelial surface.

618. Preferred embodiments are directed to methods wherein said SARS-CoV-2 virus associated blood vessel dysfunction is associated disseminated intravascular coagulation.

619. Preferred embodiments are directed to methods wherein said SARS-CoV-2 virus associated blood vessel dysfunction is associated with antiphospholipid antibody syndrome.

620. Preferred embodiments are directed to methods wherein said exosomes are used to stimulate new blood vessel formation in a patient suffering from ischemic heart failure.

621. Preferred embodiments are directed to methods wherein said exosomes are used to stimulate new blood vessel formation in a patient suffering post cardiac infarct scarring.

622. Preferred embodiments are directed to methods wherein said exosomes are used to stimulate new blood vessel formation in a patient suffering post cerebral infarct scarring.

623. Preferred embodiments are directed to methods wherein said exosomes are used to stimulate new blood vessel formation in a patient suffering post cerebral infarct scarring.

624. A method of stimulating regeneration in a tissue, wherein said in which regeneration is desired is distal from the tissue in which a regenerative means is administered through the steps of: a) identifying a diseased tissue in need of regeneration; and b) administering into said tissue a regenerative means in a manner so as to evoke a regenerative response in a tissue not treated with said regenerative means.

625. Preferred embodiments are directed to methods wherein said regeneration is desired in a tissue in which function has been lost or compromised.

626. Preferred embodiments are directed to methods wherein said tissue is comprised of one or more cells derived from the following: endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner’s gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells, bowman’s gland cells, Brunner’s gland cells, seminal vesicle cells, prostate gland cells, bulbourethral gland cells, Bartholin’s gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley’s layer, hair root sheath cells of Henle’s layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, reticulocytes, megakaryocytes, monocytes, connective tissue macrophages. epidermal Langerhans cells, dendritic cells, microglial cells, neutrophils, eosinophils, basophils, mast cell, helper T cells, suppressor T cells, cytotoxic T cell, natural Killer T cells, B cells, natural killer cells, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

627. Preferred embodiments are directed to methods wherein said regenerative means is a growth factor or combination of growth factors.

628. Preferred embodiments are directed to methods wherein said growth factors are one or more selected from a group comprising of: AM, Ang, BMP, BDNF, EGF, Epo, FGF, GNDF, G-CSF, GM-CSF, GDF-9, HGF, HDGF, IGF, migration-stimulating factor, GDF-8, GDF-11, GDF-15, MGF, NGF, P1GF, PDGF, Tpo, TGF-.alpha., TGF-.beta., TNF-.alpha., VEGF, or a Wnt protein; an interleukin; a soluble receptor for IL-1.alpha., IL-1.beta., IL-1F1, IL-1F2, IL-1F3, IL-1F4, IL-1F5, IL-1F6, IL-1F7, IL-1F8, IL-1F9, IL-2, IL-3, IL-4, IL-5, IL-6, IL-7, IL-8, IL-9, IL-10, IL-11, IL-12 35 kDa alpha subunit, IL-12 40 kDa beta subunit, IL-13, IL-14, IL-15, IL-16, IL-17A, IL-17B, IL-17C, IL-17D, IL-17E, IL-17F isoform 1, IL-17F isoform 2, IL-18, IL-19, IL-20, IL-21, IL-22, IL-23 p19 subunit, IL-23 p40 subunit, IL-24, IL-25, IL-26, IL-27B, IL-27-p28, IL-28A, IL-28B, IL-29, IL-30, IL-31, IL-32, IL-33, IL-34, IL-35, IL-36.alpha., IL-36.beta., IL-36.gamma.; an interferon (IFN); a soluble receptor for IFN-.alpha., IFN-.beta., IFN-.gamma., IFN-.lamda.1, IFN-.lamda.2, IFN-.lamda.3, IFN-K, IFN-.epsilon., IFN-.kappa., IFN-.tau., IFN-.delta., IFN-.zeta., IFN-.omega., or IFN-v; insulin or proinsulin; a receptor for insulin; leptin (LEP).

629. Preferred embodiments are directed to methods wherein said growth factor is platelet rich plasma.

630. Preferred embodiments are directed to methods wherein said platelet rich plasma is platelet lysate.

631. Preferred embodiments are directed to methods wherein said platelet rich plasma is derived from peripheral blood.

632. Preferred embodiments are directed to methods wherein said platelet rich plasma is derived from cord blood.

633. Preferred embodiments are directed to methods wherein said regenerative means comprises exosomes derived from a regenerative cell.

634. Preferred embodiments are directed to methods wherein said regenerative cell is a stem cell or a progenitor cell.

635. Preferred embodiments are directed to methods wherein said regenerative means is a mesenchymal stem cell.

636. Preferred embodiments are directed to methods wherein said mesenchymal stem cells are derived from a group of tissue sources selected from: a) foreskin; b) tummy tucks; c) placenta; d) ear lobe; e) adipose tissue; f) omentum; and g) wharton’s jelly.

637. Preferred embodiments are directed to methods wherein said mesenchymal express markers selected from a group comprising of: a) NANOG; b) OCT-4; c) SSEA-4; and d) stem cell factor receptor.

638. Preferred embodiments are directed to methods wherein said regenerative means is a regenerative cell.

639. Preferred embodiments are directed to methods wherein said stem cells are pluripotent stem cells.

640. Preferred embodiments are directed to methods wherein said pluripotent stem cells are selected from a group comprising of: a) embryonic stem cells; b) parthenogenic derived stem cells; c) inducible pluripotent stem cells; d) somatic cell nuclear transfer derived stem cells; e) cytoplasmic transfer derived stem cells; and f) stimulus-triggered acquisition of pluripotency.

641. Preferred embodiments are directed to methods wherein said stem cells are hematopoietic stem cell.

642. Preferred embodiments are directed to methods wherein said hematopoietic stem cells are capable of multi-lineage reconstitution in an immunodeficient host.

643. Preferred embodiments are directed to methods wherein said hematopoietic stem cells express the c-kit protein.

644. Preferred embodiments are directed to methods wherein said hematopoietic stem cells express the Sca-1 protein.

645. Preferred embodiments are directed to methods wherein said hematopoietic stem cells express CD34.

646. Preferred embodiments are directed to methods wherein said hematopoietic stem cells express CD133.

647. Preferred embodiments are directed to methods wherein said hematopoietic stem cells lack expression of lineage markers.

648. Preferred embodiments are directed to methods wherein said hematopoietic stem cells lack expression of CD38.

649. Preferred embodiments are directed to methods wherein said hematopoietic stem cells are positive for expression of c-kit and Sca-1 and substantially lack expression of lineage markers.

650. Preferred embodiments are directed to methods wherein said hematopoietic stem cells are derived from a group of sources, said group comprising of: a) peripheral blood; b) mobilized peripheral blood; c) bone marrow; d) cord blood; e) adipose stromal vascular fraction; and f) derived from progenitor cells.

651. Preferred embodiments are directed to methods wherein said progenitor cell is a pluripotent stem cell.

652. Preferred embodiments are directed to methods wherein said stem cells are mesenchymal stem cells.

653. Preferred embodiments are directed to methods wherein said mesenchymal stem cells are plastic adherent.

654. Preferred embodiments are directed to methods wherein said mesenchymal stem cells express a marker selected from a group comprising of: a) CD73; b) CD90; and c) CD105.

655. Preferred embodiments are directed to methods wherein said mesenchymal stem cells lack expression of a marker selected from a group comprising of: a) CD14; b) CD45; and c) CD34.

656. Preferred embodiments are directed to methods wherein said mesenchymal stem cells are derived from tissues selected from a group comprising of: a) bone marrow; b) peripheral blood; c) adipose tissue; d) mobilized peripheral blood; e) umbilical cord blood; f) Wharton’s jelly; g) umbilical cord tissue; h) skeletal muscle tissue; i) subepithelial umbilical cord; j) endometrial tissue; k) menstrual blood; and 1) fallopian tube tissue.

657. Preferred embodiments are directed to methods wherein said mesenchymal stem cells from umbilical cord tissue express markers selected from a group comprising of; a) oxidized low density lipoprotein receptor 1, b) chemokine receptor ligand 3; and c) granulocyte chemotactic protein.

658. Preferred embodiments are directed to methods wherein said mesenchymal stem cells from umbilical cord tissue do not express markers selected from a group comprising of: a) CD117; b) CD31; c) CD34; and CD45;

659. Preferred embodiments are directed to methods wherein said mesenchymal stem cells from umbilical cord tissue express, relative to a human fibroblast, increased levels of interleukin 8 and reticulon 1

660. Preferred embodiments are directed to methods wherein said mesenchymal stem cells from umbilical cord tissue have the potential to differentiate into cells of at least a skeletal muscle, vascular smooth muscle, pericyte or vascular endothelium phenotype.

661. Preferred embodiments are directed to methods wherein said mesenchymal stem cells from umbilical cord tissue express markers selected from a group comprising of: a) CD10; b) CD13; c) CD44; d) CD73; and e) CD90.

662. Preferred embodiments are directed to methods wherein said umbilical cord tissue mesenchymal stem cell is an isolated umbilical cord tissue cell isolated from umbilical cord tissue substantially free of blood that is capable of self-renewal and expansion in culture,

663. Preferred embodiments are directed to methods wherein said umbilical cord tissue mesenchymal stem cells has the potential to differentiate into cells of other phenotypes.

664. Preferred embodiments are directed to methods wherein said other phenotypes comprise: a) osteocytic; b) adipogenic; and c) chondrogenic differentiation.

665. Preferred embodiments are directed to methods wherein said cord tissue derived mesenchymal stem cells can undergo at least 20 doublings in culture.

665. Preferred embodiments are directed to methods wherein said cord tissue derived mesenchymal stem cell maintains a normal karyotype upon passaging

667. Preferred embodiments are directed to methods wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C

668. Preferred embodiments are directed to methods wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR,DP,DQ.

669. Preferred embodiments are directed to methods wherein said umbilical cord tissue-derived cell secretes factors selected from a group comprising of: a) MCP-1; b) MIP1beta; c) IL-6; d) IL-8; e) GCP-2; f) HGF; g) KGF; h) FGF; i) HB-EGF; j) BDNF; k) TPO; 1) RANTES; and m) TIMP1

670. Preferred embodiments are directed to methods wherein said umbilical cord tissue derived cells express markers selected from a group comprising of: a) TRA1-60; b) TRA1-81; c) SSEA3; d) SSEA4; and e) NANOG.

671. Preferred embodiments are directed to methods wherein said umbilical cord tissue-derived cells are positive for alkaline phosphatase staining.

672. Preferred embodiments are directed to methods wherein said umbilical cord tissue-derived cells are capable of differentiating into one or more lineages selected from a group comprising of; a) ectoderm; b) mesoderm, and; c) endoderm.

673. Preferred embodiments are directed to methods wherein said bone marrow derived mesenchymal stem cells possess markers selected from a group comprising of: a) CD73; b) CD90; and c) CD105.

674. Preferred embodiments are directed to methods wherein said bone marrow derived mesenchymal stem cells possess markers selected from a group comprising of: a) LFA-3; b) ICAM-1; c) PECAM-1; d) P-selectin; e) L-selectin; f) CD49b/CD29; g) CD49c/CD29; h) CD49d/CD29; i) CD29; j) CD18; k) CD61; 1) 6-19; m) thrombomodulin; n) telomerase; o) CD10; p) CD13; and q) integrin beta.

675. Preferred embodiments are directed to methods wherein said bone marrow derived mesenchymal stem cell is a mesenchymal stem cell progenitor cell.

676. Preferred embodiments are directed to methods wherein said mesenchymal progenitor cells are a population of bone marrow mesenchymal stem cells enriched for cells containing STRO-1

677. Preferred embodiments are directed to methods wherein said mesenchymal progenitor cells express both STRO-1 and VCAM-1.

678. Preferred embodiments are directed to methods wherein said STRO-1 expressing cells are negative for at least one marker selected from the group consisting of: a) CBFA-1; b) collagen type II; c) PPAR.gamma2; d) osteopontin; e) osteocalcin; f) parathyroid hormone receptor; g) leptin; h) H-ALBP; i) aggrecan; j) Ki67, and k) glycophorin A.

679. Preferred embodiments are directed to methods wherein said bone marrow mesenchymal stem cells lack expression of CD14, CD34, and CD45.

680. Preferred embodiments are directed to methods wherein said STRO-1 expressing cells are positive for a marker selected from a group comprising of: a) VACM-1; b) TKY-1; c) CD146 and; d) STRO-2

681. Preferred embodiments are directed to methods wherein said bone marrow mesenchymal stem cell express markers selected from a group comprising of: a) CD13; b) CD34; c) CD56 and; d) CD117

682. Preferred embodiments are directed to methods wherein said bone marrow mesenchymal stem cells do not express CD10.

683. Preferred embodiments are directed to methods wherein said bone marrow mesenchymal stem cells do not express CD2, CD5, CD14, CD19, CD33, CD45, and DRII.

684. Preferred embodiments are directed to methods wherein said bone marrow mesenchymal stem cells express CD13,CD34, CD56, CD90, CD117 and nestin, and which do not express CD2, CD3, CD10, CD14, CD16, CD31, CD33, CD45 and CD64.

685. Preferred embodiments are directed to methods wherein said skeletal muscle stem cells express markers selected from a group comprising of: a) CD13; b) CD34; c) CD56 and; d) CD117

686. Preferred embodiments are directed to methods wherein said skeletal muscle mesenchymal stem cells do not express CD10.

687. Preferred embodiments are directed to methods wherein said skeletal muscle mesenchymal stem cells do not express CD2, CD5, CD14, CD19, CD33, CD45, and DRII.

688. Preferred embodiments are directed to methods wherein said subepithelial umbilical cord derived mesenchymal stem cells possess markers selected from a group comprising of; a) CD29; b) CD73; c) CD90; d) CD166; e) SSEA4; f) CD9; g) CD44; h) CD146; and i) CD105

689. Preferred embodiments are directed to methods wherein said subepithelial umbilical cord derived mesenchymal stem cells do not express markers selected from a group comprising of; a)CD45; b) CD34; c) CD14; d) CD79; e) CD106; f) CD86; g) CD80; h) CD19; i) CD117; j) Stro-1 and k) HLA-DR.

690. Preferred embodiments are directed to methods wherein said subepithelial umbilical cord derived mesenchymal stem cells express CD29, CD73, CD90, CD166, SSEA4, CD9, CD44, CD146, and CD105.

691. Preferred embodiments are directed to methods wherein said subepithelial umbilical cord derived mesenchymal stem cells do not express CD45, CD34, CD14, CD79, CD106, CD86, CD80, CD19, CD117, Stro-1, and HLA-DR.

692. Preferred embodiments are directed to methods wherein said subepithelial umbilical cord derived mesenchymal stem cells are positive for SOX2.

693. Preferred embodiments are directed to methods wherein said subepithelial umbilical cord derived mesenchymal stem cells are positive for OCT4.

694. Preferred embodiments are directed to methods wherein said subepithelial umbilical cord derived mesenchymal stem cells are positive for OCT4 and SOX2.

695. Preferred embodiments are directed to methods wherein said regenerative means comprises umbilical cord cell derived apoptotic vesicles.

696. Preferred embodiments are directed to methods wherein said regenerative means comprises umbilical cord cell derived miRNAs.

697. Preferred embodiments are directed to methods wherein said exosomes possess a size of between 30 nm and 150 nm.

698. Preferred embodiments are directed to methods wherein said exosome possesses a size of between 2 nm and 200 nm, as determined by filtration against a 0.2 .mu.M filter and concentration against a membrane with a molecular weight cut-off of 10 kDa, or a hydrodynamic radius of below 100 nm as determined by laser diffraction or dynamic light scattering.

699. Preferred embodiments are directed to methods wherein said exosome possesses a lipid selected from the group consisting of: a) phospholipids; b) phosphatidyl serine; c) phosphatidyl inositol; d) phosphatidyl choline; e) sphingomyelin; f) ceramides; g) glycolipid; h) cerebroside; i) steroids, and j) cholesterol.

700. Preferred embodiments are directed to methods wherein said exosome possesses a lipid raft.

701. Preferred embodiments are directed to methods wherein said exosome expresses antigenic markers on surface of said exosome, wherein said antigenic markers are selected from a group comprising of: a) CD9; b) CD63; c) CD81; d) ANXA2; e) ENO1; f) HSP90AA1; g) EEF1A1; h) YWHAE; i) SDCBP; j) PDCD6IP; k) ALB; 1) YWHAZ; m) EEF2; n) ACTG1; o) LDHA; p) HSP90AB1; q) ALDOA; r) MSN; s) ANXA5; t) PGK1; and u) CFL1.

702. Preferred embodiments are directed to methods wherein enhancement of distant regenerative effect is accomplished by systemic administration of an epigenetic acting drug.

703. Preferred embodiments are directed to methods wherein said epigenetic acting drug is a histone deacetylase inhibitor.

704. Preferred embodiments are directed to methods wherein said epigenetic acting drug is a DNA methyltransferase inhibitor.

705. Preferred embodiments are directed to methods wherein said exosomes are utilized to reprogram another cell type in vivo, in vitro, or ex vivo, wherein reprogramming by said exosomes endows said cell type with a therapeutic property.

706. Preferred embodiments are directed to methods wherein said exosomes are incubated with T cells in order to generate T cells with therapeutic properties.

707. Preferred embodiments are directed to methods wherein said T cells are endowed with ability to stimulate angiogenesis after culture with said umbilical cord derived exosomes.

708. Preferred embodiments are directed to methods wherein said T cells are endowed with ability to stimulate neurogenesis after culture with said umbilical cord derived exosomes.

709. Preferred embodiments are directed to methods wherein said T cells are endowed with ability to stimulate suppression of inflammation after culture with said umbilical cord derived exosomes.

710. Preferred embodiments are directed to methods wherein said T cells are endowed with ability to stimulate neutrophil apoptosis after culture with said umbilical cord derived exosomes.

711. Preferred embodiments are directed to methods wherein said T cells are endowed with ability to suppress maturation of dendritic cells after culture with said umbilical cord derived exosomes.

712. Preferred embodiments are directed to methods wherein said T cells are endowed with ability to suppress generation of interleukin-17 after culture with said umbilical cord derived exosomes.

713. Preferred embodiments are directed to methods wherein said exosomes are utilized to reprogram a macrophage to endow onto said macrophage angiogenic activity.

714. Preferred embodiments are directed to methods wherein said exosomes are utilized to reprogram a macrophage to endow onto said macrophage antifibrotic activity.

715. Preferred embodiments are directed to methods wherein said exosomes are utilized to reprogram a macrophage to endow onto said macrophage neurogenic activity.

716. Preferred embodiments are directed to methods wherein said exosomes are utilized to reprogram a macrophage to endow onto said macrophage anti-apoptotic activity.

717. Preferred embodiments are directed to methods wherein said exosomes are utilized to reprogram a macrophage to endow onto said macrophage wound healing activity.

718. Preferred embodiments are directed to methods wherein said exosomes are utilized to reprogram a macrophage to endow onto said macrophage anti-inflammatory activity.

719. Preferred embodiments are directed to methods wherein said exosomes are utilized to reprogram a macrophage to endow onto said macrophage ability to induce neutrophil apoptosis.

720. Preferred embodiments are directed to methods wherein said exosomes are utilized to reprogram a macrophage to endow onto said macrophage ability to suppress dendritic cell maturation.

721. Preferred embodiments are directed to methods wherein said exosomes are utilized to reprogram a macrophage to endow onto said macrophage ability to suppress generation of Th1 cells.

722. Preferred embodiments are directed to methods wherein said Th1 cells express Helios.

723. Preferred embodiments are directed to methods wherein said Th1 cells express interferon gamma.

724. Preferred embodiments are directed to methods wherein said Th1 cells express STAT4.

725. Preferred embodiments are directed to methods wherein said exosomes are utilized to reprogram a macrophage to endow onto said macrophage ability to stimulate generation of T regulatory cells.

726. Preferred embodiments are directed to methods wherein said T regulatory cells are capable of inhibiting inflammation.

727. Preferred embodiments are directed to methods wherein said T regulatory cells are capable of inhibiting autoimmunity.

728. Preferred embodiments are directed to methods wherein said T regulatory cells are capable of inhibiting multiple organ failure.

729. Preferred embodiments are directed to methods wherein said T regulatory cells are capable of inhibiting acute respiratory distress syndrome.

730. Preferred embodiments are directed to methods wherein said T regulatory cells are capable of inhibiting liver failure.

731. Preferred embodiments are directed to methods wherein said T regulatory cells are capable of inhibiting pulmonary fibrosis.

732. Preferred embodiments are directed to methods wherein said exosomes are used to reprogram a macrophage, wherein said reprogrammed macrophage can be used for treatment of an orthopedic injury.

733. Preferred embodiments are directed to methods wherein said reprogrammed macrophage is a M2 macrophage.

734. Preferred embodiments are directed to methods wherein said reprogrammed macrophage expresses a substantially higher amount of VEGF as compared on a macrophage that has not been reprogrammed.

735. Preferred embodiments are directed to methods wherein said reprogrammed macrophage expresses a substantially higher amount of FGF-1 as compared on a macrophage that has not been reprogrammed.

736. Preferred embodiments are directed to methods wherein said reprogrammed macrophage expresses a substantially higher amount of FGF-2 as compared on a macrophage that has not been reprogrammed.

737. Preferred embodiments are directed to methods wherein said reprogrammed macrophage expresses a substantially higher amount of FGF-5 as compared on a macrophage that has not been reprogrammed.

738. Preferred embodiments are directed to methods wherein said reprogrammed macrophage expresses a substantially higher amount of HGF-1 as compared on a macrophage that has not been reprogrammed.

739. Preferred embodiments are directed to methods wherein said reprogrammed macrophage expresses a substantially higher amount of PDGF-1 as compared on a macrophage that has not been reprogrammed.

740. Preferred embodiments are directed to methods wherein said reprogrammed macrophage expresses a substantially higher amount of interleukin 1 receptor antagonist as compared on a macrophage that has not been reprogrammed.

741. Preferred embodiments are directed to methods wherein said reprogrammed macrophage expresses a substantially higher amount of interleukin 1 receptor antagonist as compared on a macrophage that has not been reprogrammed.

742. Preferred embodiments are directed to methods wherein said reprogrammed macrophage expresses a substantially higher amount of interleukin-10 as compared on a macrophage that has not been reprogrammed.

743. Preferred embodiments are directed to methods wherein said reprogrammed macrophage expresses a substantially higher amount of PGE-2 as compared on a macrophage that has not been reprogrammed.

744. Preferred embodiments are directed to methods wherein said reprogrammed macrophage expresses a substantially higher amount of indolamine 2,3 deoxygenase as compared on a macrophage that has not been reprogrammed.

745. Preferred embodiments are directed to methods wherein said reprogrammed macrophage expresses a substantially higher amount of Galectin 3 as compared on a macrophage that has not been reprogrammed.

746. Preferred embodiments are directed to methods wherein said reprogrammed macrophage expresses a substantially higher amount of Galectin 9 as compared on a macrophage that has not been reprogrammed.

747. Preferred embodiments are directed to methods wherein said reprogrammed macrophage expresses a substantially higher amount of HLA-G as compared on a macrophage that has not been reprogrammed.

748. Preferred embodiments are directed to methods wherein said reprogrammed macrophage expresses a substantially higher amount of ILT-3 as compared on a macrophage that has not been reprogrammed.

749. Preferred embodiments are directed to methods wherein said reprogrammed macrophage expresses a substantially higher amount of TIGIT as compared on a macrophage that has not been reprogrammed.

750. Preferred embodiments are directed to methods wherein said reprogrammed macrophage expresses a substantially higher amount of arginase as compared on a macrophage that has not been reprogrammed.

751. Preferred embodiments are directed to methods wherein said reprogrammed macrophage expresses a substantially higher amount of IL-6 as compared on a macrophage that has not been reprogrammed.

752. Preferred embodiments are directed to methods wherein said reprogrammed macrophage expresses a substantially higher amount of serpin-1 as compared on a macrophage that has not been reprogrammed.

753. Preferred embodiments are directed to methods wherein said reprogrammed macrophage expresses a substantially higher amount of PD-L1 as compared on a macrophage that has not been reprogrammed.

754. Preferred embodiments are directed to methods wherein said reprogrammed macrophage expresses a substantially higher amount of PD-L2 as compared on a macrophage that has not been reprogrammed.

755. Preferred embodiments are directed to methods wherein said reprogrammed macrophage expresses a substantially higher amount of CD206 as compared on a macrophage that has not been reprogrammed.

756. Preferred embodiments are directed to methods wherein said reprogrammed macrophage expresses a substantially lower amount of eotoxin as compared on a macrophage that has not been reprogrammed.

757. Preferred embodiments are directed to methods wherein said reprogrammed macrophage expresses a substantially lower amount of CD14 as compared on a macrophage that has not been reprogrammed.

758. Preferred embodiments are directed to methods wherein said reprogrammed macrophage expresses a substantially lower amount of M-CSF receptor as compared on a macrophage that has not been reprogrammed.

759. Preferred embodiments are directed to methods wherein said reprogrammed macrophage expresses a substantially lower amount of TGF-alpha as compared on a macrophage that has not been reprogrammed.

760. Preferred embodiments are directed to methods wherein said reprogrammed macrophage expresses a substantially lower amount of interleukin 13 as compared on a macrophage that has not been reprogrammed.

761. Preferred embodiments are directed to methods wherein said population of reprogrammed macrophage cells is administered by injection.

762. Preferred embodiments are directed to methods wherein the population of cells is administered by injection with a pharmaceutically acceptable carrier.

763. Preferred embodiments are directed to methods wherein said pharmaceutically acceptable carrier maintains viability and function of said reprogrammed macrophages.

764. Preferred embodiments are directed to methods wherein said pharmaceutically acceptable carrier is hyaluronic acid.

765. Preferred embodiments are directed to methods wherein said pharmaceutically acceptable carrier is decellularized tissue.

766. Preferred embodiments are directed to methods wherein said decellularized tissue is placental tissue.

767. Preferred embodiments are directed to methods wherein said decellularized tissue is omentum tissue.

768. Preferred embodiments are directed to methods wherein said decellularized tissue is adipose tissue.

769. Preferred embodiments are directed to methods wherein said decellularized tissue is bone marrow tissue.

770. Preferred embodiments are directed to methods wherein said decellularized tissue is subintestinal mucosa tissue.

771. Preferred embodiments are directed to methods wherein said cells are administered together with an antioxidant.

772. Preferred embodiments are directed to methods wherein said cells are administered together with a growth factor.

773. Preferred embodiments are directed to methods wherein said growth factor is TGF-beta.

774. Preferred embodiments are directed to methods wherein said growth factor is VEGF.

775. Preferred embodiments are directed to methods wherein said growth factor is EGF.

776. Preferred embodiments are directed to methods wherein said growth factor is NGF.

777. Preferred embodiments are directed to methods wherein said growth factor is HGF-1.

778. Preferred embodiments are directed to methods wherein said growth factor is bone morphogenic protein-1.

779. Preferred embodiments are directed to methods wherein said growth factor is bone morphogenic protein-2.

780. Preferred embodiments are directed to methods wherein said growth factor is platelet rich plasma.

781. Preferred embodiments are directed to methods wherein said growth factor is conditioned media from a regenerative cell treated with an inflammatory stimuli.

782. Preferred embodiments are directed to methods wherein said regenerative cell is a mesenchymal stem cell.

783. Preferred embodiments are directed to methods wherein said regenerative cell is cord blood mononuclear cells.

784. Preferred embodiments are directed to methods wherein said regenerative cell is peripheral blood mononuclear cells.

785. Preferred embodiments are directed to methods wherein said regenerative cell is bone marrow mononuclear cells.

786. Preferred embodiments are directed to methods wherein said regenerative cell is stromal vascular fraction cells.

787. Preferred embodiments are directed to methods wherein said regenerative cell is a mixed lymphocyte reaction.

788. Preferred embodiments are directed to methods wherein said regenerative cell is thymic medullary epithelial cells.

789. Preferred embodiments are directed to methods wherein said regenerative cell is dendritic cells.

790. Preferred embodiments are directed to methods wherein said inflammatory stimuli is a toll like receptor agonist.

791. Preferred embodiments are directed to methods wherein said toll like receptor is TLR-1.

792. Preferred embodiments are directed to methods wherein said activator of TLR-1 is Pam3CSK4.

793. Preferred embodiments are directed to methods wherein said toll like receptor is TLR-2.

794. Preferred embodiments are directed to methods wherein said activator of TLR-2 is HKLM.

795. Preferred embodiments are directed to methods wherein said toll like receptor is TLR-3.

796. Preferred embodiments are directed to methods wherein said activator of TLR-3 is Poly:IC.

797. Preferred embodiments are directed to methods wherein said toll like receptor is TLR-4.

798. Preferred embodiments are directed to methods wherein said activator of TLR-4 is LPS.

799. Preferred embodiments are directed to methods wherein said activator of TLR-4 is Buprenorphine.

800. Preferred embodiments are directed to methods wherein said activator of TLR-4 is Carbamazepine.

801. Preferred embodiments are directed to methods wherein said activator of TLR-4 is Fentanyl.

802. Preferred embodiments are directed to methods wherein said activator of TLR-4 is Levorphanol.

803. Preferred embodiments are directed to methods wherein said activator of TLR-4 is Methadone.

804. Preferred embodiments are directed to methods wherein said activator of TLR-4 is Cocaine.

805. Preferred embodiments are directed to methods wherein said activator of TLR-4 is Morphine.

806. Preferred embodiments are directed to methods wherein said activator of TLR-4 is Oxcarbazepine.

807. Preferred embodiments are directed to methods wherein said activator of TLR-4 is Oxycodone.

808. Preferred embodiments are directed to methods wherein said activator of TLR-4 is Pethidine.

809. Preferred embodiments are directed to methods wherein said activator of TLR-4 is Glucuronoxylomannan from Cryptococcus.

810. Preferred embodiments are directed to methods wherein said activator of TLR-4 is Morphine-3-glucuronide.

811. Preferred embodiments are directed to methods wherein said activator of TLR-4 is lipoteichoic acid.

812. Preferred embodiments are directed to methods wherein said activator of TLR-4 is β-defensin 2.

813. Preferred embodiments are directed to methods wherein said activator of TLR-4 is small molecular weight hyaluronic acid.

814. Preferred embodiments are directed to methods wherein said activator of TLR-4 is fibronectin EDA.

815. Preferred embodiments are directed to methods wherein said activator of TLR-4 is snapin.

816. Preferred embodiments are directed to methods wherein said activator of TLR-4 is tenascin C.

817. Preferred embodiments are directed to methods wherein said toll like receptor is TLR-5.

818. Preferred embodiments are directed to methods wherein said activator of TLR-5 is flagellin.

819. Preferred embodiments are directed to methods wherein said toll like receptor is TLR-6.

820. Preferred embodiments are directed to methods wherein said activator of TLR-6 is FSL-1.

821. Preferred embodiments are directed to methods wherein said toll like receptor is TLR-7.

822. Preferred embodiments are directed to methods wherein said activator of TLR-7 is imiquimod.

823. Preferred embodiments are directed to methods wherein said toll like receptor of TLR-8.

825. Preferred embodiments are directed to methods wherein said activator of TLR8 is ssRNA40/LyoVec.

826. Preferred embodiments are directed to methods wherein said toll like receptor of TLR-9.

827. Preferred embodiments are directed to methods wherein said activator of TLR-9 is a CpG oligonucleotide.

828. Preferred embodiments are directed to methods wherein said activator of TLR-9 is ODN2006.

829. Preferred embodiments are directed to methods wherein said activator of TLR-9 is Agatolimod.

830. Preferred embodiments are directed to methods wherein the population of cells is administered surgically.

831. Preferred embodiments are directed to methods wherein said orthopedic injury is selected from the group consisting of a partial tendon tear, a complete tendon tear, a partial tendon laceration, a compete tendon laceration, a partial tendon avulsion, a complete tendon avulsion, a partial ligament tear, a complete ligament tear, a partial ligament laceration, a compete ligament laceration, tendinopathy, tendinosis, tendinitis, meniscal tears, joint capsule tears.

832. Preferred embodiments are directed to methods wherein the orthopedic injury is selected from the group consisting of plantar fasciitis, tennis elbow, bicep tendinitis, and carpal tunnel syndrome.

833. Preferred embodiments are directed to methods wherein the population of reprogrammed macrophages is generated by a method comprising the step of: co-culturing a CD56 expressing umbilical cord mesenchymal stem cell with a monocyte in a manner in which said monocyte acquires anti-inflammatory properties.

834. Preferred embodiments are directed to methods wherein said culture is performed in the presence of hypoxia.

835. Preferred embodiments are directed to methods wherein said culture is performed in the presence of valproic acid.

836. Preferred embodiments are directed to methods wherein said culture is performed in the presence of valproic acid.

837. Preferred embodiments are directed to methods wherein said culture is performed in the presence of lithium.

838. Preferred embodiments are directed to methods wherein said culture is performed in the presence of GM-CSF.

839. Preferred embodiments are directed to methods wherein said culture is performed in the presence of CNTF.

840. Preferred embodiments are directed to methods wherein said culture is performed in the presence of trichostatin-A.

841. Preferred embodiments are directed to methods wherein said culture is performed in the presence of an antioxidant.

842. Preferred embodiments are directed to methods wherein said antioxidant is selected from a group comprising of: a) ascorbic acid; b) n-acetylcysteine; c) glutathione; d) superoxide dismutase; e) rutin; f) pterostilbene; g) resveratrol; h) vitamin A; i) vitamin D; j) vitamin E; k) fish oil; and 1) quercetin.

843. Preferred embodiments are directed to methods wherein said culture is performed in the presence of an inhibitor of NF-kappa B.

844. Preferred embodiments are directed to methods wherein said inhibitor of NF-kappa B is selected from a group comprising of: Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic)

845. Preferred embodiments are directed to methods wherein said culture is performed in the presence of low level laser irradiation.

846. Preferred embodiments are directed to methods wherein said low level laser irradiation is provided in at least one wavelength, said wavelength in a range between about 620 nanometers and about 1070 nanometers.

847. Preferred embodiments are directed to methods wherein said laser irradiation is administered by a light source between approximately 100 .mu.W/cm.sup.2 to approximately 10 W/cm.sup.2.

848. Preferred embodiments are directed to methods wherein said low level laser irradiation enhances growth factor production of said cells.

849. Preferred embodiments are directed to methods wherein said low level laser irradiation enhances chemotactic activityof said cells.

850. Preferred embodiments are directed to methods wherein said chemotactic ability is correlated with expression of the chemokine receptor CXCR-4, which is a membrane bound receptor whose ligand is SDF-1, otherwise known as CXCL12.

851. A method of generating therapeutic macrophages comprising the steps of: a) obtaining a monocyte or monocyte progenitor cell; and b) contacting said monocyte or monocytic progenitor cell with a umbilical cord stem cell for a sufficient time period to endow said monocyte or monocytic progenitor cell with ability to stimulate angiogenesis.

852. Preferred embodiments are directed to methods wherein said monocyte cells are derived from peripheral blood mononuclear cells.

853. Preferred embodiments are directed to methods wherein said monocyte cells express CD14.

854. Preferred embodiments are directed to methods wherein said monocyte cells express CD16.

855. Preferred embodiments are directed to methods wherein said monocyte cells are plastic adherent.

856. Preferred embodiments are directed to methods wherein said monocyte cells express CD11b.

857. Preferred embodiments are directed to methods wherein said monocytic progenitor is a myeloid progenitor cell.

858. Preferred embodiments are directed to methods wherein said monocyte cells are derived from bone marrow.

859. Preferred embodiments are directed to methods wherein said monocyte cells express CD14.

860. Preferred embodiments are directed to methods wherein said monocyte cells express CD16.

861. Preferred embodiments are directed to methods wherein said monocyte cells are plastic adherent.

862. Preferred embodiments are directed to methods wherein said monocyte cells express CD11b.

863. Preferred embodiments are directed to methods wherein said monocytic progenitor is a myeloid progenitor cell.

864. Preferred embodiments are directed to methods wherein said monocytes are derived from mobilized peripheral blood.

865. Preferred embodiments are directed to methods wherein said mobilization of peripheral blood is accomplished through pretreatment of the patient with G-CSF.

866. Preferred embodiments are directed to methods wherein said mobilization of peripheral blood is accomplished through pretreatment of the patient with flt-3 ligand.

867. Preferred embodiments are directed to methods wherein said mobilization of peripheral blood is accomplished through pretreatment of the patient with Mozibil.

868. Preferred embodiments are directed to methods wherein said mobilization of peripheral blood is accomplished through pretreatment of the patient with Mozibil.

869. Preferred embodiments are directed to methods wherein said monocyte cells express CD14.

870. Preferred embodiments are directed to methods wherein said monocyte cells express CD16.

871. Preferred embodiments are directed to methods wherein said monocyte cells are plastic adherent.

872. Preferred embodiments are directed to methods wherein said monocyte cells express CD11b.

873. Preferred embodiments are directed to methods wherein said umbilical cord cell is derived from Wharton’s Jelly and is derived from either the patient to be treated (autologous) or said donor is different from the patient to be treated (allogeneic).

874. Preferred embodiments are directed to methods wherein said umbilical cord stem cells are cultured in a media allowing for stem cell proliferation.

875. Preferred embodiments are directed to methods wherein said media allowing for umbilical cord stem cell proliferation contains one or more factors known to be mitogenic for mesenchymal stem cells.

876. Preferred embodiments are directed to methods wherein said factors known to be mitogenic for mesenchymal stem cells include one or more factors selected from a group comprising of: a) FGF-1; b) FGF-2; c) FGF-5; d) EGF; e) CNTF; f) KGF-1; g) PDGF; h) platelet rich plasma; i) TGF-alpha; and j) HGF-1.

877. Preferred embodiments are directed to methods wherein said stem cells are cultured under hypoxia.

878. Preferred embodiments are directed to methods wherein said stem cells are exposed to allogeneic T cells to induce enhancement of regenerative activity.

879. Preferred embodiments are directed to methods wherein said stem cells are exposed to extracorporeal pulse wave ultrasound in order to induce expression of stress related proteins such as hsp90..

880. Preferred embodiments are directed to methods wherein said stem cells are treated with an inflammatory stimuli to mimic a wound environment.

881. Preferred embodiments are directed to methods wherein said inflammatory stimuli is an inflammatory cytokine.

882. Preferred embodiments are directed to methods wherein said inflammatory cytokine is TNF-alpha.

883. Preferred embodiments are directed to methods wherein said inflammatory cytokine is IL-1.

884. Preferred embodiments are directed to methods wherein said inflammatory cytokine is IL-6.

885. Preferred embodiments are directed to methods wherein said inflammatory cytokine is IL-11.

886. Preferred embodiments are directed to methods wherein said inflammatory cytokine is IL-12..

887. Preferred embodiments are directed to methods wherein said inflammatory cytokine is IL-17..

888. Preferred embodiments are directed to methods wherein said inflammatory cytokine is IL-18..

889. Preferred embodiments are directed to methods wherein said inflammatory cytokine is IL-21..

890. Preferred embodiments are directed to methods wherein said inflammatory cytokine is IL-33..

891. Preferred embodiments are directed to methods wherein said inflammatory cytokine is capable of stimulating expression of genes in fibroblast cells selected from a group comprising of: IL-6, Myosin 1, IL-33, Hypoxia Inducible Factor-1, Guanylate Binding Protein Isoform I, Aminolevulinate delta synthase 2, AMP deaminase, IL-17, DNAJ-like 2 protein, Cathepsin L, Transcription factor-20, M31724, pyenylalkylamine binding protein; HEC, GA17, arylsulfatase D gene, arylaulfatase E gene, cyclin protein gene, pro-platelet basic protein gene, PDGFRA, human STS WI-12000, mannosidase, beta A, lysosomal MANBA gene, UBE2D3 gene, Human DNA for Ig gamma heavy-chain, STRL22, BHMT, homo sapiens Down syndrome critical region, FI5613 containing ZNF gene family member, IL8, ELFR, homo sapiens mRNA for dual specificity phosphatase MKP-5, homo sapiens regulator of G protein signaling 10 mRNA complete, Homo sapiens Wnt-13 Mma, homo sapiens N-terminal acetyltransferase complex ard1 subunit, ribosomal protein L15 mRNA, PCNA mRNA, ATRM gene exon 21, HR gene for hairless protein exon 2, N-terminal acetyltransferase complex ard 1 subunit, HSM801431 homo sapiens mRNA, CDNA DKFZp434N2072,RPL26, and HR gene for hairless protein, regulator of G protein signaling

892. Preferred embodiments are directed to methods wherein said monocytes or monocytic progenitors are cultured with said stem cells at a ratio of 1 monocyte or monocytic progenitor cell to 100 stem cells.

893. Preferred embodiments are directed to methods wherein said monocytes or monocytic progenitors are cultured with said stem cells at a ratio of 1 monocyte or monocytic progenitor cell to 10 stem cells.

894. Preferred embodiments are directed to methods wherein said monocytes or monocytic progenitors are cultured with said stem cells at a ratio of 1 monocyte or monocytic progenitor cell to 10 stem cells.

895. Preferred embodiments are directed to methods wherein said monocytes or monocytic progenitors are cultured with said stem cells at a ratio of 1 monocyte or monocytic progenitor cell to 1 stem cells.

896. Preferred embodiments are directed to methods wherein said monocytes or monocytic progenitors are cultured with said stem cells for a time period of 1 hour to 7 days.

897. Preferred embodiments are directed to methods wherein said monocytes or monocytic progenitors are cultured with said stem cells for a time period of 12 hours to 5 days.

898. Preferred embodiments are directed to methods wherein said monocytes or monocytic progenitors are cultured with said stem cells for a time period of 1 to 3 days.

899. Preferred embodiments are directed to methods wherein said monocytes or monocytic progenitors are cultured with said fibroblasts in a media selected from a group comprising of: Roswell Park Memorial Institute (RPMI-1640), Dublecco’s Modified Essential Media (DMEM), Dublecco’s Modified Essential Media - Low Glucose (DMEM-LG), Eagle’s Modified Essential Media (EMEM), Optimem, Iscove’s Media, or combinations thereof.

900. Preferred embodiments are directed to methods wherein said media is supplemented with growth factors selected from a group comprising of: human platelet rich plasma, platelet lysate, umbilical cord blood serum, autologous serum, human serum, serum replacement, or combinations thereof.

901. Preferred embodiments are directed to methods wherein in said therapeutic macrophages are capable of stimulating growth of new blood vessels.

902. Preferred embodiments are directed to methods wherein in said therapeutic macrophages are M2 macrophage.

903. Preferred embodiments are directed to methods wherein said stem cells are derived from the perivascular areas of Wharton’s Jelly.

904. Preferred embodiments are directed to methods wherein said stem cells are exposed to hyperthermia to enhance therapeutic activity.

905. Preferred embodiments are directed to methods wherein said therapeutic macrophages are neuroregenerative.

906. Preferred embodiments are directed to methods wherein said neuroregenerative macrophages are capable of protecting neurons from apoptosis.

907. Preferred embodiments are directed to methods wherein said neuroregenerative macrophages are capable of protecting neurons from excitotoxicity.

908. Preferred embodiments are directed to methods wherein said neuroregenerative macrophages are capable of maintaining neuronal connections.

909. Preferred embodiments are directed to methods wherein said neuroregenerative macrophages are capable of suppressing microglial activation.

910. Preferred embodiments are directed to methods wherein said neuroregenerative macrophages are capable of stimulating proliferation of endogenous neural progenitor cells.

911. Preferred embodiments are directed to methods wherein said therapeutic macrophages are hepatoprotective.

912. Preferred embodiments are directed to methods wherein said hepatoprotective macrophages induce liver regeneration.

913. Preferred embodiments are directed to methods wherein said hepatoprotective macrophages suppress liver fibrosis.

914. Preferred embodiments are directed to methods wherein said hepatoprotective macrophages inhibit production of TGF-beta.

915. Preferred embodiments are directed to methods wherein said hepatoprotective macrophages inhibit activation of the SMAD pathway.

916. Preferred embodiments are directed to methods wherein said hepatoprotective macrophages suppress oxidative stress.

917. Preferred embodiments are directed to methods wherein said hepatoprotective macrophages stimulate hepatic angiogenesis.

918. Preferred embodiments are directed to methods wherein said therapeutic macrophages are renoprotective.

919. Preferred embodiments are directed to methods wherein said therapeutic macrophages are cardioprotective.

920. Preferred embodiments are directed to methods wherein said cardioprotective macrophages preserve viability of cardiomyocytes after an infarct.

921. Preferred embodiments are directed to methods wherein said cardioprotective macrophages preserve viability of cardiac progenitor cells.

922. Preferred embodiments are directed to methods wherein said cardioprotective macrophages preserve viability of cardiac endothelial cells after an infarct.

923. Preferred embodiments are directed to methods wherein said cardioprotective macrophages reduce cardiac fibrosis.

924. Preferred embodiments are directed to methods wherein said cardioprotective macrophages inhibit pathological cardiac remodeling.

925. Preferred embodiments are directed to methods wherein said cardioprotective macrophages suppress oxidative stress in cardiac tissue.

926. Preferred embodiments are directed to methods wherein said cardioprotective macrophages recruit regenerative cells into the cardiac tissue.

927. Preferred embodiments are directed to methods wherein said regenerative cells are endogenous cardiac progenitor cells.

928. Preferred embodiments are directed to methods wherein said endogenous cardiac progenitor cells express c-kit.

929. Preferred embodiments are directed to methods wherein said endogenous cardiac progenitor cells express CD133.

930. Preferred embodiments are directed to methods wherein said endogenous cardiac progenitor cells possess ability to efflux rhodamine 231.

931. Preferred embodiments are directed to methods wherein said regenerative cells are bone marrow regenerative cells.

932. Preferred embodiments are directed to methods wherein said bone marrow regenerative cells express the marker CD34.

933. Preferred embodiments are directed to methods wherein said bone marrow regenerative cells are endothelial progenitor cells.

934. Preferred embodiments are directed to methods wherein said endothelial progenitor cells are capable of healing damaged blood vessels.

935. Preferred embodiments are directed to methods wherein said damaged blood vessel is an atherosclerotic blood vessel.

936. Preferred embodiments are directed to methods wherein said damaged blood vessel is infarct associated blood vessel.

937. Preferred embodiments are directed to methods wherein said damaged blood vessel is a blood vessel that has been exposed to disseminated intravascular coagulation.

938. Preferred embodiments are directed to methods wherein said endothelial progenitor cells are capable of forming endothelial colonies when plated on fibronectin.

939. Preferred embodiments are directed to methods wherein said endothelial progenitor cells possess CXCR4.

940. Preferred embodiments are directed to methods wherein said endothelial progenitor cells possess tie-2.

941. Preferred embodiments are directed to methods wherein said endothelial progenitor cells possess VE Cadherin.

942. Preferred embodiments are directed to methods wherein said endothelial progenitor cells possess CD31.

943. Preferred embodiments are directed to methods wherein said endothelial progenitor cells possess CD34.

944. Preferred embodiments are directed to methods wherein said endothelial progenitor cells possess CD133.

945. Preferred embodiments are directed to methods wherein said endothelial progenitor cells possess CD117.

946. Preferred embodiments are directed to methods wherein said endothelial progenitor cells possess CD105.

947. Preferred embodiments are directed to methods wherein said endothelial progenitor cells possess ETV2.

948. Preferred embodiments are directed to methods wherein said endothelial progenitor cells possess CD146.

949. Preferred embodiments are directed to methods wherein said endothelial progenitor cells possess flk-1.

950. Preferred embodiments are directed to methods wherein said endothelial progenitor cells possess flt-4.

951. Preferred embodiments are directed to methods wherein said bone marrow regenerative cell is a hematopoietic stem cell.

952. Preferred embodiments are directed to methods wherein said hematopoietic stem cell possesses IL-3 receptor.

953. Preferred embodiments are directed to methods wherein said hematopoietic stem cell possesses IL-6 receptor.

954. Preferred embodiments are directed to methods wherein said hematopoietic stem cell possesses thrombopoietin receptor.

955. Preferred embodiments are directed to methods wherein said hematopoietic stem cell expresses CD34.

956. Preferred embodiments are directed to methods wherein said hematopoietic stem cell expresses CD133.

957. Preferred embodiments are directed to methods wherein said hematopoietic stem cell does not express CD38.

958. Preferred embodiments are directed to methods wherein said umbilical cord blood stem cell expresses CD56.

959. Preferred embodiments are directed to methods wherein said umbilical cord blood stem cell expresses CD56.

960. Preferred embodiments are directed to methods wherein said umbilical cord blood stem cell expresses dopamine D3 receptor.

961. Preferred embodiments are directed to methods wherein said umbilical cord blood stem cell expresses dopamine D3 receptor.

962. Preferred embodiments are directed to methods wherein said umbilical cord blood stem cell expresses Notch.

963. Preferred embodiments are directed to methods wherein said umbilical cord blood stem cell expresses Notch 1.

964. Preferred embodiments are directed to methods wherein said umbilical cord blood stem cell expresses Jagged-1.

965. Preferred embodiments are directed to methods wherein said umbilical cord blood stem cell expresses Delta.

966. Preferred embodiments are directed to methods wherein said umbilical cord blood stem cell expresses Delta-1.

967. Preferred embodiments are directed to methods wherein said umbilical cord blood stem cell expresses Delta-4.

968. Preferred embodiments are directed to methods wherein said umbilical cord blood stem cell expresses Oct-¾.

969. Preferred embodiments are directed to methods wherein said umbilical cord blood stem cell expresses Rex-1.

970. Preferred embodiments are directed to methods wherein said umbilical cord blood stem cell expresses Nanog.

971. Preferred embodiments are directed to methods wherein said umbilical cord blood stem cell expresses LIF-STAT2.

972. Preferred embodiments are directed to methods wherein said umbilical cord blood stem cell expresses STAT5.

973. Preferred embodiments are directed to methods wherein said umbilical cord blood stem cell expresses STAT5A.

974. Preferred embodiments are directed to methods wherein said umbilical cord blood stem cell expresses sonic hedgehog.

975. Preferred embodiments are directed to methods wherein said umbilical cord blood stem cell expresses BMP2.

976. Preferred embodiments are directed to methods wherein said umbilical cord blood stem cells are treated with an agonist of one or more selected from a group comprising of: Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt10a, Wnt10b, Wnt11, Wnt14, Wnt15, or Wnt16.

977. Preferred embodiments are directed to methods wherein said umbilical cord blood stem cells are treated with an agonist of Notch.

978. Preferred embodiments are directed to methods wherein said umbilical cord blood stem cells are treated with an agonist of Delta.

979. Preferred embodiments are directed to methods wherein said umbilical cord blood stem cells are treated with an agonist of Serrate.

980. Preferred embodiments are directed to methods wherein said umbilical cord blood stem cells are treated with an agonist of Jagged.

981. Preferred embodiments are directed to methods wherein said umbilical cord blood stem cells are treated with an agonist of Mastermind.

982. Preferred embodiments are directed to methods wherein said umbilical cord blood stem cells are treated with an agonist of Enhancer of Split.

983. Preferred embodiments are directed to methods wherein said umbilical cord blood stem cells are treated with an agonist of Hes 1.

984. Preferred embodiments are directed to methods wherein said umbilical cord blood stem cells are treated with an agonist of Hairless.

985. Preferred embodiments are directed to methods wherein said umbilical cord blood stem cells are treated with an agonist of Suppressor Hairless.

986. Preferred embodiments are directed to methods wherein said umbilical cord blood stem cells are treated with an agonist of RBP-Jk.

986. Preferred embodiments are directed to methods wherein said umbilical cord blood stem cells are treated with an agonist of Desert hedgehog.

987. Preferred embodiments are directed to methods wherein said umbilical cord blood stem cells are treated with an agonist of Sonic hedgehog.

988. Preferred embodiments are directed to methods wherein said umbilical cord blood stem cells are treated with an agonist of Indian hedgehog.

989. Preferred embodiments are directed to methods wherein said umbilical cord blood stem cells are treated with an agonist of Gli.

990. Preferred embodiments are directed to methods wherein said umbilical cord blood stem cells are treated with an agonist of Gli-1.

991. Preferred embodiments are directed to methods wherein said umbilical cord blood stem cells are treated with an agonist of Gli-3.

992. Preferred embodiments are directed to methods wherein said umbilical cord blood stem cells are treated with an agonist of Patched.

993. Preferred embodiments are directed to methods wherein said umbilical cord blood stem cells are treated with an agonist of Patched-1.

### BRIEF DESCRIPTION OF THE DRAWINGS

FIG. 1 is a line graph showing treated cells suppressed production of TNF-alpha.

FIG. 2 is a line graph showing treated cells suppressed production of IL-6.

FIG. 3 is a line graph showing treated cells suppressed production of IL-8.

FIG. 4 is a bar graph showing treated cells suppress colitis.

FIG. 5 is a bar graph showing treated cells production of IL-1RA in response to LPS.

FIG. 6 is a bar graph showing treated cells production of IL-1RA in response to TNF-A.

FIG. 7 is a bar graph showing treated cells production of IL-1RA in response to IL-1B.

FIG. 8 is a bar graph showing treated cells production of IL-10 in response to LPS

FIG. 9 is a bar graph showing treated cells production of IL-10 in response to TNF-Alpha.

FIG. 10 is a bar graph showing treated cells production of IL-10 in response to IL-1B.

FIG. 11 is a bar graph showing treated cells production of VEGF in response to LPS.

FIG. 12 is a bar graph showing treated cells production of VEGF in response to TNF-A.

FIG. 13 is a bar graph showing treated cells production of VEGF in response to IL-1B.

FIG. 14 is a bar graph showing treated cells production of GM-CSF in response to LPS.

FIG. 15 is a bar graph showing treated cells production of GM-CSF in response to TNF-A.

FIG. 16 is a bar graph showing treated cells production of GM-CSF in response to IL-1B.

FIG. 17 is a bar graph showing treated cells production of HGF-1 in response to LPS.

FIG. 18 is a bar graph showing treated cells production of HGF-1 in response to TNF-A.

FIG. 19 is a bar graph showing treated cells production of HGF-1 in response to IL-1B.

### DETAILED DESCRIPTION OF THE INVENTION

The invention provides novel stem cell types, methods of manufacture, and therapeutic uses. Provided are means of deriving stem cells possessing regenerative, immune modulatory, anti-inflammatory, and angiogenic/neurogenic activity from umbilical cord tissue such as Wharton’s Jelly. In some embodiments manipulation of stem cell “potency” is disclosed through hypoxic manipulation, growth on non-xenogeneic conditions, as well as addition of epigenetic modulators.

The cells of the invention are cultured under hypoxia, in one embodiment, cultured in order to induce and/or augment expression of chemokine receptors. One such receptor is CXCR-4. The population of cells, including population of umbilical cord mesenchymal cells, may be enriched for CXCR-4, such as (or such as about) 50%, 55%, 60%, 65%, 70%, 75%, 80%, 85%, 90%, 95%, 96%, 97%, 98%, 99%, or more of the population expressing CXCR-4, CD31, CD34, or any combination thereof. In addition or alternatively, <1%, <2%, <3%, <4%, <5%, <6%, <7%, <8%, <9%, or <10% of the population of cells may express CD14 and/or CD45. The umbilical cord cells of the invention may further possess markers selected from the group consisting of STRO-1, CD105, CD54, CD56, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and a combination thereof. In some embodiments said placental cells of the invention are admixed with endothelial cells. Said endothelial cells may express one or more markers selected from the group consisting of: a) extracellular vimentin; b) CD133; c) c-kit; d) VEGF receptor; e) activated protein C receptor; and f) a combination thereof. In some embodiments, the population of endothelial cells comprises endothelial progenitor cells.

The population of cells may be allogeneic, autologous, or xenogenic to an individual, including an individual being administered the population of cells. In some embodiments, the population of cells are matched by mixed lymphocyte reaction matching.

In some embodiments, the population of cells is derived from tissue selected from the group consisting of the placental body, placenta, umbilical cord tissue, peripheral blood, hair follicle, cord blood, Wharton’s Jelly, menstrual blood, endometrium, skin, omentum, amniotic fluid, and a combination thereof. In some embodiments, the population of cells, the population of umbilical mesenchymal stem cells, or the population of endothelial cells comprises human umbilical cord derived adherent cells. The human umbilical cord derived adherent cells may express a cytokines selected from the group consisting of ) FGF-1; b) FGF-2; c) HGF; d) interleukin-1 receptor antagonist; and e) a combination thereof. In some embodiments, the population of cells, the population of umbilical cord cells express arginase, indoleamine 2,3 deoxygenase, interleukin-10, and/or interleukin 35. In some embodiments, the population of cells, the population of umbilical cord cells, or the population of endothelial cells express hTERT and Oct-4 but does not express a STRO-1 marker.

In some embodiments, the population of cells, the population of umbilical cord cells has an ability to undergo cell division in less than 36 hours in a growth medium. In some embodiments, the population of cells, the population of umbilical cord cells has an ability to proliferate at a rate of 0.9-1.2 doublings per 36 hours in growth media. In some embodiments, the population of cells, the population of umbilical cord cells has an ability to proliferate at a rate of 0.9, 1.0, 1.1, or 1.2 doublings per 36 hours in growth media. The population of cells, population of umbilical cord cells may produce exosomes capable of inducing more than 50% proliferation when the exosomes are cultured with human umbilical cord endothelial cells. The induction of proliferation may occur when the exosomes are cultured with the human umbilical cord endothelial cells at a concentration of 10, 20, 30, 40, 50, 60, 70, 80, 90, 100, 110, 120, 130, 140, 150, 160, 170, 180, 190, 200, or more exosomes per cell.

In some embodiments, a population of cells, including a population of umbilical cells alone, are administered to an individual, including an individual having and acute or chronic pathology, wherein the population of cells may be administered via any suitable route, including as non-limiting examples, intramuscularly and/or intravenously.

In some embodiments, a population of umbilical cord cells is optionally obtained, the population is then optionally contacted via culturing with a population of progenitor for T regulatory cells, wherein the culturing conditions allow for the generation of T regulatory cells, then the generated T regulatory cells are administered to an individual.

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

Chemical Modification: As used herein, “chemical modification” refers to the process wherein a chemical or biochemical is used to induce genomic changes in the donor cell, or nucleus thereof, that allow the donor cell, or nucleus thereof, to be responsive during maturation and receptive to the host cell cytoplasm.

Committed: As used herein, “committed” refers to cells which are considered to be permanently committed to a specific function. Committed cells are also referred to as “terminally differentiated cells.”

Cytoplast Extract Modification: As used herein, “cytoplast extract modification” refers to the process wherein a cellular extract consisting of the cytoplasmic contents of a cell are used to induce genomic changes in the donor cell, or nucleus thereof, that allow the donor cell, or nucleus thereof, to be responsive during maturation and receptive to the host cell cytoplasm.

Dedifferentiation: As used herein, “dedifferentiation” refers to loss of specialization in form or function. In cells, dedifferentiation leads to an a less committed cell.

Differentiation: As used herein, “differentiation” refers to the adaptation of cells for a particular form or function. In cells, differentiation leads to a more committed cell.

Donor Cell: As used herein, “donor cell” refers to any diploid (2N) cell derived from a pre-embryonic, embryonic, fetal, or post-natal multi-cellular organism or a primordial sex cell which contributes its nuclear genetic material to the hybrid stem cell. The donor cell is not limited to those cells that are terminally differentiated or cells in the process of differentiation. For the purposes of this invention, donor cell refers to both the entire cell or the nucleus alone.

Donor Cell Preparation: As used herein, “donor cell preparation” refers to the process wherein the donor cell, or nucleus thereof, is prepared to undergo maturation or prepared to be receptive to a host cell cytoplasm and/or responsive within a post-natal environment.

Germ Cell: As used herein, “germ cell” refers to a reproductive cell such as a spermatocyte or an oocyte, or a cell that will develop into a reproductive cell.

Host Cell: As used herein, “host cell” refers to any multipotent stem cell derived from a pre-embryonic, embryonic, fetal, or post-natal multicellular organism that contributes the cytoplasm to a hybrid stem cell.

Host Cell Preparation: As used herein, “host cell preparation” refers to the process wherein the host cell is enucleated.

Hybrid Stem Cell: As used herein, “hybrid stem cell” refers to any cell that is multipotent and is derived from an enucleated host cell and a donor cell, or nucleus thereof, of a multicellular organism. Hybrid stem cells are further disclosed in co-pending U.S. Pat. application Ser. No. 10/864,788.

Karyoplast Extract Modification: As used herein, “karyoplast extract modification” refers to the process wherein a cellular extract consisting of the nuclear contents of a cell, lacking the DNA, are used to induce genomic changes in the donor cell, or nucleus thereof, that allow the donor cell, or nucleus thereof, to be responsive during maturation or receptive to the host cell cytoplasm.

Maturation: As used herein, “maturation” refers to a process of coordinated steps either forward or backward in the differentiation pathway and can refer to both differentiation or de-differentiation. As used herein, maturation is synonymous with the terms develop or development when applied to the process described herein.

Modified Germ Cell: As used herein, “modified germ cell” refers to a cell comprised of a host enucleated ovum and a donor nucleus from a spermatogonia, oogonia or a primordial sex cell. The host enucleated ovum and donor nucleus can be from the same or different species. A modified germ cell can also be called a “hybrid germ cell.”

Multipotent: As used herein, “multipotent” refers to cells that can give rise to several other cell types, but those cell types are limited in number. An example of a multipotent cells is hematopoietic cells-blood stem cells that can develop into several types of blood cells but cannot develop into brain cells.

Multipotent Adult Progenitor Cells: As used herein, “multipotent adult progenitor cells” refers to multipotent cells isolated from the bone marrow which have the potential to differentiate into mesenchymal, endothelial and endodermal lineage cells.

Pre-embryo: As used herein, “pre-embryo” refers to a fertilized egg in the early stage of development prior to cell division. During the pre-embryonic stage the initial stages of cleavage are occurring.

Pre-embryonic Stem Cell: See “Embryonic Stem Cell” above.

Post-natal Stem Cell: As used herein, “post-natal stem cell” refers to any cell that is multipotent and derived from a multi-cellular organism after birth.

Pluripotent: As used herein, “pluripotent” refers to cells that can give rise to any cell type except the cells of the placenta or other supporting cells of the uterus.

Primordial Sex Cell: As used herein, “primordial sex cell” refers to any diploid cell that is derived from the male or female mature or developing gonad, is able to generate cells that propagate a species and contains a diploid genomic state. Primordial sex cells can be quiescent or actively dividing. These cells include male gonocytes, female gonocytes, spermatogonial stem cells, ovarian stem cells, oogonia, type-A spermatogonia, Type-B spermatogonia. Also known as germ-line stem cells.

Primordial Germ Cell: As used herein, “primordial germ cell” refers to cells present in early embryogenesis that are destined to become germ cells.

Reprogamming: As used herein “reprogramming” refers to the resetting of the genetic program of a cell such that the cell exhibits pluripotency and has the potential to produce a fully developed organism.

Responsive: As used herein, “responsive” refers to the condition of a cell, or group of cells, wherein they are susceptible to and can function accordingly within a cellular environment. Responsive cells are capable of responding to and functioning in a particular cellular environment, tissue, organ and/or organ system.

Somatic Stem Cells: As used herein, “somatic stem cells” refers to diploid multipotent or pluripotent stem cells. Somatic stem cells are not totipotent stem cells. Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of multipotent cells is hematopoietic cells-blood cells that can develop into several types of blood cells, but cannot develop into brain cells. At the end of the long chain of cell divisions that make up the embryo are “terminally differentiated” cells-cells that are considered to be permanently committed to a specific function.

Therapeutic Cloning: As used herein, “therapeutic cloning” refers to the cloning of cells using nuclear transfer methods including replacing the nucleus of an ovum with the nucleus of another cell and stem cells derived from the inner cell mass.

Therapeutic Reprogramming: As used herein, “therapeutic reprogramming” refers to the process of maturation wherein a stem cell is exposed to stimulatory factors according to the teachings of the present invention to yield either pluripotent, multipotent or tissue-specific committed cells. Therapeutically reprogrammed cells are useful for implantation into a host to replace or repair diseased, damaged, defective or genetically impaired tissue. The therapeutically reprogrammed cells of the present invention do not possess non-human sialic acid residues.

Totipotent: As used herein, “totipotent” refers to cells that contain all the genetic information needed to create all the cells of the body plus the placenta. Human cells have the capacity to be totipotent only during the first few divisions of a fertilized egg.

Whole Cell Extract Modification: As used herein, “whole cell extract modification” refers to the process wherein a cellular extract consisting of the cytoplasmic and nuclear contents of a cell are used to induce genomic changes in the donor cell, or nucleus thereof, that allow the donor cell, or nucleus thereof, to be responsive during maturation and receptive to the host cell cytoplasm.

In one embodiment the invention teaches phenotypically defined MSC which can be isolated from the Wharton’s jelly of umbilical cord segments and defined morphologically and by cell surface markers. By dissecting out the veins and arteries of cord segments and exposing the Wharton’s jelly, the cells of invention, of one embodiment of the invention, may be obtained. An approximately 1-5 cm cord segment is placed in collagenase solution (1 mg/ml, Sigma) for approximately 18 hrs at room temperature. After incubation, the remaining tissue is removed and the cell suspension is diluted with PBS into two 50 ml tubes and centrifuged. Cells are then washed in PBS and counted using hematocytometer. 5-20.times.10.sup.6 cells were then plated in a 6 cm tissue culture plate in low-glucose DMEM (Gibco) with 10% FBS (Hyclone), 2 mM L-Glutamine (Gibco), 100 U/ml penicillin/100 ug/ml streptomycin/0.025 ug/ml amphotericin B (Gibco). At this step of the purification process, cells are exposed to hypoxia. The amount of hypoxia needed is the sufficient amount to induce activagion of HIF-1 alpha. In one embodiment cells are cultured for 24 hours at 2% oxygen. After 48 hrs cells are washed with PBS and given fresh media. Cells were given new media twice weekly. After 7 days, cells are approximately 70-80% confluent and are passed using HyQTase (Hyclone) into a 10 cm plate. Cells are then regularly passed 1:2 every 7 days or upon reaching 80% confluence.

In another embodiment of the invention, biologically useful stem cells are disclosed, of the mesenchymal or related lineages, which are therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover the therapeutically reprogrammed cells of the present invention are immunologically privileged and therefore suitable for therapeutic applications. Additional methods of the present invention provide for the generation of hybrid stem cells. Furthermore, the present invention includes related methods for maturing stem cells made in accordance with the teachings of the present invention into specific host tissues. For use in the current invention, the practitioner is thought that ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied. During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G0/G1 phase of the cell cycle. In mice and rats these gonocytes resume division within a few days after birth to generate spermatogonial stem cells and eventually undergo differentiation and meiosis related to spermatogenesis. It is known that embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has lead to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage. Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory. During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be effected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandemly repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress. In one teaching, or embodiment, of the invention, therapeutically reprogrammed cells, in some embodiments mesenchymal stem cells, are provided. Therapeutic reprogramming refers to a maturation process wherein a stem cell is exposed to stimulatory factors according the teachings of the present invention to yield enhanced therapeutic activity. In some embodiments, enhancement of therapeutic activity may be increase proliferation, in other embodiments, it may be enhanced chemotaxis. Other therapeutic characteristics include ability to under resistance to apoptosis, ability to overcome senescence, ability to differentiate into a variety of different cell types effectively, and ability to secrete therapeutic growth factors which enhance viability/activity, of endogenous stem cells. In order to induce therapeutic reprogramming of cells, in some cases, as disclosed herein, of wharton’s jelly originating cells, the invention teaches the utilization of stimulatory factors, including without limitation, chemicals, biochemicals and cellular extracts to change the epigenetic programming of cells. These stimulatory factors induce, among other results, genomic methylation changes in the donor DNA. Embodiments of the present invention include methods for preparing cellular extracts from whole cells, cytoplasts, and karyplasts, although other types of cellular extracts are contemplated as being within the scope of the present invention. In a non-limiting example, the cellular extracts of the present invention are prepared from stem cells, specifically embryonic stem cells. Donor cells are incubated with the chemicals, biochemicals or cellular extracts for defined periods of time, in a non-limiting example for approximately one hour to approximately two hours, and those reprogrammed cells that express embryonic stem cell markers, such as Oct4, after a culture period are then ready for transplantation, cryopreservation or further maturation. In another embodiment of the present invention, hybrid stem cells are provided which can be used for cellular regenerative/reparative therapy. The hybrid stem cells of the present invention are pluripotent and customized for the intended recipient so that they are immunologically compatible with the recipient. Hybrid stem cells are a fusion product between a donor cell, or nucleus thereof, and a host cell. Typically the fusion occurs between a donor nucleus and an enucleated host cell. The donor cell can be any diploid cell, including but not limited to, cells from pre-embryos, embryos, fetuses and post-natal organisms. More specifically, the donor cell can be a primordial sex cell, including but not limited to, oogonium or differentiated or undifferentiated spermatogonium, or an embryonic stem cell. Other non-limiting examples of donor cells are therapeutically reprogrammed cells, embryonic stem cells, fetal stem cells and multipotent adult progenitor cells. Preferably the donor cell has the phenotype of the intended recipient. The host cell can be isolated from tissues including, but not limited to, pre-embryos, embryos, fetuses and post-natal organisms and more specifically can include, but is not limited to, embryonic stem cells, fetal stem cells, multipotent adult progenitor cells and adipose-derived stem cells. In a non-limiting example, cultured cell lines can be used as donor cells. The donor and host cells can be from the same individual or different individuals. In one embodiment of the present invention, lymphocytes are used as donor cells and a two-step method is used to purify the donor cells. After the tissues was disassociated, an adhesion step was performed to remove any possible contaminating adherent cells followed by a density gradient purification step. The majority of lymphocytes are quiescent (in G0 phase) and therefore can have a methylation status than conveys greater plasticity for reprogramming. Multipotent or pluripotent stem cells or cell lines useful as donor cells in embodiments of the present invention are functionally defined as stem cells by their ability to undergo differentiation into a variety of cell types including, but not limited to, adipogenic, neurogenic, osteogenic, chondrogenic and cardiogenic cell.

In some embodiments, host cell enucleation for the generation of hybrid stem cells according to the teachings of the present invention can be conducted using a variety of means. In a non-limiting example, ADSCs were plated onto fibronectin coated tissue culture slides and treated with cells with either cytochalasin D or cytochalasin B. After treatment, the cells can be trypsinized, re-plated and are viable for about 72 hours post enucleation. Host cells and donor nuclei can be fused using one of a number of fusion methods known to those of skill in the art, including but not limited to electrofusion, microinjection, chemical fusion or virus-based fusion, and all methods of cellular fusion are envisioned as being within the scope of the present invention. The hybrid stem cells made according to the teachings of the present invention possess surface antigens and receptors from the enucleated host cell but has a nucleus from a developmentally younger cell. Consequently, the hybrid stem cells of the present invention will be receptive to cytokines, chemokines and other cell signaling agents, yet possess a nucleus free from age-related DNA damage. The therapeutically reprogrammed cells and hybrid stem cells made in accordance with the teachings of the present invention are useful in a wide range of therapeutic applications for cellular regenerative/reparative therapy. For example, and not intended as a limitation, the therapeutically reprogrammed cells and hybrid stem cells of the present invention can be used to replenish stem cells in animals whose natural stem cells have been depleted due to age or ablation therapy such as cancer radiotherapy and chemotherapy. In another non-limiting example, the therapeutically reprogrammed cells and hybrid stem cells of the present invention are useful in organ regeneration and tissue repair. In one embodiment of the present invention, therapeutically reprogrammed cells and hybrid stem cells can be used to reinvigorate damaged muscle tissue including dystrophic muscles and muscles damaged by ischemic events such as myocardial infarcts. In another embodiment of the present invention, the therapeutically reprogrammed cells and hybrid stem cells disclosed herein can be used to ameliorate scarring in animals, including humans, following a traumatic injury or surgery. In this embodiment, the therapeutically reprogrammed cells and hybrid stem cells of the present invention are administered systemically, such as intravenously, and migrate to the site of the freshly traumatized tissue recruited by circulating cytokines secreted by the damaged cells. In another embodiment of the present invention, the therapeutically reprogrammed cells and hybrid stem cells can be administered locally to a treatment site in need or repair or regeneration.

In one embodiment, umbilical cord samples were obtained following the delivery of normal term babies with Institutional Review Board approval. A portion of the umbilical cord was then cut into approximately 3 cm long segments. The segments were then placed immediately into 25 ml of phosphate buffered saline without calcium and magnesium (PBS) and 1.times. antibiotics (100 U/ml penicillin, 100 ug/ml streptomycin, 0.025 ug/ml amphotericin B). The tubes were then brought to the lab for dissection within 6 hours. Each 3 cm umbilical cord segment was dissected longitudinally utilizing aseptic technique. The tissue was carefully undermined and the umbilical vein and both umbilical arteries were removed. The remaining segment was sutured inside out and incubated in 25 ml of PBS, 1.times. antibiotic, and 1 mg/ml of collagenase at room temperature. After 16-18 hours the remaining suture and connective tissue was removed and discarded. The cell suspension was separated equally into two tubes, the cells were washed 3.times. by diluting with PBS to yield a final volume of 50 ml per tube, and then centrifuged. Red blood cells were then lysed using a hypotonic solution. Cells were plated onto 6-well plates at a concentration of 5-20.times.10.sup.6 cells per well. UC-MSC were cultured in low-glucose DMEM (Gibco) with 10% FBS (Hyclone), 2 mM L-Glutamine (Gibco), 100 U/ml penicillin, 100 ug/ml streptomycin, 0.025 ug/ml amphotericin B (Gibco). Cells were washed 48 hours after the initial plating with PBS and given fresh media. Cell culture media were subsequently changed twice a week through half media changes. After 7 days or approximately 70-80% confluence, cells were passed using HyQTase (Hyclone) into a 10 cm plate. Cells were then regularly passed 1:2 every 7 days or upon reaching 80% confluence. Alternatively, 0.25% HQ trypsin/EDTA (Hyclone) was used to passage cells in a similar manner.

In some embodiments of the invention, administration of cells of the invention is performed for suppression of an inflammatory and/or autoimmune disease. In these situations, it may be necessary to utilize an immune suppressive/or therapeutic adjuvant. Immune suppressants are known in the art and can be selected from a group comprising of: cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc. In another embodiment, the use of stem cell conditioned media may be used to potentiate an existing anti-inflammatory agent. Anti-inflammatory agents may comprise one or more agents including NSAIDs, interleukin-1 antagonists, dihydroorotate synthase inhibitors, p38 MAP kinase inhibitors, TNF-α inhibitors, TNF-α sequestration agents, and methotrexate. More specifically, anti-inflammatory agents may comprise one or more of, e.g., anti-TNF-α, lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric .acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid.

The cells of the invention may be useful for numerous types of indications. Below are some examples of indications and rationale that they may be used for

In one embodiment, cells of the invention may be utilized for treatment of Amyotrophic lateral sclerosis (ALS). Thisis a progressive neurodegenerative condition causing muscular atrophy and death within 3-5 years after its onset [1]. In the majority of patients (90%) the cause of ALS idiopathic, however in about 10% of the patients a familial form of the disease is presented [2]. Specific muscular degeneration is exclusive to motor neurons and begins focally and spreads, leading to weakness of limb, respiratory, and bulbar muscles. Immediately preceding death, there is a near total loss of limb and respiratory function, as well as a loss of the ability to chew, swallow, and speak. In the USA, ALS is defined as an “orphan disease,” with approximately 2 per 100,000 new cases per year and a prevalence of about 5 per 100,000 total cases each year [3]. In the United States [4] and Europe [5], ALS is diagnosed in about 1 in 500 to 1 in 1,000 adult deaths, implying that 500,000 people in the United States will develop this disease in their lifetimes. About 10% of ALS cases are inherited, usually as dominant traits [6]. Both familial ALS (fALS) and sporadic ALS (sALS) can develop concurrently with frontotemporal lobar dementia (FTLD). By contrast with the dementia of Alzheimer disease (AD), in which the cardinal finding is memory loss, FTLD is characterized by behavioral changes and progressive aphasia, sometimes accompanied by movement disorders. While AD involves prominent pathology in the hippocampus, the essential finding in FTLD is, as the name suggests, early atrophy of the frontal and temporal lobes. Four recurring themes have emerged from the pathological analysis of autopsied cases with sALS, fALS, or ALS-FTLD with diverse genetic causes. First, the motor neuron death usually entails deposition of aggregated proteins, often ubiquitinated and predominantly cytoplasmic. Second, in ALS, the levels and functions of RNA and RNA-binding proteins are abnormal. Aggregates of protein and RNA are detected both in motor neurons and non-neuronal cells, such as astrocytes and microglia. Third, most cases entail some disturbance of neuronal cytoskeletal architecture and function. Additionally, in almost all cases, motor neuron death is influenced by non-neuronal cells, including oligodendroglia and cells involved in neuroinflamation (e.g., astroglia and microglia).

We incorporate be reference trials of other types of MSC in order to provide guidance for one of skill in the art how to administer the cells of the current invention, and how to designed clinical trials based on previous types of cell therapies.

One of the first clinical interventions using mesenchymal stem cells in ALS was a report by Mazzin et al [7], who treated ALS patients with bone marrow ex vivo expanded MSC. Specifically, bone marrow collection was performed according to the standard procedure by aspiration from the posterior iliac crest. Ex vivo expansion of mesenchymal stem cells was induced according to Pittenger’s protocol [8]. The cells were suspended in 2 ml of autologous cerebrospinal fluid and transplanted into the spinal cord by a micrometric pump injector. No patient manifested major adverse events such as respiratory failure or death. Minor adverse events were intercostal pain irradiation (4 patients) which was reversible after a mean period of three days after surgery, and leg sensory dysesthesia (5 patients) which was reversible after a mean period of six weeks after surgery. No modification of the spinal cord volume or other signs of abnormal cell proliferation were observed. The authors concluded by stating that it appears that the procedures of ex vivo expansion of autologous mesenchymal stem cells and of transplantation into the spinal cord of humans are safe and well tolerated by ALS patients. The same group reported 3 year follow up of the initial patients treated. Seven patients affected by definite ALS were enrolled in the study and two patients were treated for compassionate use. No patient manifested major adverse events such as respiratory failure or death. Minor adverse events were intercostal pain irradiation and leg sensory dysesthesia, both reversible after a mean period of 6 weeks. No modification of the spinal cord volume or other signs of abnormal cell proliferation were observed. A significant slowing down of the linear decline of the forced vital capacity was evident in four patients 36 months after MSCs transplantation [9]. An additional two studies where performed by the same group on 10 and 19 patients. The longest observation of treated patients was performed at 9 years after treatment. No long term adverse effects were detected and marginal therapeutic effects were seen [10, 11] .

A study by an independent group evaluated the safety of two repeated intrathecal injections of autologous bone marrow (BM)-derived mesenchymal stromal cells (MSCs) in ALS patients. Eight patients with definite or probable ALS were enrolled. After a 3-month lead-in period, autologous MSCs were isolated two times from the BM at an interval of 26 days and were then expanded in vitro for 28 days and suspended in autologous cerebrospinal fluid. Of the 8 patients, 7 received 2 intrathecal injections of autologous MSCs (1 × 10(6) cells per kg) 26 days apart. Clinical or laboratory measurements were recorded to evaluate the safety 12 months after the first MSC injection. The ALS Functional Rating Scale-Revised (ALSFRS-R), the Appel ALS score, and forced vital capacity were used to evaluate the patients’ disease status. One patient died before treatment and was withdrawn from the study. The death was not study related, and was attributable to natural progression of disease. With the exception of that patient, no serious adverse events were observed during the 12-month follow-up period. Most of the adverse events were self-limited or subsided after supportive treatment within 4 days. Decline in the ALSFRS-R score was not accelerated during the 6-month follow-up period. Two repeated intrathecal injections of autologous MSCs were safe and feasible throughout the duration of the 12-month follow-up period [12]. A subsequent study from Belarus utilized autologous mesenchymal stem cells were injected intravenously (intact cells) or via lumbar puncture (cells committed to neuronal differentiation). Evaluation of the results of cell therapy after 12-month follow-up revealed slowing down of the disease progression, as assessed by ALSFRS-R score was observed in 10 patients that were treated with cells. In comparison, in a control group that was matched for age and disease status, no slowing down of progression was observed. The Control group consisted of 15 patients. The study reported no adverse effects associated with administration of mesenchymal stem cells intravenously or intrathecally [13].

Although the above clinical studies support a possible benefit for reducing progression of ALS, as determined by slowing down advancement of disease as quantified by measures such as ALSFRS-R score, none of the studies reversed the disease progression. One possible reason is that when the stem cells where introduced via lumbar injection (intrathecally), the cells hypothetically would tend to sink downwards rather than ascending to the brain and cervical and thoracic spinal cord. Therefore, a study by Baek et al. [14], assessed the ability to utilize intraventricular injections directly into the brain by using an Ommaya reservoir to administer cells. The Ommaya reservoir is catheter system that is typically used for the delivery of drugs directly into the ventricles of the brain. It consists of a catheter in one lateral ventricle attached to a reservoir implanted under the scalp. It is typically used to treat brain tumors, leukemia/lymphoma or leptomeningeal disease, as well as for intracerebroventricular (ICV) injection of morphine [15]. Others have previously used the Ommaya reservoir to deliver cell therapy into the brain. To give an indication of the relative safety of this approach, in one study in glioma patients, autologous tumor infiltrating lymphocytes that were expanded ex vivo we administered in 6 patients by use of the Ommaya reservoir. One patient had complete response, 2 had partial responses, and 3 succumbed to disease. Most interestingly, no serious adverse effects were noted, despite the fact that activated lymphocytes were directly injected into the brain, an area typically classified as very sensitive in inflammation [16]. With the rational this, and other studies have successfully administered cells in the brain [17-19], and mesenchymal stem cells are generally considered anti-inflammatory, Baek et al attempted to adopt this procedure for use in ALS in a patient. Bone marrow mesenchymal stem cells were isolated from the bone marrow of a male patient with ALS who underwent insertion of an Ommaya reservoir. Expanded MSCs (hBM-MSCs: dose of 1 X 106 cells/kg) were suspended in autologous CSF and directly transplanted into the ALS patient’s lateral ventricle via the Ommaya reservoir. Clinical, laboratory, and radiographic evaluation of the patient revealed no serious adverse effects related to the stem cell therapy. The authors concluded that intraventricular injection with an optimized number of cells is safe, and is a potential route for stem cell therapy in patients with ALS. Intraventricular injection via an Ommaya reservoir makes repetitive injection of stem cells easy and reliable even in far advanced ALS patients. Unfortunately, no discussion on impact on disease progression was given in the publication.

In another attempt to increase therapeutic efficacy of mesenchymal stem cells in ALS, researchers have explored in vitro means of augmenting neurotrophic factor production by manipulation of culture conditions. A series of studies from the Hadassah Medical Center in Jerusalem, Israel attempted to treat ALS by in vitro manipulated MSC that are validated to produce higher amounts of neurotrophic factors. In the studies, all patients were followed up for 3 months before transplantation and 6 months after transplantation. In the phase ½ part of the trial, 6 patients with early-stage ALS were injected intramuscularly (IM) and 6 patients with more advanced disease were transplanted intrathecally (IT). In the second stage, a phase 2a dose-escalating study, 14 patients with early-stage ALS received a combined IM and IT transplantation of autologous MSC-NTF cells. It was reported that among the 12 patients in the phase ½ trial and the 14 patients in the phase 2a trial aged 20 and 75 years, the administration of mesenchymal stem cells was found to be safe and well tolerated over the study follow-up period. Most of the adverse effects were mild and transient, not including any treatment-related serious adverse event. The rate of progression of the forced vital capacity and of the ALSFRS-R score in the IT (or IT+IM)-treated patients was reduced (from -5.1% to -1.2%/month percentage predicted forced vital capacity, P < 0.04 and from -1.2 to 0.6 ALSFRS-R points/month, P = 0.052) during the 6 months following MSC-NTF cell transplantation vs the pretreatment period. Of these patients, 13 (87%) were defined as responders to either ALSFRS-R or forced vital capacity, having at least 25% improvement at 6 months after treatment in the slope of progression. A subsequent report on MSC-NTF cells described observations after these cells where delivered by combined intrathecal and intramuscular administration to participants with amyotrophic lateral sclerosis (ALS) in a phase 2 randomized controlled trial. The study enrolled 48 participants randomized 3:1 (treatment: placebo). After a 3-month pretransplant period, participants received 1 dose of MSC-NTF cells (n = 36) or placebo (n = 12) and were followed for 6 months. CSF was collected before and 2 weeks after transplantation. The study met its primary safety endpoint. The rate of disease progression (Revised ALS Functional Rating Scale [ALSFRS-R] slope change) in the overall study population was similar in treated and placebo participants. In a prespecified rapid progressor subgroup (n = 21), rate of disease progression was improved at early time points (p < 0.05). To address heterogeneity, a responder analysis showed that a higher proportion of treated participants experienced ≥1.5 points/month ALSFRS-R slope improvement compared to placebo at all time points, and was significant in rapid progressors at 4 and 12 weeks (p = 0.004 and 0.046, respectively). CSF neurotrophic factors increased and CSF inflammatory biomarkers decreased in treated participants (p < 0.05) post-transplantation. CSF monocyte chemoattractant protein-1 levels correlated with ALSFRS-R slope improvement up to 24 weeks (p < 0.05). Thus the authors summarized that a single-dose transplantation of MSC-NTF cells is safe and demonstrated early promising signs of efficacy. This establishes a clear path forward for a multidose randomized clinical trial of intrathecal autologous MSC-NTF cell transplantation in ALS [20].

In conclusion, for the use of the current invention in the treatment of ALS, there are means of enhancing efficacy. The cells of the current invention allow for a) increasing frequency of dosing, in part because the cells are smaller in size than typical MSC, higher number of cells may be given without fear of lung pathology.; b) further augmentation of mesenchymal stem cell regenerative activity through culture conditions or gene manipulation, for example, addition of neurotrophic factors in vitro to enhance activity may be performed; c) the use of other growth/neurotrophic factors as adjuvants. For example, it is known that endogenous neuronal stem cells exist, whose activity may be modified by administration of various compounds, some compounds already clinically available. Well-known examples of approved drugs that augment endogenous neural stem cell activity include lithium [21, 22], valproic acid [23], and human chorionic gonadotropin [24], these agents are disclosed for use within the context of the current invention. Interestingly, the stem cell modifier combination of lithium and valproic acid was already assessed on its own in a small trial which suggested some possible efficacy. The study recruited 18 patients that were treated with the combination and compared them to 31 controls that were carefully paired by age, gender, evolution rate and time of the disease, who never received treatment with lithium and/or valproate. Assessment of disease by ALSFRS-R was performed before treatment (baseline), 1 month after treatment, and every 4 months until the outcome (death or an adverse event). The investigators reported that lithium and valproate cotreatment significantly increased survival, and this treatment also exerted neuroprotection in our patients because all biochemical markers reached levels normal levels in the ALS patients that were treated. The biochemical markers were Cu/Zn superoxide dismutase and glutathione peroxidase activity, and reduced glutathione [25].

In another embodiment of the invention, cells described may be used for treatment of conditions associated with immunological attack on the bone marrow or hematopoitic systems. On such condition, which may be treated by the current inventions, is aplastic anemia (AA) which is a orphan disorder characterized by a slowing or cessation of blood cell production caused by the destruction of stem cells in the bone marrow. The resulting deficit in red cells, white cells, and platelets leads to fatigue and increased risk of infection and uncontrolled bleeding, which are potentially life-threatening complications. The majority of cases of AA are acquired and immune mediated, whereby an aberrant immune response mediates destruction of hematopoietic stem cells by T lymphocytes [26-28]. Support for autoimmune pathophysiology of AA is derived from observations of a high rate of response of AA patients to immunosuppressive therapy [29-32]. In fact, in a majority of treatment situations cyclosporine is utilized as a frontline treatment. Other causes of AA include exposure to toxic chemicals (including inhaled solvents), chemotherapy and other drugs, radiation, and viruses (e.g., hepatitis, Epstein-Barr virus, cytomegalovirus, parvovirus B 19, and HIV), although in many cases no cause is identified [33]. Autoimmune diseases, diseases of the bone marrow, and, rarely, pregnancy are also associated with AA. Hereditary AA is rare, occurring with inherited conditions such as Fanconi anemia, Shwachman-Diamond syndrome, and dyskeratosis congenital [34]. AA affects an estimated 2-3 people per million in the United States, with approximately 600 to 900 new diagnoses annually. The incidence of AA is at least two to three times higher in Asian countries, with about 6,000 to 7,000 new diagnoses reported annually worldwide. This disease is usually diagnosed in 2-5 year old children, young adults 20-25 years of age, and a smaller peak of incidence in adults 55-60 years of age. Current treatments for aplastic anemia include transfusion of blood or blood components (red cells or platelets), antibiotics/anti-infective drugs, immune-suppressing drugs, bone marrow transplantation, colony stimulating factors, and erythropoietin [35]. Analysis of bone marrow cellularity is performed for grading the severity of AA as non-severe, severe, or very severe on the basis of the levels of neutrophils, platelets and reticulocytes. In biopsy specimens, patients with AA show a loss of hematopoietic cells (<30%) in the bone marrow, increased fat space, no extensive marrow fibrosis, no extensive iron deposition, and no evidence of malignant disease. Patients with severe or very severe aplastic anemia are at risk for life-threatening infections or bleeding. While patients with moderate AA often respond to immune suppressive agents, there are no treatment options for patients with severe AA who lack a suitable bone marrow donor. Rational for utilization of mesenchymal stem cells in treatment of AA derives from previous studies that have shown mesenchymal stem cells are immune modulatory and have ability to control autoimmune conditions and associated pathological immune responses [36-40]. Additionally, in many patients with AA a deficiency of T regulatory cells is known to occur [41-43]. T regulatory cells are positively selected for in thymic selection so that adults possess a repertoire of autoreactive cells that instead of cause pathology, instruct conventional T cells not to attack self-tissue. In AA, it is postulated that the lack of T regulatory cells allows for initiation and maintenance of autoreactive immunity to bone marrow elements. It is known that mesenchymal stem cells not only directly suppress pathological immunity, but also induce generation of T regulatory cells [44-48]. Additionally, mesenchymal stem cells produce growth factors that stimulate hematopoiesis [49-51]. Use of mesenchymal stem cells for stimulation of hematopoietic stem cell is discussed in more detail in the chapter “Hematopoietic Reconstitution”

In one study, Wang et al. reported on a 3-year-old girl with severe AA that was previously unresponsive to steroid, cyclosporine and filgrastim treatments. The patient had experienced repeated bacterial infections and received 44 blood transfusions during 8 months after diagnosis. In an effort to “reprogram” her immune system, hematopoietic stem cells of her father were administered after myeloablative therapy. The concept being that the defect causing AA found in the patient’s bone marrow compartment would be healed after introduction of her father’s stem cell compartment. The patient received 1.25 × 10(6)/kg bone marrow derived mesenchymal stem cells from her father, as well as mobilized BM and peripheral blood stem cell grafts from her father. The conditioning regimen consisted of fludarabine, cyclophosphamide and busulfan, and prophylaxis of acute graft-versus-host disease (GvHD) was performed by administration of anti-CD25 monoclonal antibody, cyclosporine A, methotrexate, mycophenolate mofetil and anti-thymocyte globulin. The patient achieved rapid hematopoietic engraftment of donor origin and no acute or chronic GvHD was observed. She was alive at time of publication (4 years subsequent to procedure and with good performance status [52].

Based on the initial positive findings with the first patient treated on this protocol, the same group performed an expanded study using a similar treatment regimen. In the study there were 6 patients recruited with severe AA ranging in age from 3 to 16 years, and the median time from diagnosis to transplantation was 32 months (range: 3-156 months). The hematopoietic stem cell grafts were granulocyte colony-stimulating factor-mobilized bone marrow and peripheral blood from HLA antigen-haploidentical donors (3 cases) or peripheral blood only from unrelated HLA antigen-identical donors (3 cases). Mesenchymal stem cells were intravenously injected at a median dose of 1.43 × 10(6)/kg (range: 0.85-2.5 × 10(6)/kg). The mean time for neutrophil and platelet recovery was 12.3 and 13.8 days, respectively. Acute GvHD grade I and II developed in 2 cases, and no chronic GvHD was documented. All patients were alive and transfusion independent at a median follow-up of 15 months (range: 6-29 months). The efficacy in this trial was superior to similar case reports of pediatric aplastic anemia who received hematopoietic stem cell grafts only without co-administration of mesenchymal stem cells [53].

The principle of using allogeneic mesenchymal stem cells to accelerate hematopoietic engraftment in patients with AA after transplant has been also reported in several case reports. For example, Luan et al. reported a case of severe aplastic anemia treated by umbilical cord hematopoietic cell transplant combined with mesenchymal stem cells. Our case reveals that infusing mesenchymal stem cells early (about 40 days) after UCBT may promote hematopoietic recovery [54]. In another case report a 26-year-old patient with aplastic anemia complicated by invasive sino-orbital aspergillosis. The patient was treated with MSCs to benefit from the dual effects of MSCs in immune reconstitution: suppression against alloreactive T cells and facilitation of the re-engraftment process. The patient did not develop acute or chronic graft-versus-host disease. The aspergillus infection healed completely. The engraftment failure was also ended without any complications. During his last visit in his fourth year after transplantation, the patient was in hematological remission [55]. A clinical trial evaluated the possibility of using umbilical cord derived mesenchymal stem cells to support umbilical cord hematopoietic stem cell grafts. Five young patients with severe AA who had failed initial CSA treatment and lacked a HLAmatched sibling donor, underwent co-transplantation of unrelated donor peripheral blood stem cells (UD-PBSCs) and umbilical cord mesenchymal/stroma stem cells (UC-MSCs). After conditioning, all patients received UD-PBSCs and UC-MSCs. There were no side effects attributable to the infused MSCs, and no severe complications or infections were observed in any patient after transplantation. After transplantation, one patient experienced primary graft failure, the reason for which may be related to a long history (>17 years) of SAA. The other four patients achieved complete hematopoietic recovery and complete donor hematopoietic chimerism. The authors did not observe severe aGVHD or cGVHD [56]. The use of mesenchymal stem cells in patients without a hematopoietic stem cell transplant has also been performed. In a study in 9 patients refractory to standard immune suppression, two to five weekly intravenous infusions of allogeneic unrelated non-human leukocyte antigen-matched bone marrow-derived mesenchymal stromal cells was performed. The median does of mesenchymal stem cells per infusion was 2.7 × 10(6) cells/kg (range, 1.3-4.5). Outcomes were compared to age and disease matched patients that were treated with standard rabbit ATG and cyclosporine. After a median follow-up of 20 months, no infusion-related adverse event was observed, but four deaths occurred as the result of heart failure and bacterial or invasive fungal infections; only two patients achieved partial hematologic responses at 6 months. The authors were not able to demonstrate by fluorescence in situ hybridization or variable number tandem repeat any MSC engraftment in patient marrow 30, 90 or 180 days after infusions [57]. The use of mesenchymal stem cells, such as the novel, enhanced, type of mesenchymal stem cells disclosed in the invention, for treatment of AA offers the possibility of simultaneously addressing the autoimmune/inflammatory basis of the condition, as well as stimulating hematopoiesis. Unfortunately, relatively little has been published in the utilization of mesenchymal stem cells alone in AA patients. Combination therapies disclosed in the current patent include the utilization of the novel mesenchymal stem cells described herein, together with other stem/progenitor cells such as endothelial progenitor cells. Given the importance of endothelial cells in so many aspects of biological systems, it would be conceptually feasible to believe that endothelial cells provide support, if not control, for hematopoietic processes. The original experiments highlighting this were studies which attempted to recapitulate hematopoiesis in vitro. Early experiments demonstrated that an endothelial cell layer was essential as part of the “stroma” for in vitro hematopoiesis [58, 59]. Interestingly, soluble factors generated by endothelial cells that supported in vitro hematopoiesis were not only identified [60], but it was demonstrated that their production was inducible by various agents such as lipopolysaccharide [61]. This suggested that the endothelium was an inducible source of factors stimulating hematopoiesis when physiologically necessary, such as during infection [62]. Detailed characterization of the role of endothelium in hematopoiesis was performed initially by anatomical studies which defined the sinusoidal aspects of the bone marrow hematopoietic endothelium [63, 64]. Morphological changes of this specialized endothelium have been identified during times of excessive production of various blood cells [65], hinting at a possible involvement in the process of hematopoiesis [66]. The concept that bone marrow resident endothelial cells support proliferation and granulocytic differentiation of hematopoietic stem cells was demonstrated decades ago in tissue culture studies [67]. Quesenberry and Gimbrone observed that not only that endothelial cells capable of stimulating hematopoiesis and granulopoiesis, but also that pretreatment of the endothelial cells with either endotoxin or granulocytes, augmented the ability of endothelial cells to promote granulopoiesis [61]. Further studies revealed that treatment of endothelial cells with TNF-alpha, a potent mediator of inflammation, actually resulted in augmentation of hematopoietic stimulatory activity, in part through production of a protein that was later identified as GM-CSF [68]. A similar finding was demonstrated with the inflammatory cytokine IL-1 [69]. Ascensao et al. sought to identify other mechanisms responsible for endothelial cell stimulation of bone marrow hematopoiesis. In their system they identified not only stimulation of granulopoiesis but also erythropoiesis. Specifically, they found that endothelial cells produce proteins of approximately 30,000 daltons, with isoelectric focusing points of 4.5 and 7.2, which stimulate the growth of human BFU-E and CFU-mix, as well as a heat-labile protein(s) of 30,000 and 15,000 daltons stimulated the proliferation and differentiation of granulocyte-macrophage (CFU-GM) colonies. Interestingly, no erythropoietin was found in the culture [70]. More detailed examination revealed that GM-CSF was one of these proteins [71]. Several other studies have confirmed GM-CSF production from endothelial cells. Malone et al used adipose derived endothelial cells and demonstrated GM-CSF production in an unstimulated state [72].

Thus in one embodiment, the invention teaches combination of endothelial cells, or endothelial progenitor cells together with the novel type of mesenchymal stem cells to obtain responses in this condition that, to date, has been relatively resilient to numerous therapeutic interventions. One potential source of endothelial progenitor cells could be derived from umbilical cords [73, 74], such as HUVEC cells. In another embodiment of the invention, trophoblast cells [75], may be utilized together with the stem cells of the invention, and/or also with administration of endothelial cells in order to enhance hematopoietic reconstitution together with inhibition of immunotoxicity. This approach faces the obvious issue of alloreactivity, however, alloreactivity would be small if at all present. The current fear of host versus graft is alleviated by the previously mentioned study as well as animal studies in which HUVEC cells were administered for the purpose of stimulating cancer immunity. In humans, the administration of allogeneic lymphocytes has been performed at up to 10(8) cells in patients with recurrent spontaneous abortions [76, 77]. Additionally, HUVEC administration has been performed at up to 10(9) cells without adverse effects associated with injection [78].

In another embodiment the invention teaches the use of the novel MSC described herein in the treatment of chronic traumatic encephalopathy (CTE). CTE is typically defined after the patient dies, based on autopsy examination of the brain. According to more recent criteria, there are 4 stages of CTE, all with increasing neuropathology [79-86].

In Stage 1 CTE, at the macroscopic level, the brain appears normal, however, immunohistochemistry reveals the presence of phosphorylated tau in a limited number of places in the brain, usually in lateral and frontal cortices, as well as proximal to small blood vessels in the depth of sulci. Although unclear, it is believed at a clinical level, patients with Stage 1 CTE appear generally asymptomatic, or in some situations exhibit short term memory deficiency In some cases mild depression and/or concurrent aggressiveness is exhibited. In State 2 CTE, there are some distinct anatomical deviations that may be seen such as enlargement of lateral ventricles, cavum septum pellucidum with or without fenestration, as well as pallor of the locus coeruleus and substantia nigra. Using immunohistochemistry, depositions of phosphorylated tau can be seen deep in the sulci, and there is an emergent spreading pattern. Behaviorally, Stage 2 CTE is characterized by mood and behavioral symptoms which could include behavioral outbursts and more severe depressive symptoms. In Stage 3 CTE, macroscopic abnormalities are highly visible. Also, global brain weight loss, mild frontal lobe and temporal lobe atrophy, and dilation of the ventricles is observed. In these patients, one half display septal abnormalities, including cavum septum pellucidum. Furthermore, immunocytochemistry reveals that tau pathology spreads, involving the frontal, temporal, parietal and insular cortices. At a clinical level these patients present with more cognitive deficits, including memory loss, executive functioning deficits, visuospatial dysfunction, and apathy. In Stage 4 CTE, there is a major reduction in brain weight, with brains weighing up to 30% less than control brains when “age-matched”. Severe atrophy of the frontal, medial temporal lobes, as well as anterior thalami is observed, along with atrophy of the white matter tracts. The majority of Stage 4 patients have septal abnormalities. The spread of the p-tau affects most regions, including the calcarine cortex. At a clinical level, patients present with advanced language deficits, psychotic symptoms which include paranoia, motor deficits, and parkinsonism.

The concept of CTE was originally described in an 1928 article in the Journal of the American Medical Association, in which he observed “fight fans and promoters have recognized a peculiar condition occurring among prize fighters which, in ring parlance, they speak of as″ punch drunk.” [87]. The name “punch drunk” remained until in 1937. Millspaugh developed the much more medical sounding term “dementia pugilistica” [88, 89]. In more recent times the term CTE was adopted [81, 90]. Modern day study of CTE was publicized by the pioneer work of pathologist Bennett Omalu, when in 2005 he reported a representative case of a retired National Football (NFL) player with progressive neurological dysfunction [91]. According to Omalu, the term CTE includes dementia pugilistica and supplants the use of the term dementia pugilistica. Due to initial controversy, and implications of CTE on various sports, it has been said that CTE is a very peculiar condition, in part because according to some authors “it is unique among brain diseases in having a history of decades of organized opposition to its codification as an authentic or valid entity [92].” About one-third of CTE cases are progressive, but clinical progression is not always sequential or predictable. The clinical symptoms vary extensively, which is probably due to multiple damage sites among athletes with the condition [84]. The severity varies from mild complaints, to severe deficits accompanied by dementia, Parkinson-like symptoms, and behavioral changes. Clinical symptoms include neurological and cognitive complaints together with psychiatric and behavioral disturbances. Early neurological symptoms may include speech problems and impaired balance, while later symptoms include ataxia, spasticity, impaired coordination, and extrapyramidal symptoms, with slowness of movements and tremor [84, 93]. Cognitive problems, such as attention deficits and memory disturbances, often become major factors in later stages of the disease, although may occur at varying times throughout the course of CTE. Psychiatric and behavioral problems include lack of insight and judgment, depression, disinhibition, euphoria, hypomania, irritability, aggressiveness and suicidal tendencies. A concussion is a type of traumatic brain injury which affects brain function. These effects are usually temporary but can include headaches and problems with concentration, memory, balance and coordination. Concussions are usually caused by a blow to the head. Violently shaking the head and upper body also can cause concussions. In some concussions the patient loses consciousness, but most do not. It’s possible to have a concussion and not realize it [90, 94, 95].

Concussion has been recognized as a clinical entity for more than 1,000 years. Throughout the 20th century it was studied extensively in boxers, but it did not pique the interest of the general population because it is the accepted goal of the boxer to inflict such an injury on their opponent. In 2002, however, the possibility that repetitive concussions could result in chronic brain damage and a progressive neurologic disorder was raised by a postmortem evaluation of a retired player in the most popular sports institution in the United States, the National Football League. Since that time, concussion has been a frequent topic of conversation in homes, schools, and throughout the media. It has become a major focus of sports programs in communities and schools at all levels [94]. Concussive injuries are also a problem in the military and industrial worksites. In the case of the former, traumatic brain injury resulting from exposure to the force of a detonation trigger, similar neuropathological mechanisms leading to neuropathology and sequelae indistinguishable to chronic traumatic encephalopathy is observed. In some cases, concussion causes no gross pathology, such as hemorrhage, and no abnormalities on structural brain imaging. There also may be no loss of consciousness. Many other complaints such as dizziness, nausea, reduced attention and concentration, memory problems, and headache have been reported. A greater likelihood of unconsciousness occurs with more severe concussions. These types of concussive head impacts are very frequent in American football whose athletes, especially linemen and linebackers, may be exposed to more than 1,000 impacts per season [96]. The effects of multiple concussions are becoming better recognized in these professional athletes, but much less is known about the long term-effects of repeated concussion in the brains of amateur athletes, teenagers and adolescents. Moreover, the amateur codes of football are less regulated than the professional codes, and the adolescent brain may be more vulnerable to concussion. The better-developed neck musculature of the professional football player, the more strictly controlled tackling and the better aftercare of the concussed professional means that the long-term public health problem of concussion in sport is grossly underestimated. Military personnel who have experienced concussion experience a range of detrimental and chronic medical conditions. Concussion occurring among soldiers deployed in Iraq is strongly associated with Post Traumatic Stress Disease (PTSD) and physical health problems 3 to 4 months after the soldiers return home. PTSD and depression are important mediators of the relationship between mild traumatic brain injury and physical health problems. PTSD was strongly associated with mild traumatic brain injury. It was reported that overall, 43.9% of soldiers who reported loss of consciousness met the criteria for PTSD, as compared with 27.3% of those with altered mental status, 16.2% of those with other injuries, and 9.1% of those with no injuries [97]. Also, more than 1 in 3 returning military troops who have sustained a deployment-related concussion have headaches which meet criteria for post traumatic headache [98]. It has been shown that nearly 15% of combat personnel sustained concussion while on duty [97]. Repeated concussion is a serious issue for combat personnel. A study showed that a majority of concussion incidents were blast related. The median time between events was 40 days, with 20% experiencing a second event within 2 weeks of the first and 87% within 3 months [99]. While an isolated concussion has been widely considered to be an innocuous event, recent studies [82, 93] have suggested that repeated concussion is associated with the development of a neurodegenerative disorder known as chronic traumatic encephalopathy (CTE). CTE is regarded as a disorder which often occurs in midlife, years or decades after the sports or military career has ended [82, 84, 90]. It is believed that in England at least 17% of boxers have CTE as judged by disturbed gait and coordination, slurred speech and tremors, as well as cerebral dysfunction causing cognitive impairments and neuro-behavioural disturbances [100]. In one study, diffusion tensor imaging (DTI), which is sensitive to microscopic white matter changes when routine MR imaging is unrevealing [101, 102], was used together with tract-based spatial statistics (TBSS) together with neuropsychological examination of executive functions and memory to investigate a collective of 31 male amateur boxers and 31 age-matched controls as well as a subgroup of 19 individuals, respectively, who were additionally matched for intellectual performance (IQ). It was found that participants had normal findings in neurological examination and conventional MR. Amateur boxers did not show deficits in neuropsychological tests when their IQ was taken into account. Fractional anisotropy was significantly reduced, while diffusivity measures were increased along central white matter tracts in the boxers’ group. These changes were in part associated with the number of fights. This study demonstrated that TBSS revealed widespread white matter disturbance partially related to the individual fighting history in amateur boxers. These findings closely resemble those in patients with accidental TBI and indicate similar histological changes in amateur boxers [103]. In addition to boxing, Jockeys have also been reported to suffer from CTE, in a 1976 publication, Foster et al reported Five National Hunt jockeys have been found to have post-traumatic encephalopathy- three with epilepsy and two with significant intellectual and psychological deterioration [104]. Other reports of jockey’s having similar situations have been described [105]. Numerous other causes of CTE have been described including whiplash [106], shaken baby syndrome [107], wrestling [108], military combat [109, 110], football [91, 111-114], rugby [115], soccer [116, 117], jail head trauma [118], shotgun injury [119] and mixed martial arts [120].

One study in the Journal of the American Medical Association (JAMA) examined a case series of 202 football players whose brains were donated for research. Neuropathological evaluations and retrospective telephone clinical assessments (including head trauma history) with informants were performed blinded. Online questionnaires ascertained athletic and military history. Neuropathological diagnoses of neurodegenerative diseases, including CTE, was based on defined diagnostic criteria. These included CTE neuropathological severity (stages I to IV or dichotomized into mild [stages I and II] and severe [stages III and IV]); informant-reported athletic history and, for players who died in 2014 or later, clinical presentation, including behavior, mood, and cognitive symptoms and dementia. Among 202 deceased former football players (median age at death, 66 years [interquartile range, 47-76 years]), CTE was neuropathologically diagnosed in 177 players (87%; median age at death, 67 years [interquartile range, 52-77 years]; mean years of football participation, 15.1 [SD, 5.2]), including 0 of 2 pre-high school, 3 of 14 high school (21%), 48 of 53 college (91%), 9 of 14 semiprofessional (64%), 7 of 8 Canadian Football League (88%), and 110 of 111 National Football League (99%) players. Neuropathological severity of CTE was distributed across the highest level of play, with all 3 former high school players having mild pathology and the majority of former college (27 [56%]), semiprofessional (5 [56%]), and professional (101 [86%]) players having severe pathology. Among 27 participants with mild CTE pathology, 26 (96%) had behavioral or mood symptoms or both, 23 (85%) had cognitive symptoms, and 9 (33%) had signs of dementia. Among 84 participants with severe CTE pathology, 75 (89%) had behavioral or mood symptoms or both, 80 (95%) had cognitive symptoms, and 71 (85%) had signs of dementia. In a sample of deceased football players who donated their brains for research, a high proportion had neuropathological evidence of CTE, suggesting that CTE may be related to prior participation in football [121]. In another study, the authors examined the effect of age of first exposure to tackle football on chronic traumatic encephalopathy (CTE) pathological severity and age of neurobehavioral symptom onset in tackle football players with neuropathologically confirmed CTE. The sample included 246 tackle football players who donated their brains for neuropathological examination. Two hundred eleven were diagnosed with CTE (126 of 211 were without comorbid neurodegenerative diseases), and 35 were without CTE. Informant interviews ascertained age of first exposure and age of cognitive and behavioral/mood symptom onset. Analyses accounted for decade and duration of play. Age of exposure was not associated with CTE pathological severity, Alzheimer’s disease or Lewy body pathology. In the 211 participants with CTE, every 1-year younger participants began to play tackle football predicted earlier reported cognitive symptom onset by 2.44 years (p < 0.0001) and behavioral/mood symptoms by 2.50 years (p < 0.0001). Age of exposure before 12 predicted earlier cognitive (p < 0.0001) and behavioral/mood (p < 0.0001) symptom onset by 13.39 and 13.28 years, respectively. In participants with dementia, the younger age of exposure corresponded to earlier functional impairment onset. Similar effects were observed in the 126 CTE-only participants. Effect sizes were comparable in participants without CTE. In this sample of deceased football players, the younger age of exposure to tackle football was not associated with CTE pathological severity, but predicted earlier neurobehavioral symptom onset. Youth exposure to football may reduce resiliency to late-life neuropathology [122]. One of the major observations found in patients with CTE is the extensive presence of neurofibrillary tangles [82, 84, 123-126]. Tangles are found intracellularly in the cytoplasm of neurons and consist of threadlike aggregates of hyperphosphorylated tau protein. In some cases, peripheral levels of Tau are reported to be elevated [127]. Tau is a normal axonal protein that binds to microtubules via their microtubule binding domains, thus promoting microtubule assembly and stability [128-132]. The hyperphosphorylated form of tau causes disassembly of microtubules and thus impaired axonal transport, leading to compromised neuronal and synaptic function, increased propensity of tau aggregation, and subsequent formation of insoluble fibrils and tangles [133, 134]. Unlike in Alzheimer’s disease, tangles in athletes with CTE tend to accumulate perivascularly within the superficial neocortical layers, particularly at the base of the sulci. Tau pathology in CTE is also patchy and irregularly distributed, possibly related to the many different directions of mechanical force induced by physical trauma [82]. It is the accumulation of hyperphosphorylated tau protein that is thought to result in the development of CTE and its associated psychiatric and behavioral disturbances.

How does tau become hyperphosphorylated in CTE? One hypothesis is that brain damage is associated with activation of caspase-3, which cleaves tau in a manner predisposing it to phosphorylation, as well as taking abnormal and potentially pathological confirmations [123, 135, 136]. Another proposed mechanism relates to decreased alkaline phosphatase that occurs as a result of various head injuries. For example, in one study, researchers used blast or weight drop models of traumatic brain injury (TBI) in rats, and observed pTau accumulation in the brain as early as 6 hours post-injury and further accumulation which varied regionally by 24h post-injury. The pTau accumulation was accompanied by reduced tissue non-specific alkaline phosphatase (TNAP) expression, activity in the injured brain regions and a significantly decreased plasma total alkaline phosphatase activity after the weight drop. These results reveal that both blast- and impact acceleration-induced head injuries cause an acute decrease in the level/activity of TNAP in the brain, which potentially contributes to trauma-induced accumulation of pTau and the resultant tauopathy. The regional changes in the level/activity of TNAP or accumulation of pTau after these injuries did not correlate with the accumulation of amyloid precursor protein, suggesting that the basic mechanism underlying tauopathy in TBI might be distinct from that associated with AD [137]. One of the interesting properties of the tau associated pathology is what appears to be the ability of phosphorylated tau to “spread” throughout the brain in a manner that has been previously compared to prion disease. One of the first possibilities of this type of tau propagation was suggested in a brain study. The brain extracted from deceased individuals with PiD, a neurodegenerative disorder characterized by three-repeat (3R) tau prions, were used to infect HEK293T cells expressing 3R tau fused to yellow fluorescent protein (YFP). Extracts from patient samples, which contain four-repeat (4R) tau prions, were transmitted to HEK293 cells expressing 4R tau fused to YFP. These studies demonstrated that prion propagation in HEK cells requires isoform pairing between the infecting prion and the recipient substrate. Interestingly, tau aggregates in AD and CTE, containing both 3R and 4R isoforms, were unable to robustly infect either 3R- or 4R-expressing cells. However, AD and CTE prions were able to replicate in HEK293T cells expressing both 3R and 4R tau. Unexpectedly, increasing the level of 4R isoform expression alone supported the propagation of both AD and CTE prions. These results allowed us to determine the levels of tau prions in AD and CTE brain extracts [138]. In a more definitive animal study, scientists evaluated whether moderate to severe TBI can trigger the initial formation of pathological tau that would induce the development of the pathology throughout the brain. To this end, the authors subjected tau transgenic mice to TBI and assessed tau phosphorylation and aggregation pattern to create a spatial heat map of tau deposition and spreading in the brain. The results suggest that brain injured tau transgenic mice have an accelerated tau pathology in different brain regions that increases over time compared to sham mice. The appearance of pathological tau occurs in regions distant to the injury area which are synaptically connected, indicating the spreading of tau aggregates spreading in a prion-like manner [139]. A more comprehensive study examined the ability of aggregated tau to spread. Scientists demonstrated a single severe brain trauma is associated with the emergence of widespread hyperphosphorylated tau pathology in a proportion of humans surviving after injury. In parallel experimental studies, a model of severe traumatic brain injury in wild-type mice, found progressive and widespread tau pathology, replicating the findings in humans. Brain homogenates from these mice, when inoculated into the hippocampus and overlying cerebral cortex of naïve mice, induced widespread tau pathology, synaptic loss, and persistent memory deficits. Accordingly, this data provides evidence that experimental brain trauma induces a self-propagating tau pathology, which can be transmitted between mice, and call for future studies aimed at investigating the potential transmissibility of trauma associated tau pathology in humans [140]. The ability of the pathological tau to spread has been postulated as one of the mechanisms by which brain pathology advances in patients with CTE years, if not decades, after cessation of repetitive injuries [141]. Interestingly some studies have demonstrated the possibility of using antibodies to inhibit pathological tau [142].

The invention provides means of inhibiting CTE through suppression of neuroinflammation. It is widely known that one result of a head injury is inflammation. However, the concept of propagating inflammation and self-maintaining inflammation is something relatively new. In contrast to traditional TBI, in which there is one major acute insult, CTE is characterized by multiple smaller insults, and in some cases progression of pathology increases despite large periods of time during after which the damaging agent has been removed. One of the cardinal features of CTE, which initiates with the concussive or subconcussive brain injury is the activation of the microglia. The microglia cells are brain residing macrophage lineage cells whose main physiological function is the phagocytosis of debris, as well as protection of the CNS from various pathogens. In one study, immunohistochemistry for reactive microglia (CD68 and CR3/43) was performed on human autopsy brain tissue and assessed ‘blind’ by quantitative image analysis. Head injury cases were compared with age matched controls, and within the traumatic brain injury group cases with diffuse traumatic axonal injury were compared with cases without diffuse traumatic axonal injury. The study found a neuroinflammatory response that develops within the first week and persists for several months after traumatic brain injury [143]. In a CTE study, the effects of repetitive head impacts (RHI) on the development of neuroinflammation and its relationship to CTE where examined. Specifically, the investigation aimed to determine the relationship between RHI exposure, neuroinflammation, and the development of hyperphosphorylated tau (pTau) pathology and dementia risk in CTE. A cohort of 66 deceased American football athletes from the Boston University-Veteran’s Affairs-Concussion Legacy Foundation Brain Bank as well as 16 non-athlete controls where utilized for the investigation. Subjects with a neurodegenerative disease other than CTE were excluded. Counts of total and activated microglia, astrocytes, and phosphorylated tau pathology were performed in the dorsolateral frontal cortex (DLF). Binary logistic and simultaneous equation regression models were used to test associations between RHI exposure, microglia, pTau pathology, and dementia. Duration of RHI exposure and the development and severity of CTE were associated with reactive microglial morphology and increased numbers of CD68 immunoreactive microglia in the DLF. The invention discloses means of inhibiting CTE, in part, through suppression of microglial activation by administration of mesenchymal stem cells, particularly, the novel mesenchymal stem cells disclosed in the current invention. A simultaneous equation regression model demonstrated that RHI exposure had a significant direct effect on CD68 cell density (p < 0.0001) and pTau pathology (p < 0.0001) independent of age at death. The effect of RHI on pTau pathology was partially mediated through increased CD68 positive cell density. A binary logistic regression demonstrated that a diagnosis of dementia was significantly predicted by CD68 cell density (OR = 1.010, p = 0.011) independent of age (OR = 1.055, p = 0.007), but this effect disappeared when pTau pathology was included in the model. In conclusion, RHI is associated with chronic activation of microglia, which may partially mediate the effect of RHI on the development of pTau pathology and dementia in CTE. The authors concluded that inflammatory molecules may be important diagnostic or predictive biomarkers as well as promising therapeutic targets in CTE [144].

In one embodiment the invention teaches the use of the novel mesenchymal stem cells to suppress generation of kynurenine through inhibition of macrophage activation and/or microglial activation. It is known that activated microglia produce kynurenine, in part through upregulation of the enzyme indolamine 2,3, deoxygenase [145-149]. An imbalance of neuroactive kynurenine pathway metabolites has been proposed as one mechanism behind the neuropsychiatric sequelae of certain neurological disorders. It has been hypothesized that concussed football players would have elevated plasma levels of neurotoxic kynurenine metabolites and reduced levels of neuroprotective metabolites relative to healthy football players and that altered kynurenine levels would correlate with post-concussion mood symptoms. In one study, Mood scales and plasma concentrations of kynurenine metabolites were assessed in concussed (N=18; 1.61 days post-injury) and healthy football players (N=18). A subset of football players returned at 1-week (N=14; 9.29 days) and 1-month post-concussion (N=14, 30.93 days).

Concussed athletes had significantly elevated levels of quinolinic acid (QUIN) and significantly lower ratios of kynurenic acid (KYNA) to QUIN at all time points compared with healthy athletes (p’s<0.05), with no longitudinal evidence of normalization of KYNA or KYNA/QUIN. At 1-day post-injury, concussed athletes with lower levels of the putatively neuroprotective KYNA/QUIN ratio reported significantly worse depressive symptoms (p=0.04), and a trend toward worse anxiety symptoms (p=0.06), while at 1-month higher QUIN levels were associated with worse mood symptoms (p’s<0.01). Finally, concussed athletes with worse concussion outcome, defined as number of days until return-to-play, had higher QUIN and lower KYNA/QUIN at 1-month post-injury (p’s<0.05). The authors concluded that the results converge with existing kynurenine literature on psychiatric patients and provide the first evidence of altered peripheral levels of kynurenine metabolites following sports-related concussion [150].

Direct monitoring of brain inflammation in vivo has been reported in a pilot study in which former National Football League (NFL) players were examined by new neuroimaging techniques and clinical measures of cognitive functioning. It was hypothesized that former NFL players would show molecular and structural changes in medial temporal and parietal lobe structures as well as specific cognitive deficits, namely those of verbal learning and memory. A significant increase in binding of [(11)C]DPA-713 to the translocator protein (TSPO), a marker of brain injury and repair, in several brain regions, such as the supramarginal gyrus and right amygdala, in 9 former NFL players compared to 9 age-matched, healthy controls was observed. Additionally, significant atrophy of the right hippocampus was seen. Finally, these same former players had varied performance on a test of verbal learning and memory, suggesting that these molecular and pathologic changes may play a role in cognitive decline. These results suggest that localized brain injury and repair, indicated by increased [(11)C]DPA-713 binding to TSPO, may be linked to history of NFL play. [(11)C]DPA-713 PET is a promising new tool that can be used in future study design to examine further the relationship between TSPO expression in brain injury and repair, selective regional brain atrophy, and the potential link to deficits in verbal learning and memory after NFL play [151]. In one embodiment, the invention teaches the suppression of brain atrophy through administration of mesenchymal stem cells, and in one embodiment the mesenchymal stem cells of the invention are utilized to prevention and/or reverse brain atrophy.

The invention also discloses treatments for traumatic brain injury.

In traumatic brain injury Wang et al reported on 40 patients with sequelae of TBI were randomly assigned to the stem cell treatment group (culture expanded allogeneic umbilical cord mesenchymal stem cells) or the control group. The patients in the stem cell treatment group underwent 4 stem cell transplantations via lumbar puncture. All patients of the group were also evaluated using Fugl-Meyer Assessments (FMA) and Functional Independence Measures (FIM) before and at 6 months after the stem cell transplantation. The patients in the control group did not receive any medical treatment (i.e., neither surgery nor medical intervention), and their FMA and FIM scores were determined on the day of the visit to the clinic and at 6 months after that clinical observation. The FMA results demonstrated an improvement in upper extremity motor sub-score, lower extremity motor sub-score, sensation sub-score and balance sub-score in the stem cell transplantation group at 6 months after the transplantation (P<0.05). The FIM results also exhibited significant improvement (P<0.05) in the patient self-care sub-score, sphincter control sub-score, mobility sub-score, locomotion sub-score, communication sub-score and social cognition sub-score. The control group exhibited no improvements after 6 months (P>0.05). All in all, the study results confirmed that the umbilical cord mesenchymal stem cell transplantation improved the neurological function and self-care in patients with TBI sequels [152].

Another study examined a total of 97 patients (24 with persistent vegetative state and 73 with disturbance motor activity) who developed a complex cerebral lesion after traumatic brain injury. Administration of autologous bone marrow ex vivo expanded mesenchymal stem cells was performed by lumbar puncture. Fourteen days after cell therapy, no serious complications or adverse events were reported. Thirty eight of 97 patients (39.2%) improved in the function of brain after transplant. Eleven of 24 patients (45.8%) with persistent vegetative state showed posttherapeutic improvements in consciousness (P = 0.024). Twenty-seven of 73 patients (37.0%) with a motor disorder began to show improvements in motor functions (P = 0.025). The age of patients and the time elapsed between injury and therapy had effects on the outcomes of the cellular therapy (P < 0.05) [153]. In another embodment, the invention teaches the use of the disclosed mesenchymal stem cells for the treatment of Multiple system atrophy (MSA). This is a a neurodegenerative disorder characterized by autonomic failure (cardiovascular and/or urinary), parkinsonism, cerebellar impairment and corticospinal signs with a median survival of 6-9 years [154]. The hallmark of its neuropathology is glial cytoplasmic inclusions composed of filamentous α-synuclein proteins in the striato-nigral and olivo-ponto-cerebellar structures [155]. Prevalence ranges from 1/50,000-1/20,000. MSA-parkinsonian type (MSA-p) predominates in the Western Hemisphere and MSA-cerebellar type (MSA-c) predominates in the Eastern Hemisphere. Genders are equally distributed [156]. Onset of MSA occurs in adulthood (>30 years, mean age 55-60 years). Clinical manifestations include autonomic failure (orthostatic hypotension, syncope, respiratory disturbances (sleep apnea, stridor and inspiratory sighs), constipation, bladder dysfunction (early urinary incontinence), erectile dysfunction in males and Raynaud syndrome) [157]. There are two main types of MSA: MSA-p and MSA-c [158]. MSA-p, possesses predominant parkinsonian features, including bradykinesia, rigidity, irregular jerky postural tremor and abnormal postures (camptocormia, Pisa syndrome and disproportionate antecollis). Patients with MSA-p may develop levodopa-induced orofacial and craniocervical dystonia. Classic pill-rolling rest tremor is uncommon .

MSA-c is a type of MSA with predominant cerebellar features such as gait and limb ataxia, oculomotor dysfunction and dysarthria. The predominant motor feature can change with time and patients with cerebellar ataxia can develop increasingly severe parkinsonian features which dominate the clinical presentation. Neuropsychiatric features, oculomotor dysfunction and sleep disturbances are also observed in MSA and include apathy, anxiety, depression, rapid eye movement sleep behavior disorder and periodic limb movements in sleep.

Lee et al utilized autologous bone marrow mesenchymal stem cells (MSC) to treat 33 patients with MSA-cerebellar type (MSA-C). Patients with baseline unified MSA rating scale (UMSARS) scores ranging from 30 to 50 were randomly assigned to receive MSC (4 × 10(7) /injection) via intra-arterial and intravenous routes or placebo. The primary outcome was change in the total UMSARS scores from baseline throughout a 360-day follow-up period between groups. Secondary outcomes were changes in the UMSARS part II scores, cerebral glucose metabolism, gray matter density, and cognitive performance over a 360-day period. The mixed model analysis of neurological deficits revealed a significant interaction effect between treatment group and time, suggesting that the MSC group had a smaller increase in total and part II UMSARS scores compared with the placebo group (p = 0.047 and p = 0.008, respectively). Cerebral glucose metabolism and gray matter density at 360 days relative to the baseline were more extensively decreased in the cerebellum and the cerebral cortical areas, along with greater deterioration of frontal cognition in the placebo group compared with the MSC group. The investigators found no serious adverse effects that were directly related to MSC treatment [159]. The same group also published one bone marrow mesenchymal stem cells administered through consecutively intra-arterial and three repeated intravenous injections and compared the long-term prognosis between mesenchymal stem cell -treated (n=11) and control MSA) patients (n=18). The mesenchymal stem cell-treated patients showed significantly greater improvement on the UMSARS than the control patients at all visits throughout the 12-month study period. Orthostasis in UMSARS I items and cerebellar dysfunction-related items of UMSARS II items were significantly different in favor of mesenchymal stem cell treatment compared to controls. Serial positron emission tomography scan in the mesenchymal stem cell treated group showed that increased fluorodeoxyglucose uptake from baseline was noted in cerebellum and frontal white matters. No serious adverse effects related to mesenchymal stem cell therapy occurred. This study demonstrated that cellular therapy in patients with MSA was safe and delayed the progression of neurological deficits with achievement of functional improvement in the follow-up period [160]. Another study exampled umbilical cord mesenchymal stromal cells in the treatment of spinocerebellar ataxia (SCA) and multiple system atrophy-cerebellar type (MSA-C). Specifically, 14 cases of SCA and 10 cases of MSA-C were given UC-MSC by weekly intrathecal injection, at a dose of 1 × 10(6)/kg four times as one course. All the patients received one course of treatment, except three patients who received two courses. The movement ability and quality of daily life were evaluated with the International Cooperative Ataxia Rating Scale (ICARS) and Activity of Daily Living Scale (ADL) and the scores compared with those before cell therapy. A follow-up of 6-15 months was carried out for all of the patients. The results showed that the ICARS and ADL scores were significantly decreased 1 month after treatment (P < 0.01). The symptoms, including unstable walking and standing, slow movement, fine motor disorders of the upper limbs, writing difficulties and dysarthria, were greatly improved except for one patient, who had no response. The observed side-effects included dizziness (four patients), back pain (two cases) and headache (one case), which disappeared within 1-3 days. During the follow-up, 10 cases remained stable for half a year or longer, while 14 cases had regressed to the status prior to the treatment within 1-14 months (an average of 3 months) [161].

Xi et al reported 10 patients suffering from multiple system atrophy who were treated by multiple cell transplantations from August 2005 to March 2011. They were six males and four females, with an average age of 51.90 ± 12.92 years (23-66 years). Multiple cell types were transplanted by intravenous, intrathecal, and intracranial routes; for example, 0.4-0.5 × 10(6)/kg umbilical cord mesenchymal cells by intravenous drip, intrathecal implantation of 2.0 × 10(6) Schwann cells and 2.0-5.0 × 10(6) neural progenitor cells through cerebellar cistern puncture, or 2 × 10(6) olfactory ensheathing cells and 4 × 10(6) neural progenitor cells injected into key points for neural network restoration (KPNNR). The neurological function was assessed before and after treatment with the International Cooperative Ataxia Rating Scale (ICARS) by UMSARS. The patients achieved neurological function amelioration after treatment, which included improvements in walking ability, gaits, standing, speech, and muscular tension; the ICARS score decreased from a preoperative 46.30 ± 14.50 points to postoperative 41.90 ± 18.40 points (p = 0.049). The UMSARS score decreased from preoperative 50.00 ± 20.65 points to postoperative 46.56 ± 23.05 points (p = 0.037). Among them, two patients remained stable and underwent a second treatment 0.5-1 year after the first therapy. After treatment, five patients were followed up for more than 6 months. Balance and walking ability improved further in four patients, while one patient remained stable for over 6 months. In conclusion, a strategy of comprehensive cell-based neurorestorative therapy for patients with multiple system atrophy is safe and appears to be beneficial [162]. In one embodiment the stem cells of the invention are utilized to treat Parkinson’s. Progressive supranuclear palsy (PSP), Steele-Richardson-Olszewsky syndrome (SR) type, is a progressive neurodegenerative disorder belonging to the group of taupathies, with motor, cognitive and behavioral symptoms. Its prevalence is about 6.5 cases per 100,000 people and its incidence is about 5.3 new cases every 100,000 people. Canesi et al used bone marrow derived mesenchymal stem cells as a therapeutic a pilot phase-I study for patients affected by progressive supranuclear palsy (PSP), a rare, severe and no-option form of Parkinsonism. Five patients received the cells by infusion into the cerebral arteries. Effects were assessed using the best available motor function rating scales (UPDRS, Hoehn and Yahr, PSP rating scale), as well as neuropsychological assessments, gait analysis and brain imaging before and after cell administration. One year after cell infusion, all treated patients were alive, except one, who died 9 months after the infusion for reasons not related to cell administration or to disease progression (accidental fall). In all treated patients motor function rating scales remained stable for at least six-months during the one-year follow-up. The authors claimed that disease stabilization is encouraging and suggest continued exploration of this intervention [163]. A study in classical Parkinson’s disease was performed in 7 patients aged 22 to 62 years with a mean duration of disease 14.7+/-7.56 years. Patients received a single-dose of autologous bone-marrow-derived mesenchymal stem cells implanted into the sublateral ventricular zone by stereotaxic surgery. Patients were followed up for a period that ranged from 10 to 36 months. The mean baseline “off” score was 65+/-22.06, and the mean baseline “on” score was 50.6+/-15.85. Three of 7 patients have shown a steady improvement in their “off″/“on” Unified Parkinson’s Disease Rating Scale (UPDRS). The mean “off” score at their last follow-up was 43.3 with an improvement of 22.9% from the baseline. The mean “on” score at their last follow-up was 31.7, with an improvement of 38%. Hoehn and Yahr (H&Y) and Schwab and England (S&E) scores showed similar improvements from 2.7 and 2.5 in H&Y and 14% improvement in S&E scores, respectively. A subjective improvement was found in symptoms like facial expression, gait, and freezing episodes; 2 patients have significantly reduced the dosages of PD medicine. The authors concluded that the protocol seems to be safe, and no serious adverse events [164].

Inflammation inhibition by cells of the invention, as well as growth factor release and regenerative activities, support the use of the cells for treatment of autoimmune conditions. One example is the treatment of Crohn’s Disease. Crohn’s disease is an autoimmune condition that in advanced stages is refractory to treatment. In less advanced stages treatments such as systemic immune suppression carry unintended consequences. The rationale for treatment of Crohn’s disease by systemic administration of mesenchymal stem cells is based on the established immune modulatory potential of the cells, but directly, as well as indirectly through stimulation of T regulatory cells and inhibition of dendritic cell maturation. One clinical trial treated adult patients with refractory Crohn’s disease (eight females and two males) with autologous ex vivo expanded bone marrow mesenchymal stem cells. 9 patients received two doses of 1-2×10(6) cells/kg body weight, intravenously, 7 days apart. During follow-up, possible side effects and changes in patients’ Crohn’s disease activity index (CDAI) scores were monitored. Colonoscopies were performed at weeks 0 and 6, and mucosal inflammation was assessed by using the Crohn’s disease endoscopic index of severity. The investigators reported that mesenchymal stem cells isolated from patients with Crohn’s disease showed similar morphology, phenotype and growth potential compared to cells from healthy donors. Importantly, immunomodulatory capacity was intact, as Crohn’s disease derived mesenchymal stem cells possessed ability to significantly reduce peripheral blood mononuclear cell proliferation in vitro. Infusion of cells was without side effects, besides a mild allergic reaction probably due to the cryopreservant DMSO in one patient. Baseline median CDAI was 326 (224-378). Three patients showed clinical response (CDAI decrease ≥70 from baseline) 6 weeks post-treatment; conversely three patients required surgery due to disease worsening. Disease worsening was anticipated in all the patients treated based on severity of the disease [165].

In order to augment therapeutic efficacy, treatment of bone marrow mesenchymal stem cells with interferon gamma was performed in a case study of a patient suffering from childhood-onset, multidrug resistant and steroid-dependent Crohn’s disease who underwent systemic infusions of interferon gamma pretreated mesenchymal stem cells, which led to a temporary reduction in CCR4, CCR7 and CXCR4 expression by T-cells, and a temporary decrease in switched memory B-cells, In addition, following mesenchymal stem cell infusion, lower doses of steroids were needed to inhibit proliferation of the patient’s peripheral blood mononuclear cells. Despite these changes, no significant clinical benefit was observed, and the patient required rescue therapy with infliximab and subsequent autologous hematopoietic stem cell transplantation [166]. In another attempt to increase efficacy of autologous bone marrow mesenchymal stem cells, investigators assessed 3 doses of cells that propagated for 2-3 weeks with fibrinogen depleted human platelet lysate and subsequently administered to subjects without interval cryobanking. Twelve subjects received a single mesenchymal stromal cell intravenous infusion of 2, 5 or 10 million cells/kg BW(n = 4/group). All patients tolerated the mesenchymal stromal cell infusion well and no dose limiting toxicity was seen. Seven patients had serious adverse events of which five were hospitalisations for Crohn’s disease flare. Two of these serious adverse events were possibly related to the mesenchymal stromal cells infusion. Five subjects showed clinical response 2 weeks after the infusion. Mesenchymal stromal cell phenotype, cytokine responsiveness, and peripheral blood mononuclear cell proliferation blockade were not different among the patients [167]. A complication of Crohn’s disease is complex perianal fistulas. A perianal fistula is an abnormal connection between the perianal space and outside skin surface which causes a significant negative impact on quality of life. A fistula is considered to be complex when its treatment involves a high risk of causing loss of anal continence, the fistulous tract crosses more than 30% of external sphincter, several tracts are found, it is recurrent, or when the patient has incontinence, local irritation, or Crohn’s disease. Current treatment for complex fistula in patients with Crohn’s disease is marked by poor efficacy and high costs. Patients with complex fistula but no inflammatory disease require surgery, with its attendant risk of faecal incontinence. The effective management of fistulas in patients with Crohn’s disease presents an extremely challenging problem. While surgical options exist, numerous patients are not eligible candidates, and those who are often need subsequent surgeries due to presence of the underlying cause. A Phase I clinical trial was conducted involving 4 patients with Crohn’s disease, to test the feasibility and safety of autologous stem cells transplantation in the treatment of fistulas. The researchers also studied the expression of various cell markers and the growth rates of the lipoaspirate-derived cells that were used for transplantation. Nine fistulas in four patients with autologous adipose tissue-derived stem cells at Passage 3 or earlier. Eight inoculated fistulas were followed weekly for at least eight weeks. In six fistulas, the external opening was covered with epithelium at the end of Week 8, and, thus, these fistulas were considered healed (75 percent). In the other two fistulas, there was only incomplete closure of the external opening, with a decrease in output flow (not healed; 25 percent). No adverse effects were observed in any patient at the end of the follow-up period (minimum follow-up, 12 months; maximum follow-up, 30 months; follow-up average, 22 months) [168].

Another study evaluated ex vivo expanded autologous adipose derived mesenchymal stem cells. This study was substantially larger being a multicenter, randomized, single-blind, add-on clinical trial, in which 200 adult patients from 19 centers were randomly assigned to receive 20 million stem cells (group A, 64 patients), 20 million adipose-derived stem cells plus fibrin glue (group B, 60 patients), or fibrin glue (group C, 59 patients) after closure of the internal opening. Fistula healing was defined as reepithelization of the external opening and absence of collection >2 cm by MRI. If the fistula had not healed at 12 weeks, a second dose (40 million stem cells in groups A and B) was administered. Patients were evaluated at 24 to 26 weeks (primary end point) and at 1 year (long-term follow-up). All results where reported according to the “blinded evaluator” assessment. After 24 to 26 weeks, the healing rate was 39.1%, 43.3%, 37.3% in groups A, B, and C (p = 0.79). At 1 year, the healing rates were 57.1%, 52.4%, and 37.3 % (p = 0.13). On analysis of the subpopulation treated at the technique’s pioneer center, healing rates were 54.55%, 83.33%, and 18.18%, at 24 to 26 weeks (p < 0.001). No SAEs were reported [169]. The difference for the discrepancy in therapeutic efficacy between all the centers in the study versus the original center was not provided in the publication. Use of autologous bone marrow mesenchymal stem cells was also attempted in Crohn’s fistulas. In one clinical study, Ten patients received intrafistular cell injections (median 4) scheduled every 4 weeks, and were monitored by surgical, MRI and endoscopic evaluation for 12 months afterwards. The feasibility of obtaining at least 50×10⁶ mesenchymal stem cells from each patient, the appearance of adverse events, and the efficacy in terms of fistula healing and reduction of both Crohn’s disease and perianal disease activity indexes were evaluated. In addition, the percentage of both mucosal and circulating regulatory T cells expressing FoxP3, and the ability of mesenchymal stem cells to influence mucosal T cell apoptosis were investigated. The authors reported that expansion of mesenchymal stem cells was successful in all cases; sustained complete closure (seven cases) or incomplete closure (three cases) of fistula tracks with a parallel reduction of Crohn’s disease and perianal disease activity indexes (p < 0.01 for both), and rectal mucosal healing were induced by treatment without any adverse effects. The percentage of mucosal and circulating regulatory T cells significantly increased during the treatment and remained stable until the end of follow up (p < 0.0001 and p < 0.01, respectively). Furthermore, mesenchymal stem cells have been proven to affect mucosal T cell apoptotic rate [170]. Another study reported long-term follow up of patients treated with autologous bone marrow mesenchymal stem cells. Starting from Jan. 10, 2007, through Jun. 30, 2014, clinical evaluation, calculation of the Crohn disease activity index (CDAI), therapeutic management, and documentation of adverse events in 8 of the 10 patients (5 men; median age, 37 years) who had been injected locally with MSCs were prospectively recorded for 72 months. Cumulative probabilities of fistula recurrence and medical or surgical treatment were estimated using a Kaplan-Meier method, whereas differences among the pre- and post-MSC CDAI values were calculated with the Mann-Whitney U test. Following disease remission observed after 12 months from MSC treatment (P<0.001), the mean CDAI score increased significantly during the subsequent 2 years (P=0.007), and was then followed by a gradual decrease, with the patients achieving remission again (P=0.02) at the end of the 5-year follow-up. The probability of fistula relapse-free survival was 88% at 1 year, 50% at 2 years, and 37% during the following 4 years, and the cumulative probabilities of surgery- and medical-free survival were 100% and 88% at 1 year, 75% and 25% at 2, 3, and 4 years, and 63% and 25% at 5 and 6 years, respectively. No adverse events were recorded [171]. Given that numerous clinical trials have demonstrated therapeutic signals of allogeneic mesenchymal stem cells, as well as the fact that allogeneic cells are commercially more attractive, a study was conducted attempting to heal Crohn’s fistulas using allogeneic mesenchymal stem cells. An open-label, single-arm clinical trial was conducted at six Spanish hospitals. Twenty-four patients were administered intralesionally with 20 million allogeneic expanded adipose derived mesenchymal stem cells in one draining fistula tract. A subsequent administration of 40 million cells was performed if fistula closure was incomplete at week 12. Subjects were followed until week 24 after the initial administration. Treatment-related adverse events did not indicate any clinical safety concerns after 6 months follow-up. The full analysis of efficacy data at week 24 showed 69.2 % of the patients with a reduction in the number of draining fistulas, 56.3 % of the patients achieved complete closure of the treated fistula achieved, and 30 % of the cases presenting complete closure of all existing fistula tracts. Of note, closure was strictly defined as: absence of suppuration through the external orifice and complete re-epithelization, plus absence of collections measured by magnetic resonance image scan (MRI). Furthermore, MRI Score of Severity showed statistically significant differences at week 12 with a marked reduction at week 24 [172].

A randomised, double-blind, parallel-group, placebo-controlled study at 49 hospitals in seven European countries and Israel from Jul. 6, 2012, to Jul. 27, 2015 was performed using allogeneic adipose derived mesenchymal stem cells. Adult patients (≥18 years) with Crohn’s disease and treatment-refractory, draining complex perianal fistulas were randomly assigned (1:1) using a pre-established randomisation list to a single intralesional injection of 120 million cells or 24 mL saline solution (placebo), with stratification according to concomitant baseline treatment. Treatment was administered by an unmasked surgeon, with a masked gastroenterologist and radiologist assessing the therapeutic effect. The primary endpoint was combined remission at week 24 (ie, clinical assessment of closure of all treated external openings that were draining at baseline, and absence of collections >2 cm of the treated perianal fistulas confirmed by masked central MRI). Efficacy was assessed in the intention-to-treat (ITT) and modified ITT populations; safety was assessed in the safety population. 212 patients were randomly assigned: 107 to cells and 105 to placebo. A significantly greater proportion of patients treated with cells versus placebo achieved combined remission in the ITT (53 of 107 [50%] vs 36 of 105 [34%]; difference 15-2%, 97-5% CI0.2-30.3; p=0.024) and modified ITT populations (53 of 103 [51%] vs 36 of 101 [36%]; 15.8%, 0.5-31.2; p=0.021). 18 (17%) of 103 patients in the cell treated group versus 30 (29%) of 103 in the placebo group experienced treatment-related adverse events, the most common of which were anal abscess (six in the cell group vs nine in the placebo group) and proctalgia (five vs nine) [173]. A subsequent study from an independent group also examined allogeneic bone marrow mesenchymal stem cells in patients with Crohn’s fistulas. Twenty-one patients with refractory perianal fistulizing Crohn’s disease were randomly assigned to groups given injections of 1 × 10(7) (n = 5, group 1), 3 ×10(7) (n = 5, group 2), or 9 × 10(7) (n = 5, group 3) MSCs, or placebo (solution with no cells, n = 6), into the wall of curettaged fistula, around the trimmed and closed internal opening. The primary outcome, fistula healing, was determined by physical examination 6, 12, and 24 weeks later; healing was defined as absence of discharge and <2 cm of fluid collection-the latter determined by magnetic resonance imaging at week 12. All procedures were performed at Leiden University Medical Center, The Netherlands, from June 2012 through July 2014. No adverse events were associated with local injection of any dose of MSCs. Healing at week 6 was observed in 3 patients in group 1 (60.0%), 4 patients in group 2 (80.0%), and 1 patient in group 3 (20.0%), vs 1 patient in the placebo group (16.7%) (P = 0.08 for group 2 vs placebo). At week 12, healing was observed in 2 patients in group 1 (40.0%), 4 patients in group 2 (80.0%), and 1 patient in group 3 (20.0%), vs 2 patients in the placebo group (33.3%); these effects were maintained until week 24 and even increased to 4 (80.0%) in group 1. At week six, 4 of 9 individual fistulas had healed in group 1 (44.4%), 6 of 7 had healed in group 2 (85.7%), and 2 of 7 had healed in group 3 (28.6%) vs 2 of 9 (22.2%) in the placebo group (P = 0.04 for group 2 vs placebo). At week twelve, 3 of 9 individual fistulas had healed in group 1 (33.3%), 6 of 7 had healed in group 2 (85.7%), 2 of 7 had healed in group 3 (28.6%), and 3 of 9 had healed in the placebo group (33.3%). These effects were stable through week 24 and even increased to 6 of 9 (66.7%) in group 1 (P = 0.06 group 2 vs placebo, weeks 12 and 24) [174].

Cells of the invention are potently anti-inflammatory. Accordingly, they may be used to suppress one of the major inflammatory conditions called Graft Versus Host Disease (GVHD). A Lancet publication in 2004 described transplantation of haploidentical mesenchymal stem cells in a patient with severe treatment-resistant grade IV acute graft-versus-host disease of the gut and liver. Clinical response was striking. The patient is now well after 1 year. The authors postulated that mesenchymal stem cells have a potent immunosuppressive effect in vivo which induced remission of GVHD [175].

A subsequent publication by the same group administered bone marrow derived MSC to eight patients with steroid-refractory grades III-IV GVHD and one who had extensive chronic GVHD. The MSC dose was median 1.0 (range 0.7 to 9)×10(6)/kg. No acute side-effects occurred after the MSC infusions. Six patients were treated once and three patients twice. Two patients received MSC from HLA-identical siblings, six from haplo-identical family donors and four from unrelated mismatched donors. Acute GVHD disappeared completely in six of eight patients. One of these developed cytomegalovirus gastroenteritis. Complete resolution was seen in gut (6), liver (1) and skin (1). Two died soon after MSC treatment with no obvious response. One of them had MSC donor DNA in the colon and a lymph node. Five patients are still alive between 2 months and 3 years after the transplantation. Their survival rate was significantly better than that of 16 patients with steroid-resistant biopsy-proven gastrointestinal GVHD, not treated with MSC during the same period (P = 0.03). One patient treated for extensive chronic GVHD showed a transient response in the liver, but not in the skin and he died of Epstein-Barr virus lymphoma [176].

Expanding on these clinical experiences, LeBlanc’s group reported on 55 patients with steroid-resistant, severe, acute GVHD were treated with mesenchymal stem cells, derived with the European Group for Blood and Marrow Transplantation ex-vivo expansion procedure, in a multicentre, phase II experimental study. We recorded response, transplantation-related deaths, and other adverse events for up to 60 months’ follow-up from infusion of the cells. The median dose of bone-marrow derived mesenchymal stem cells was 1.4×10(6) (min-max range 0.4-9×10(6)) cells per kg bodyweight. 27 patients received one dose, 22 received two doses, and six three to five doses of cells obtained from HLA-identical sibling donors (n=5), haploidentical donors (n=18), and third-party HLA-mismatched donors (n=69). 30 patients had a complete response and nine showed improvement. No patients had side-effects during or immediately after infusions of mesenchymal stem cells. Response rate was not related to donor HLA-match. Three patients had recurrent malignant disease and one developed de-novo acute myeloid leukaemia of recipient origin. Complete responders had lower transplantation-related mortality 1 year after infusion than did patients with partial or no response (11 [37%] of 30 vs 18 [72%] of 25; p=0.002) and higher overall survival 2 years after hematopoietic-stem-cell transplantation (16 [53%] of 30 vs four [16%] of 25; p=0.018) [177].

Other similar studies have been conducted, for example, a report on two cases of the patients with acute steroid-refractory GVHD who underwent allogeneic HSCT. These patients received infusions of third-party clonal mesenchymal stem cells (cMSCs), which were isolated by a subfractionation culturing method (SCM) developed recently by our group, and showed marked improvement of the disease. MSCs represent a new potential therapeutic option in the treatment of steroid-refractory acute GVHD. They showed safety and improved clinical findings of the acute GVHD patients [178].

A repetition of MSC for steroid refractory GVHD was performed by Herrmann et al. The primary end point was safety, and secondary end points were best response achieved and overall survival. A median of two infusions per patient were administered. The response rate overall for AGVHD was complete in seven, partial in four and no response in one patient. Of the seven patients who achieved a complete response, six are alive. The actuarial survival for the overall group of AGVHD was 55% at 30 months. Two patients with CGVHD achieved complete response with two partial responses and three with no response. The survival for those with AGVHD who achieved a complete response compared with those who did not was significant (p = 0.03) [179].

Bone marrow multipotent mesenchymal stem cells are generated by selecting cells expressing STRO-4, which are the therapeutic product of Mesoblast Inc. Assessment of these cells in GVHD was performed by Muroi et al. who published a phase II/III study using the cells focused on steroid-refractory grade III or IV aGVHD was conducted. The number of infused MSCs and the number of MSC infusions were the same as the phase I/II study. No additional immunosuppressant was given for steroid-refractory aGVHD during the course of MSC infusions. Twenty-five patients (grade III, 22 patients and grade IV, 3 patients) were enrolled in this study. At 4 weeks after the first MSC infusions, six (24 %) and nine patients (36 %) achieved a complete response (CR) and partial response (PR), respectively. Durable CR by 24 weeks, which was the primary end-point, was obtained in 12 of 25 patients (48 %). At 52 weeks, 12 patients (48 %) treated with MSCs only (six patients) and MSCs plus additional treatments (six patients) were alive in CR. The survival was significantly better in patients showing overall response (OR; CR+PR) than in those showing no OR at 4 weeks. Adverse effects commonly associated with MSC infusions were not observed. Taken together, our two clinical trials suggest multipotent MSC to be effective for steroid-refractory aGVHD [180].

An interesting approach to 3^{rd} party mesenchymal stem cell transplantation that was assessed involved pooling bone marrow mononuclear cells of 8 healthy “3(rd)-party” donors. Generated cells were frozen in 209 vials and designated as mesenchymal stromal cell bank. These vials served as a source for generation of clinical grade mesenchymal stromal cell end-products, which exhibited typical mesenchymal stromal cell phenotype, trilineage differentiation potential and at later passages expressed replicative senescence-related markers (p21 and p16). Genetic analysis demonstrated their genomic stability (normal karyotype and a diploid pattern). Importantly, clinical end-products exerted a significantly higher allosuppressive potential than the mean allosuppressive potential of mesenchymal stromal cells generated from the same donors individually. Administration of 81 mesenchymal stromal cell end-products to 26 patients with severe steroid-resistant acute graft-versus-host disease in 7 stem cell transplant centers who were refractory to many lines of treatment, induced a 77% overall response at the primary end point (day 28). Remarkably, although the cohort of patients was highly challenging (96% grade III/IV and only 4% grade II graft-versus-host disease), after treatment with mesenchymal stromal cell end-products the overall survival rate at two years follow up was 71±11% for the entire patient cohort, compared to 51.4±9.0% in graft-versus-host disease clinical studies, in which mesenchymal stromal cells were derived from single donors. Mesenchymal stromal cell end-products may, therefore, provide a novel therapeutic tool for the effective treatment of severe acute graft-versus-host disease [181].

### Serum Free MSC Expansion for GVHD

In order to decrease possible immunogenic effects of fetal calf serum used in expansion of mesenchymal stem cells, investigators have been attempting to use serum-free mesenchymal stem cell expansion media. A publication reported on a case series of 13 patients with steroid-refractory aGVHD who received BM-derived MSC expanded in platelet lysate-containing medium from unrelated HLA disparate donors. MSC were characterized by their morphological, phenotypical and functional properties. All tested preparations suppressed the proliferation of in vitro activated CD4+ T cells. MSC were transfused at a median dosage of 0.9 x 10(6)/kg (range 0.6-1.1). The median number of MSC applications was 2 (range 1-5). Only two patients (15%) responded and did not require any further escalation of immunosuppressive therapy. Eleven patients received additional salvage immunosuppressive therapy concomitant to further MSC transfusions, and after 28 days, five of them (45%) showed a response. Four patients (31%) are alive after a median follow-up of 257 days, including one patient who initially responded to MSC treatment [182].

The use of serum free media for expansion of mesenchymal stem cells was also demonstrated in a pediatric population. Eleven pediatric patients diagnosed with acute or chronic GVHD (aGVHD, cGVHD) treated for compassionate use with GMP-grade unrelated HLA-disparate donors’ bone marrow-derived MSCs, expanded in platelet-lysate (PL)-containing medium. Eleven patients (aged 4-15 years) received intravenous (i.v.) MSCs for aGVHD or cGVHD, which was resistant to multiple lines of immunosuppression. The median dose was 1.2 × 10(6)/kg (range: 0.7-3.7 × 10(6)/kg). No acute side effects were observed, and no late side effects were reported at a median follow-up of 8 months (range: 4-18 months). Overall response was obtained in 71.4% of patients, with complete response in 23.8% of cases. None of our patients presented GVHD progression upon MSC administration, but 4 patients presented GVHD recurrence 2 to 5 months after infusion. Two patients developed chronic limited GVHD. This study underlines the safety of PL-expanded MSC use in children. MSC efficacy seems to be greater in aGVHD than in cGVHD, even after failure of multiple lines of immunosuppression [183].

Another study used platelet lysate for expansion of MSC. The study evaluated 58 adult patients suffering from steroid-resistant acute GVHD (aGVHD) treated with mesenchymal stromal cells (MSCs) as salvage therapy for steroid-refractory aGvHD. Third-party MSCs expanded in platelet lysate-containing medium were transfused at a median dose of 0.99 × 10(6) cells per kg b.wt. A median of two MSC infusions were administered to each patient. Median time between the onset of aGvHD and the first infusion of MSCs was 12 days (range, 6-62 days). Most patients (79%) had grade IV aGvHD. Five patients showed complete response, five showed very good partial response, 17 showed partial response, and 31 showed no response. The estimated probability of survival after 1 year was 19%, and median survival was 69 days. Overall survival was not significantly different from that of a historical cohort of patients receiving alternative salvage therapy and no MSC infusions. In conclusion, MSC treatment on top of conventional immunosuppression was associated with an overall response rate of 47% but improved outcome in terms of survival remains to be shown [184].

An alternative to platelet lysate for MSC expansion is utilization of human plasma. A clinical trial evaluated the feasibility and efficacy of the infusion of mesenchymal stem cells expanded using human serum for the treatment of refractory acute or chronic graft-versus-host disease. Twenty-eight expansions were started. In 22, a minimum of more than 1 × 10⁶ mesenchymal stem cells/kg were obtained after a median of 26 days; this threshold was not obtained in the remaining cases. Ten patients received cells for the treatment of refractory or relapsed acute graft-versus-host disease and 8 for chronic disease. One patient treated for acute graft-versus-host disease obtained a complete response, 6 had a partial response and 3 did not respond. One of the chronic patients achieved complete remission, 3 patients a partial response, and 4 patients did not respond. The current study supports the use of this approach in less heavily treated patients for both acute and chronic graft-versus-host disease [184].

Muroi et al conducted a multicenter phase I/II study using mesenchymal stem cells (MSCs) manufactured from the bone marrow of healthy unrelated volunteers to treat steroid-refractory acute graft-versus-host disease (aGVHD). Fourteen patients with hematological malignancies who suffered from grade II (9 patients) or III aGVHD (5) were treated. Affected organs were gut (10 patients), skin (9 patients), and liver (3 patients). Seven patients had two involved organs. The median age was 52. No other second-line agents were given. MSCs were given at a dose of 2 × 10(6) cells/kg for each infusion twice a week for 4 weeks. If needed, patients were continuously given MSCs weekly for an additional 4 weeks. By week 4, 13 of 14 patients (92.9 %) had responded to MSC therapy with a complete response (CR; n = 8) or partial response (PR; n = 5). At 24 weeks, 11 patients (10 with CR and 1 with PR) were alive. At 96 weeks, 8 patients were alive in CR. A total of 6 patients died, attributable to the following: underlying disease relapse (2 patients), breast cancer relapse (1), veno-occlusive disease (1), ischemic cholangiopathy (1), and pneumonia (1). No clear adverse effects associated with MSC infusion were observed. Third party-derived bone marrow MSCs may be safe and effective for patients with steroid-refractory aGVHD [185].

Cotransplantation of MSC at the time of hematopoietic stem cell grafting also appears to reduce GVHD. In 13 children with hematological disorders (median age 2 years) undergoing UCBT, investigators co-transplanted paternal, HLA-disparate MSCs with the aim of improving hematological recovery and reducing rejection. No differences in hematological recovery or rejection rates compared with 39 matched historical controls, most of whom received G-CSF after UCBT. However, the rate of grade III and IV acute GVHD was significantly decreased in the study cohort when compared with controls (P=0.05), thus resulting in reduced early transplant related mortality (TRM). Although these data do not support the use of MSCs in UCBT to support hematopoietic engraftment, they suggest that MSCs, possibly because of their immunosuppressive effect, may abrogate life-threatening acute GVHD and reduce early TRM [186].

Administration of MSC for prophylaxis of GVHD was assessed in a prospective clinical trial was based on the random patient allocation to the following two groups receiving (1) standard GVHD prophylaxis and (2) standard GVHD prophylaxis combined with MMSCs infusion. Bone marrow MMSCs from hematopoietic stem cell donors were cultured and administered to the recipients at doses of 0.9-1.3 ×10(6)/kg when the blood counts indicated recovery. aGVHD of stage II-IV developed in 38.9% and 5.3% of patients in group 1 and group 2, respectively, (P = 0.002). There were no differences in the graft rejection rates, chronic GVHD development, or infectious complications. Overall mortality was 16.7% for patients in group 1 and 5.3% for patients in group 2. The efficacy and the safety of MMSC administration for aGVHD prophylaxis were demonstrated in this study [187]

A pediatric study reported on premanufactured, universal donor, formulation of hMSCs (Prochymal) in children (n = 12; 10 boys; 9 Caucasian; age range: 0.4-15 years) with treatment-resistant grade III and IV aGVHD who received therapy on compassionate use basis between July 2005 and June 2007 at 5 transplant centers. All patients had stage III or IV gut (GI) symptoms and half had additional liver and/or skin involvement. Disease was refractory to steroids in all cases and additionally to a median of 3 other immunosuppressive therapies. The hMSCs (8 × 10(6)cells/kg/dose in 2 patients and 2 × 10(6)cells/kg/dose in the rest) were infused intravenously over 1 hour twice a week for 4 weeks. Partial and mixed responders received subsequent weekly therapy for 4 weeks. HLA or other matching was not needed. The hMSCs were started at a median of 98 days (range: 45-237) posttransplant. A total of 124 doses were administered, with a median of 8 doses (range: 2-21) per patient. Overall, 7 (58%) patients had complete response, 2 (17%) partial response, and 3 (25%) mixed response. Complete resolution of GI symptoms occurred in 9 (75%) patients. Two patients relapsed after initial response and showed partial response to retreatment. The cumulative incidence of survival at 100 days from the initiation of Prochymal therapy was 58%. Five of 12 patients (42%) were still alive after a median follow-up of 611 days (range: 427-1111) in surviving patients. No infusional or other identifiable acute toxicity was seen in any patient. Multiple infusions of hMSCs were well tolerated and appeared to be safe in children. Clinical responses, particularly in the GI system, were seen in the majority of children with severe refractory aGVHD. Given the favorable results observed in a patient population with an otherwise grave prognosis, we conclude that hMSCs hold potential for the treatment of aGVHD, and should be further studied in phase III trials in pediatric and adult patients [188].

Another pediatric study retrospectively analysed a cohort of 37 children (aged 3 months-17 years) treated with MSCs for steroid-refractory grade III-IV aGvHD. All patients but three received multiple MSC infusions. Complete response (CR) was observed in 24 children (65%), while 13 children had either partial (n = 8) or no response (n = 5). Cumulative incidence of transplantation-related mortality (TRM) in patients who did or did not achieve CR was 17% and 69%, respectively (P = 0.001). After a median follow-up of 2.9 years, overall survival (OS) was 37%; it was 65% vs. 0% in patients who did or did not achieve CR, respectively (P = 0.001). The median time from starting steroids for GvHD treatment to first MSC infusion was 13 d (range 5-85). Children treated between 5 and 12 d after steroid initiation showed a trend for better OS (56%) and lower TRM (17%) as compared with patients receiving MSCs 13-85 d after steroids (25% and 53%, respectively; P = 0.22 and 0.06, respectively). The authors concluded that multiple MSC infusions are safe and effective for children with steroid-refractory aGvHD, especially when employed early in the disease course [189].

Kurtzburg et al. described use of remestemcel-L (Prochymal), a third-party, off-the-shelf source of hMSCs, as a rescue agent for treatment-resistant aGVHD in pediatric patients. Children with grade B-D aGVHD failing steroids and, in most cases, other immunosuppressive agents were eligible for enrollment. Patients received 8 biweekly i.v. infusions of 2 × 10(6) hMSCs/kg for 4 weeks, with an additional 4 weekly infusions after day +28 for patients who achieved either a partial or mixed response. The enrolled patients compose a very challenging population with severe disease that was nonresponsive to the standard of care, with 88% of the patients experiencing severe aGVHD (grade C or D). Seventy-five patients (median age, 8 yr; 58.7% male; and 61.3% Caucasian) were treated in this study. Sixty-four patients (85.3%) had received an unrelated hematopoietic stem cell graft, and 28 patients (37.3%) had received a cord blood graft. At baseline, the distribution of aGVHD grades B, C, and D was 12.0%, 28.0%, and 60.0%, respectively. The median duration of aGVHD before enrollment was 30 d (range, 2 to 1639 d), and patients failed a median of 3 immunosuppressive agents. Organ involvement at baseline was 86.7% gastrointestinal, 54.7% skin, and 36.0% liver. Thirty-six patients (48.0%) had 2 organs involved, and 11 patients (14.7%) had all 3 organs involved. When stratified by aGVHD grade at baseline, the rate of overall response (complete and partial response) at day +28 was 66.7% for aGVHD grade B, 76.2% for grade C, and 53.3% for grade D. Overall response for individual organs at day +28 was 58.5% for the gastrointestinal system, 75.6% for skin, and 44.4% for liver. Collectively, overall response at day +28 for patients treated for severe refractory aGVHD was 61.3%, and this response was correlated with statistically significant improved survival at day +100 after hMSC infusion. Patients who responded to therapy by day +28 had a higher Kaplan-Meier estimated probability of 100-d survival compared with patients who did not respond (78.1% versus 31.0%; P < 0.001). Prochymal infusions were generally well tolerated, with no evidence of ectopic tissue formation [190].

A study reported the first experience using third party bone marrow MSCs to treat refractory aGVHD in 33 pediatric patients undergoing allogeneic HSCT. Totally, 68 doses of bone marrow derived MSCs were infused. The median dose of MSC was 1.18 × 10(6) cells per kg body weight. Overall, complete response (CR) was documented in 18 patients, partial response (PR) was documented in 7 patients, and no response (NR) was documented in 8 patients. The 2-year estimated probability of overall survival (OS) for patients achieving CR and PR/NR was 63.8% and 29.4%, respectively (p = 0.0002). While the cumulative incidence of transplant related mortality (TRM) at day 100 after first MSC infusion was 46.6% in PR/NR patients, there was no any TRM at day 100 after first MSC infusion in CR patients (p = 0.001). Twelve patients developed chronic GVHD (cGVHD); eight of them were alive, with five having extensive disease and three having limited disease. In conclusion, MSCs appear to be safe and effective treatment option for pediatric patients with steroid refractory aGVHD [191].

Another study assessing adult and pediatric patients assessed the feasibility and safety of intravenous administration of third party bone marrow-derived mesenchymal stromal cells (MSC) expanded in platelet lysate in 40 patients (15 children and 25 adults), experiencing steroid-resistant grade II to IV graft-versus-host disease (GVHD). Patients received a median of 3 MSC infusions after having failed conventional immunosuppressive therapy. A median cell dose of 1.5 × 10(6)/kg per infusion was administered. No acute toxicity was reported. Overall, 86 adverse events and serious adverse events were reported in the study, most of which (72.1%) were of infectious nature. Overall response rate, measured at 28 days after the last MSC injection, was 67.5%, with 27.5% complete response. The latter was significantly more frequent in patients exhibiting grade II GVHD as compared with higher grades (61.5% versus 11.1%, P = 0.002) and was borderline significant in children as compared with adults (46.7 versus 16.0%, P = 0.065). Overall survival at 1 and 2 years from the first MSC administration was 50.0% and 38.6%, with a median survival time of 1.1 years. In conclusion, MSC can be safely administered on top of conventional immunosuppression for steroid resistant GVHD treatment [192].

Another use of MSC in GVHD has been to add the cells as an adjuvant to standard corticosteroid therapy. Patients with grades II-IV GVHD were randomized to receive 2 treatments of human MSCs (Prochymal(R)) at a dose of either 2 or 8 million MSCs/kg in combination with corticosteroids. Patients received GVHD prophylaxis with tacrolimus, cyclosporine, (CsA) or mycophenolate mofetil (MMF). Study endpoints included safety of Prochymal administration, induction of response to Prochymal, and overall response of aGVHD by day 28, and long-term safety. Thirty-two patients were enrolled, with 31 evaluable: 21 males, 10 females; median age 52 years (range: 34-67). Twenty-one patients had grade II, 8 had grade III, and 3 had grade IV aGVHD. Ninety-four percent of patients had an initial response to Prochymal (77% complete response [CR] and 16% partial response [PR]). No infusional toxicities or ectopic tissue formations were reported. There was no difference with respect to safety or efficacy between the low and high Prochymal dose. In conclusion, Prochymal can be infused safely into patients with aGVHD and induces response in a high proportion of GVHD patients [193].

Types of GVHD that are resistant to treatment include sclerodermous GVHD. A publication described 4 patients with sclerodermatous GVHD (ScGVHD) who received MSCs expanded ex vivo from unrelated donors by intra-BM injection. After MSC infusion, the ratio of helper T lymphocyte (Th) 1 cells to Th2 cells was dramatically reversed, with an increase in Th1 and a decrease in Th2 achieving a new balance. Correspondingly, symptoms gradually improved in all 4 patients. During the course of MSC treatment, the patients’ vital signs and laboratory results remained normal. At the time of this report, none of the 4 patients had experienced recurrence of leukemia. Although this study alone cannot guarantee the application of MSCs in ScGVHD, our findings strongly suggest that this treatment is therapeutically practicable, with no detectable side effects. This approach may provide new insight into the clinical treatment of ScGVHD, with the aim of greatly increasing the survival rate in patients with leukemia who undergo allogeneic BM transplantation [194]. Complications of GVHD are known to occur. This was addressed in an interesting case series in which ten consecutive patients, treated with MSC due to tissue toxicity following allogeneic hematopoietic stem cell transplantation, (ASCT) were included. Their median age was 48 (13-64) years. Seven had hemorrhagic cystitis grades 2-5, two had pneumomediastinum and one had perforated colon and peritonitis. MSC donors were mainly third-party, HLA-mismatched (n=11), HLA-haploidentical (n=3) and, in two cases, the HLA-identical ASCT sibling donors. MSC were given intravenously, the median cell dose was 1.0 (range 0.7-2)x10(6)/kg. In five patients, the severe hemorrhagic cystitis cleared after MSC infusion. Gross hematuria disappeared after median 3 (1-14) days. Two patients had reduced transfusion requirements after MSC infusion, but died of multiorgan failure. In one of them, MSC donor DNA was demonstrated in the urinary bladder. In two patients, pneumomediastinum disappeared after MSC infusions. A patient with steroid-resistant graft-versus-host disease of the gut experienced perforated diverticulitis and peritonitis that was reversed twice by MSC. MSC is a novel treatment for therapy-induced tissue toxicity [195].

Dry eye is another completion of GVHD. A total of 22 patients with refractory dry eye secondary to cGVHD were enrolled. The symptoms of 12 out of 22 patients abated after MSCs transplantation by intravenous injection, improving in the dry eye scores, ocular surface disease index scores and the Schirmer test results. The clinical improvements were accompanied by increasing level of CD8(+)CD28(-) T cells, but not CD4(+)CD25(+) T cells, in the 12 patients who were treated effectively. They had significantly higher levels of Th1 cytokines (interleukin (IL)-2 and interferon-y) and lower levels of Th2 cytokines (IL-10 and IL-4). In addition, CD8(+) T cells were prone to differentiation into CD8(+)CD28(-) T cells after co-culture with MSCs in vitro. In conclusion, transfusion of MSCs improved the clinical symptoms in patients (54.55%) with refractory dry eye secondary to cGVHD. MSCs appear to exert their effects by triggering the generation of CD8(+)CD28(-) T cells, which may regulate the balance between Th1 and Th2 [196]. The use of adipose MSC was reported in 43-year-old woman with chronic hepatic graft-versus-host disease who failed previous immunosuppressive therapy with cyclosporine and prednisone was treated with tacrolimus starting on day 165 after allogeneic hematopoietic stem cell transplantation. Fifteen days later, tacrolimus was discontinued because of progressive deterioration of renal function. However, after changing treatment to human adipose tissue-derived mesenchymal stem cells (AMSC), we observed rapid and complete resolution of hepatic GVHD and renal toxicity. We concluded that it is worthwhile to administer AMSC as a treatment for common hepatic GVHD, particularly for atypical cases presenting as acute hepatitis [197]. Another paper from the same group reported two pediatric patients who developed severe refractory acute GVHD following ASCT and were successfully treated with AMSC from HLA-mismatched unrelated donors [198]. In addition to GVHD, severe late-onset hemorrhagic cystitis (LO-HC) is another adverse effect of allogeneic hematopoietic stem cell transplantation. Investigators retrospectively analyzed the efficacy and safety of allogeneic MSC infusions in 7 of 33 patients with severe LO-HC after allogeneic HSCT. During treatment, each patient received at least one MSC infusion of Wharton’s jelly derived from the umbilical cord of a third-party donor. In 6 patients, MSC treatment was initiated within 3 days of gross hematuria onset, while the 7th patient received an infusion 40 days later. The median dose was 1.0 (0.8-1.6) × 10(6)/kg. Five of 7 patients responded to treatment. Notably, gross hematuria promptly disappeared in 3 patients after 1 infusion, with a time to remission not seen in patients without MSC infusion. Two patients showed no response even after several infusions. No acute or late complications were recorded. Our findings indicate that MSC transfusion might be a feasible and safe supplemental therapy for patients with severe LO-HC after allogeneic hematopoietic stem cell transplantation [199].

A study assessed cotransplantation of culture-expanded third-party donor-derived umbilical cord MSCs (UC-MSCs) in 50 people with refractory/relapsed hematologic malignancy undergoing haplo-HSCT with myeloablative conditioning. They observed that all patients given MSCs showed sustained hematopoietic engraftment without any adverse UC-MSC infusion-related reaction. The median times to neutrophil >0.50 × 10(9)/L and platelet >20 × 10(9)/L engraftment were 12.0 and 15.0 days, respectively. No increase in severe acute GVHD (aGVHD) and extensive chronic GVHD (cGVHD), was seen. Grade II-IV aGVHD was observed in 12 of 50 (24.0 %) patients. cGVHD was observed in 17 of 45 (37.7 %) patients and was extensive in 3 patients. Additionally, only five patients (10.0 %) experienced relapse at a median time to progression of 192 days. The probability that patients would attain progression-free survival at 2 years was 66.0 %. The results indicate that this new strategy is effective in improving donor engraftment and reducing severe GVHD, which will provide a feasible option for the therapy of high-risk hematologic malignancy [200].

A study was performed to investigate the curative effect of third-party umbilical cord blood-derived human MSCs (UCB-hMSCs) on GVHD patients after allogeneic hematopoietic stem cell transplantation (allo-HSCT) and their immune regulatory mechanism. Twenty-four refractory GVHD patients after allo-HSCT were treated with UCB-hMSCs. Immune cells including T lymphocyte subsets, NK cells, Treg cells and dendritic cells (DCs) and cytokines including interleukin-17 (IL-17) and tumor necrosis factor-alpha (TNF-α) were monitored before and after MSCs transfusion. The results showed that the symptoms of GVHD were alleviated significantly without increased relapse of primary disease and transplant-related complications after MSCs transfusion. The number of CD3(+), CD3(+)CD4(+) and CD3(+)CD8(+) cells decreased significantly, and that of NK cells remained unchanged, whereas the number of CD4(+) and CD8(+) Tregs increased and reached a peak at 4 weeks; the number of mature DCs, and the levels of TNF-α and IL-17 decreased and reached a trough at 2 weeks. It was concluded that MSCs ameliorate GVHD and spare GVL effect via immunoregulation [201].

A multicenter, double-blind, randomized controlled trial, we investigated the incidence and severity of cGVHD among patients, and the changes in T, B, and natural killer (NK) cells after the repeated infusion of umbilical cord MSC. The 2-year cumulative incidence of cGVHD in the MSCs group was 27.4% (95% CI, 16.2% to 38.6%), compared with 49.0% (95% CI, 36.5% to 61.5%) in the non-MSCs control group (P = 0.021). Seven patients in the non-MSCs control group had severe lung cGVHD, but no patients in the MSCs group developed typical lung cGVHD (P = 0.047). After the MSC infusions, increasing memory B lymphocytes and regulatory T cells, as well as the ratio of type 1 T helper to type 2 T helper cells, were observed, whereas the number of NK cells decreased. Our findings suggest that the repeated infusion of umbilical cord MSCs might inhibit cGVHD symptoms in patients after HLA-haplo hematopoietic stem cell transplant, accompanied by changes in the numbers and subtypes of T, B, and NK cells, leading to the acquisition of immune tolerance [202]. A phase I trial using third party, early passage BMSCs for patients with steroid-refractory GVHD, tissue injury, or marrow failure following SCT to investigate safety and efficacy was performed. To identify mechanisms of BMSC immunomodulation and tissue repair, patients were serially monitored for plasma GVHD biomarkers, cytokines, and lymphocyte phenotype. Ten subjects were infused a fixed dose of 2 × 10(6) BMSCs/kg intravenously weekly for three doses. There was no treatment-related toxicity (primary endpoint). Eight subjects were evaluable for response at 4 weeks after the last infusion. Five of the seven patients with steroid-refractory acute GVHD achieved a complete response, two of two patients with tissue injury (pneumomediastinum/pneumothorax) achieved resolution but there was no response in two subjects with delayed marrow failure. Rapid reductions in inflammatory cytokines were observed. Clinical responses correlated with a fall in biomarkers (Reg 3α, CK18, and Elafin) relevant for the site of GVHD or tissue injury. The GVHD complete responders survived significantly longer and had higher baseline absolute lymphocyte and central memory CD4 and CD8 counts. Cytokine changes also segregated with survival. These results confirm that BMSCs are associated with rapid clinical and biomarker responses in GVHD and tissue injury. However, BMSCs were ineffective in patients with prolonged GVHD with lower lymphocyte counts, which suggest that effective GVHD control by BMSCs requires a relatively intact immune system [203].

Another study investigating potential markers associated with remission from GVHD by MSC assessed the numbers, phenotypes, and subpopulations of B lymphocytes in cGVHD patients who showed a complete response (CR), partial response (PR), or no response (NR) after MSC treatment. We found that the frequencies and numbers of CD27+ memory and pre-germinal center B lymphocytes were significantly increased in the CR and PR cGVHD patients after MSC treatment but decreased in the NR patients. A further analysis of CR/PR cGVHD patients showed that MSC treatment led to a decrease in the plasma levels of B cell-activating factor (BAFF) and increased expression of the BAFF receptor (BAFF-R) on peripheral B lymphocytes but no changes in plasma BAFF levels or BAFF-R expression on B lymphocytes in NR patients. Overall, these findings imply that MSCs might exert therapeutic effects in cGVHD patients, accompanied by alteration of naive and memory B-cell subsets, modulating plasma BAFF levels and BAFF-R expression on B lymphocytes [204].

Further supporting a possible role of B cell modulation by MSC was a prospective clinical study, 20 of 23 cGVHD patients that had a complete response or partial response to MSC therapy in a 12-month follow-up study. The most marked improvements in cGVHD symptoms were observed in the skin, oral mucosa and liver. Clinical improvement was accompanied by a significantly increased number of interleukin (IL)-10-producing CD5+ B cells. Importantly, CD5+ B cells from cGVHD patients showed increased IL-10 expression after MSCs treatment, which was associated with reduced inflammatory cytokine production by T cells. Mechanistically, MSCs could promote the survival and proliferation of CD5+ regulatory B cells (Bregs), and indoleamine 2, 3-dioxygenase partially participates in the MSC-mediated effects on Breg cells. Thus, CD5+ Breg cells may have an important role in the process of MSC-induced amelioration of refractory cGVHD and may provide new clues to reveal novel mechanisms of action for MSCs [205].

The possibility of T regulatory cells mediating tolerogenic responses in GVHD after MSC administration was explored in a clinical study. Forty-seven patients with refractory aGVHD were enrolled: 28 patients receiving third party bone marrow derived MSC and 19 patients without MSC treatment. MSCs were given at a median dose of 1 × 10(6) cells/kg weekly until patients got complete response or received 8 doses of MSCs. After 125 doses of MSCs were administered, with a median of 4 doses (range, 2 to 8) per patient, overall response rate was 75% in the MSC group compared with 42.1% in the non-MSC group (P = 0.023). The incidence of cytomegalovirus, Epstein-Barr virus infections, and tumor relapse was not different between the 2 groups during aGVHD treatment and follow-up. The incidence and severity of chronic GVHD in the MSC group were lower than those in the non-MSC group (P = 0.045 and P = 0.005). The ratio of CD3(+)CD4(+)/CD3(+)CD8(+) T cells, the frequencies of CD4(+)CD25(+)Foxp3(+) regulatory T cells (Tregs), and the levels of signal joint T cell-receptor excision DNA circles (sjTRECs) after MSCs treatment were higher than those pretreatment. MSC-treated patients exhibited higher Tregs frequencies and sjTRECs levels than those in the non-MSC group at 8 and 12 weeks after treatment. MSCs derived from bone marrow of a third-party donor are effective to refractory aGVHD. It might reduce the incidence and severity of chronic GVHD in aGVHD patients by improving thymic function and induction of Tregs but not increase the risks of infections and tumor relapse [206].

In another study, genes on the MSC were evaluated for correlation to suppression of GVHD. A randomized study was performed on 77 patients. They were randomized into two groups - those receiving standard prophylaxis of aGVHD and those who were additionally infused with MMSC derived from the bone marrow of hematopoietic stem cell donors. It was found that the infusion of MMSC halves the incidence of aGVHD and increases the overall survival of patients. Four of 39 MMSC samples were ineffective for preventing aGVHD. Analysis of individual donor characteristics (gender, age, body mass index) and the MMSC properties of these donors (growth parameters, level of expression of 30 genes involved in proliferation, differentiation, and immunomodulation) revealed no significant difference between the MMSC that were effective or ineffective for preventing aGVHD. The authors used multiple logistic regression to establish a combination of features that characterize the most suitable MMSC samples for the prevention of aGVHD. A model predicting MMSC sample success for aGVHD prophylaxis was constructed. Significant model parameters were increased relative expression of the FGFR1 gene in combination with reduced expression levels of the PPARG and IGF1 genes. Depending on the chosen margin for probability of successful application of MMSC, this model correctly predicts the outcome of the use of MMSC in 82-94% of cases. The proposed model of prospective evaluation of the effectiveness of MMSC samples will enable prevention of the development of0 aGVHD in the maximal number of patients [207]. In another study, assessment of MSC properties that correlate with ability to inhibit GVHD were explored by quantifying growth parameters of MSCs and the relative expression levels (REL) of different regeneration/immunology associated genes. Specifically, in a clinical trial of allogeneic hematopoietic stem cell transplants MSCs infusion induced a significant threefold decrease in aGvHD development and improved overall survival compared with the standard prophylaxis group. In ineffective MSC samples (9.4%), a significant decrease in total cell growth and the REL of CSF1, FGFR1, and PDGFRB was observed [208].Given the immune suppressive role of MSC, as well as the fact that GVHD is associated graft versus leukemia, and MSC suppress GVHD, it would seem logical that MSC may increase risk of leukemic relapse post-transplant. This fear was first highlighted in a paper by Ning et al. who performed an open-label randomized clinical trial in which HLA-identical sibling-matched hematopoietic stem cells (HSC) were transplanted (non-MSCs group, n=15) or cotransplanted with mesenchymal stem cells (MSCs) (MSCs group, n=10) in hematologic malignancy patients. The median number of MSCs infused was 3.4 × 10(5) kg(-1) (range, 0.3-15.3 x 10(5) kg(-1)). MSCs infusions were well tolerated. The median time to neutrophil engraftment (absolute neutrophil count >0.5 × 10(9) 1(-1)) was 16 days for MSCs group and 15 days for non-MSCs group. The median time to platelet engraftment (platelet count >50 × 10(9) 1(-1)) was 30 and 27 days, respectively. Grades II-IV acute graft-versus-host disease (GVHD) was observed respectively, in one (11.1%) and eight (53.3%) evaluable patients. Chronic GVHD was found in one (14.3%) and four (28.6%) evaluable patients. The number of patients who relapsed were six (60.0%) and three (20.0%), and the 3-year disease-free survivals were 30.0 and 66.7%, respectively. Thus cotransplantation of MSCs and HSCs may prevent GVHD, but the relapse rate is obviously higher than the control group [209].

Another study examined 31 patients treated with mesenchymal stromal cells (MSCs) for acute graft-versus-host disease (aGVHD) or hemorrhagic cystitis between 2002 and 2007, who were followed to investigate predictors of outcome, immunologic effects in vivo, and long-term survival. There was no correlation between in vitro suppression by MSCs in mixed lymphocyte cultures and outcome. Soluble IL-2 receptors were measured in blood before and after MSC infusion and declined significantly during the first week after MSC infusion (P = 0.03). Levels of interleukin-6 and HLA-G were unaffected. Infectious complications occurred several years after recovery from aGVHD. Cytomegalovirus viral load was high, and cytomegalovirus disease was common. Among patients recovering from aGVHD, 54% died of late infections, between 4 months and 2 years after MSC treatment. No increase in leukemia relapse or graft rejection was found. Children had a better survival rate than adults (P = 0.005). In GVHD patients, 1-year survival was 75% in patients who received early-passage MSCs (from passages 1-2) in contrast to 21% using later passage MSCs (from passages 3-4) (P < 0.01). The authors concluded that treatment with early-passage MSCs improved survival in patients with therapy-resistant GVHD. Death from infection was common in MSC-treated patients, but there was no increase in leukemia relapse [210].

Another study assessed ability of MSC to reduce GVHD versus suppression of graft versus leukemia (GVL). It was tested whether MSC infusion before HCT could allow nonmyeloablative (NMA) HCT (a transplant strategy based nearly exclusively on graft-versus-tumor effects for tumor eradication) from HLA-mismatched donors to be performed safely. Twenty patients with hematologic malignancies were given MSCs from third party unrelated donors 30-120 minutes before peripheral blood stem cells (PBSCs) from HLA-mismatched unrelated donors, after conditioning with 2 Gy total body irradiation (TBI) and fludarabine. The primary endpoint was safety, defined as a 100-day incidence of nonrelapse mortality (NRM) <35%. One patient had primary graft rejection, whereas the remaining 19 patients had sustained engraftment. The 100-day cumulative incidence of grade II-IV acute GVHD (aGVHD) was 35%, whereas 65% of the patients experienced moderate/severe chronic GVHD (cGVHD). One-year NRM (10%), relapse (30%), overall survival (OS) (80%) and progression-free survival (PFS) (60%), and 1-year incidence of death from GVHD or infection with GVHD (10%) were encouraging. These figures compare favorably with those observed in a historic group of 16 patients given HLA-mismatched PBSCs (but no MSCs) after NMA conditioning, which had a 1-year incidence of NRM of 37% (P = 0.02), a 1-year incidence of relapse of 25% (NS), a 1-year OS and PFS of 44% (P = 0.02), and 38% (P = 0.1), respectively, and a 1-year rate of death from GVHD or infection with GVHD of 31% (P = 0.04). In conclusion, these data suggest that HLA-mismatched NMA HCT with MSC coinfusion appeared to be safe without diminishing of GVL [211].

In one embodiment, cells of the invention are utilized to treat inflammation associated diseases. In one particular embodiment, cells are utilized with the concept of treating heart conditions. Chronic inflammation both locally and systemically is part of the cascade leading to heart failure. Acute inflammation occurs during infarction as a result of tissue damage; however, chronic inflammatory markers are present in post-infarct patients, as well as ischemic heart failure patients, and patients with congenital defects. In general, a positive correlation between advanced heart failure and levels of the inflammatory marker, the pentraxin C-reactive protein (CRP) has been reported [212, 213]. While CRP elevation is conventionally seen as a marker of ongoing inflammation, produced by the liver in response to cytokines such as IL-1, IL-6, and TNF-alpha [214], it also plays an active role in cardiac deterioration through induction of endothelial dysfunction [215, 216], as well as exacerbation of inflammatory processes through activation of complement [217, 218]. In addition to CRP, elevated levels of inflammatory cytokines are also noted in CHF patients [219]. Inflammatory mediators are produced not only as a result of cardiomyocyte ischemia, but also stretch injury as a result of hypertrophic accommodation [220, 221] and systemic activation of immune cells including T cells [222] and monocytes [223]. Functionally, inflammatory mediators induce direct apoptosis of cardiomyocytes. For example TNF-alpha is known to induce reduction of bcl-2 gene expression and activate a caspase-dependent apoptosis in cardiac cells at physiological concentrations [224]. Reduction of TNF-alpha activity using soluble receptors has demonstrated beneficial effects in animal models of heart failure [225]. Accordingly, in some embodiments of the invention, administration of cells of the invention is guided by reduction of inflammation observed subsequent to administration. Assessment of inflammation can be determined using markers known in the art, and also markers discussed herein such as CRP. The importance of inflammatory stimuli in heart failure can be seen in animal models in which activators of inflammatory agents, such as toll-like receptors (TLRs) are knocked-out. Generally, TLRs particularly TLR 2 and 4, recognize endogenous “danger signals” associated with damaged tissue such as extracellular matrix degradation products [226, 227], and heat shock proteins [228]. Doxorubicin induced heart failure is substantially attenuated in animals lacking TLR-2 [229] or TLR-4 [230]. TLR-2 knockout mice have a substantially better clinical outcome after experimental infarction, including reduction in remodeling, wall thinning, and preservation of LVEF as compared to wild-type controls [231]. Clinically, expression of TLR-4 is associated with poor prognosis in post-infarct patients [232]. In some embodiments of the invention, the administration of cells of the invention is performed so as to decrease TLR signalling. In other embodiments, cells of the invention are used in combination with other types of stem cells. Other stem cells including MSC-like populations that have been isolated from a diverse range of sources such as adipose [233], heart [234], Wharton’s Jelly [235], dental pulp [236], peripheral blood [237], cord blood [238], and more recently menstrual blood [239-241]. In some embodiments are stimulated to have potent anti-inflammatory activities which appear to be present regardless of tissue of origin [242, 243] through exposure to mild inflammatory stimuli such as CpG motifs. Mechanistically, MSC appear to suppress inflammation through secretion of anti-inflammatory cytokines such as IL-10 [244], TGF-beta [245], LIF [246], soluble HLA-G [47] and IL-1 receptor antagonist [247], expression of immune regulatory enzyme such as cycloxygenase [248] and indolamine 2,3 deoxygenase [249], and ability to induce generation of anti-inflammatory T regulatory cells [46]. The in vivo anti-inflammatory effects of MSC may be witnessed by success in treating animal models of immune mediate/inflammatory pathologies such as multiple sclerosis [250], colitis [251], graft versus host [252], rheumatoid arthritis [253], and ischemia/reperfusion injury [254]. In heart failure administration of MSC post infarct has been demonstrated to decrease production of TNF-alpha and IL-6, but upregulate generation of the anti-inflammatory cytokine IL-10, which correlated with therapeutic benefit [255]. Clinically, MSC have demonstrated repeatedly potent therapeutic activity at suppressing graft versus host (GVHD), for which Phase III FDA-registration trials are currently ongoing [175-177, 209, 256, 257].

In one embodiment of the invention, utility of the disclosed stem cells to prevent cardiomyocyte apoptosis is described. Cardiomyocyte death, either by apoptosis [258], or other types of death such as autophagy and programmed necrosis is part of the self-perpetuating cascade leading to heart failure [259, 260]. Thus the manipulation of these death pathways and upregulation of endogenous repair mechanisms in the heart could be a possible method of decreasing the progression to heart failure. For example, suppression of apoptotic machinery such as the transgenic expression of a dominant negative form of Mammalian sterile 20-like kinase-1 (Mst1) inhibits post infarct remodeling, through suppression of cardiomyocyte death [261], similar protective effects can be attained by transfection of anti-apoptotic genes such as IAP-2 [262]. Transfection of genes such as hepatocyte growth factor (HGF-1) into cardiac cells has also been demonstrated to reduce progression to heart failure in animal models [263]. ACE inhibitors have been postulate to have some beneficial effects through inhibition of cardiomyocyte apoptosis [264]. Thus one attractive method of addressing the progression to heart failure would be identification of methods to prevent ongoing cell death. Cell death in the heart causes some level of replacement by resident cardiac stem cells (CSC). In one embodiment of the invention, cells of the invention are utilized to activate endogenous cardiac stem cells. These cells are relatively rare and are believed to respond to signals associated with damage to the myocardium. Fransioli et al generated a transgenic mouse expressing GFP under control of the c-kit promoter. Subsequent to infarct, increased proliferation of c-kit positive cells was seen in the myocardium [265]. In humans Urbanek et al examined 20 human hearts from patients who died after acute infarct, 20 hearts with chronic infarct that were transplanted and 12 control hearts. A population of cells expressing c-kit, MDR1 and Sca-1 were seen to enter cell cycle from a basal rate of 1.5% cycling cells in controls, to 28% and 14% in acute and chronic infarcts, respectively. The cells expressing the phenotype were demonstrated to be capable of differentiating into myocyte, smooth muscle, and endothelial cell lineages [266]. Isolated CSC have been successfully expanded ex vivo and administered via the intracoronary route in rats post-infarct. Successful transmigration of the CSC across the endothelium and active regeneration of myocardium was demonstrated [267]. Thus it appears that a functional population of stem cells exists in the heart that can, to some extent cause regeneration post injury.

Both hematopoietic stem cells (HSC) and MSC are capable of secreting factors that on the one hand inhibit apoptosis [268-270] and on the other hand stimulate activation of CSC [267]. Accordingly, in one embodiment of the invention, administration of cells of the invention is performed to enhance activation and regenerative ability of HSC and/or other MSC. For example, it was demonstrated that administration of non-fractionated bone marrow cells containing both cell populations protects against apoptosis in a doxorubin induced cardiomyopathy model [271]. Furthermore bone marrow cells are known to produce HGF [272] and IGF-1 [273] that are anti-apoptotic and activate endogenous cardiomyocyte stem cells [274] have been reported. Interestingly production of these factors is upregulated in response to inflammatory mediators associated with heart failure such as TNF-alpha [270]. Therefore it may be possible to believe that MSC not only migrate to injured tissue but can “sense” inflammatory stimuli such as TNF-alpha and actually try to grade the level of their therapeutic response according to the level of damage sensed. Another means by which stem cells may repair the heart is through actually differentiating into new heart muscle. Reports exist of both hematopoietic [275] and mesenchymal stem cells [276, 277] differentiating into cardiac-like cells, although this is controversial and some groups have reported this to be a product of cell fusion [278]. Additionally, stem cells promote angiogenesis, thus providing nutrients to ischemic areas and potentially allowing regeneration [279, 280].

The cells of the invention possess numerous activities. In one embodiment, said cells are utilized for the purpose of replacing dead or deficient cardiac muscle with injected therapeutic stem cells might augment contractile function in the heart[281]. Various experimental studies provided evidence that the infusion or injection of stem or progenitor cells may reduce scar formation and fibrosis. The most promising approach for regeneration of necrotic myocardium is cell transplantation; the implantation of muscle cells, progenitor cells, or pluripotent stem cells in infarcted myocardium has been reported by many investigators [281-285] Initial encouraging results from animal studies [286-289] and several small, uncontrolled clinical trials, [290-292] have prompted scientists and clinicians to focus more on critical evaluation of the scientific basis for myocyte regeneration and the efficacy of such proposed cell therapy in clinical practice. Moreover, Li et al. have showed in cryoinjury-induced myocardial infarction in the rat, that the intramyocardial injection of fetal cardiomyocytes improved systolic and diastolic function up to two months after transplantation, as assessed by ex vivo Langendorff perfusion studies[293]. In a reperfused infarcted area in rats, fetal cardiomyocytes were intramyocardially implanted and, one month later, function was found to be significantly improved in transplanted animals, compared with controls[294]. Furthermore, in a mouse model of anthracycline induced toxic cardiomyopathy, it was possible to show that injected fetal cardiomyocytes also improved cardiac function one month after transplantation, as compared with control nontransplanted animals indicating cellular transplantation could effectively improve function of ischemically damaged myocardium.

However, issues relating to availability, ethical problems regarding the fetal source of myocytes and the necessity for immunosuppressive therapy limit the potential clinical application of this allograft technique. In the search of alternative cellular types, investigators have refocused on the clinical use of other cell types such as skeletal myoblasts marrow-derived stromal cells and peripheral blood derived precursor cells to be used in cell therapy for cardiac ischemia.

In some embodiments of the invention, cells of the invention are administered to allow enhanced activity of myocytes. In some embodiments cells of the invention provide an immune modulatory activity which allows for myocytes to be transplanted in an allogeneic and/or xenogeneic manner, with minimal or no immune suppression.. The precursors of skeletal muscle fibers, the myoblasts, present in adult animals as quiescent cells and may become activated, proliferate and differentiate upon muscle injury (in vivo) or following tissue dissociation (in vitro) in culture. Working on primary myoblast transplantations in a cryo-injury model of myocardial infarction in dogs, Chiu et al. (1995) were able to characterize the donor cells in the myocardium 14 weeks after injection[295]. Murry et al. (1996) observed the formation of myotubes and skeletal muscle fibers within the cardiac tissue but they were unable to identify cardiac specific markers within the tissue formed by the injection of myoblasts[296]. Taylor et al. (1998) assessed transplanted myoblasts in a model of rabbit heart cryo-infarction and observed a functional improvement of the skeletal muscle cells within the scar tissue following transplantation by sonomicrometry[285]. More importantly, in this study, the functional improvement was only seen in those animals in which implanted cells were histologically identified, thereby bringing strong evidence for a causal relationship between the presence of engrafted cells and the functional outcome. On the other hand, Murry et al. (1996) could not identify changes in the myoblast transplanted phenotype[296]. In a myocardial infarction model in rats created by coronary artery ligation, Scorsin et al. (1998, 2000) showed that one month after myoblast transplantation the treated group displayed a significant improvement of function, primarily manifested as a limitation of post infarction ventricular remodeling, compared with non-transplanted controls[294, 297]. No gap-junction was detected between transplanted cells as demonstrated by the negative staining for connexin-43. Further experimental studies have shown that when implanted into post-infarction scar tissue, skeletal myoblasts (satellite cells) improved the left ventricular hemodynamic parameters, including contractile function[297]. The results of experimental studies [298] [299]have encouraged Menasché et al. [300] to perform the first autologous skeletal myoblast transplantation in a patient, a survivor of myocardial infarction, during CABG. This was shortly followed by a similar procedure by Siminiak et al. [301] and many other investigators[286, 302].Despite promising results in animal models and initial human studies, skeletal myoblast transplantation into damaged myocardium remains an unproven technology. A number of technical issues remain unresolved, including optimum cell type, ideal number of cells, factors that promote engraftment, surgical delivery method and patient selection criteria. Several studies are geared towards addressing these issues by many investigators [286-289] and several encouraging small, uncontrolled clinical trials [290-292]. In 2007, Genezyme in association with Medtronic’s MAGIC clinical trial on evaluation of skeletal myoblast transplant for treating ischemic heart failure was stopped due to lack of efficacy. The long-term viability and functionality of the transplanted cells has not been proven. Additionally, none of the studies have demonstrated an improvement in patient functional status or survival. Thus, transplantation of skeletal myoblasts remains experimental for the treatment of damaged myocardium. The above studies clearly demonstrate that skeletal myoblasts does not fulfill the major criteria required for a true cardiac regeneration: a coupling of the grafted cells with those of the recipient myocardium and the subsequent generation of a contractile force. These observations provide strong rationale to explore alternative cell populations, among which marrow-derived stem cells are particularly attractive.

Cardiomyocytes formation from circulating BM cells has been first demonstrated by Bittner et al.[303]. Goodel’s group then demonstrated that in infarcted hearts of adult mice, cardiomyocytes and vascular cells can be generated from stem cell population isolated from mouse BM cells, termed side population (SP) cells,[304] . The BM-derived SP cells that express a hematopoietic stem cell antigen Sca-1 and a VEGF receptor Flt-1 have a capacity to form cardiomyocytes and vascular cells in damaged hearts, whereas the exact origin for these cells in the SP compartment has not been confirmed yet[305]. Cardiomyocytes are also formed after direct injection of BM derived cells such as hematopoietic lineage marker-negative (Lin⁻) and c-kit⁺ (the receptor for stem cell factor) similar to HSCs [306]or MSCs [307] in to damaged heart tissue. Recent work documented that there is a close relationship between hematopoietic stem cells (HSCs) and Endothelial Progenitor Cells (EPC) [308], as well as between mesenchymal stem cells (MSCs) and EPCs[309, 310]. Recently, implantation of BM mononuclear cells was shown to provide angiogenesis and enhance regional function of the ischemic myocardium in the pig. Despite the very interesting and promising option to restore myocardial viability, the use of BM stem cells raises two important questions. Firstly, they have not yet shown the potential to multiply and produce a very large number of differentiated cells in vitro, the first condition to fully recolonize diseased myocardium and thus improving ventricular function. Secondly, there is a risk of developing other types of tissues including severe calcification [311] if undifferentiated BM stem cells are used.

One of the original descriptions of clinical stem cell use in the cardiac space was Strauer et al who reported a case in which a 46 year old patient received autologous bone marrow mononuclear cells by a percutaneous transluminal catheter placed in the infarct-related artery. 10 weeks after administration the transmural infarct area had been reduced from 24.6 % to 15.7 % of left ventricular circumference, while ejection fraction, cardiac index and stroke volume had increased by 20-30 % [312]. A subsequent paper in the same year reported administration of similar cells in 5 patients with advanced ischemia undergoing coronary artery bypass grafting. Cells were administered intramuscularly into areas deemed ungraftable and perfusion was assessed by imaging. Specific improvement in areas injected was documented in 3 of the 5 patients. Perhaps more importantly, no ectopic growths or adverse effects were reported at 1 year follow-up [313]. Since these pioneering studies, cardiac stem cell therapy has been used by numerous groups for numerous conditions causing heart failure. These can be broken down into: a) inhibiting post acute myocardial infarction remodeling; b) stimulation of regeneration in chronically injured hearts and c) induction of angiogenesis in coronary artery disease. The methods of administering stem cells have included the intracoronary, epicardial, and intravenous routes. Stem cells used to date are bone marrow mononuclear cells, mobilized peripheral blood stem cells, purified CD34 or CD133 cells, autologous mesenchymal stem cells, and allogeneic bone marrow and placental mesenchymal stem cells.

Several meta-analysis of ongoing clinical trials performed indicated that both hematopoietic and mesenchymal cells have promising clinical effects in various types of heart failure. Briefly, Abdel-Latif et al described 999 patients enrolled 18 independent controlled cardiac trials in which patients were treated with either unfractionated bone marrow cells, bone marrow mesenchymal, or mobilized peripheral blood [314]. They found that in comparison to controls, there was a statistically significant improvement in ejection fraction, reduction in infarct size and left ventricular end-systolic volume. Importantly, no safety issues or serious treatment associated adverse events were noted. In another such comprehensive review, Martin-Rendon et al focused on bone marrow therapy for post acute infarction trials. Of 13 randomized studies conducted, encompassing 811 participants, the authors of the review stated that more trials are needed to establish efficacy in terms of clinical endpoints such as death. However that authors of the review did observe a consistent improvement in LVEF, as well as trends for decrease in left ventricular end systolic and end diastolic volumes, and infarct size [315]. Three other meta-analysis of randomized trials in the area of bone marrow stem cell infusions also supported the conclusion of safety and mild but statistically significant improvement in LVEF [316-318]. These data suggest that stem cell therapy, both hematopoietic and mesenchymal have clinical effects in various types of heart failure. Theoretically the leap between these clinical trials and widespread implementation is more of a business question than a medical question. In order to postulate on the future of cardiac stem cell therapy, we will discuss several possible means of optimizing existing work.

However, the lack of concordance between emerging animal data showing low or undetectable contributions of BM cells to new cardiomyocyte formation [319, 320] and the contrasting clinical benefits derived in early uncontrolled trials [321-323] raises the question of what mechanism underlies contractile-function improvement following human BM cell therapy. Mechanisms of cell benefit in addition to myogenesis might involve intrinsic remodeling of the ventricle in the absence of a direct cellular effect, improvement in contractile function and myocardial perfusion secondary to the primary revascularization procedure (in the case of stent placement or coronary bypass surgery) or impairment of myocyte death. Additional nonmyogenic contributions to BM cell improvements in cardiac function might include vasculogenic effects of angioblastic, myeloid, mesenchymal or other stem cells resident in the marrow and heart, or paracrine effects of these cells through the release of pro-angiogenic growth and survival factors. Moreover, hematopoietic stem cells characterized by CD133+ and/or CD34+ expression might contribute directly or indirectly to cardiac remodeling, myocyte survival or long-term improvements in contractile function.

CD133+ EPC are of particular interest in studies directed to therapeutic angiogenesis. Reports by Asahara and other groups indicate that these cells differentiate into endothelial cells after short-term culture[324]. Stamm et al. injected with high dose of autologous BM-derived CD133+ cells in six patients in an akinetic infarcted area not amenable to revascularization at the time of CABG[323]. Four patients demonstrated improved ejection fraction (EF), and five patients demonstrated decreased ischemic defect on nuclear scintigraphy [323] A larger phase I trial with 12 patients demonstrated improvement in ventricular perfusion and dimensions by scintigraphic imaging and no episodes of ventricular arrhythmias[325]. In patients with recent myocardial infarction, Bartunek et al. performed intracoronary administration of enriched CD133+ cells. Among 35 patients with acute myocardial infarction treated with stenting, 19 underwent intracoronary administration of CD133+ progenitor cells. These authors noted that intracoronary infusion of selected CD133+ EPC is associated with improved left ventricular performance paralleled with increased myocardial perfusion and viability[326]. Taken together, these data support the efficacy of patient-derived CD133+ EPC in mediating vasculogenesis in response to ischemia and lay the basis for our Umbilical Cord Blood (UCB) derived CD133+ EPC or individual patient-derived CD133+ EPC [327-330] cells as a source for cellular therapy for cardiac ischemia. Attempts at increasing efficacy of stem cells for cardiac indications have taken several avenues of investigation: increasing trafficking efficacy; enhancing plasticity of administered cells; and increasing growth factor production. Endowment of these features as been performed by gene transfection or modification of culture conditions such as exposure to cytokines or hypoxia. Another interesting approach is addition of chemotactic agents to the area of tissue injury to enhance trafficking. These approaches will be discussed below.

Mesenchymal stem cells are known to migrate to injured tissue and hypoxic tissue through expression of receptors such as CD44 [331-333] and CXCR-4 [334], respectively. One method of increase efficacy of these cells is to increase their ability to traffic to where they are needed. This has been performed using various approaches. Cheng et al used retroviral transfection to over express CXCR-4 on rat bone marrow derived MSC. These cells were functionally competent as judged by similar growth profiles and differentiation ability when compared to control transfected MSC. Intravenous administration of the modified cells in a rat model of myocardial infarction led to a significant improvement in migration to the area of infarct, and LVEF, as well as decreased wall thinning and fibrosis when compared to animals receiving control MSC [317]. Although many fears exist surrounding genetically modified cells, current advances in delivery vectors have for increased safety features which may allow such modified MSC to become a clinical reality [335]. An alternative and perhaps easier way of inducing MSC to expression CXCR-4 is simply “pulsing” them with a brief period of hypoxia [336], or exposure to cytokines such as SCF, IL-6, Flt-3 ligand, HGF and IL-3 [337].

Instead of increasing affinity of the stem cells to the chemoattractant, the other way to achieve the same result is to increase the concentration of the chemoattractant. One way is to provide an exogenous depot of angiogenic cytokines in proximity to the area where stem cell migration is desired. Tang et al administered a SDF-1 expressing plasmid into the ischemic border zone 2 weeks after induction of infarct in BALB/c mice. To determine whether the expressed chemoattractant actually caused stem cell homing, syngeneic labeled bone marrow cells were intravenously injected 3 days after SDF-1 plasmid administration. A significantly increased number of labeled cells were observed in the group receiving the plasmid, in the area whether the plasmid was injected [338]. This data suggest that it is feasible to reproduce mobilization induced by infarcts through the administration of homologous cytokines. However, the authors did not describe therapeutic benefit. In another experiment, a more clinically-translatable approach was taken. Fibrin glue, fibrinogen and thrombin mixed at the point of care, is used in surgery to control bleeding [339]. Zhang et al used pegylation technology to covalently bind recombinant SDF-1 to fibrinogen and demonstrated that subsequent to mixing with thrombin, the resultant “patch” could serve as a means of controlled release of SDF-1. The patch was placed on the infarct area of the left ventricle of mice after ligation of the left anterior descending coronary artery. In comparison to control mice receiving a fibrin patch lacking SDF-1, an increase in cells with a stem cell antigen and c-kit positive phenotype was observed in the experimental group. Additionally, at completion of experiment, an increased LVEF was observed in the treatment mice [340]. Since endogenous cardiac stem cells also express a similar phenotype [341], and cell labeling was not performed, it is difficult to determine whether the therapeutic effect was mediated by mobilization of bone marrow progenitors or cardiac resident stem cells. One interesting way of enhancing activity of such a localization of chemoattractant is to concurrently administer exogenous stem cells, or to mobilize endogenous bone marrow stem cells. In fact, the latter was performed in a study where fibroblasts expressing SDF-1 were injected into the hindlimbs of mice after femoral ligation. A synergistic induction of angiogenesis was detected when endogenous bone marrow derived stem cells were mobilized with G-CSF [342]. Other clinically used methods may be implemented to enhance stem cell trafficking. For example, erythropoietin (EPO), in addition to its well-known anti-apoptotic effects on cardiomyocytes [343], has actually been shown to stimulate responsiveness of bone marrow derived stem cells to SDF-1 when administered in vivo [344]. Combination therapies of this sort will be interesting to evaluate clinically, especially when the various components are already approved. Once we can make sure that stem cells arrive to the site where they are needed to stimulate regeneration, how do we know that they can do this effectively? For example, we do know that in general, stem cell activity diminishes with age [345], and specifically, in patients with cardiovascular risk factors stem cell activity is additionally suppressed as compared to healthy age-matched controls [346]. There are several issues that must be taken into consideration. Perhaps, most importantly, is how do the stem cells mediate their therapeutic effects? On the one hand, people will state that adult stem cells, such as hematopoietic [347] and even in some cases mesenchymal stem cells [348], do not differentiate into functional cardiomyocytes, so therefore therapy with these cells is a futile endeavor. As we discussed above, efficacy of cardiac stem cell therapy does not rely on cell replacement but could be, and most likely is, mediated by trophic, angiogenic, anti-inflammatory and anti-apoptotic effects. Regardless of this, the concept of “revitalizing” an adult stem cell so to be able to actually replace cardiac cells is very exciting.

One method of such “revitalization” is involves making the stem cells take a more primitive, embryonic stem cell-like phenotype. It is known that the more differentiated cells become, the less plasticity they have, and the more restricted epigenetically, they become. Perhaps this was associated with the reason why DNA methyltransferase inhibitors such as 5-azacytidine were initially added to stem cells before implantation into infracted hearts [284, 349]. Other agents that act epigenetically, such as the histone deacetylase inhibitor valproic acid have been demonstrated to enhance hematopoietic stem cell self renewal capacity in vitro [350, 351], and have a positive effect on post infarct remodeling in vivo, although it is not clear whether stem cell activation is implicated [352]. Instead of using agents such as these that upregulate factors associated with pluripotency such as Nanog [353], an alternative approach is to simply transfect the cells with such genes. For example Go et al transfected bone marrow derived MSC with Nanog and reported superior expansion potential and ability to differentiate as compared to control transfected cells [354]. Transfection of such “retrodifferentiation” genes is particularly exciting in light of the recent discovery that fibroblasts can be induced to pluripotency through introduction of the pluripotency genes Oct¾, Sox2, c-Myc, and Klf4 in mice [355] and humans [356]. These “inducible pluripotent stem cells” (iPS) appear to be functional, not only by gene transcription profile, but also ability to reconstitute animals hematopoietically [357]. Theoretically, it would make sense that retrodifferentiation of an adult stem cell into an iPS would be easier than a skin fibroblast. Indeed, Kim et al demonstrated that in order to derived iPS cells from neural stem cells, only the factors Oct-4 and klf-4 or c-Myc are needed [358]. Furthermore, newer transfection methods of generating iPS through non-retroviral means have been reported, giving the possibility of generating clinically applicable therapies from these cells [359]. Unfortunately, carcinogenesis associated with the viral vectors is not the main limitation. It is known in general that ES cells are carcinogenic [360]. Additionally, the very transcriptional profile associated with cancer stem cells appears to be related to that of pluripotent cells, regardless if they are generated by iPS or from ES cells [361].

Thus one way of increasing potency of MSC-based therapy is through induction of such “rejuvenation” unfortunately; too much rejuvenation leads to the possibility of carcinogenesis, and additionally may have implications on ability of the cells to evade immune responsiveness and/or migration to the area of injury. For example, it is known that embryonic stem cells are hypoimmunogenic, as seen by weak ability to stimulate allogeneic lymphocyte proliferation [362]. However it remains an open question whether ES cells can actively suppress ongoing immune responses as is the case with MSC both in animal models [251] and clinically [363, 364]. In terms of migratory ability, it is known that functionally various adult stem cells play a protective role in the physiological response to injury. Although the effects in clinical situations are minor, there is suggestive evidence, for example in stroke patients that a correlation between endogenous stem cell mobilization and positive outcome exists [365, 366]. While in cardiac infarct cases we do know that mobilization occurs [367], but correlation with infarct recovery have not been made. Regardless, the question of what stage of differentiation the best cell population is for treatment of cardiac indications remains unclear. While the approach of utilizing the patient’s own HSCs has the advantage of avoiding potential immune response relative to allogeneic cells, it has several disadvantages as well. Patients with acute myocardial infarction may experience significant morbidity with attempted large volume BM harvest or attempted cytokine-induced BM stem cell mobilization. Moreover, a majority of cardiac patients are of advanced age. Increasing evidence suggests reduced potential and regenerative capacity of marrow-derived circulating EPC with increasing age [368, 369], and compromised capability of responding to inflammatory signals and cytokines released from the ischemic bed. [370] Thus the ready availability of an “off-the shelf” allogeneic EPC cellular infusion is optimal.

Cord blood stem cells have been used successfully for more than 20 years. Currently, they are used to treat approximately 70 diseases including immunodeficiencies; genetic and neurological disorders; certain types of cancers; and blood disorders, such as leukemia, lymphoma, sickle cell anemia, and aplastic anemia. [371] Today, physicians have performed more than 8,000 cord blood stem cell transplants worldwide. These stem cells hold vast therapeutic promise to address major unmet medical needs and are increasingly being used in medical therapies to improve - and save - lives. Cord blood stem cell treatments differ from bone marrow stem cell treatments in three key areas: increased tolerance of HLA-mismatching, decreased risk of graft-versus-host disease, and enhanced proliferation ability.[372]

Studies in a clinical setting of leukemia have shown that Umbilical Cord Blood Transplants (UCBT) are more useful than peripheral blood or bone marrow derived stem cell transplants. [373] The UCBT tend to need less HLA matching for graft survival. Lubin and Greene[373] found that a 4/6 and ⅚ HLA match was still useful if the stem cells were UCB derived. UCBT are also more advantageous because it is easier and less painful for the donors and is associated with a decreased risk of viral infections after transplantation. UCBT also has a larger number of potential donors, longer telomeres, and is more quickly available.[372-377] Barker et al. found that individuals receiving UCBT received their cord blood transplant an average of 25 days sooner than individuals receiving Bone Marrow derived stem cell transplants.[378]

Umbilical cord blood (UCB) can be utilized as a source of potential therapeutic allogeneic endothelial progenitor cells (EPC). The advantages of cord blood include: collection at no risk to the donor, easy storage, low inherent viral contamination risk, and decreased donor discovery time. [372] However, in the setting of vascular regeneration, allogeneic cord blood EPCs are expected to be detected by the immune system of an immune competent patient. Few in vitro studies have shown that UCB EPC cells express HLA class I and II surface molecules and elicit allogeneic T-cell proliferation by immune competent adult mononuclear cells in mixed lymphocyte cultures (MLR). It is important to address whether the immunogenicity of a UCB EPC allogeneic cellular product is advantageous (i.e. augmenting a vasculogenesis response by recipient cells in situ) or potentially deleterious (i.e. dampening vasculogenesis response or worsening vascular ischemia via allogeneic inflammatory responses). More importantly, pre-clinical studies by our group and others to date have identified that in vascular injury rodent models, augmentation of murine endogenous microvascular collateralization is not completely due to anatomic incorporation of infused human EPC into the murine vascular endothelium.[379] These observations suggest possible paracrine-mediated angiogenic effects elicited by the infused human EPC responding to inflammatory signals, cytokines and growth factors released from the ischemic region[380] suggesting that stimulation of inflammatory signaling molecules by allogeneic cells in the injured area augment growth factor-induced neovascularization.

While various stem cell sources have been studied to induce myogenesis, recent interest has focused on promoting cardiac angiogenesis by proangiogeneic factors [381]. The existence of angiogenic factors such as acidic and basic fibroblast growth factor (FGF1 and 2), VEGF, PDGF, insulin-like growth factor-1 (IGF-1), angiogenin, transforming growth factor (TGF-α and TGF-β), tumor necrosis factor (TNF-α), hepatocyte growth factors (HGF), granulocyte colony-stimulating factor (G-CSF), placental growth factor (PGF), interleukin 8 to be mitogenic for endothelial cells[382-388]. Of the large number of angiogenesis factors that have been described, the FGF and VEGF families have been most intensively studied [389, 390]. Although several elements are likely to be involved in the process of angiogenesis, in vivo studies have amply demonstrated that the simple administration of an angiogenic growth factor is sufficient to stimulate the cascade of events that lead to angiogenesis and to the augmentation of blood delivery. However, administration of growth factors in the normal heart does not result in angiogenesis [391]. These techniques retain an important potential as an adjunct to cellular transplantation in inducing angiogenesis in injured myocardium when revascularization cannot be achieved by standard techniques, namely percutaneous angioplasty and coronary artery bypass grafting.

Although a number of angiogenesis studies using gene therapy technique to deliver growth factors are in clinical trials, the resident population of vascular endothelial cells in adults competent to respond to available angiogenic growth factors limits these interventions due to age-related diminution of vascular endothelial cell number. Additionally, vascular endothelial cell function may limit the efficacy of patient-derived progenitor cells in mediating neo-vascularization [392-394]. This data supports the concept that an exogenous source of EPC such as UCB-derived HSCs, rather than autologous patient-derived cells, may be optimal for cellular therapeutics intended to enhance angiogenesis and collateralization around stenosed or occluded vessels to relieve ischemia. UCB-derived HSC offer distinct advantages as a cell source, including greater potential lifespan and reparative proliferation, relative to existing models of therapeutic angiogenesis derived from patient peripheral blood or marrow.

The available data thus far indicates that there is no proven “off-the-shelf” stem therapy to repair or regenerate heart after acute myocardial infarction (MI) or congestive heart failure (CHI). The limited capacity of heart cells to regenerate and also the available alternative source of stem cells for myocyte regeneration and cardiac revascularization is also limited[395] Human UCB contains several different type of stem cells including HSC, EPC and MSCs.[276, 396-398]. These studies suggest human UCB derived stem cells as a potential alternative source of stem cells for a potent cardiomyocyte regenerative and revascularization. Although more studies are needed to prove the full clinical benefit of UCB derived stem cells, scientists and clinicians believe the human UCB have the most immediate benefit as ideal source for stem cells. Wharton’s Jelly MSC (WJ-MSC) are a potent type of MSC that possesses superior angiogenic and regenerative activities compared to other types of MSC [399, 400]. Clinical studies have shown that UC-MSC may be safely administered intravenously [401-407]. Animal studies have demonstrated UC-MSC are effective in models of CHF [399, 400, 408-410]. Mechanistically UC-MSC mediate therapeutic activity on CHF through angiogenic [411], anti-inflammatory [412], and cardioregenerative effects [413]. The endometrium is a unique tissue that undergoes approximately 500 cycles of highly vascularized tissue growth and regression within a tightly controlled manner in the lifetime of the average female. This has triggered the concept that a self-renewing cell capable of differentiating into various tissues may be present in the endometrium. One of the first series of data describing the possibility of stem cells in the endometrium came from Prianishnikov in 1978 who reported that three types of stem cells can be isolated from this tissue: estradiol-sensitive cells, estradiol- and progesterone-sensitive cells and progesterone-sensitive cells [414]. A subsequent study in 1982 demonstrated that cells in the endometrium destined to generate the decidual portion of the placenta are bone marrow derived [415], which prompted the speculation of the existence of a stem cell like cell in the endometrium. Further hinting at this possibility were seen in the studies showing expression of telomerase in endometrial tissue collected during the proliferative phase [416, 417].

The first demonstration of pluripotent stem cells derived from the endometrium occurred almost simultaneously by two independent groups. Meng et al [418], used the process of cloning rapidly proliferating adherence cells derived from menstrual blood and generated a homogenous cell population expressing CD9, CD29, CD41a, CD44, CD59, CD73, CD90, and CD105 and lacking CD14, CD34, CD45 and STRO-1 expression. In contrast to Cui et al, the authors demonstrated that the cells had substantially faster replicative potential as compared to bone marrow MSC, a unique cytokine and matrix metalloprotease (MMP) profile, as well as ability to differentiate into cardiomyocytic, respiratory epithelial, neurocytic, myocytic, endothelial, pancreatic, hepatic, adipocytic, and osteogenic lineages. Interestingly, the identified cells expressed telomerase and OCT-4 but lacked expression of NANOG-1. Given the pluripotent nature of these cells, the authors named them “Endometrial Regenerative Cells” (ERC). Shortly after, Patel et al [419] reported a population of cells isolated using c-kit selection of menstrual blood mononuclear cells. The cells had a similar phenotype, proliferative capacity, and ability to be expanded for over 68 doublings without induction of karyotypic abnormalities. It is worth noting that both groups found expression of the pluripotency gene OCT-4 but not NANOG.

More recent investigations have confirmed these initial findings. For example, Park et al demonstrated that endometrial cells are significantly more potent originating sources for dedifferentiation into inducible pluripotent cells as compared to other cell populations [420]. Specifically, human endometrial cells displayed accelerated expression of endogenous NANOG and OCT4 during reprogramming compared with neonatal skin fibroblasts. Additionally, the reprogramming resulted in an average colony-forming iPS efficiency of 0.49 ± 0.10%, with a range from 0.31-0.66%, compared with the neonatal skin fibroblasts, resulting in an average efficiency of 0.03 ± 0.00% per transduction, with a range from 0.02-0.03%. Suggesting pluripotency within the endometrium compartment, another study demonstrated that purification of side population (eg rhodamine effluxing) cells from the endometrium results in a population (identification?) of cells expressing transdifferentiation potential with a genetic signature similar to other types of somatic stem cells [421].

Given the high degree of angiogenesis occurring monthly in the endometrium, it is hypothesized that ERC possess a physiological role in this process. This is supported by the high concentrations of angiogenic cytokines and MMPs expressed in ERC compared to other MSC types [418]. The therapeutic angiogenesis promoting cytokine, vascular endothelial growth factor (VEGF) is naturally found in the endometrium and its production is stimulated monthly by estradiol [422]. Further confirmation of ovary-derived hormones’ role in endometrial vasculogenesis came from the studies in which ovarectomy resulted in lack of endometrial VEGF production [423] and angiogenesis [424]. While the cellular mechanisms of angiogenesis appear to be multifactorial and involve neutrophils [425, 426], uterine natural killer (NK) cells [427], and circulating endothelial progenitor cells (EPC) [428-430], the possibility of ERC playing a significant role in this process is plausible, given that bone marrow derived cells are known to form the endometrium [431], and that other types of MSC have been shown to be potently angiogeneic.[432]. Additionally, it was shown that CD34 and VEGFR2 positive cells of bone marrow donor origin are found in the endometrium [433], thus suggesting that bone marrow derived EPCs control endometrial angiogenesis. Mobilization of bone marrow CD34 stem cells with G-CSF during menstruation was reported to be significantly lower than can be accounted for by blood loss, further supporting the possibility that a bone marrow originating population contributes to endometrial tissue turnover and angiogenesis [434]. Following up on the notion that ERC play a key role in angiogenesis, Murphy et al utilized an aggressive hindlimb ischemia model combining femoral artery ligation with nerve excision in order to induce ischemic injury that results in limb loss. ERC administration resulted in prevention of limb loss in all treated animals, whereas control animals suffered limb necrosis [435]. In the same study, ERC were demonstrated to inhibit ongoing mixed lymphocyte reaction, stimulate production of the anti-inflammatory cytokine IL-4 and inhibit production of IFN-g and TNF-alpha. It is important to note that the animal model involved administration of human ERC into immunocompetent BALB/c mice.

The relationship between angiogenesis and post myocardial infarct healing is well-known. Expanding on previous work by Umezawa’s group demonstrating myocytic differentiation of ERC-like cells [436], administration of ERC into a model of post infarct cardiac injury was performed [437]. Recovery was compared to bone marrow MSC. A superior rate of post-infarct recovery of ejection fraction, as well as reduction in fibrosis was observed with the ERC-like cells. Furthermore, it was demonstrated that the cells were capable of functionally integrating with existing cardiomyocytes and exerted effects through direct differentiation. The investigators also demonstrated in vitro generation of cardiomyocyte cells that had functional properties. The stimulation of neurogenesis in post-stroke injury models has been demonstrated to be linked directly to angiogenesis [438]. Borlongan et al [439], injected ERC intracerebrally, or intravenously into immune competent rats subsequent to middle cerebral artery ligation. They demonstrated substantial increases in functional recovery of the rats receiving ERC. Additionally, it was shown that ERC secreted significant amounts of neurotrophic growth factors including VEGF, BDNF, and NT-3. Using ERC-like cells derived from endometrial explants, Hugh Taylor’s group used the rat MPTP model of Parkinson’s Disease. The cells administered were shown to express CD90, platelet derived growth factor (PDGF)-Rβ and CD146 but not CD45 or CD31. Administration of the human cells into immune competent mice resulted in restoration of dopamine production, differentiation into dopaminergic neurons, and functional improvement [440]. Although we have demonstrated differentiation of ERC into cells capable of producing insulin in vitro, a study by Li et al [441]demonstrated that growth in spheroid cultures of ERC could induce differentiation into islet-like clusters when cultured under appropriate differentiation media. Transplantation of the cells into mice made diabetic by administration of the beta cell toxin streptozotocin resulted in improved glucose control and production of insulin in response to glucose challenge. One of the most interesting aspects of ERC published to date is their apparent anti-neoplastic activity. The field of MSC modulation of oncogenesis is controversial with reports demonstrating cancer promotion and cancer inhibition. We observed that intracerebral or intravenous administration of ERC was capable of reducing size of C6 gliomas, which was associated with reduced angiogenesis [442].

A clinical trial examining ERC, in patients with end stage heart failure was initiated Non-Revascularizable IschEmic Cardiomyopathy treated with Retrograde COronary Sinus Venous DElivery of Cell TheRapy (RECOVER-ERC) trial. The trial was aimed at assessing safety and efficacy of the company’s Endometrial Regenerative Cell (ERC) stem cell product in 60 heart failure patients who have no available treatment options. The ERC will be administered using a novel catheter-based retrograde administration methodology that directly implants cells in a simple, 30 minute, procedure. To date 17 patients have been treated. A review of the trial was previously published [443]. Given that we do not know the best stage of differentiation to administer the stem cells, as well as the various drawbacks of transfection and reprogramming approaches, one possible way of advancing efficacy of stem cell therapy would be to combine various stem cell types that we know have trophic activity. One interesting combination would be the use of CD34 cells, which are primarily hematopoietic, but also angiogenic, together with allogeneic mesenchymal stem cells, which have trophic, angiogenic, and potent anti-inflammatory potential. The rationale for combining these two approaches come from several perspectives: a) after tissue injury both mesenchymal [331, 444, 445] and hematopoietic stem cells [446-448] are mobilized thus potentially both cells may have therapeutic synergistic activity in a physiological sense; b) In vivo MSC provide a microenvironment for CD34 stem cells both embryonically [449], and postnatally [450], in vitro MSC promote expansion of CD34 stem cells [451, 452]; and c) animal models suggest synergy of function [453].

The combination of MSC and HSC has been previously published to save end-stage patient suffering from dilated cardiomyopathy which underwent a profound improvement in ejection fraction after receiving a combination of cord blood expanded CD34 cells and placental matrix derived mesenchymal stem cells [454].

The use of the cells of the invention in treatment of peripheral artery disease is disclosed. A case report described 3 intravenous infusions of expanded bone marrow autologous mesenchymal stem cells in one 34 year old patient with critical limb ischemia due to systemic sclerosis. After 1 month areas of necrotic skin were reduced after the first mesenchymal stem-cell infusion. After the third infusion, angiography showed revascularization of the patient’s extremities. Skin section analysis revealed cell clusters with tubelike structures, and angiogenic factors were strongly expressed [455].

Although bone marrow mononuclear cells have been used in critical limb ischemia, comparison with mesenchymal stem cells has not been made. A pilot study was conducted to identify better cells for the treatment of diabetic critical limb ischemia (CLI) and foot ulcer in a pilot trial. Under ordinary treatment, the limbs of 41 type 2 diabetic patients with bilateral CLI and foot ulcer were injected intramuscularly with bone marrow mesenchymal stem cells (BMMSCs), bone marrow-derived mononuclear cells (BMMNCs), or normal saline (NS). The ulcer healing rate of the BMMSC group was significantly higher than that of BMMNCs at 6 weeks after injection (P=0.022), and reached 100% 4 weeks earlier than BMMNC group. After 24 weeks of follow-up, the improvements in limb perfusion induced by the BMMSCs transplantation were more significant than those by BMMNCs in terms of painless walking time (P=0.040), ankle-brachial index (ABI) (P=0.017), transcutaneous oxygen pressure (TcO(2)) (P=0.001), and magnetic resonance angiography (MRA) analysis (P=0.018). There was no significant difference between the groups in terms of pain relief and amputation and there was no serious adverse events related to both cell injections. The authors concluded that BMMSCs therapy may be better tolerated and more effective than BMMNCs for increasing lower limb perfusion and promoting foot ulcer healing in diabetic patients with CLI [456].

Given the potential superiority of bone marrow mesenchymal stem cells to mononuclear cells, a study was performed in a double-blind manner to further elucidate therapeutic potential of the mesenchymal stem cells. The study was conducted in patients with established CLI as per Rutherford classification in category II-4, III-5, or III-6 with infra-inguinal arterial occlusive disease and were not suitable for or had failed revascularization treatment. The primary end point was incidence of treatment - related adverse events (AE). Exploratory efficacy end points were improvement in rest pain, increase in Ankle Brachial Pressure Index (ABPI), ankle pressure, healing of ulcers, and amputation rates. Twenty patients (BM-MSC: Placebo = 1:1) were administered with allogeneic BM-MSCs at a dose of 2 million cells/kg or placebo (PlasmaLyte A) at the gastrocnemius muscle of the ischemic limb. Improvement was observed in the rest pain scores in both the arms. Significant increase in ABPI and ankle pressure was seen in BM-MSC arm compared to the placebo group. Incidence of AEs in the BM-MSC arm was 13 vs. 45 in the placebo arm where as serious adverse events (SAE) were similar in both the arms (5 in BM-MSC and 4 in the placebo group). SAEs resulted in death, infected gangrene, amputations in these patients. It was observed that the SAEs were related to disease progression and not related to stem cells. The authors concluded that BM-MSCs are safe when injected IM at a dose of 2 million cells/kg body weight. Efficacy parameters such as ABPI and ankle pressure showed positive trend warranting further studies [457].

In another study, 7 patients were consecutively enrolled, on the basis of the following criteria: (i) lower-limb rest pain or ulcer; (ii) ankle systolic oxygen pressure <50 or 70 mm Hg for non-diabetic and diabetic patients, respectively, or first-toe systolic oxygen pressure <30 mm Hg or 50 mm Hg for non-diabetic and diabetic patients, respectively; (iii) not suitable for revascularization. ASCs from abdominal fat were grown for 2 weeks and were then characterized. More than 200 million cells were obtained, with almost total homogeneity and no karyotype abnormality. The expressions of sternness markers Oct4 and Nanog were very low, whereas expression of telomerase was undetectable in human ASCs compared with human embryonic stem cells. ASCs (10(8)) were then intramuscularly injected into the ischemic leg of patients, with no complication, as judged by an independent committee. Trans-cutaneous oxygen pressure tended to increase in most patients. Ulcer evolution and wound healing showed improvement [458].

Assessment of autologous adipose mesenchymal stem cells was performed in another 15 patients by another group, 15 male CLI patients with ischemic resting pain in 1 limb with/without non-healing ulcers and necrotic foot. ATMSC were isolated from adipose tissue of thromboangiitis obliterans (TAO) patients (B-ATMSC), diabetes patients (D-ATMSC), and healthy donors (control ATMSC). In a colony-forming unit assay, the stromal vascular fraction of TAO and diabetic patients yielded lesser colonies than that of healthy donors. D-ATMSC showed lower proliferation abilitythan B-ATMSC and control ATMSC, but they showed similar angiogenic factor expression with control ATMSC and B-ATMSC. Multiple intramuscular ATMSC injections cause no complications during the follow-up period (mean follow-up time: 6 months). Clinical improvement occurred in 66.7% of patients. Five patients required minor amputation during follow-up, and all amputation sites healed completely. At 6 months, significant improvement was noted on pain rating scales and in claudication walking distance. Digital subtraction angiography before and 6 months after ATMSC implantation showed formation of numerous vascular collateral networks across affected arteries [459].

In order to investigate efficacy of other routes of bone marrow mesenchymal stem cell infusion, as well as the use of allogeneic cells, a study evaluated 13 patients for a phase I trial to investigate the safety and efficacy of intra-arterial MSCs in CLI patients. Eight patients with ten affected limbs were recruited for the study. As two patients (three limbs) died of ischemic cardiac events during the 6-month follow-up period, seven limbs were finally evaluated for the study. There was significant pain relief. Visual analog scale (VAS) scores decreased from 2.29 ± 0.29 to 0.5 ± 0.34 (p < 0.05), ankle brachial pressure index (ABPI) increased significantly from 0.56 ± 0.02 to 0.67 ± 0.021 (p < 0.01), and transcutaneous oxygen pressure (TcPO2) also increased significantly in the foot from 13.57 ± 3.63 to 38 ± 3.47. Similar improvement was seen in the leg as well as the thigh. There was 86 % limb salvage and six of seven ulcers showed complete or partial healing [460].

A total of eight patients (all male, median age 52 years, range 31 ~77) with CLI were enrolled in this phase I trial. All patients were considered ineligible for further revascularization to improve CLI. We injected 1×10(7) hUCB-MSCs per single dose intramuscularly into the affected limb. The primary end points of safety were occurrence of adverse events (procedure-related complication, allergic reaction to hUCB-MSCs, graft-versus-host disease, cardiovascular and cerebrovascular events) and improvement of symptoms/clinical parameters (healing of foot ulcer, ankle-brachial index, and pain-free walking distance). Angiogenesis was measured with conventional angiography and scored by an independent reviewer. There were four adverse events in three patients. One patient, developed whole body urticaria after injection on treatment day, which disappeared after one day of antihistamine treatment. The other adverse events included diarrhea, oral ulceration, and elevation of serum creatinine level; all conditions improved without treatment. Abnormal results of laboratory parameters were not detected in any patients. Three of four ulcerations (75%) healed completely. Angiographic scores increased in three of eight patients. This phase I study demonstrates that intramuscular hUCB-MSC injection is a safe and well tolerated treatment for patients with end-stage CLI due to ASO and TAO [461]. Thus the cells of the invention may be utilized for stimulation of neovascularization, reduction of inflammation and stimulation of muscle regeneration in patients with critical limb ischemia and/or other types of peripheral artery disease.

Cells of the invention are useful for the treatment of liver failure and other hepatic pathologies. We present examples from use of other types of stem cells, such as mesenchymal stem cells, which are provided to guide one of skill in the art in the practice of the invention. In one study, eight patients (four hepatitis B, one hepatitis C, one alcoholic, and two cryptogenic) with end-stage liver disease having Model for End-Stage Liver Disease score > or =10 were treated with autologous bone marrow MSCs. Approximately 30-50 million MSCs were administered into peripheral or the portal vein. Treatment was well tolerated by all patients. Liver function improved as verified by the Model for End-Stage Liver Disease score, which decreased from 17.9+/-5.6 to 10.7+/-6.3 (P<0.05) and prothrombin complex from international normalized ratio 1.9+/-0.4 to 1.4+/-0.5 (P<0.05). Serum creatinine decreased from 114+/-35 to 80+/-18 micromol/l (P<0.05). Serum albumin changed from 30+/-5 to 33+/-5 g/l and bilirubin from 46+/-29 to 41+/-31 micromol/l. No adverse effects were noted [462]. A larger study examined efficacy and safety of single transplantation with autologous marrow mesenchymal stem cells (MMSCs) in patients with hepatitis B associated end stage liver failure. Treatment was performed in 53 patients and a total of 105 patients matched for age, sex, and biochemical indexes, including alanine aminotransferase (ALT), albumin, total bilirubin (TBIL), prothrombin time (PT), and Model for End-Stage Liver Disease (MELD), comprised the control group. A total of 120 mL of bone marrow was obtained from each patient and then diluted and separated. Then, the MMSC suspension was slowly transfused into the liver through the proper hepatic artery. The success rate of transplantation was 100%, without serious side effects or complications. Levels of ALB, TBIL, and PT and MELD score of patients in the transplantation group were markedly improved from 2-3 weeks after transplantation, compared with those in the control group. At 192 weeks of follow-up, there were no dramatic differences in incidence of hepatocellular carcinoma (HCC) or mortality between the two groups. Additionally, there were no significant differences in the incidence of HCC or mortality between patients with and without cirrhosis in the transplantation group [463].

To increase efficacy, an assessment was performed in another clinical trial in which autologous MSC were either administered in an undifferentiated or hepatic differentiated state. Twenty-five patients with hepatitis C induced Child C liver cirrhosis, MELD score >12 were included. They were divided into 2 groups. Group I, the MSCs group (n=15), this group was subdivided into two subgroups: Ia & Ib (undifferentiated and differentiated respectively). Group II (control group; n=10) involved patients with cirrhotic liver under conventional supportive treatment. Ninety ml BM was aspirated from the iliac bone for separation of MSCs. Surface expression of CD271, CD29 and CD34 were analyzed using flowcytometry. Hepatogenesis was assessed by immunohistochemical expression of OV6, AFP and albumin. Finally approximately 1 million MSCs/Kg were suspended in saline and were placed in blood bag and injected slowly intravenously over 15 min at a rate of 5 drops/min in one session. Follow up of patients at 3 and 6 months postinfusion revealed partial improvement of liver function tests with elevation of prothrombin concentration and serum albumin levels, decline of elevated bilirubin and MELD score in MSCs group. Statistical comparisons between the two subgroups (group Ia & Ib) did not merit any significant difference regarding clinical and laboratory findings [464].

A Turkish study recruited 25 patients with biopsy-proven liver cirrhosis. Patients received 1×106 autologous mesenchymal stem cells/kg via a peripheral vein. Biochemical parameters were checked monthly. Periodical radiological screening and liver biopsies before mesenchymal stem cell transplantation were performed after 6 months. Liver specimens were assessed by a pathologist. No side effect was observed and the mesenchymal stem cell transplantation procedure was well tolerated. Twelve patients completed the study. In 8 patients, improvements in Model for End-Stage Liver Disease (MELD) scores were observed. Serum albumin levels markedly increased in the third month. In patients with non-responder hepatitis C, HCV RNA levels both became negative after mesenchymal stem cell transplantation. Histopathological examinations of liver tissues before and at 6 months after transplantation revealed no change in liver tissue regeneration or fibrosis. However, in 5 patients, hepatitis activity index scores decreased. The authors concluded that peripheral vein is safe and feasible. Improvement in patients and clearance of HCV RNA may have occurred through immunomodulatory mediators secreted by transplanted mesenchymal stem cells, namely the “endocrine” effect [465].

An Egyptian study assessed peripheral vein infusion of autologous BM-MSC in end state liver failure patients with hepatitis C. Forty patients with post-hepatitis C virus (HCV) end-stage liver disease were randomized into two groups: Group 1 (GI): 20 patients who received granulocyte colony-stimulating factor (G-CSF) for 5 days followed by autologous MSCs peripheral-vein infusion and group 2 (GII): 20 patients who received regular liver-supportive treatment only (control group). In MSC-infused patients (GI), 54% showed near normalization of liver enzymes and improvement in liver synthetic function. Significant changes were reported in albumin (P = 0.000), bilirubin (P = 0.002), increased international normalized ratio (INR) (P = 0.017), prothrombin concentration (P = 0.029) and alanine transaminase (ALT) levels (P = 0.029), with stabilization of clinical and biochemical status in 13% of cases. None of the patients in GII showed any significant improvement. Hepatic fibrosis was assessed in GI by detection of procollagen IIIC peptide level (PIIICP) and procollagen III N peptide level (PIIINP). The pretreatment values of s-PIIICP and s-PIIINP were 9.4 ± 4.2 and 440 ± 189, respectively, with a decrease to 8.1 ± 2.6 and 388 ± 102, respectively, 3 months after MSC therapy. However, the difference was statistically nonsignificant (P = 0.7). A significant correlation coefficient was reported after 3 months between the s-PIIINP and prothrombin concentration (P = -0.5) and between s-PIIICP and ascites (P = 0.550) [466].

Another study examined autologous BM-MSC in a randomized placebo controlled study. The enrolled patients with decompensated cirrhosis were randomly assigned to receive MSC or placebo infusions. A median of 195 million (range: 120-295 million) cultured MSCs were infused through a peripheral vein. The primary outcome was absolute changes in MELD score. Secondary outcomes were absolute changes in Child score, liver function tests and liver volumes between the MSC and placebo group 12 months after infusion. A total of 27 patients were enrolled. Of these, 15 patients received MSC and 12 patients received placebo. One patient in the MSC group and one patient in the placebo group were lost to follow-up. Three patients in the MSC group died of liver failure 3 months (one patient), or 5 months (two patients) after cellular infusion. The baseline MELD scores of the deceased patients were significantly higher than those who remained alive in either group (20.0 vs. 15.1; P = 0.02). The absolute changes in Child scores, MELD scores, serum albumin, INR, serum transaminases and liver volumes did not differ significantly between the MSC and placebo groups at 12 months of follow-up [467].

Although the majority of studies on liver failure used patients whose hepatic pathology was induced by viral infections, a recent study assessed effects in patients with alcohol induced cirrhosis. Seventy-two patients with baseline biopsy-proven alcoholic cirrhosis who had been alcohol-abstinent for more than 6 months underwent a multicenter, randomized, open-label, phase 2 trial. Patients were randomly assigned to three groups: one control group and two autologous BM-MSC groups that underwent either one-time or two-time hepatic arterial injections of 5 × 10⁷ BM-MSCs 30 days after BM aspiration. A follow-up biopsy was performed 6 months after enrollment, and adverse events were monitored for 12 months. The primary endpoint was improvement in fibrosis quantification based on picrosirius red staining. The secondary endpoints included liver function tests, Child-Pugh score, and Model for End-stage Liver Disease score. Outcomes were analyzed by per-protocol analysis. In terms of fibrosis quantification (before versus after), the one-time and two-time BM-MSC groups were associated with 25% (19.5 ± 9.5% versus 14.5 ± 7.1%) and 37% (21.1 ± 8.9% versus 13.2 ± 6.7%) reductions in the proportion of collagen, respectively (P < 0.001). In the intergroup comparison, two-time BM-MSC transplantation in comparison with one-time BM-MSC transplantation was not associated with improved results in fibrosis quantification (P > 0.05). The Child-Pugh scores of both BM-MSC groups (one-time 7.6 ± 1.0 versus 6.3 ± 1.3 and two-time 7.8 ± 1.2 versus 6.8 ± 1.6) were also significantly improved following BM-MSC transplantation (P < 0.05). The proportion of patients with adverse events did not differ among the three groups. The authors concluded that autologous BM-MSC transplantation safely improved histologic fibrosis and liver function in patients with alcoholic cirrhosis [468]. A study by another group also assessed ability of autologous BM-MSC to repair liver after alcoholic cirrhosis. Twelve patients (11 males, 1 female) with baseline biopsy-proven alcoholic cirrhosis who had been alcohol free for at least 6 months were enrolled. BM-MSCs were isolated from each patient’s BM and amplified for 1 month, and 5 × 10(7) cells were then injected twice, at weeks 4 and 8, through the hepatic artery. One patient was withdrawn because of ingestion of alcohol. Finally, 11 patients completed the follow-up biopsy and laboratory tests at 12 weeks after the second injection. The primary outcome was improvement in the patients’ histological features. According to the Laennec fibrosis system, histological improvement was observed in 6 of 11 patients (54.5%). The Child-Pugh score improved in ten patients (90.9%) and the levels of transforming growth factor-β1, type 1 collagen and α-smooth muscle actin significantly decreased (as assessed by real-time reverse transcriptase polymerase chain reaction) after BM-MSCs therapy (P < 0.05). The authors concluded that BM-MSC therapy in alcoholic cirrhosis induces a histological and quantitative improvement of hepatic fibrosis [469].

In an effort to augment therapeutic efficacy, the PPAR-gamma agonist pioglitozone was administered in a 2 patient pilot study of liver failure patients receiving autologous BM-MSC. Intraportal autologous bone marrow-derived MSCs were transplanted twice (6 months interval) to the w patients. Meanwhile, 30 mg/day pioglitazone was prescribed for 12 months. Patients were assessed at baseline and months 1, 3, 6, and 12 post-infusion. Procedural complications or any major adverse effects did not occur in this pilot study. The patients’ clinical conditions remained stable with no evidence of deterioration during the course of the study. A transient improvement in the Model for End-Stage Liver Disease (MELD) score was observed at month 3 post-infusion in one patient, which eventually returned to baseline at month 12 [470].

As with other indications, the use of allogeneic umbilical cord MSC (UC-MSC) was attempted in decompensated liver failure. A total of 45 chronic hepatitis B patients with decompensated liver cirrhosis, including 30 patients receiving UC-MSC transfusion, and 15 patients receiving saline as the control, were recruited; clinical parameters were detected during a 1-year follow-up period. No significant side-effects and complications were observed in either group. There was a significant reduction in the volume of ascites in patients treated with UC-MSC transfusion compared with controls (P < 0.05). UC-MSC therapy also significantly improved liver function, as indicated by the increase of serum albumin levels, decrease in total serum bilirubin levels, and decrease in the sodium model for end-stage liver disease scores. UC-MSC transfusion is clinically safe and could improve liver function and reduce ascites in patients with decompensated liver cirrhosis. UC-MSC transfusion, therefore, might present a novel therapeutic approach for patients with decompensated liver cirrhosis [407].

Another allogeneic trial utilized similar UC-MSC for a type of liver condition called acute-on-chronic liver failure (ACLF). A total of 43 hepatitis B ACLF patients were enrolled for this open-labeled and controlled study; 24 patients were treated with UC-MSCs, and 19 patients were treated with saline as controls. UC-MSC therapy was given three times at 4-week intervals. The liver function, adverse events, and survival rates were evaluated during the 48-week or 72-week follow-up period. No significant side effects were observed during the trial. The UC-MSC transfusions significantly increased the survival rates in ACLF patients; reduced the model for end-stage liver disease scores; increased serum albumin, cholinesterase, and prothrombin activity; and increased platelet counts. Serum total bilirubin and alanine aminotransferase levels were significantly decreased after the UC-MSC transfusions. The authors concluded that UC-MSC transfusions are safe in the clinic and may serve as a novel therapeutic approach for HBV-associated ACLF patients [406].

The use of UC-MSC was also investigated in patients with primary biliary cirrhosis (PBC). A single-arm trial that included seven PBC patients with a suboptimal response to standard treatment. UC-MSCs were first cultured, and then 0.5 × 10(6) cells/kg body weights were infused through a peripheral vein. UC-MSCs were given three times at 4-week intervals, and patients were followed up for 48 weeks. Primary outcomes were to evaluate the safety and feasibility of UC-MSC treatment, and secondary outcomes were to evaluate liver functions and patient’s quality of life. No obvious side-effects were found in the patients treated with UC-MSCs. Symptoms such as fatigue and pruritus were obviously alleviated in most patients after UC-MSC treatment. There was a significant decrease in serum alkaline phosphatase and γ-glutamyltransferase levels at the end of the follow-up period as compared with baseline. No significant changes were observed in serum alanine aminotransferase, aspartate aminotransferase, total bilirubin, albumin, prothrombin time activity, international normalized ratio, or immunoglobulin M levels. The Mayo risk score, a prognostic index, was also stable during the treatment and follow-up period. The authors concluded that UC-MSC transfusion is feasible and well tolerated in patients with PBC who respond only partially to UDCA treatment, thus representing a novel therapeutic approach for patients in this subgroup. A larger, randomized controlled cohort study is warranted to confirm the clinical efficacy of UC-MSC transfusion [471].

Treatment of PBC was also explored with allogeneic BM-MSC. Ten patients were enrolled in this trial of BM-MSCT. All patients were permitted to concurrently continue their previous UDCA treatment. The efficacy of BM-MSCT in UDCA-resistant PBC was assessed at various time points throughout the 12-month follow up. No transplantation-related side effects were observed. The life quality of the patients was improved after BM-MSCT as demonstrated by responses to the PBC-40 questionnaire. Serum levels of ALT, AST, γ-GT, and IgM significantly decreased from baseline after BM-MSCT. In addition, the percentage of CD8+ T cells was reduced, while that of CD4+CD25+Foxp3+ T cells was increased in peripheral lymphocytic subsets. Serum levels of IL-10 were also elevated. Notably, the optimal therapeutic outcome was acquired in 3 to 6 months and could be maintained for 12 months after BM-MSCT. The authors concluded that, allogeneic BM-MSCT in UDCA-resistant PBC is safe and appears to be effective [472].

Guo et al explored the immunomodulatory effects of bone marrow mesenchymal stem cells (BMSCs) on peripheral blood T lym-phocytes in patients with decompensation stage, hepatitis B-associated cirrhosis. MSCs from nine patients were analyzed by flow cytometry. Peripheral blood lymphocytes were isolated for fluorescent staining. Following stimulation by phytohemagglutinin (PHA), peripheral blood lymphocytes were co-cultured with BMSCs in serum and divided into four groups: (1) BMSC + lymphocyte + PHA contact culture group; (2) BMSC + lymphocyte + PHA non-contact culture group; (3) lym-phocyte + PHA positive control group; and (4) lymphocyte-only negative control group. Lymphocyte proliferation and frequencies of CD4(+)CD25(+)CD127(-) Tregs and CD4(+)CD8(-)IL-17(+) (Th17) cells were de-tected. Cell proliferation in groups 1 and 2 declined compared with group 3 (P < 0.01), and was notably higher than in group 4 (P < 0.01). CD4(+)CD25(+)CD127(-) Tregs frequencies in groups 1 and 2 were higher than in groups 3 and 4. In an intra-group comparison before and after culture, Th17 cell frequencies in groups 1 and 2 were higher than in group 4 (P < 0.01), but lower than in group 3 (P < 0.01). The Treg/Th17 ratio in groups 1 and 2 increased (P < 0.01), but did not change significantly in groups 3 and 4 (P > 0.05). In a comparison between groups after culture, the Treg/Th17 ratio in groups 1 and 2 increased more than in groups 3 and 4 (P < 0.01). BMSCs from cirrhotic patients can inhibit the proliferation of peripheral blood T lymphocytes, upregulate the ex-pression of CD4(+)CD25(+)CD127(-) Tregs, and improve Treg/Th17 imbalance. The mechanism by which this takes place may be associated with immunomodulatory effects induced by the secretion of soluble factors [473].

Another study also supported the possibility of immune modulation as one of the mechanisms of action of MSC. 56 patients were enrolled and randomly assigned to the MSC transplantation group and control group. After 24-week follow-up, 39 patients completed the study (20 cases in transplantation group and 19 cases in control group). The Model for End-Stage Liver Disease scores, liver function, changes of Treg/Th17 cells, as well as related transcription factors and serum cytokines, were determined. Although patients in both groups showed significant improvement after Entecavir treatment, ABMSC transplantation further improved patients’ liver function. Moreover, there was a significant increase in Treg cells and a marked decrease in Th17 cells in the transplantation group compared with control, leading to an increased Treg/Th17 ratio. Furthermore, mRNA levels of Treg-related transcription factor (Foxp3) and Th17-related transcription factor (RORyt) were increased and decreased, respectively. In addition, serum transforming growth factor-β levels were significantly higher at early weeks of transplantation, while serum levels of interleukin-17, tumor necrosis factor-α, and interleukin-6 were significantly lower in patients in the transplantation group compared with control. The authors concluded that BM-MSCs transplantation was effective in improving liver function in patients with HBV-LC, which was mediated, at least in part, through the regulation of Treg/Th17 cell balance [474].

In some embodiments, the invention teaches the treatment of lower back pain utilizing the novel described stem cells. Particularly, in some embodiments the cells are utilized as a means of treating disc degenerative disease. We provide means of teaching those in the art to utilize the disclosed stem cells by providing examples from other studies. One of the first reports of utilizing bone marrow MSC for treatment of lower disc degenerative disease (DDD) is a description of two women aged 70 and 67 years; both patients had lumbago, leg pain, and numbness. Myelography and magnetic resonance imaging showed lumbar spinal canal stenosis, and radiograph confirmed the vacuum phenomenon with instability. From the ilium of each patient, marrow fluid was collected, and MSCs were cultured using the medium containing autogenous serum. In surgery, fenestration was performed on the stenosed spinal canal and then pieces of collagen sponge containing autologous MSCs were grafted percutaneously to degenerated intervertebral discs. At 2 years after implantation, radiograph and computed tomography showed improvements in the vacuum phenomenon in both patients. On T2-weighted magnetic resonance imaging, signal intensity of intervertebral discs with cell grafts was high, thus indicating high moisture contents. X-rays showed that lumbar disc instability improved. Symptom was alleviated in both patients [475]. Based on this initial success, a larger study was performed on 10 patients with chronic back pain diagnosed with lumbar disc degeneration with intact annulus fibrosus were treated with autologous expanded bone marrow MSC injected into the nucleus pulposus area. Clinical evolution was followed for 1 year and included evaluation of back pain, disability, and quality of life. Magnetic resonance imaging measurements of disc height and fluid content were also performed. Feasibility and safety were confirmed and strong indications of clinical efficacy identified. Patients exhibited rapid improvement of pain and disability (85% of maximum in 3 months) that approached 71% of optimal efficacy. This outcome compares favorably with the results of other procedures such as spinal fusion or total disc replacement. Although disc height was not recovered, water content was significantly elevated at 12 months [476]. Twenty-two consecutive patients, who suffered of spine DDD, were treated: in 11 cases the MSCs were harvested from red bone marrow, 11 from fat tissue. The red bone marrow withdrawal was performed from the vertebral bodies; processed by a fully-automated, mobile system. The fat tissue withdrawal was acted from the subcutaneous adipose tissue; processed through a microfluidic fractioning procedure. MSCs were implanted in the central part of the nucleus pulposus of the DDD or added to bone chips to accelerate posterolateral arthrodesis. All the 14 posterolateral fusions and MSCs implantations showed at three months a complete bone bridge, stable at follow-up. The one intersomatic implantation gained a complete interbody fusion after 1 month; while 80% black discs treated with MSCs presented a new T2-W hyperintensity at postoperative MRI. The mean VAS pain score improved from 70±20 to 10±5 at 12 months, as the ODI score from 70±5% to 20±10% [477].

In order to augment therapeutic efficacy of MSC, treatment of cells with hypoxia before implantation was performed. Five patients diagnosed with degenerative disc disease received an intra-discal injection of autologous, hypoxic cultured, bone marrow-derived mesenchymal stem cells (15.1-51.6 million cells) as part of a previous study. These patients were re-consented to participate in this study in order to assess long-term safety and feasibility of intra-discal injection of autologous, hypoxic cultured, bone marrow-derived mesenchymal stem cells 4-6 years post mesenchymal stem cell infusion. The follow-up study consisted of a physical examination, a low back MRI, and a quality of life questionnaire. Patients’ lower back MRI showed absence of neoplasms or abnormalities surrounding the treated region. Based on the physical examination and the quality of life questionnaire, no adverse events were reported due to the procedure or to the stem cell treatment 4-6 years post autologous, hypoxic cultured mesenchymal stem cell infusion. All patients self-reported overall improvement, as well as improvement in strength, post stem cell treatment, and four out of five patients reported improvement in mobility [478].

Allogeneic MSC offer the potential for a standardized “drug-like” cellular therapy. A study randomized 24 patients with chronic back pain diagnosed with lumbar disk degeneration and unresponsive to conservative treatments into 2 groups. The test group received allogeneic bone marrow MSCs by intradiscal injection of 25×10 cells per segment under local anesthesia. The control group received a sham infiltration of paravertebral musculature with the anesthetic. Feasibility and safety were confirmed and indications of clinical efficacy were identified. MSC-treated patients displayed a quick and significant improvement in algofunctional indices versus the controls. This improvement seemed restricted to a group of responders that included 40% of the cohort. Degeneration, quantified by Pfirrmann grading, improved in the MSC-treated patients and worsened in the controls [479].

Another study used allogeneic human umbilical cord tissue-derived mesenchymal stem cells (HUC-MSCs) contain stem cells and possess the ability to regenerate degenerative discs. Two patients with chronic discogenic low back pain were treated with HUC-MSC transplantation. An 11-point visual analog scale (VAS, 0-10) and Oswestry Disability Index (ODI, 0-100) were used to assess the back pain symptoms and the lumbar function, respectively. After transplantation, the pain and function improved immediately in the 2 patients. The VAS and ODI scores decreased obviously during a 2-year follow-up period [480].

It is believed that matrices can enhance therapeutic activities of MSC. One approach is the implantation of live mesenchymal stem cell (MSC) allograft-containing allogeneic bone grafts used in anterior cervical discectomy and fusion (ACDF). Two matched cohorts of adult patients who underwent ACDF with MSC or standard allograft were included. A consecutive series of 57 patients who underwent a one- or two-level instrumented ACDF procedure between 2010 and 2012 were retrospectively analyzed. All fusion constructs comprised an interbody allograft, an anterior plate, and Osteocel (NuVasive, San Diego, CA, USA). These patients were matched to a control group of 57 patients. Of the 57 cases in both cohorts, 29 (50.9%) were single-level, and 28 (49.1%) were two-level instrumented ACDFs. There were no significant differences in patient age (p=0.71), gender, comorbidity burden (Charlson Comorbidity Index [CCI]: 1.95; 2.42, p=0.71) or body mass index (p=0.79). At the 1-year follow-up, 50 of 57 (87.7%) patients in the Osteocel cohort demonstrated a solid fusion compared with 54 of 57 (94.7%) in the control group (p=0.19). Seven (12.3%) patients in the Osteocel cohort were reported as having a failed fusion at 1 year. The authors concluded that patients treated with MSC allografts demonstrated lower fusion rates compared with a matched non-MSC cohort [481].

The use of the novel stem cells disclosed can be applied for treatment of Chronic Obstructive Pulmonary Disease (COPD), an umbrella term covering chronic bronchitis and emphysema, is the fourth largest cause of death in the United States and is projected to be the third by 2020 [482]. COPD is associated with an exaggerated chronic inflammatory response which is responsible for the airway abnormalities such as constriction and architectural distortion of the lung parenchyma. Patients generally undergo a progression of declining lung function, characterized by intensification of cough, shortness of breath, and sputum production. Extrapulmonary manifestations of COPD include osteoporosis, cardiovascular disease, skeletal muscle abnormalities, and depression [483]. Current treatments for COPD are primarily palliative and are based on severity of disease. According to the Global Strategy for the Diagnosis, Management, and Prevention of COPD (GOLD) guidelines, the following treatments are recommended: Stage I, which is characterized by mild obstruction, the aim is to reduce risk factors associated with exacerbations, for example by providing flu vaccine and use of short-acting bronchodilator as needed. Stage II patients are classified as moderate obstruction, where risk factors are to be reduced by vaccination, and the use of long-acting bronchodilators, as well as cardiopulmonary rehabilitation is advised in addition to short-acting bronchodilators. Patients with Stage III disease are considered to suffer from severe obstruction, in which inhaled glucocorticoids are added to the regime of Stage II. In Stage IV, which is considered very severe obstruction or moderate obstruction with evidence of chronic respiratory failure, long-term oxygen therapy is added, as well as consideration of surgical options such as lung volume reduction surgery and lung transplantation [484].

Bone marrow stem cells have been used in over a thousand cardiac patients with some indication of efficacy [314, 485]. Adipose-based stem cell therapies have been successfully used in thousands of race-horses and companion animals without adverse effects [486], as well as numerous clinical trials are ongoing and published human data reports no adverse effects (reviewed in ref [487]). Unfortunately, evaluation of stem cell therapy in COPD has lagged behind other areas of regenerative investigation, with no published clinical trial results to date; b) the underlying cause of COPD appears to be inflammatory and/or immunologically mediated[488]. The destruction of alveolar tissue is associated with T cell reactivity [489, 490], pathological pulmonary macrophage activation [491], and auto-antibody production [492]. Mesenchymal stem cells have been demonstrated to potently suppress autoreactive T cells [493, 494], inhibit macrophage activation [495], and autoantibody responses [496]. Additionally, mesenchymal stem cells can be purified in high concentrations from adipose stromal vascular tissue together with high concentrations of T regulatory cells [487], which in animal models are approximately 100 more potent than peripheral T cells at secreting cytokines therapeutic for COPD such as IL-10 [497, 498]. The use of adipose derived cells has yielded promising clinical results in autoimmune conditions such as multiple sclerosis [487]; and c) Pulmonary stem cells capable of regenerating damaged parenchymal tissue have been reported [499]. Administration of mesenchymal stem cells into neonatal oxygen-damaged lungs, which results in COPD-like alveoli dysplasia, has been demonstrated to yield improvements in two recent publications [500, 501].

The disclosed stem cells are capable of suppressing innate immune activation in the cases of COPD and other pulmonary issues. Initiation of COPD is believed to occur in many cases as result of noxious agents, particularly, but not exclusively in cigarette smoke. One established mechanism of initial alveolar injury involves smoke induced activation of inducible nitric oxide synthase (iNOS), which in turn produces cytotoxic free radicals such as peroxynitrite (ONOO⁻), which cause in mice a condition resembling emphesyma. Interestingly, mice lacking iNOS, or treated with a chemical inhibitor had some degree of protection from cigarette smoke induced pathology [502]. Studies using human neutrophils have shown that nicotine itself stimulates neutrophils to produce the inflammatory cytokine interleukin-8, in an iNOS-dependent manner [503]. Indeed the same study demonstrated that smokers possessed higher systemic levels of interleukin-8 as compared to non-smokers. This is correlated in patients with COPD which have higher inflammatory markers compared to controls, including TNF-alpha and IL-8 [504]. Another mechanism associated with COPD initiation is the generation of collagen degradation products such as the tripeptide chemoattractant N-acetyl Pro-Gly-Pro (PGP), which potently elicits neutrophil retention and activation. PGP is found in significantly higher concentrations in lavage samples of COPD patients as compared to controls, and also has been demonstrated to induce a COPD-like condition when administered into experimental animals [505]. Matrix metalloprotease (MMP)-9 has been demonstrated to be involved in the generation of PGP from collagen, and treatment of neutrophils with this agent stimulates their activation of MMP-9, thus suggesting an auto stimulatory loop [506].

The invention teaches the utilization of the novel stem cells for suppressing pulmonary inflammation. Inflammatory conditions stimulated by free radical stress and extracellular matrix degradation products stimulate various receptors within the lung to cause damage, and/or inhibit regeneration. For example, RTP801, is a protein that is inducible by HIF-1alpha, which causes death of alveolar cells in smoke induced lung injury models [507]. Yoshida et al demonstrated that Rtp801 transcript and protein are overexpressed in human emphysematous lungs and in lungs of mice exposed to cigarette smoke. Mechanistically, they found that Rtp801 was necessary and sufficient for NF-kB activation in cultured pulmonary cells and, when artificially expressed in mouse lungs by gene transfection, the protein promoted NF-kB activation, alveolar inflammation, oxidative stress and apoptosis of alveolar septal cells. Experiments furthermore demonstrated that mice lacking Rtp801 by means of gene knock-out were protected against acute cigarette smoke-induced lung injury. Protection was associated with increased mTOR signaling. Furthermore, the authors found that Rtp801 knockout mice were protected against emphysema when exposed chronically to cigarette smoke [508]. The mechanism of pulmonary damage associated with Rtp801 involves not only NF-kB associated induction of inflammatory cytokines but also ceramide-dependent apoptotic pathways. Specifically, it was demonstrated that direct lung instillation of either RTP801 expression plasmid or ceramides in mice triggered alveolar cell apoptosis and oxidative stress. RTP801 overexpression up-regulated lung ceramide levels 2.6-fold as compared to administration of a control plasmid. In turn, instillation of lung ceramides doubled the lung content of RTP801. Cell sorting after lung tissue dissociation into single-cell suspension showed that ceramide triggers both endothelial and epithelial cell apoptosis in vivo. It may be possible that endothelial apoptosis triggers a cascade of enhanced hypoxia, which in turn further augments HIF-1 alpha activation, thus self-perpetuating expression of RTP801. Interestingly, mice lacking rtp801 were protected against ceramide-induced apoptosis of epithelial type II cells, but not type I or endothelial cells [509]. This is of interest for two reasons, firstly, epithelial type II cells are known to be capable of acting as “regenerative cells” in the lung, which start proliferating after various injury signals [510], and secondly, the ceramide apoptotic pathway is triggered by various inflammatory signals associated with COPD such as TNF-alpha produced by neutrophils and monocytes [511-513].

Globally, RTP801 may be seen as a damage “sensor” molecule, amongst which other molecules such as toll like receptors (TLRs), and other activators of innate immune system play similar roles [514]. For example, TLR2, TLR3, and TLR4 have been found to be expressed in airway smooth muscle cells, which were activated by ligands found in inflammatory conditions associated with COPD and pulmonary remodeling such as extracellular matrix degradation products [515]. A clinical study performed mini-bronchoalveolar lavage (mini-BAL) on ten nonsmoker subjects without COPD, six smokers without COPD, and fifteen smokers with COPD. COPD mini-BAL showed increased neutrophil numbers, reduced neutrophil apoptosis, which was associated with increased TLR4 expression, compared with those in nonsmoker subjects without COPD. Demonstrating the importance of TLR4 was that in vitro administration of blocking antibodies to TLR4 resulted in increased neutrophil apoptosis [516]. Specific genetic variants of TLR4 have been associated with development of COPD, thus suggesting this molecule to also be a link to initiation and progression of the inflammatory state of this condition [517].

In addition to neutrophils, other cells of the innate immune system are associated with COPD. For example, natural killer (NK) cells have between found to be associated with initiation and progression of the disease. Wang et al performed a 124 patient study in smokers with COPD. They found systemic NK cell activation correlated with number of cigarettes smoked. Additionally, in induced sputum, the proportion of activated killer cells was related to disease state rather than current smoking status, with current and ex-smokers with COPD having significantly higher rates of activation than healthy smokers and healthy non-smokers [518]. NK activation is associated with production of cytotoxic factors such as granzyme, as well as various inflammatory cytokines including interferon gamma, which sensitize cells to inflammatory and immunologically mediated damage [519]. NK cell activation appears to be associated with recognition by the NK activating receptor NKG2D of the ligand RAET1ε, which is expressed on injured and stressed tissues. In fact, mice lacking NKG2D have been demonstrated to have a resistance to development of COPD-like pathology after exposure to cigarette smoke or viral infection [520]. While the natural function of NK cells in the lungs appears to be control of various infections [521], in the case of COPD it appears that these cells are “misguided” towards augmentation and self-perpetuation of the ongoing inflammatory cascade [522].

The cells of the invention, in some embodiments, may be utilized to suppress adaoptive immune responses in pulmonary pathologies. Contributions of adaptive immune cells to ongoing inflammatory processes has becoming increasingly recognized in situations such as ischemia/reperfusion injury [523], liver injury [524], and cancer [525], the situation of COPD is no exception. Suggesting a role for T cells in COPD was an early study in 1987 in which T lymphocytes were found to be significantly present in lavage fluid of patients with COPD but not controls. Furthermore, numbers of T cells were significantly reduced in responders to thiol drug tiopronin [526]. Indeed, other studies have confirmed the presence of various types of T cells, both CD4 and CD8 are present in abnormally high levels in COPD patients as compared to controls, with smoking augmenting levels of these cells [527-530]. Suggestive of a possible autoimmune activity of intrapulmonary T cells came from studies showing activated state of T cells in lungs of COPD patients. A study by Glader et al. examine peripheral blood lymphocytes from six never-smokers, eight smokers and 17 smokers with COPD. The number of lymphocytes per millilitre was higher in smokers than in never-smokers. No differences were found between the three groups in regard to proportions of lymphocyte populations, but the number of CD4+ T-cells in smokers was higher than in both never-smokers and COPD patients. The degree of T-cell activation was similar in all patient groups; however, a clear correlation between CD69 expression on CD4+ T-cells and lung function (FEV(1)% of predicted) was found when examining current smokers, with or without COPD [531]. Another study examined the Th1 associated transcription factor, STAT4, expression in lungs of patients with COPD. Th1 cells are associated with interferon gamma production and stimulation of inflammatory cascades, in part through macrophage activation and specifically stimulation of iNOS, as well as augmentation of NK activity. The study examined expression of STAT4, phospho-STAT4, IFN-gamma and T-box expressed in T-cells (T-bet) proteins in bronchial biopsies and bronchoalveolar lavage (BAL)-derived lymphocytes, obtained from 12 smokers with mild/moderate chronic obstructive pulmonary disease (COPD) (forced expiratory volume in one second (FEV1) 59 +/- 16% predicted), 14 smokers with normal lung function (FEV1 106 +/- 12% pred) and 12 nonsmoking subjects (FEV1 111 +/- 14% pred). In bronchial biopsies of COPD patients, the number of submucosal phospho-STAT4+ cells was increased (240 (22-406) versus 125 (0-492) versus 29 (0-511) cells mm(-2)) when compared with both healthy smokers and control nonsmokers, respectively. In smokers, phospho-STAT4+ cells correlated with the degree of airflow obstruction and the number of IFN-gamma+ cells. Similar results were seen in BAL (2.8 (0.2-5.9) versus 1.03 (0.09-1.6) versus 0.69 (0-2.3) lymphocytes × mL(-1) × 10(3)). In all smokers who underwent lavage, phospho-STAT4+ lymphocytes correlated with airflow obstruction and the number of IFNgamma+ lymphocytes [532].

In addition to CD4 activation, activation of CD8 T cells has been reported in COPD. An investigation of bronchoscopy with airway lavages and endobronchial mucosal biopsy sampling was performed in 35 patients with COPD, 21 healthy never-smokers and 16 smokers with normal lung function. Epithelial CD8+ lymphocyte numbers were higher in the COPD group compared to never-smoking controls. Among gated CD3+cells in BAL, the percentage of CD8+ NKG2D+ cells was enhanced in patients with COPD and smokers with normal lung function, compared to never-smokers. NKG2D is a receptor associated with stimulation of cytotoxic function by both NK cells and CD8 T cells. The percentage of CD8+ CD69+ cells and cell surface expression of CD69 were enhanced in patients with COPD and smokers with normal lung function, compared to never-smokers [533]. Given that both NKG2D and CD69 are associated with activation of CD8 cells, it is reasonable to believe that COPD is associated with an abnormality in the activation status of these cells.

In addition to activation of CD4 and CD8 T cells, there appears to be a deficiency in the T cells that are required to suppress rampant T cell activation, the T regulatory (Treg) cells. Hou et al. examined Blood samples from 57 never-smokers, 32 smokers with normal lung function and 66 patients with COPD, as well as bronchoalveolar lavage samples were taken from 12 never-smokers, 12 smokers and 18 patients with COPD. They found In peripheral blood, increased proportions of rTregs, aTregs and Fr III cells in smokers compared with never-smokers, whereas patients with COPD showed decreased rTregs and aTregs, and significantly increased Fr III cells compared with smokers. The changes in Treg subpopulations, with an overall decrease in the (aTreg+rTreg):(Fr III) ratio, indicated that immune homeostasis favoured inflammation and correlated with enhanced CD8 T-cell activation (r=-0.399, p<0.001) and forced expiratory volume in 1 s (FEV₁) % predicted value (r=0.435, p<0.001).The BAL (aTreg+rTreg):(Fr III) ratios displayed more robust correlations with FEV₁% predicted value (r=0.741, p<0.01) and activation of effector T cells(r=-0.763, p<0.001) [534]. Abnormalities of reduced Treg in COPD have also been described in animal models [535, 536].

Examples of using cell therapy for COPD may be seen in previous studies. For example, Sixty-two patients at six sites were randomized to double-blinded IV infusions of either allogeneic MSCs (Prochymal; Osiris Therapeutics Inc) or vehicle control. Patients received four monthly infusions (100 × 10⁶ cells/infusion) and were subsequently followed for 2 years after the first infusion. End points included comprehensive safety evaluation, pulmonary function testing (PFT), and quality-of-life indicators including questionnaires, 6MWT, and assessments of systemic inflammation. All study patients completed the full infusion protocol, and 74% completed the 2-year follow-up. There were no infusional toxicities and no deaths or serious adverse events deemed related to MSC administration. There were no significant differences in the overall number of adverse events, frequency of COPD exacerbations, or worsening of disease in patients treated with MSCs. There were no significant differences in PFTs or quality-of-life indicators; however, an early, significant decrease in levels of circulating C-reactive protein (CRP) was observed in patients treated with MSCs who had elevated CRP levels at study entry [537].

A phase I, prospective open-label study examined autologous BM MSC in 7 patients that had lung volume reduction surgery (LVRS) on two separate occasions. During the first LVRS bone marrow was collected, from which MSCs were isolated and expanded ex vivo. After 8 weeks, patients received two autologous MSC infusions 1 week apart, followed by the second LVRS procedure at 3 weeks after the second BM-MSC infusion. No adverse events related to MSC infusions occurred and lung tissue showed no fibrotic responses. After LVRS and MSC infusions alveolar septa showed a 3-fold increased expression of the endothelial marker CD31. The authors concluded that autologous MSC treatment in severe emphysema is feasible and safe. The increase in CD31 expression after LVRS and MSC treatment suggests responsiveness of microvascular endothelial cells in the most severely affected parts of the lung [538].

The invention provides the use of novel stem cells for treatments of bronchopulmonary dysplasia. In a phase I dose-escalation trial, the safety and feasibility of a single, intratracheal transplantation of hUCB-derived MSCs in preterm infants at high risk for bronchopulmonary dysplasia (BPD). The first 3 patients were given a low dose (1 × 10(7) cells/kg) of cells, and the next 6 patients were given a high dose (2 × 10(7) cells/kg). We compared their adverse outcomes, including BPD severity, with those of historical case-matched comparison group. Intratracheal MSC transplantation was performed in 9 preterm infants, with a mean gestational age of 25.3 ± 0.9 weeks and a mean birth weight of 793 ± 127 g, at a mean of 10.4 ± 2.6 days after birth. The treatments were well tolerated, without serious adverse effects or dose-limiting toxicity attributable to the transplantation. Levels of interleukin-6, interleukin-8, matrix metalloproteinase-9, tumor necrosis factor α, and transforming growth factor β1 in tracheal aspirates at day 7 were significantly reduced compared with those at baseline or at day 3 posttransplantation. BPD severity was lower in the transplant recipients, and rates of other adverse outcomes did not differ between the comparison group and transplant recipients. Intratracheal transplantation of allogeneic hUCB-derived MSCs in preterm infants is safe and feasible, and warrants a larger and controlled phase II study [539].

In one embodiment, the invention teaches the use of the novel stem cells in treatment of ARDS. A case report was presented of 59-yr-old man who was treated with umbilical cord MSCs (UC-MSC) in the course of ARDS and subsequent pulmonary fibrosis. He received a long period of mechanical ventilation and weaning proved difficult. On hospital day 114, he underwent the intratracheal administration of UCB-derived MSCs at a dose of 1 × 10(6)/kg. After cell infusion, an immediate improvement was shown in his mental status, his lung compliance (from 22.7 mL/cmH2O to 27.9 mL/cmH2O), PaO2/FiO2 ratio (from 191 mmHg to 334 mmHg) and his chest radiography over the course of three days. Even though he finally died of repeated pulmonary infection, our current findings suggest the possibility of using MSCs therapy in an ARDS patient [540]. Another clinical study evaluated twelve adult patients meeting the Berlin definition of ARDS with a PaO2/FiO2 ratio of < 20.0. Patients were randomized to receive allogeneic adipose-derived MSCs or placebo in a 1:1 fashion. Patients received one intravenous dose of 1 × 106 cells/kg of body weight or saline. There were no infusion toxicities or serious adverse events related to MSCs administration and there were no significant differences in the overall number of adverse events between the two groups. Length of hospital stay, ventilator-free days and ICU-free days at day 28 after treatment were similar. There were no changes in biomarkers examined in the placebo group. In the MSCs group, serum surfactant protein-D levels at day 5 were significantly lower than those at day 0 (p = 0.027) while the changes in IL-8 levels were not significant. The IL-6 levels at day 5 showed a trend towards lower levels as compared with day 0, but this trend was not statistically significant (p = 0.06). Although safety was demonstrated, clinical effect with the doses of MSCs used was weak [541].

Differences have been published between adipose and BM MSC. Accordingly, a multicentre, open-label, dose-escalation, phase 1 clinical trial was performed. Patients were included if they had moderate-to-severe ARDS as defined by the acute onset of the need for positive pressure ventilation by an endotracheal or tracheal tube, a PaO2:FiO2 less than 200 mm Hg with at least 8 cm H2O positive end-expiratory airway pressure (PEEP), and bilateral infiltrates consistent with pulmonary oedema on frontal chest radiograph. The first three patients were treated with low dose MSCs (1 million cells/kg predicted bodyweight [PBW]), the next three patients received intermediate dose MSCs (5 million cells/kg PBW), and the final three patients received high dose MSCs (10 million cells/kg PBW). No infusion-associated events or treatment-related adverse events were reported in any of the nine patients. Serious adverse events were subsequently noted in three patients during the weeks after the infusion: one patient died on study day 9, one patient died on study day 31, and one patient was discovered to have multiple embolic infarcts of the spleen, kidneys, and brain that were age-indeterminate, but thought to have occurred before the MSC infusion based on MRI results. None of these severe adverse events were thought to be MSC-related[542] .

A detailed analysis of the immunomodulatory properties and proteomic profile of MSCs systemically administered to two patients with refractory ARDS. Both patients received 2×10(6) cells per kilogram, and each subsequently improved with resolution of respiratory, hemodynamic, and multiorgan failure. In parallel, a decrease was seen in multiple pulmonary and systemic markers of inflammation, including epithelial apoptosis, alveolar-capillary fluid leakage, and proinflammatory cytokines, microRNAs, and chemokines. In vitro studies of the MSCs demonstrated a broad anti-inflammatory capacity, including suppression of T-cell responses and induction of regulatory phenotypes in T cells, monocytes, and neutrophils. Some of these in vitro potency assessments correlated with, and were relevant to, the observed in vivo actions. These experiences highlight both the mechanistic information that can be gained from clinical experience and the value of correlating in vitro potency assessments with clinical effects. The findings also suggest, but do not prove, a beneficial effect of lung protective strategies using adoptively transferred MSCs in ARDS [543].

The invention provides the use of novel stem cells to treat interstitial lung disease. case report described a patient of ILD treated with allogeneic BM-MSC infusion. The index patient had end-stage ILD due to a combination of insults including treatment with radiotherapy and a tyrosine kinase inhibitor Erlotinib. He was oxygen-dependent and this was hampering his quality of life. He tolerated the first infusion stem cells without any problem. During the second infusion he developed anaphylactic shock, which was appropriately managed. At 6-months follow-up he had no improvement in oxygenation, pulmonary function or CT scan parameters. To our knowledge, this is one of the only cases of anaphylaxis associated with allogeneic MSC infusion [544] .

The invention provides the use of novel stem cells to treat idiopathic pulmonary fibrosis. Autologous BM-MSC were used 8 idiopathic pulmonary fibrosis (IPF) patients in a single centre, non-randomized, dose escalation phase 1b trial, patients with moderately severe IPF (diffusing capacity for carbon monoxide (DLCO) ≥ 25% and forced vital capacity (FVC) ≥ 50%) received either 1 × 10(6) (n = 4) or 2 × 10(6) (n = 4) unrelated-donor, placenta-derived MSC/kg via a peripheral vein and were followed for 6 months with lung function (FVC and DLCO ), 6-min walk distance (6MWD) and computed tomography (CT) chest. The patients (4 female, aged 63.5 (57-75) years) with median (interquartile range) FVC 60 (52.5-74.5)% and DLCO 34.5 (29.5-40)% predicted were treated. Both dose schedules were well tolerated with only minor and transient acute adverse effects. MSC infusion was associated with a transient (1% (0-2%)) fall in SaO2 after 15 min, but no changes in haemodynamics. At 6 months FVC, DLCO, 6MWD and CT fibrosis score were unchanged compared with baseline. There was no evidence of worsening fibrosis [545].

The invention provides the use of novel stem cells to treat sarcoidosis. Placental MSC termed PDA-001 (made by Celgene Inc) were culture-expanded in vitro as a plastic-adherent, undifferentiated cell population that expresses the nominal phenotype CD34-, CD10+, CD105+ and CD200+. Four patients with refractory pulmonary sarcoidosis received two infusions of 150 million PDA-001 cells in 240 ml dextran-40 solution one week apart. During and for two hours after the first infusion, the pulmonary artery pressure was monitored. Prior to first infusion and within 24 hours after the second infusion, bronchoscopy and bronchoalveolar lavage (BAL) were performed. After the first infusion, all patients had a mild, non-clinically significant increase in mean pulmonary artery pressure, but none exhibited right heart failure or volume overload. In the year following treatment, there was no significant change in the FVC, but two patients had improvement in their chest x-ray and had prednisone withdrawn. BAL samples after the second infusion were sufficiently viable to undergo FACS analysis in three cases and in two patients, CD10+CD49c+C105+ cells (indicative of PDA-001 cells) were found. Two of four patients demonstrated some steroid sparing benefit, including one patient with prolonged remission. [546]

The invention provides the use of novel stem cells to treat tuberculosis. A large study described 108 Belarussians with multi drug MDR-TB patients receiving antibiotics. Thirty-six patients (“cases”) also had bone marrow MSCs extracted, cultured and re-infused (average time from antibiotic start to infusion was 49 days); another 36 patients were “study controls”. We identified another control group: 36 patients from the Belarussian surveillance database (“surveillance controls”) 1: 1 matched to cases. Of the cases, 81% had successful outcomes versus 42% of surveillance controls and 39% of study controls. Successful outcome odds were 6.5 (95% Confidence Interval: 1.2-36.2, p=0.032) times greater for cases than surveillance controls (age-adjusted). Radiological improvement was more likely in cases than study controls. Culture analysis prior to infusion demonstrated a poorer initial prognosis in cases, yet despite this they had better outcomes than the control groups. MSC treatment could vastly improve outcomes for MDR-TB patients [547].

In another study, 30 patients with MDR and extensively drug-resistant (XDR) tuberculosis, were treated with single-dose autologous bone marrow-derived MSCs (aimed for 1×10(6) cells per kg), within 4 weeks of the start of antituberculosis-drug treatment. Inclusion patients were those with pulmonary tuberculosis confirmed by sputum smear microscopy, culture, or both; MDR or XDR tuberculosis confirmed by drug-susceptibility testing to first-line and second-line drugs; age older than 21 years to 65 years or younger; and absence of lesion compatible with a malignant process or ongoing tuberculosis in organs other than the lungs and pleura. The most common (grade 1 or 2) adverse events were high cholesterol levels (14 of 30 patients), nausea (11 of 30 patients), and lymphopenia or diarrhea (ten of 30 patients). There were no serious adverse events reported. We recorded two grade 3 events that were transitory-ie, increased plasma potassium ion concentrations in one patient and a transitory grade 3 γ-glutamyltransferase elevation in another patient. It is unclear if the adverse effects where related to treatment intervention given the advanced disease of these patients [548].

Another utilization of the invention is for the treatment of systemic lupus erythromatosis. The initial publication using MSC for lupus described 4 patients who were non-responsive to CTX/glucocorticoid treatment using allogenic BM-MSC and showed a stable 12-18 months disease remission in all treated patients. The patients benefited an amelioration of disease activity, improvement in serologic markers and renal function [496].

A subsequent study using autologous BM-MSC found no effect on disease activity but an increase in T regulatory cells. The study described safety and efficacy of bone marrow (BM)-derived MSCs in two SLE patients; the suppressor effect of these cells in-vitro and the change in CD4+CD25+FoxP3+ T regulatory (Treg) cells in response to treatment. Two females (JQ and SA) of 19 and 25 years of age, fulfilling the 1997 American College of Rheumatology (ACR) criteria for SLE were infused with autologous BM-derived MSCs. Disease activity indexes and immunological parameters were assessed at baseline, 1, 2, 7 and 14 weeks. Peripheral blood lymphocyte (PBL) subsets and Treg cells were quantitated by flow cytometry, and MSCs tested for in-vitro suppression of activation and proliferation of normal PBLs. No adverse effects or change in disease activity indexes were noted during 14 weeks of follow-up, although circulating Treg cells increased markedly. Patient MSCs effectively suppressed in-vitro PBL function. However, JQ developed overt renal disease 4 months after infusion. MSC infusion was without adverse effects, but did not modify initial disease activity in spite of increasing CD4+CD25+FoxP3+ cell counts. One patient subsequently had a renal flare [549].

Umbilical cord MSC (UC-MSC) were utilized in a larger study in an allogeneic setting. The single-arm trial involved 16 SLE patients whose disease was refractory to standard treatment or who had life-threatening visceral involvement. The median follow-up time after UC-MSC therapy was 8.25 months (range 3-28 months). Significant improvements in the SLEDAI score, levels of serum ANA, anti-dsDNA antibody, serum albumin, and complement C3, and renal function were observed. Clinical remission was accompanied by an increase in peripheral Treg cells and a re-established balance between Th1- and Th2-related cytokines. Significant reduction in disease activity was achieved in all patients, and there has been no recurrence to date and no treatment-related deaths [550].

A subsequent study by the same group addressed SLE patients who have diffuse alveolar hemorrhage (DAH) is a rare complication of SLE with a high mortality usually over 50%. Four SLE patients complicated with DAH, who underwent UC-MSC. All the four patients showed dramatic improvements of their clinical manifestations. Hemoglobin was elevated after UC-MSC and was sustained at a normal level 6 months after UC-MSC in the four patients. Platelet level was upregulated in two patients who had thrombocytopenia at baseline. Oxygen saturation appeared to be normal at 1 month after UC-MSC, and this result was confirmed by the scan of the chest. Serum albumin elevated to 3.5 g/dl 6 months after transplantation. These findings suggest that UC-MSC results in amelioration of oxygen saturation as well as hematological and serologic changes, which revealed that UC-MSC could be applied as a salvage strategy for DAH patients [551].

In an attempt to increase efficacy, the same group attempted to ascertain whether a double infusion of UC-MSC can enhance therapeutic efficacy. Fifty-eight refractory SLE patients were enrolled in this study, in which 30 were randomly given single UC-MSC, and the other 28 were given double UC-MSC. Patients were followed up for rates of survival, disease remission, and relapse, as well as transplantation-related adverse events. SLE disease activity index (SLEDAI) and serologic features were evaluated. The results showed that no remarkable differences between single and double allogenic MSCT were found in terms of disease remission and relapse, amelioration of disease activity, and serum indexes in an SLE clinical trial with more than one year followup. This study demonstrated that single MSCs transplantation at the dose of one million MSCs per kilogram of body weight was sufficient to induce disease remission for refractory SLE patients [552].

Longer follow-up of treated patients with allogeneic UC-MSC or BM-MSC was reported. Eighty-seven patients with persistently active SLE who were refractory to standard treatment or had life-threatening visceral involvement were enrolled. Allogeneic bone marrow or umbilical cord-derived MSCs were harvested and infused intravenously (1 × 10(6) cells/kg of body weight). Primary outcomes were rates of survival, disease remission and relapse, as well as transplantation-related adverse events. Secondary outcomes included SLE disease activity index (SLEDAI) and serologic features. During the 4-year follow-up and with a mean follow-up period of 27 months, the overall rate of survival was 94% (82/87). Complete clinical remission rate was 28% at 1 year (23/83), 31% at 2 years (12/39), 42% at 3 years (5/12), and 50% at 4 years (3/6). Rates of relapse were 12% (10/83) at 1 year, 18% (7/39) at 2 years, 17% (2/12) at 3 years, and 17% (⅙) at 4 years. The overall rate of relapse was 23% (20/87). Disease activity declined as revealed by significant changes in the SLEDAI score, levels of serum autoantibodies, albumin, and complements. A total of five patients (6%) died after MSCT from non-treatment-related events in the 4-year follow-up, and no transplantation-related adverse event was observed. Allogeneic MSCT resulted in the induction of clinical remission and improvement in organ dysfunction in drug-resistant SLE patients [553].

A subsequent multi-center study evaluated UC-MSC in refractory SLE. Forty patients with active SLE were recruited from four clinical centers. Allogeneic UC MSCs were infused intravenously on days 0 and 7. The primary endpoints were safety profiles. The secondary endpoints included major clinical response (MCR), partial clinical response (PCR) and relapse. Clinical indices, including Systemic Lupus Erythematosus Disease Activity Index (SLEDAI) score, British Isles Lupus Assessment Group (BILAG) score and renal functional indices, were also taken into account. The overall survival rate was 92.5% (37 of 40 patients). UC-MSCT was well tolerated, and no transplantation-related adverse events were observed. Thirteen and eleven patients achieved MCR (13 of 40, 32.5%) and PCR (11 of 40, 27.5%), respectively, during 12 months of follow up. Three and four patients experienced disease relapse at 9 months (12.5%) and 12 months (16.7%) of follow-up, respectively, after a prior clinical response. SLEDAI scores significantly decreased at 3, 6, 9 and 12 months follow-up. Total BILAG scores markedly decreased at 3 months and continued to decrease at subsequent follow-up visits. BILAG scores for renal, hematopoietic and cutaneous systems significantly improved. Among those patients with lupus nephritis, 24-hour proteinuria declined after transplantation, with statistically differences at 9 and 12 months. Serum creatinine and urea nitrogen decreased to the lowest level at 6 months, but these values slightly increased at 9 and 12 months in seven relapse cases. In addition, serum levels of albumin and complement 3 increased after MSCT, peaked at 6 months and then slightly declined by the 9- and 12-month follow-up examinations. Serum antinuclear antibody and anti-double-stranded DNA antibody decreased after MSCT, with statistically significant differences at 3-month follow-up examinations. The authors concluded that there may be a need for UC-MSC re-administration at 6 months due to observations of relapse [554].

Lupus nephritis (LN) is a complication of SLE. In an open-label and single-center clinical trial 81 Chinese patients with active and refractory LN were enrolled. Allogeneic bone marrow- or umbilical cord-derived mesenchymal stem cells (MSCs) were administered intravenously at the dose of 1 million cells per kilogram of bodyweight. All patients were then monitored over the course of 12 months with periodic follow-up visits to evaluate renal remission, as well as possible adverse events. The primary outcome was complete renal remission (CR) and partial remission (PR) at each follow-up, as well as renal flares. The secondary outcome included renal activity score, total disease activity score, renal function, and serologic index. During the 12-month follow-up, the overall rate of survival was 95 % (77/81). Totally, 60.5 % (49/81) patients achieved renal remission during 12-month visit by MSCT. Eleven of 49 (22.4 %) patients experienced renal flare by the end of 12 months after a previous remission. Renal activity evaluated by British Isles Lupus Assessment Group (BILAG) scores significantly declined after MSCT (mean ± SD, from 4.48 ± 2.60 at baseline to 1.09 ± 0.83 at 12 months), in parallel with the obvious amelioration of renal function. Glomerular filtration rate (GFR) improved significantly 12 months after MSCT (mean ± SD, from 58.55 ± 19.16 to 69.51 ± 27.93 mL/min). Total disease activity evaluated by Systemic Lupus Erythematosus Disease Activity Index (SLEDAI) scores also decreased after treatment (mean ± SD, from 13.11 ± 4.20 at baseline to 5.48 ± 2.77 at 12 months). Additionally, the doses of concomitant prednisone and immunosuppressive drugs were tapered. No transplantation-related adverse event was observed. Allogeneic MSCT resulted in renal remission for active LN patients within 12-month visit, confirming its use as a potential therapy for refractory LN [555].

One of the important aspects of MSC clinical trials is generally lack of long term follow-ups. To address this, 9 SLE patients, who were refractory to steroid and immunosuppressive drugs treatment and underwent MSCs transplantation in 2009, were enrolled. One million allogeneic UC MSCs per kilogram of body weight were infused intravenously at days 0 and 7. The possible adverse events, including immediately after MSCs infusions, as well as the long-term safety profiles were observed. Blood and urine routine test, liver function, electrocardiogram, chest radiography and serum levels of tumor markers, including alpha fetal protein (AFP), cancer embryo antigen (CEA), carbohydrate antigen 155 (CA155) and CA199, were assayed before and 1, 2, 4 and 6 years after MSCs transplantation. All the patients completed two times of MSCs infusions. One patient had mild dizzy and warm sensation 5 min after MSCs infusion, and the symptoms disappeared quickly. No other adverse event, including fluster, headache, nausea or vomit, was observed. There was no change in peripheral white blood cell count, red blood cell count and platelet number in these patients after followed up for 6 years. Liver functional analysis showed that serum alanine aminotransferase, glutamic-oxalacetic transaminase, total bilirubin and direct bilirubin remained in normal range after MSCs infusions. No newly onset abnormality was detected on electrocardiogram and chest radiography. Moreover, the investigators found no rise of serum tumor markers, including AFP, CEA, CA125 and CA199, before and 6 years after MSCs infusions [556].

Immunological mechanisms by which UC-MSC may suppress SLE include downregulation of Th17 cells and upregulation of Treg. Thirty patients with active SLE, refractory to conventional therapies, were given UC MSCs infusions. The percentages of peripheral blood CD4+CD25+Foxp3+ regulatory T cells (Treg) and CD3+CD8-IL17A+Th17 cells and the mean fluorescence intensities (MFI) of Foxp3 and IL-17 were measured at 1 week, 1 month, 3 months, 6 months, and 12 months after MSCs transplantation (MSCT). Serum cytokines, including transforming growth factor beta (TGF-β), tumor necrosis factor alpha (TNF-α), interleukin 6 (IL-6), and IL- 17A were detected using ELISA. Peripheral blood mononuclear cells from patients were collected and co-cultured with UC MSCs at ratios of 1:1, 10: 1, and 50:1, respectively, for 72 h to detect the proportions of Treg and Th17 cells and the MFIs of Foxp3 and IL-17 were determined by flow cytometry. The cytokines in the supernatant solution were detected using ELISA. Inhibitors targeting TGF-β, IL-6, indoleamine 2,3-dioxygenase (IDO), and prostaglandin E2 were added to the co-culture system, and the percentages of Treg and Th17 cells were observed. The percentage of peripheral Treg and Foxp3 MFI increased 1 week, 1 month, and 3 months after UC MSCs transplantation, while the Th17 proportion and MFI of IL-17 decreased 3 months, 6 months, and 12 months after the treatment, along with an increase in serum TGF-β at 1 week, 3 months, and 12 months and a decrease in serum TNF-α beginning at 1 week. There were no alterations in serums IL-6 and IL-17A before or after MSCT. In vitro studies showed that the UC MSCs dose-dependently up-regulated peripheral Treg proportion in SLE patients, which was not depended on cell-cell contact. However, the down-regulation of Th17 cells was not dose-dependently and also not depended on cell-cell contact. Supernatant TGF-β and IL-6 levels significantly increased, TNF-α significantly decreased, but IL-17A had no change after the co-culture. The addition of anti-TGF-β antibody significantly abrogated the up-regulation of Treg, and the addition of PGE2 inhibitor significantly abrogated the down-regulation of Th17 cells. Both anti-IL-6 antibody and IDO inhibitor had no effects on Treg and Th17 cells [557].

The invention teaches the use of novel MSC for treatment of multiple sclerosis. Multiple sclerosis (MS) is an autoimmune condition in which the immune system attacks the central nervous system (CNS), leading to demyelination. It may cause numerous physical and mental symptoms, and often progresses to physical and cognitive disability. Disease onset usually occurs in young adults, and is more common in women [558]. MS affects the areas of the brain and spinal cord known as the white matter. Specifically, MS destroys oligodendrocytes, which are the cells responsible for creating and maintaining the myelin sheath, which helps the neurons carry electrical signals. MS results in a thinning or complete loss of myelin and, less frequently, transection of axons [559]. Current therapies for MS include steroids, immune suppressants (cyclosporine, azathioprine, methotrexate), immune modulators (interferons, glatiramer acetate), and immune modulating antibodies (natalizumab). At present none of the MS treatment available on the market selectively inhibit the immune attack against the nervous system, nor do they stimulate regeneration of previously damaged tissue.

Induction of remission in MS has been associated with stimulation of T regulatory cells. For example, patients responding to the clinically used immune modulatory drug glatiramer acetate have been reported to have increased levels of CD4+, CD25+, FoxP3+ Treg cells in peripheral blood and cerebral spinal fluid [560]. Interferon beta, another clinically used drug for MS induces a renormalization of Treg activity after initiation of therapy through stimulation of de novo regulatory cell generation [561]. In the animal model of MS, experimental allergic encephalomyelitis (EAE), disease progression is exacerbated by Treg depletion [562], and natural protection against disease in certain models of EAE is associated with antigen-specific Treg [563]. Thus there is some reason to believe that stimulation of the Treg compartment may be therapeutically beneficial in MS.

In addition to immune damage, MS patients are known to have a certain degree of recovery based on endogenous repair processes. Pregnancy associated MS remission has been demonstrated to be associated with increased white matter plasticity and oligodendrocyte repair activity [564]. Functional MRI (fMRI) studies have suggested that various behavioral modifications may augment repair processes at least in a subset of MS patients [565]. Endogenous stem cells in the sub-ventricular zone of brains of mice and humans with MS have been demonstrated to possess ability to differentiate into oligodendrocytes and to some extent assist in remyelination [565]. For example, an 8-fold increase in de novo differentiating sub-ventricular zone derived cells was observed in autopsy samples of MS patients in active as compared to non-active lesions [566].

The therapeutic effects of MSC in MS have been demonstrated in several animal studies. In one of the first studies of immune modulation, Zappia et al. demonstrated administration of MSC subsequent to immunization with encephalomyelitis-inducing bovine myelin prevented onset of the mouse MS-like disease EAE. The investigators attributed the therapeutic effects to stimulation of Treg cells, deviation of cytokine profile, and apoptosis of activated T cells [567]. It is interesting to note that the MSC were injected intravenously. Several other studies have shown inhibition of EAE using various MSC injection protocols [250, 568].

Ten patients with progressive MS that had not responded to disease modifying agents including Mitoxantrone where treated with autologous. Their Expanded Disability Status Scale (EDSS) score ranged from 3.5 to 6. Patients were injected intrathecally with culture expanded MSCs. They were followed with monthly neurological assessment and a MRI scan at the end of the first year. During 13 to 26 months of follow up (mean: 19 months), the EDSS of one patient improved from 5 to 2.5 score. Four patients showed no change in EDSS. Five patients’ EDSS increased from 0.5 to 2.5. In the functional system assessment, six patients showed some degree of improvement in their sensory, pyramidal, and cerebellar functions. One showed no difference in clinical assessment and three deteriorated. The result of MRI assessment after 12 months was as following: seven patients with no difference, two showed an extra plaque, and one patient showed decrease in the number of plaques [569].

In another study, autologous BM-MSC where administered in 7 patients with advanced multiple sclerosis (MS). Patients were assessed at 3, 6 and 12 months. Assessment at 3-6 months revealed Expanded Disability Scale Score (EDSS) improvement in 5/7, stabilization in ⅐, and worsening in ⅐ patients. MRI at 3 months revealed new or enlarging lesions in 5/7 and Gadolinium (Gd+) enhancing lesions in 3/7 patients. Vision and low contrast sensitivity testing at 3 months showed improvement in ⅚ and worsening in ⅙ patients. Early results show hints of clinical but not radiological efficacy and evidence of safety with no serious adverse events [570].

Another autologous BM-MSC study recruited 15 patients with MS (mean [SD] Expanded Disability Status Scale [EDSS] score, 6.7 [1.0]). After culture, a mean (SD) of 63.2 × 10(6) (2.5 × 10(6)) MSCs was injected intrathecally (n = 34) and intravenously (n = 14). No major adverse effects were reported during follow-up. Mean EDSS score improved from 6.7 (1.0) to 5.9 (1.6). Magnetic resonance imaging visualized the MSCs in the occipital horns of the ventricles, indicating the possible migration of ferumoxides-labeled cells in the meninges, subarachnoid space, and spinal cord. Immunological analysis revealed an increase in the proportion of CD4(+)CD25(+) regulatory T cells, a decrease in the proliferative responses of lymphocytes, and the expression of CD40(+), CD83(+), CD86(+), and HLA-DR on myeloid dendritic cells at 24 hours after MSC transplantation [571].

In a larger study, patients with secondary progressive multiple sclerosis involving the visual pathways (expanded disability status score 5·5-6·5) were treated by intravenous infusion of autologous bone-marrow-derived mesenchymal stem cells. The primary objective was to assess feasibility and safety. Adverse events from up to 20 months before treatment until up to 10 months after the infusion where compared. As a secondary objective, efficacy the efficacy outcome was the anterior visual pathway as a model of wider disease. Masked endpoint analyses was used for electrophysiological and selected imaging outcomes. The mean dose was 1·6×10(6) cells per kg body weight (range 1·1-2·0). One patient developed a transient rash shortly after treatment; two patients had self-limiting bacterial infections 3-4 weeks after treatment. The authors did not identify any serious adverse events. Improvement after treatment in visual acuity (difference in monthly rates of change -0·02 logMAR units, 95% CI -0·03 to -0·01; p=0·003) and visual evoked response latency (-1·33 ms, -2·44 to -0·21; p=0·020), with an increase in optic nerve area (difference in monthly rates of change 0·13 mm(2), 0·04 to 0·22; p=0·006) where observed. No significant effects on colour vision, visual fields, macular volume, retinal nerve fibre layer thickness, or optic nerve magnetisation transfer ratio [572].

In another study, 25 patients with progressive MS (expanded disability status scale score: 4.0 -6.50) unresponsive to conventional treatments were recruited for this study. BM-MSC were administered by a single intrathecal injection. Associated short-term adverse events of injection consisted of transient low-grade fever, nausea /vomiting, weakness in the lower limbs and headache. No major delayed adverse effect was reported. 3 patients left the study for personal reasons. The mean (SD) expanded disability status scale (EDSS) score of 22 patients changed from 6.1 (0.6) to 6.3 (0.4). Clinical course of the disease (measured by EDSS) improved in 4, deteriorated in 6 and had no change in 12 patients. In MRI evaluation, 15 patients showed no change, whereas 6 patients showed new T2 or gadolinium enhanced lesions (1 lost to follow-up). The authors concluded that MSC therapy can improve/stabilize the course of the disease in progressive MS in the first year after injection with no serious adverse effects [573].

A small placebo controlled study assessed 9 patients unresponsive to conventional therapy, defined by at least 1 relapse and/or GEL on MRI scan in past 12 months, disease duration 2 to 10 years and Expanded Disability Status Scale (EDSS) 3.0-6.5 were randomized to receive IV 1-2×10(6) bone-marrow-derived-MSCs/Kg or placebo. After 6 months, the treatment was reversed and patients were followed-up for another 6 months. Secondary endpoints were clinical outcomes (relapses and disability by EDSS and MS Functional Composite), and several brain MRI and optical coherence tomography measures. Immunological tests were explored to assess the immunomodulatory effects. At baseline 9 patients were randomized to receive MSCs (n=5) or placebo (n=4). One patient on placebo withdrew after having 3 relapses in the first 5 months. We did not identify any serious adverse events. At 6 months, patients treated with MSCs had a trend to lower mean cumulative number of GEL (3.1, 95% CI=1.1-8.8 vs 12.3, 95% CI=4.4-34.5, p=0.064), and at the end of study to reduced mean GEL (-2.8±5.9 vs 3±5.4, p=0.075) [574].

A 15 patients study evaluated the safety and efficacy of autologous bone marrow-derived mesenchymal stem cells (MSCs) as a potential treatment for neuromyelitis optica spectrum disorder (NMOSD), a complication of MS. Fifteen patients with NMOSD were recruited. All patients received a single intravenous infusion of 1.0 × 10(8) autologous MSC within 3-4 generations derived from bone marrow. The primary endpoints of the study were efficacy as reflected by reduction in annualized relapse rates (ARRs) and inflammatory lesions observed by MRI. At 12 months after MSC infusion, the mean ARR was reduced (1.1 vs. 0.3, P = 0.002), and the T2 or gadolinium-enhancing T1 lesions decreased in the optic nerve and spinal cord. Disability in these patients was reduced (EDSS, 4.3 vs. 4.9, P = 0.021; visual acuity, 0.4 vs. 0.5, P = 0.007). The patients had an increase in retinal nerve fiber layer thickness, optic nerve diameters and upper cervical cord area. The authors did not identify any serious MSC-related adverse events. At 24 months of MSC infusion, of 15 patients, 13 patients (87%) remained relapse-free, the mean ARR decreased to 0.1; the disability of 6 patients (40%) was improved, and the mean EDSS decreased to 4.0. The study demonstrates that MSC infusion is safe, reduces the relapse frequency, and mitigates neurological disability with neural structures in the optic nerve and spinal cord recover in patients with NMOSD. The beneficial effect of MSC infusion on NMOSD was maintained, at least to some degree, throughout a 2-year observational period [575].

A unique case reported described a 46-year-old male diagnosed with neuromyelitis optica (NMO) (an autoimmune, demyelinating CNS disorder) who had relapses with paraplegia despite treatment and developed two stage IV pressure ulcers (PUs) on his legs. The patient consented for local application of autologous MSCs on PUs. MSCs isolated from the patient’s bone marrow aspirate were multiplied in vitro during three passages and embedded in a tridimensional collagen-rich matrix which was applied on the PUs. Eight days after MSCs application the patient showed a progressive healing of PUs and improvement of disability. Two months later the patient was able to walk 20 m with bilateral assistance and one year later he started to walk without assistance. For 76 months the patient had no relapse and no adverse event was reported. The original method of local application of autologous BM-MSCs contributed to healing of PUs. For 6 years the patient was free of relapses and showed an improvement of disability. The association of cutaneous repair, sustained remission of NMO and improvement of disability might be explained by a promotion/optimization of recovery mechanisms in the central nervous system even if alternative hypothesis should be considered [576]. This report is particularly interestingly because it implies the potential of achieving systemic immune modulatory/disease modulatory responses with local applications of MSC. The suggests that administration of readily available sources of MSC such as amniotic membrane grafts could be useful in treatment of autoimmune conditions.

The group of Dr. Sun from China reported administration of 1 million/kg UC-MSC in a patient with refractory progressive MS, and the disease course was stabilized after the transplantation. Stabilization was documented by EDSS score and reduction of plaques by MRI [401].

A case report described the treatment of aggressive multiple sclerosis with multiple allogenic human umbilical cord-derived mesenchymal stem cell and autologous bone marrow-derived mesenchymal stem cells over a 4 y period. The treatments were tolerated well with no significant adverse events. Clinical and radiological disease appeared to be suppressed following the treatments and support the expansion of mesenchymal stem cell transplantation into clinical trials as a potential novel therapy for patients with aggressive multiple sclerosis [577].

Twenty-three patients were enrolled in a study evaluating UC-MSC in refractory MS. Thirteen patients where infused with UC-MSC at the same time as anti-inflammatory treatment, whereas 10 control patients received the anti-inflammatory treatment only. Treatment schedule included 1,000 mg/kg of methylprednisolone intravenously (IV) daily for 3 days and then 500 mg/kg for 2 days, followed by oral prednisone 1 mg/kg/day for 10 days. The dosage of prednisone was then reduced by 5 mg every 2 weeks until reaching a 5-mg/day maintenance dosage. Intravenous infusion of UC-MSCs was applied three times in a 6-week period for each patient. The overall symptoms of the UC-MSC-treated patients improved compared to patients in the control group. Both the EDSS scores and relapse occurrence were significantly lower than those of the control patients. Inflammatory cytokines were assessed, and the data demonstrated a shift from Th1 to Th2 immunity in UC-MSC-treated patients [578].

Sixteen patients with relapsing-remitting multiple sclerosis or secondary progressive multiple sclerosis were randomized 3:1 to receive 2 low-dose infusions of placental MSC generated by Celgene Corporation (PDA-001) at a concentration of (150×10(6) cells) or placebo, given 1 week apart. After completing this cohort, subsequent patients received high-dose PDA-001 (600×10(6) cells) or placebo. Monthly brain magnetic resonance imaging scans were performed. The primary end point was ruling out the possibility of paradoxical worsening of MS disease activity. This was monitored using Cutter’s rule (≥5 new gadolinium lesions on 2 consecutive scans) by brain magnetic resonance imaging on a monthly basis for six months and also the frequency of multiple sclerosis relapse. Ten patients with relapsing-remitting multiple sclerosis and 6 with secondary progressive multiple sclerosis were randomly assigned to treatment: 6 to low-dose PDA-001, 6 to high-dose PDA-001, and 4 to placebo. No patient met Cutter’s rule. One patient receiving high-dose PDA-001 had an increase in T2 and gadolinium lesions and in Expanded Disability Status Scale score during a multiple sclerosis flare 5 months after receiving PDA-001. No other patient had an increase in Expanded Disability Status Scale score>0.5, and most had stable or decreasing Expanded Disability Status Scale scores. With high-dose PDA-001, 1 patient experienced a grade 1 anaphylactoid reaction and 1 had grade 2 superficial thrombophlebitis. Other adverse events were mild to moderate and included headache, fatigue, infusion site reactions, and urinary tract infection. The authors concluded that PDA-001 infusions are safe and well tolerated in relapsing-remitting multiple sclerosis and secondary progressive multiple sclerosis patients [579].

The invention discloses means of treating rheumatoid arthritis by administration of novel MSC. Rheumatoid arthritis (RA) is a chronic inflammatory disorder affecting approximately 0.5-1% of the global population [580], characterized by immune-mediated synovial inflammation and joint deterioration. In general, because of the critical role of inflammation in the pathology of RA, patients have usually been started on NSAIDS, however more recent practice has been concurrent initiation of disease modifying antirheumatic drugs (DMARDs). These agents are slow acting but have been demonstrated to inhibit radiological progression of RA. Such agents typically include: 1) hydroxychloroquine, which acts in part as a toll like receptor (TLR) 7/9 antagonist, thus decreasing innate immune activation [581]; 2) Leflunomide, an antimetabolite that inhibits pyrimidine synthesis and protein tyrosine kinase activity [582], which results in suppression of T cell responses [583], and has been also demonstrated to inhibit dendritic cell (DC) activation [584]; 3) Injectable gold compounds such as auranofin which directly or through metabolites such as dicyanogold(i) have been demonstrated to inhibit T cell and antigen presenting cell activation [585, 586], as well as cause Th2 deviation [587]; 4) Sulfasalazine, was used since 1950, acts primarily through inhibition of cycloxygenase and lipoxygenase [588]; and 5) Methotrexate, an antifolate that inhibits T cell activation and proliferation, that has been one of the golden standards for RA [589]. Typically combinations of DMARDs with glucocorticoids are used, or alternatively pulse of high dose glucocorticoids are administered to cause a general inhibition of inflammation [590].

The TNF-alpha targeting agents, Remicade, Enbrel, and Humira, sometimes referred to as “biological DMARDs” are used primarily after response to conventional DMARDs has failed [591]. Although improvement in quality of life has occurred as a result of biological DMARDs, substantial progress remains to be made. For example, TNF-alpha blockers have been associated with reactivation of infectious disease, autoantibody formation and the possibility of increased lymphoma risk [592, 593]. Thus to date, one of the major limitations to RA therapy has been lack of ability to specifically inhibit autoreactive responses while allowing other immune components to remain intact.

### Ideal Therapy for RA Would Be Tolerance Induction

The autoimmune nature of RA suggests the possibility of specifically inhibiting the pathological response through “reprogramming” of immune effectors. However, in order to evoke antigen-specific immune modulation, it is necessary to have knowledge of autoantigens that are present in a majority of the population and contribute to disease. Collagen II is an extracellular matrix component found primarily in the synovial tissue that is usually sequestered from immunological attack. Induction of a RA-like disease has been reported in inbred strains following immunization of collagen II in the presence of adjuvant [594]. Autoimmunity was not induced by collagen I or III, nor by denatured collagen II protein. Supporting a causative immunopathological effect of collagen II specific T cells were experiments in which the RA-like disease could be transferred to naive recipients by administration of lymph node cells [595]. Subsequent work cloning T cell lines from synovial membranes of patients with RA demonstrated existence of collagen II-specific cells that persisted for a period of 3 years in vivo [596]. Subsequent PCR-studies of T cell receptor beta chains confirmed the oligoclonal expansion of collagen II-reactive cells in patients [597]. In 1993 Weiner’s group reported a double blind, placebo-controlled trial of 60 patients with advanced RA treated by oral administration of chicken collagen II for a period of 3 months. Responses in terms of decreased number of swollen joints were observed in the treated population but not placebo controls. Of the treated patients four presented with complete remission of disease. No treatment-associated adverse effects were noted [598]. Unfortunately, Phase III trials using oral tolerance in RA have not met primary efficacy endpoints [599].

Given the general failure of oral tolerance in RA, more specific approaches have involved stimulation of tolerogenic responses using ex vivo manipulated DC. Dendritic cells (DC) under physiological conditions promote tolerance, and when exposed to injury/damage signals mature and induce T cell activation. By ex vivo manipulating antigen pulsed/donor specific DC, we have previously been able to induce antigen-specific suppression of immunity and generation of T regulatory (Treg) cells. Tolerogenic modifications of DC performed by our group have included exposure of the DC to small molecule immune suppressants [600-602], gene transfection with tolerogenic genes [603, 604] and gene silencing of immune activatory genes [605-608]. In our previous work, we have demonstrated ability to prevent RA induction by pulsing DC with collagen II (CII) and suppressing DC maturation with chemical or genetic means. Limitations of these data, however, have been the lack of robust inhibition of inflammatory responses when administration of manipulated DC was performed at various time points subsequent to disease onset. The general failure of antigen specific approaches, both in oral tolerance, as well as DC-based approaches may be the result of underlying inflammatory reactions.

### MSC as Inducers of Tolerance

Specifically, the possibility of using systemically-administered mesenchymal stem cells (MSC) as a cellular therapy for RA has several conceptual advantages that address the previously mentioned drawbacks of current approaches. One such advantage is that the MSC may be viewed as a “smart” immune modulator. In contrast to current therapies, which globally cause immune suppression, production of anti-inflammatory factors by MSC appears to be dependent on their environment, with upregulation of factors such as TGF-b, HLA-G, IL-10, and neuropilin-A ligands galectin-1 and Semaphorin-3A in response to immune/inflammatory stimuli but little in the basal state [245, 246, 494, 609, 610]. Additionally, systemically administered MSC possess ability to selectively home to injured/hypoxic areas by recognition of signals such as HMGB1 or CXCR1, respectively [334, 337, 611, 612]. The ability to home to injury, combined with selective induction of immune modulation only in response to inflammatory/danger signals suggests the possibility that systemically administered MSC do not cause global immune suppression. This is supported by clinical studies using MSC for other inflammatory conditions, which to date, have not reported immune suppression associated adverse effects [193, 613, 614]. Another important aspect of MSC therapy is their ability to regenerate injured tissue through direct differentiation into articular tissue [615], as well as ability to secret growth factors capable of augmenting endogenous regenerative processes [616].

Physiologically, the role of MSC in RA is a matter of debate. Nakagawa et al used radiolabeling of bone marrow cells to demonstrate migration of bone marrow stromal cells into synovium of rats suffering from CIA. While inference was made to contribution of the MSC to synovial proliferation, a causal relationship was not demonstrated [617]. Subsequently, it was reported that MSC differentiate into nurse-like cells that promote adhesion of lymphocytes to the synovium [618]. Indeed, in patients with RA, but not healthy controls, bone marrow MSC-generating capacity is markedly reduced [619], whether this is due to systemic TNF-alpha suppression of bone marrow [620], or exhaustion of MSC precursors by heightened demand is not known. However, there are suggestions of the latter based on observations of shorter telomeres in MSC derived from RA patients [619]. The concept of MSC contributing to pathology was demonstrated in the CIA model by Djouad et al who reported administration of MSC resulted in upregulation of Th1 immunity and worsening of symptoms [621]. The investigators attributed this to their observations that TNF-alpha abrogates immune regulatory activities of MSC. This study however was contradicted by several more recent studies in which inhibition of arthritogenesis, or even regression of disease was observed. Mao et al demonstrated administration of rat MSC intravenously into DBA mice with full-blow CIA resulted in regression of disease, which was correlated with decreased production of TNF-alpha and IL-17 [622]. Gonzalez et al administered ex vivo expanded human adipose-derived MSC into the same animal model. Inhibition of disease progression was observed, which correlated with increased Treg numbers that were specific for CII. This study supports the previous principle discussed that an antigen-nonspecific tolerizing event may contribute to development of antigen specific suppression [623]. In addition to immune modulation, it is possible that cartilage tissue generated de novo from MSC possesses a decreased level of immunogenicity [624]. The overall anti-inflammatory/immune modulatory effects of MSC have been demonstrated in a variety of settings including the mouse model of multiple sclerosis [567, 625], transplant rejection [46], diabetes [626], the mouse model of SLE [627], and autoimmune enteropathy [251].

### Clinical Trials of MSC in RA

One of the first studies was aimed to determine the safety and efficacy of allogeneic mesenchymal stem cells transplantation (MSCT) in refractory rheumatoid arthritis (RA). Four patients with persistently active RA underwent MSCT. The outcome was evaluated by changes in the visual analog scale (VAS 100 mm) pain score, C-reactive protein (CRP) and erythrocyte sedimentation rate (ESR), and 28-joint disease activity score (DAS-28). Three of four patients received a reduction in ESR, DAS-28, and pain VAS score at 1 and 6 months after transplantation. Two of the three had a European League Against Rheumatism (EULAR) moderate response at 6 months but experienced a relapse at 7 and 23 months, respectively. Two patients had no EULAR response to MSCT. No one had achieved the DAS-28-defined remission in the follow-up period. No serious adverse events were reported. Allogeneic MSCT is a safe treatment in severe and resistant RA, but the effectiveness needs to be clarified [628].

A subsequent study used human umbilical cord mesenchymal stem cells (UC-MSCs) in the treatment of rheumatoid arthritis (RA). In this ongoing cohort, 172 patients with active RA who had inadequate responses to traditional medication were enrolled. Patients were divided into two groups for different treatment: disease-modifying anti-rheumatic drugs (DMARDs) plus medium without UC-MSCs, or DMARDs plus UC-MSCs group (4×10(7) cells per time) via intravenous injection. Adverse events and the clinical information were recorded. Tests for serological markers to assess safety and disease activity were conducted. Serum levels of inflammatory chemokines/cytokines were measured, and lymphocyte subsets in peripheral blood were analyzed. No serious adverse effects were observed during or after infusion. The serum levels of tumor necrosis factor-alpha and interleukin-6 decreased after the first UC-MSCs treatment (P<0.05). The percentage of CD4(+)CD25(+)Foxp3(+) regulatory T cells of peripheral blood was increased (P<0.05). The treatment induced a significant remission of disease according to the American College of Rheumatology improvement criteria, the 28-joint disease activity score, and the Health Assessment Questionnaire. The therapeutic effects maintained for 3-6 months without continuous administration, correlating with the increased percentage of regulatory T cells of peripheral blood. Repeated infusion after this period can enhance the therapeutic efficacy. In comparison, there were no such benefits observed in control group of DMARDS plus medium without UC-MSCs. Thus, our data indicate that treatment with DMARDs plus UC-MSCs may provide safe, significant, and persistent clinical benefits for patients with active RA.[629]. Another UC-MSC trial aimed to treat juvenile idiopathic arthritis (JIA), known as Juvenile rheumatoid arthritis, is the most common type of arthritis in children aged under 17. It may cause sequelae due to lack of effective treatment. The goal of this study is to explore the therapeutic effect of umbilical cord mesenchymal stem cells (UC-MSCs) for JIA. Ten JIA patients were treated with UC-MSCs and received second infusion three months later. Some key values such as 28-joint disease activity score (DAS28), TNF-α, IL-6, and regulatory T cells (Tregs) were evaluated. Data were collected at 3 months and 6 months after first treatment. DAS28 score of 10 patients was between 2.6 and 3.2 at three months after infusion. WBC, ESR, and CRP were significantly decreased while Tregs were remarkably increased and IL-6 and TNF-α were declined. Similar changes of above values were found after 6 months. At the same time, the amount of NSAIDS and steroid usage in patients was reduced. However, no significant changes were found comparing the data from 3 and 6 months. These results suggest that UC-MSCs can reduce inflammatory cytokines, improve immune network effects, adjust immune tolerance, and effectively alleviate the symptoms and they might provide a safe and novel approach for JIA treatment [630].

An interesting perspective is the use of expanded allogeneic adipose MSC. It is a multicentre, dose escalation, randomised, single-blind (double-blind for efficacy), placebo-controlled, phase Ib/IIa clinical trial. Patients with active refractory RA (failure to at least two biologicals) were randomised to receive three intravenous infusions of Cx611: 1 million/kg (cohort A), 2 million/kg (cohort B), 4 million/kg (cohort C) or placebo, on days 1, 8 and 15, and they were followed for therapy assessment for 24 weeks. Fifty-three patients were treated (20 in cohort A, 20 in cohort B, 6 in cohort C and 7 in placebo group). A total of 141 adverse events (AEs) were reported. Seventeen patients from the group A (85%), 15 from the group B (75%), 6 from the group C (100%) and 4 from the placebo group (57%) experienced at least one AE.Eight AEs from 6 patients were grade 3 in intensity (severe), 5 in cohort A (lacunar infarction, diarrhoea, tendon rupture, rheumatoid nodule and arthritis), 2 in cohort B (sciatica and RA) and 1 in the placebo group (asthenia). Only one of the grade 3 AEs was serious (the lacunar infarction). American College of Rheumatology 20 responses for cohorts A, B, C and placebo were 45%, 20%, 33% and 29%, respectively, at month 1, and 25%, 15%, 17% and 0%, respectively, at month 3. The authors concluded that intravenous infusion of Cx611 was in general well tolerated, without evidence of dose-related toxicity at the dose range and time period studied. In addition, a trend for clinical efficacy was observed. These data, in our opinion, justify further investigation of this innovative therapy in patients with RA[631].

In the current disclosure, the use of novel stem cells for treatment of stroke is disclosed. Stroke is the third leading cause of death and disability in adults in the US. Thrombolytic therapy only benefits about 2% of the ischemic stroke patients. Reduction of tissue plasminogen activator induced hemorrhage and brain injury by free radical spin trapping after embolic focal cerebral ischemia in rats.. The dismal record of neurorestorative regimens for stroke both in the laboratory and the clinic solicits an urgent need to develop novel therapies. Because the secondary cellular death that ensues after the initial stroke episode occurs over an extended time. Treatment strategies directed at rescuing these ischemic neurons have the potential to retard the disease progression and even afford restoration of function. The recognition of this delay in secondary stroke-induced pathophysiologic alterations has prompted investigations on neurorestorative treatments, including cell therapy, to salvage the ischemic penumbra and promote functional recovery from stroke. Cell therapy thus offers a new avenue for the treatment and management of stroke.

### Autologous MSC for Stroke

A study prospectively and randomly allocated 30 patients with cerebral infarcts within the middle cerebral arterial territory and with severe neurological deficits into one of two treatment groups: the MSC group (n = 5) received intravenous infusion of 1 x 10(8) autologous MSCs, whereas the control group (n = 25) did not receive MSCs. Changes in neurological deficits and improvements in function were compared between the groups for 1 year after symptom onset. Neuroimaging was performed serially in five patients from each group. Outcomes improved in MSC-treated patients compared with the control patients: the Barthel index (p = 0.011, 0.017, and 0.115 at 3, 6, and 12 months, respectively) and modified Rankin score (p = 0.076, 0.171, and 0.286 at 3, 6, and 12 months, respectively) of the MSC group improved consistently during the follow-up period. Serial evaluations showed no adverse cell-related, serological, or imaging-defined effects. The authors concluded that for patients with severe cerebral infarcts, the intravenous infusion of autologous MSCs appears to be a feasible and safe therapy that may improve functional recovery [632].

A follow up to this study by the same group reported on an open-label, observer-blinded clinical trial of 85 patients with severe middle cerebral artery territory infarct. Patients were randomly allocated to one of two groups, those who received i.v. autologous ex vivo cultured MSCs (MSC group) or those who did not (control group), and followed for up to 5 years. Mortality of any cause, long-term side effects, and new-onset comorbidities were monitored. Of the 52 patients who were finally included in this study, 16 were the MSC group and 36 were the control group. Four (25%) patients in the MSC group and 21 (58.3%) in the control group died during the follow-up period, and the cumulative surviving portion at 260 weeks was 0.72 in the MSC group and 0.34 in the control group (log-rank; p = 0.058). Significant side effects were not observed following MSC treatment. The occurrence of comorbidities including seizures and recurrent vascular episodes did not differ between groups. When compared with the control group, the follow-up modified Rankin Scale (mRS) score was decreased, whereas the number of patients with a mRS of 0-3 increased in the MSC group (p = 0.046). Clinical improvement in the MSC group was associated with serum levels of stromal cell-derived factor-1 and the degree of involvement of the subventricular region of the lateral ventricle. Intravenous autologous MSCs transplantation was safe for stroke patients during long-term follow-up. This therapy may improve recovery after stroke depending on the specific characteristics of the patients [633].

Another study used autologous BM-MSC in 12 patients who had stroke lasting 3 months to 1 year, motor strength of hand muscles of at least 2, and NIHSS of 4-15, and patients had to be conscious and able to comprehend. Fugl Meyer (FM), modified Barthel index (mBI), MRC, Ashworth tone grade scale scores and functional imaging scans were assessed at baseline, and after 8 and 24 weeks. Bone marrow was aspirated under aseptic conditions and expansion of MSC took 3 weeks with animal serum-free media (Stem Pro SFM). Six patients were administered a mean of 50-60 × 10(6) cells i.v. followed by 8 weeks of physiotherapy. Six patients served as controls. This was a non-randomized experimental controlled trial. Clinical and radiological scanning was normal for the stem cell group patients. There was no mortality or cell-related adverse reaction. The laboratory tests on days 1, 3, 5 and 7 were also normal in the MSC group till the last follow-up. The FM and mBI showed a modest increase in the stem cell group compared to controls. There was an increased number of cluster activation of Brodmann areas BA 4 and BA 6 after stem cell infusion compared to controls, indicating neural plasticity [634].

Another study used serum free media similar to the above study, with exception that they used patient’s own sera for expansion of autologous BM-MSC. The publication described an unblinded study on 12 patients with ischaemic grey matter, white matter and mixed lesions, in contrast to a prior study on autologous mesenchymal stem cells expanded in foetal calf serum that focused on grey matter lesions. Cells cultured in human serum expanded more rapidly than in foetal calf serum, reducing cell preparation time and risk of transmissible disorders such as bovine spongiform encephalomyelitis. Autologous mesenchymal stem cells were delivered intravenously 36-133 days post-stroke. All patients had magnetic resonance angiography to identify vascular lesions, and magnetic resonance imaging prior to cell infusion and at intervals up to 1 year after. Magnetic resonance perfusion-imaging and 3D-tractography were carried out in some patients. Neurological status was scored using the National Institutes of Health Stroke Scale and modified Rankin scores. We did not observe any central nervous system tumours, abnormal cell growths or neurological deterioration, and there was no evidence for venous thromboembolism, systemic malignancy or systemic infection in any of the patients following stem cell infusion. The median daily rate of National Institutes of Health Stroke Scale change was 0.36 during the first week post-infusion, compared with a median daily rate of change of 0.04 from the first day of testing to immediately before infusion. Daily rates of change in National Institutes of Health Stroke Scale scores during longer post-infusion intervals that more closely matched the interval between initial scoring and cell infusion also showed an increase following cell infusion. Mean lesion volume as assessed by magnetic resonance imaging was reduced by >20% at 1 week post-cell infusion [635].

### Allogeneic MSC for Stroke

One of the first publications regarding cerebral infarcts treated by MSC was a case report in which a 17-year-old Korean man who was diagnosed with basilar artery dissection. Infarction of the bilateral pons, midbrain and right superior cerebellum due to his basilar artery dissection was partially recanalized by intrathecal injection of human umbilical cord blood-derived mesenchymal stem cells. No immunosuppressants were given to our patient, and human leukocyte antigen alloantibodies were not detected after cell therapy. A subjective response was reported [636].

In an attempt to increase efficacy and leverage potential synergies between MSC and tissue specific progenitors, a study investigated combination of MSC together with neural progenitor cells. Eight patients were enrolled in this study. All patients had a hemisphere with infarct lesions located on one side of the territories of the cerebral middle or anterior arteries as revealed with cranial magnetic resonance imaging (MRI). The patients received one of the following two types of treatment: the first treatment involved four intravenous injections of MSCs at 0.5 × 10(6)/kg body weight; the second treatment involved one intravenous injection of MSCs at 0.5 × 10(6)/kg weight followed by three injections of MSCs at 5 × 10(6)/patient and NSPCs at 6 × 10(6)/patient through the cerebellomedullary cistern. The patients’ clinical statuses were evaluated with the National Institutes of Health Stroke Scale (NIHSS), the modified Rankin Scale (mRS), and the Barthel index (BI). Six patients were given four cell transplantations. The most common side effect of stem cell transplantation in these six cases was low fever that usually lasted 2-4 days after each therapy. One patient exhibited minor dizziness. All side effects appeared within the first 2-24 h of cell transplantation, and they resolved without special treatment. There was no evidence of neurological deterioration or neurological infection. Most importantly, no tumorigenesis was found at a 2-year follow-up. The neurological functions, disability levels, and daily living abilities of the patients in this study were improved [637].

Another attempt to augment potency of allogeneic MSC involves transfection with neurotrophic factors. Eighteen patients with stable, chronic stroke were enrolled in a 2-year, open-label, single-arm study to evaluate the safety and clinical outcomes of surgical transplantation of modified bone marrow-derived mesenchymal stem cells (SB623).All patients in the safety population (N=18) experienced at least 1 treatment-emergent adverse event. Six patients experienced 6 serious treatment-emergent adverse events; 2 were probably or definitely related to surgical procedure; none were related to cell treatment. All serious treatment-emergent adverse events resolved without sequelae. There were no dose-limiting toxicities or deaths. Sixteen patients completed 12 months of follow-up at the time of this analysis. Significant improvement from baseline (mean) was reported for: (1) European Stroke Scale: mean increase 6.88 (95% confidence interval, 3.5-10.3; P<0.001), (2) National Institutes of Health Stroke Scale: mean decrease 2.00 (95% confidence interval, -2.7 to -1.3; P<0.001), (3) Fugl-Meyer total score: mean increase 19.20 (95% confidence interval, 11.4-27.0; P<0.001), and (4) Fugl-Meyer motor function total score: mean increase 11.40 (95% confidence interval, 4.6-18.2; P<0.001). No changes were observed in modified Rankin Scale. The area of magnetic resonance T2 fluid-attenuated inversion recovery signal change in the ipsilateral cortex 1 week after implantation significantly correlated with clinical improvement at 12 months (P<0.001\ for European Stroke Scale) [638].

The invention further discloses the utilization of new stem cells for treatment of type 1 diabetes. Diabetes mellitus refers to disorders in which the body has trouble regulating its blood glucose levels. There are two major types of diabetes: type 1 and type 2. Type 1 diabetes mellitus (T1D), the indication being studied in this trial, is also called juvenile diabetes or insulin-dependent diabetes.

Diabetes annually accounts for more than $130 billion in health care costs in the U.S. alone [639].Type 1 diabetes affects ~15-30 million people globally and 1.4 million in the United States [640, 641]. The incidence is increasing significantly in many populations, especially among young children. In general, most people are diagnosed with type 1 diabetes before the age of 30. Not only will these people be insulin-dependent for life, but devastating life-limiting and life-shortening complications can occur. Insulin is the primary method of controlling diabetes by regulating blood glucose levels, but it may not reverse or prevent disease progression.

T1D is an autoimmune condition characterized by immunological destruction of insulin producing pancreatic beta cells resulting in dependency on insulin injections for survival [642]. Additionally, patients are required to maintain tight glucose control through diet and monitoring to prevent secondary complications of diabetes such as blindness, kidney failure and cardiovascular disease [643]. To date no treatment has been effective in the treatment of T1D with exception of pancreas or pancreatic islet transplantation. Unfortunately these approaches are limited by the number of donor pancreas’s available, as well as the need for life-long immune suppression [644]. A clinical trial involving autologous hematopoietic stem cell therapy in T1D has yielded promising results with 20 of 23 patients becoming insulin independent and 12 of the patients maintaining insulin independence for a mean of 31 months [645]. Unfortunately the immune suppressive regimen required for autologous hematopoietic stem cell transplant, as well as invasiveness of stem cell extraction limits the wide-spread applicability of this procedure. Accordingly, novel means of intervention in T1D are urgently needed.

### Mesenchymal Stem Cell Activities Relevant to Type 1 Diabetes

Mesenchymal stem cells (MSC) are a population of stem cells originally discovered in the bone marrow as playing a role in supporting the hematopoietic stem cell function through paracrine effects, as well as suppressing local inflammation [646, 647]. MSC have a history of safe clinical use with over 1000 patients having been treated in clinical trials with no adverse events reported [648]. MSC, whether derived from the bone marrow, adipose, or other sources, have been demonstrated to exert inhibit autoimmunity [233-237]. Mechanistically MSC inhibit innate immune activation by blocking dendritic cell (DC) maturation [649, 650], by suppressing macrophage activation [651], and by producing agents such as IL-1 receptor antagonist [247] and IL-10 [244] that directly block stimulation of antigen presentation and perpetuation of autoimmunity. Perhaps the strongest example of MSC inhibiting the innate immune response is the recent publication of Nemeth et al, which demonstrated that administration of MSC can block onset of sepsis in the aggressive cecal ligation and puncture model through suppressing macrophage activation [651].

Through inhibiting DC activation, MSC suppress subsequent adaptive immunity by generating T regulatory (Treg) cells [46], as well as blocking cytotoxic activities of CD8 cells. The overall anti-inflammatory/immune modulatory effects of MSC have been demonstrated in a variety of settings including the mouse model of multiple sclerosis [567, 625], transplant rejection [46], diabetes [626], the mouse model of SLE [627], and autoimmune enteropathy[251]. To date, clinical trials have suggested safety and signals of efficacy of MSC in the following autoimmune conditions: systemic lupus erythromatosus[550, 652], Type 1 Diabetes [653], multiple sclerosis [572], and Sjögren syndrome[654].

### Pathophysiology of T1D

Clinical manifestation of T1D reflects the consequence of an underlying, sustained autoimmune process involving T cells, dendritic cells and B cells. For instance, autoantibodies against islet antigens are detected before the clinical onset of T1D. This suggests that a sequence of inciting events precedes the hyperglycemia for at least months, but most likely several years. The possibility exists that inciting agents may be of a “hit-and-run” type, leaving no detectable molecular trace. Alternatively, it may take multiple environmental insults to unleash autoimmunity, and different patients may incur them in divergent combinations [655, 656].

Several immune events occur before the clinical symptoms of type 1 diabetes become apparent. Most importantly, autoantibodies are produced and self-reactive lymphocytes become activated and infiltrate the pancreas to destroy the insulin-producing beta-cells in the islets of Langerhans. The main autoantibodies in T1D are reactive to 4 islet autoantigens (islet cell autoantibodies or ICA): insulinoma-associated antigen-2 (I-A2, ICA512), insulin (micro IAA or mIAA), glutamic acid decarboxylase 65 (GAD65), and zinc transporter 8 (ZnT8) [657]. These antibodies cause persistent, targeted destruction that may go undetected for many years, and the first clinical symptoms only become apparent after a majority of the beta-cells have been destroyed or rendered dysfunctional, making the individual dependent on insulin for survival [658]. The importance of B cells and antibodies in the progression of T1D is illustrated by studies demonstrating in the NOD model of diabetes that depletion of B cells protects from disease onset [659, 660]. Clinical trials have also demonstrated a level of protection of insulin production in patients treated with B cell depleting anti-CD20 antibodies [661].

Perhaps an earlier event in the pathogenesis of T1D progression is the infiltration of the pancreas by dendritic cells (DC). In animal models such as the BB rat or the NOD mouse, associated with the pre-diabetic insulitis is infiltration of dendritic cells [662, 663]. Using transgenic models of T1D it has been demonstrated that islet-specific expression of antigens on DC leads to breaking of tolerance and islet-destroying autoimmunity [663]. Additionally, in the NOD model it has been demonstrated that an initial wave of islet cell death during development causes release of antigens that are taken up by DC, presented to T cells, resulting in the autoimmune cascade. Furthermore, deletion of DC subsets revealed a specific phenotype associated with stimulation of autoreactivity [664].

T cells are considered to be the final executors of beta-cell destruction. This is evidenced by the precipitation or prevention of diabetes by transfer or elimination of CD4 or CD8 T cells, respectively. CD8 T cell-mediated beta-cell killing is likely a major mechanism of beta-cell destruction. CD8 T cells, found in insulitic lesions in NOD mice and in human can destroy beta cells upon activation via MHC class I expressed on beta cells. Indeed, deficiency in MHC class I due to lack of beta-2 microglobulin, or beta cell-restricted MHC class I deficiency is sufficient to arrest diabetes development and prevent beta-cell destruction in NOD [665]. This is similarly supported by studies in which mice lacking CD8 cells that are crossed with the NOD strain do not develop diabetes [666]. Mechanistically, beta-cell destruction can involve the release by CD8 T cells of cytolytic granules containing perforin and granzyme, or through Fas and Fas ligand-dependent interactions. CD4 T cells mostly provide help to both B cells and CD8 T cells by providing cytokines, such as IL-21, and a positive-feedback loop via CD40L-CD40 interactions to antigen-presenting cells, culminating in a proficient autoreactive CD8 T-cell response. Their presence in insulitic lesions suggests a beacon role and direct inflammatory properties [642].

The major autoantibody targets: proinsulin (PI), GAD65, I-A2, and ZnT8 [667] appear to also represent antigens recognized by T cells. So far, practical issues have made it difficult to determine systematically when autoantibodies and autoreactive T cells arise in the periphery relative to each other. Additionally, smaller contributions from heat shock protein (HSP)-60 and islet-specific glucose-6-phosphatase catalytic subunit-related protein (IGRP), as well as HSP70 in humans, complete the known range of CD4 epitopes. On the other hand, autoreactive CD8 epitopes in humans come mainly from preproinsulin signal peptide [668] and to a lesser extent IA2, human islet amyloid polypeptide (IAPP) precursor protein [669], IGRP, cation efflux transporter ZnT8 (Slc30A8) [657], and GAD65 [670]. In mice, the CD8 epitopes are mainly derived from IGRP and GAD65/67, and ~30% from PI and 10% from dystrophia myotonica kinase (DMK). Interestingly, CD8 peptide epitopes were identified from the human preproinsulin signal peptide by elution from HLA-A2 molecules [668]. This has led to the concept that beta-cells unwittingly contribute to their own demise, because they are targeted even more when stimulated to produce more insulin. Together, these data have provided strong evidence that CD8 T-cell autoreactivity is associated with beta-cell destruction in T1D in humans.

### Immune Modulation Therapeutically Benefits T1D

Based on the knowledge that T1D is an autoimmune, T cell mediated attack on the pancreatic beta cells, therapeutic interventions have been developed that block various components of the immune response in order to delay progression of diabetes or induce reversal of disease. Early studies utilized the global immune suppressant agent cyclosporine to patients who were diagnosed with T1D. Of 30 patients that were treated within 6 weeks of diagnosis, 16 became insulin independent, which was accompanied by reduction in anti-islet antibodies [671]. There promising results were subsequently repeated in a larger double blind placebo controlled study [672]. While these important clinical experiments demonstrated unequivocally the importance of T cells in contributing to T1D pathology, the use of cyclosporine is impractical due to predisposition to infectious agents as well as induction of kidney failure, which was actually hinted at by the increased creatinine in the treated children. Additionally, long-term follow-up of patients treated with cyclosporine who subsequently discontinued it, revealed relapse of diabetes and insulin requirements similar to pre-intervention levels [673].

More specific immune modulators were subsequently attempted in trials of early-onset T1D patients. For example, blockade of CD80 and CD86 using soluble CTLA4-Ig (abatacept) was used in a double blind, placebo controlled trial of 112 patients who were assigned to receive Abatacept (77) and placebo (35) [674]. While the trial consisted of 2 year administration, response as assessed by preserved stimulated C peptide levels lasted for only 9.5 months.

Other immune modulatory interventions included use of monoclonal antibody against the T cell receptor associated molecule CD3. Data from phase IIrecent-onset T1D trials with anti-CD3 have demonstrated positive therapeutic efficacy and benefit. For example, a 42 recent onset T1D patient study administered a 12 or 14 day course of humanized anti-CD3. Treatment, within the first 6 weeks after diagnosis, preserved C-peptide responses to a mixed meal for 1 year after diagnosis (97 +/- 9.6% of response at study entry in drug-treated patients vs. 53 +/- 7.6% in control subjects, P < 0.01), with significant improvement in C-peptide responses to a mixed meal even 2 years after treatment (P < 0.02). The improved C-peptide responses were accompanied by reduced HbA(1c) and insulin requirements [675]. In a subsequent multicenter study, 80 patients with new-onset type 1 diabetes were randomly assigned to receive placebo or anti-CD3 for six consecutive days. At 6, 12, and 18 months, residual beta-cell function was better maintained with anti-CD3 than with placebo. The insulin dose increased in the placebo group but not in the anti-CD3 group. This effect of anti-CD3 was most pronounced among patients with initial residual beta-cell function at or above the 50th percentile of the 80 patients. In this subgroup, the mean insulin dose at 18 months was 0.22 IU per kilogram of body weight per day with anti-CD3, as compared with 0.61 IU per kilogram with placebo (P<0.001). In this subgroup, 12 of 16 patients who received anti-CD3 (75 percent) received minimal doses of insulin (< or =0.25 IU per kilogram per day) as compared with none of the 21 patients who received placebo [676]. Continuation of this work led to Phase III pivotal studies for two types of anti-CD3 antibodies, teplizumab and otelixizumab, unfortunately both trials failed to meet their primary endpoints [677, 678]. While antigen nonspecific immune modulatory appear to show some promise, besides their clinical failure, the possibility of non-specifically suppressing immune responses necessary for resistance to opportunistic infections also exists. In fact this has been seen in the anti-CD3 trials in which reactivation of Epstein Barr virus was observed in one of the treated pateints [679].

A more recent Phase III study involving antigen nonspecific immune modulation involved administration of anti-IL1 antibody or recombinant interleukin 1 receptor antagonist in recent onset T1D patients. As compared to placebo, no efficacy was found using either IL-1 blocking approach [680].

The possibility of antigen-specifically inducing tolerance in T1D is substantially more tantalizing from a therapeutics development perspective. Numerous examples of immunological tolerance exist, for example, ingestion of antigen, including the RA autoantigen collagen II [681], has been shown to induce inhibition of both T and B cell responses in a specific manner [682, 683]. Remission of disease in animal models of RA [684], multiple sclerosis [685], and type I diabetes [686], has been reported by oral administration of autoantigens. Anterior chamber associated immune deviation (ACAID) is a phenomena in which local implantation of antigen results in a systemic immune modulation towards the antigen. Commonly this is demonstrated by antigen-specific suppression of DTH responses after intra-chamber administration of antigen [687]. Induction of ACAID has been used therapeutically in treatment of a mouse model of pulmonary inflammation: pretreatment with anterior chamber antigen injection resulted in systemic production from pulmonary damage [688]. In the context of T1D, not only has it been shown that antigen-specific tolerance is possible and protects from diabetes, but also that the tolerogenic effect can “spread” so that tolerance to one beta cell antigen can result in tolerance to other antigens [689, 690].

Unfortunately, clinical trials attempting to induce antigen-specific tolerance in T1D have primarily failed. One of the most well-known studies was that of DiaMyd, which administered the autoantigen GAD65 peptide in alum in a double blind pivotal trial. The trial consisted of 334 patients, 10 to 20 years of age, with type 1 diabetes, fasting C-peptide levels of more than 0.3 ng per milliliter (0.1 nmol per liter), and detectable serum GAD65 autoantibodies. Within 3 months after diagnosis, patients were randomly assigned to receive one of three study treatments: four doses of GAD-alum, two doses of GAD-alum followed by two doses of placebo, or four doses of placebo. The primary outcome was the change in the stimulated serum C-peptide level (after a mixed-meal tolerance test) between the baseline visit and the 15-month visit. Secondary outcomes included the glycated hemoglobin level, mean daily insulin dose, rate of hypoglycemia, and fasting and maximum stimulated C-peptide levels. The stimulated C-peptide level declined to a similar degree in all study groups, and the primary outcome at 15 months did not differ significantly between the combined active-drug groups and the placebo group (P=0.10). The use of GAD-alum as compared with placebo did not affect the insulin dose, glycated hemoglobin level, or hypoglycemia rate [691]. Another double blind study using GAD peptide in alum reported similar negative results [692].

### Mesenchymal Stem Cells as Immune Modulators

Mesenchymal stem cells (MSC) are classically defined as adherent, non-hematopoietic cells expressing markers such as CD90, CD105, and CD73, while lacking expression of CD14, CD34, and CD45, and being able to differentiate into adipocytes, chondrocytes, and osteocytes in vitro after treatment with differentiation inducing agents [693]. Although early studies in the late 1960s initially identified MSC in the bone marrow [694], more recent studies have reported these cells can be purified from various tissues such as adipose [233], heart [234], Wharton’s Jelly [235], dental pulp [236], peripheral blood [237], cord blood [238], and more recently menstrual blood [239-241].

A unique property of MSC is their apparent hypoimmunogenicity and actually immune modulatory activity [242], which is present in MSC derived from various sources [243]. This is believed to account for the ability to achieve therapeutic effects in an allogeneic manner. Allogeneic bone marrow derived MSC have been used by academic investigators with clinical benefit treatment of diseases such as graft versus host (GVHD) [175-177, 209, 256, 257], osteogenesis imperfecta [695], Hurler syndrome, metachromatic leukodystrophy [696], and acceleration of hematopoietic stem cell engraftment [697-699]. The company Osiris has successfully completed Phase I safety studies using allogeneic bone marrow MSCs is now in efficacy finding clinical trials (Phase II and Phase III) for Type I Diabetes, Crohn’s Disease, and Graft Versus Host Disease using allogeneic bone marrow derived MSC.

MSC modulate the immune system at several levels, we will discuss below the effects of MSC on various aspects of immune response induction from antigen presentation to effector function.

Dendritic cells (DC) are considered the primary sentinels of the immune response, playing a key role in determining whether productive immunity will ensure, versus stimulation of T regulatory cells and suppression of immunity [700, 701]. Although various subtypes of DC exist, with varying specialized functions, one of the common themes appears to be that immature myeloid type DC reside in an immature state in the periphery, which engulf antigens and present in a tolerogenic manner to T cells in the lymph nodes. This is one of the mechanisms by which self tolerance is maintained. Specifically, although small numbers of autoreactive T cells escape the thymic selection process, these T cells are either anergized, or their activity suppressed by T regulatory cells generated as a result of immature dendritic cells presenting self antigens to autoreactive T cells. In contrast, in the presence of “danger” signals, such as toll like receptor agonists, immature DC take a mature phenotype, characterized by high expression of costimulatory molecules, and subsequently induce T cell activation [702-704]. In the context of T1D it has previously been demonstrated that targeting of diabetogenic autoantigens to immature DC leads to prevention of disease [705]. Administration of immature DC into 10 T1D patients resulted in increased C-peptide levels with some evidence of immunomodulatory activity[706].

Given the fundamental role of the DC in controlling immunity versus tolerance, the manipulation of DC maturation by MSC would strongly support an immune modulatory role of MSC. Early studies suggested that MSC may inhibit the ability of DC to stimulate CD4 and CD8 cells using in vitro systems, however, it was demonstrated that MSC also inhibited T cell activation directly [707]. Subsequently, Zhang et al performed a definitive study in which bone marrow MSC were cultured directly with monocytes which were stimulated to differentiate into DC using a standard IL-4 and GM-CSF protocol in the murine system. It was found that MSCs inhibit the up-regulation of CD1a, CD40, CD80, CD86, and HLA-DR during DC differentiation and prevent an increase of CD40, CD86, and CD83 expression during DC maturation. MSCs supernatants had no effect on DCs differentiation, but they inhibited the up-regulation of CD83 during maturation. Both MSCs and their supernatants interfered with endocytosis of DCs, decreased their capacity to secret IL-12 and activate alloreactive T cells [708]. Using the human system, Aggarwal et al cultured Prochymal BM derived MSC together with DC that were polarized to generate Th1 promoting cytokines (DC1) and DC polarized to generate Th2 cytokines (DC2). MSC were demonstrated to inhibit production of TNF-alpha and IL-12 in DC 1 cells while increase production of IL-10 in DC2 cells [708]. The concept of MSC inducing immaturity in DC was further demonstrated in mixed lymphocyte reactions where it was shown that addition of MSC would suppress MLR only in the presence of antigen presenting cells [709]. Interestingly, the addition of DC maturation agents such as LPS or antiCD40 antibody was capable of overcoming MSC mediated suppression, thus implying that inhibition of DC immunogenic activities by MSC is a reversible process. The general ability of MSC to suppress DC immune stimulatory activities, through inhibition of maturation and costimulatory molecule expression was replicated in other studies [649, 710-714]. Other methods by which MSC inhibit DC activity include blocking the physical interaction with T cells [710], blocking DC progenitor entry into cell cycle [710], production of TSG-6 [715], induction of Notch in DC progenitors [716], and arresting DC migration into lymph nodes in vivo [650, 717, 718].

Some of the immune suppressive effects of MSC appear to be inducible by the presence of local inflammation. For example, a recent study showed that TLR activation on MSC increases ability of the MSC to suppress T cell activation through blockade of DC maturation [719]. Other studies have shown that treatment of MSC with inflammatory mediators such as IL-1 beta actually stimulates production of cytokines such as IL-10 that block DC maturation. IL-1 treated MSC possess superior in vivo ability to suppress inflammatory diseases such as DSS induced colitis [720]. Similar augmentation of anti-inflammatory activity of MSC by pretreatment with inflammatory cytokines was also reported by treatment with IFN-gamma [721-723]. On a cellular level it has been reported that coculture of MSC with monocytes leads to enhanced immune suppressive activities of the MSC, in part through monocyte produced IL-1 [724].

Inhibition of T cell reactivity by MSC has been widely described. One of the initial publications supporting this assessed baboon MSCs in vitro for their ability to elicit a proliferative response from allogeneic lymphocytes, to inhibit an ongoing allogeneic response, and to inhibit a proliferative response to potent T-cell mitogens. It was found that the MSCs failed to elicit a proliferative response from allogeneic lymphocytes. MSCs added into a mixed lymphocyte reaction, either on day 0 or on day 3, or to mitogen-stimulated lymphocytes, led to a greater than 50% reduction in proliferative activity. This effect could be maximized by escalating the dose of MSCs and could be reduced with the addition of exogenous IL-2. In vivo administration of MSCs led to prolonged skin graft survival when compared to control animals [725, 726]. Inhibition of T cell proliferation could not be restored by costimulation or pretreatment of the MSC with IFN-gamma [727], which is intriguing given that the previous study mentioned showed IL-2 could overcome MSC mediated suppression. In vivo studies using humanized mice demonstrated that human MSC were capable of suppressing human T cell responses in vivo, both allogenic and antigen-specific responses [728]. Inhibition of T cell activity seems to be not limited to proliferation but also was demonstrated to include suppression of cytotoxic activity of CD8 T cells [729, 730].

Several mechanisms have been reported for MSC suppression of T cell activation including inhibition of IL-2 receptor alpha (CD25) [731], induction of division arrest [732, 733], induction of T cell anergy directly [567] or via immature DC [714], stimulation of apoptosis of activated T cells [734, 735], blockade of IL-2 signaling and induction of PGE2 production [736-741], induction of TGF-beta[244], production of HLA-G [742], expression of serine protease inhibitor 6 [743], stimulation of nitric oxide release [744-746], stimulation of indolamine 2,3 deoxygenase [747-750], expression of adenosine generating ectoenzymes such as CD39 and CD73 [751, 752], Galectin expression[753, 754], induction of hemoxygenase 1[755, 756], activation of the PD1 pathway [753, 757-759], Fas ligand expression [760, 761], CD200 expression [762], Th2 deviation [763-765], inhibition of Th17 differentiation [766-770], TSG-6 expression [771], NOTCH-1 expression [772], stimulation of Treg cell generation [45, 46, 48, 773-777].

### Clinical Trials of Mesenchymal Stem Cells in Type 1 Diabetes

A clinical trial assessed the long-term effects of the implantation of Wharton’s jelly-derived mesenchymal stem cells (WJ-MSCs) from the umbilical cord for Newly-onset T1DM. Twenty-nine patients with newly onset T1DM were randomly divided into two groups, patients in group I were treated with WJ-MSCs and patients in group II were treated with normal saline based on insulin intensive therapy. Patients were followed-up after the operation at monthly intervals for the first 3 months and thereafter every 3 months for the next 21 months, the occurrence of any side effects and results of laboratory examinations were evaluated. There were no reported acute or chronic side effects in group I compared with group II, both the HbAlc and C peptide in group I patients were significantly better than either pretherapy values or group II patients during the follow-up period. These data suggested that the implantation of WJ-MSCs for the treatment of newly-onset T1DM is safe and effective [404].

In order to attempt to increase efficacy, Cai et al administered a combination of autologous bone marrow mononuclear cells together with umbilical cord mesenchymal stem cells in 21 type 1 diabetes patients. Another group of 21 patients were randomized to receive standard of care. Cells were administered at a concentration of (1.1 × 10(6)/kg UC-MSC, 106.8 × 10(6)/kg aBM-MNC through supraselective pancreatic artery cannulation). Patients were followed for 1 year at 3-month intervals. The primary end point was C-peptide area under the curve (AUC(C-Pep)) during an oral glucose tolerance test at 1 year. Additional end points were safety and tolerability of the procedure, metabolic control, and quality of life. The treatment was well tolerated. At 1 year, metabolic measures improved in treated patients: AUCC-Pep increased 105.7% (6.6 ± 6.1 to 13.6 ± 8.1 pmol/mL/180 min, P = 0.00012) in 20 of 21 responders, whereas it decreased 7.7% in control subjects (8.4 ± 6.8 to 7.7 ± 4.5 pmol/mL/180 min, P = 0.013 vs. SCT); insulin area under the curve increased 49.3% (1,477.8.00 ± 1,012.8.00 to 2,205.5.00 ± 1,194 \.0 mmol/mL/180 min, P = 0.01), whereas it decreased 5.7% in control subjects (1,517.7.00 ± 630.2 to 1,431.7.00 ± 441.6 mmol/mL/180 min, P = 0.027 vs. SCT). HbAlc decreased 12.6% (8.6 ± 0.81% [70.0 ± 7.1 mmol/mol] to 7.5 ± 1.0% [58.0 ± 8.6 mmol/mol], P < 0.01) in the treated group, whereas it increased 1.2% in the control group (8.7 ± 0.9% [72.0 ± 7.5 mmol/mol] to 8.8 ± 0.9% [73 ± 7.5 mmol/mol], P < 0.01 vs. SCT). Fasting glycemia decreased 24.4% (200.0 ± 51.1 to 151.2 ± 22.1 mg/dL, P < 0.002) and 4.3% in control subjects (192.4 ± 35.3 to 184.2 ± 34.3 mg/dL, P < 0.042). Daily insulin requirements decreased 29.2% in only the treated group (0.9 ± 0.2 to 0.6 ± 0.2 IU/day/kg, P = 0.001), with no change found in control subjects (0.9 ± 0.2 to 0.9 ± 0.2 IU/day/kg, P < 0.01 vs. SCT).The authors concluded that transplantation of UC-MSC and aBM-MNC was safe and associated with moderate improvement of metabolic measures in patients with established T1D [778].

Another combination therapy clinical exploration was performed by Dave et al. who reported on two children: a 5-year-old girl on exogenous insulin therapy of 30 IU/day and a 9-year-old boy on short-acting insulin 30 IU/day, long-acting insulin 70 IU/day, with IDDM since 4 and 7 years, respectively. They infused in vitro-generated donor bone marrow (BM)-derived haematopoietic stem cells (HSC) in patient 1 and insulin-secreting cells trans-differentiated from autologous adipose tissue-derived mesenchymal stem cells along with BM-HSC in patient 2 under non-myeloablative conditioning. Patient 1 improved during the initial 6 months, but then again lost metabolic control with increased blood sugar levels and insulin requirement of 32 IU/day; the patient was lost to follow-up after 18 months. Patient 2, over follow-up of 24.87 months, has stable blood sugar levels with glycosylated haemoglobin of 6.4% and present insulin requirement of 15 IU/day [779]. The same group reported a variation of the protocol in a case report describing a 30-year-old-man with T1DM since 15 years and ESRD since 2 years, who underwent living donor RT and co-infusion of in vitro generated insulin-making cells differentiated from donor adipose tissue derived mesenchymal stem cells and bone marrow -derived haematopoietic SC into subcutaneous tissue, portal and thymic circulation under non-myeloablative conditioning. Over follow-up of 13 months he has stable graft function with serum creatinine, 1.2 mg/dl, zero rejection and glycosylated haemoglobin level of 6.1% on calcineurin-inhibitor based therapy [780].

The mesenchymal stem cell pioneer, Dr. Katherine Le Blanc, led a study on 20 adult patients with newly diagnosed type 1 diabetes who were enrolled and randomized to bone marrow mesenchymal stem cell treatment or to the control group. Residual β-cell function was analyzed as C-peptide concentrations in blood in response to a mixed-meal tolerance test (MMTT) at 1-year follow-up. In contrast to the patients in the control arm, who showed loss in both C-peptide peak values and C-peptide when calculated as area under the curve during the 1st year, these responses were preserved or even increased in the MSC-treated patients. Importantly, no side effects of MSC treatment were observed. The authors concluded that autologous MSC treatment in new-onset type 1 diabetes constitutes a safe and promising strategy to intervene in disease progression and preserve β-cell function [781].

In other embodiments of the invention, the invention teaches the use of novel stem cells for treatment of type 2 diabetes. Diabetes is a disease of hyperglycemia. There are two main forms of diabetes, Type 1 diabetes, and Type 2. In Type 1 diabetes, also known as insulin-dependent diabetes mellitus (IDDM), or juvenile diabetes, the patient’s pancreas produces little or no insulin, believed to be in part the result of autoimmune attached on the insulin producing beta-cells in the pancreas. It’s one of the most costly, chronic diseases of childhood and one you never outgrow. It is believed that more than one million Americans have IDDM. Patients with full-blown IDDM must take multiple insulin injections daily or continually infuse insulin through a pump, and test their blood sugar by pricking their fingers for blood six or more times per day. Neither dietary therapy nor treatment with an oral hypoglycemic agent is effective, and only treatment with insulin is effective. Ketonemia and acidosis due to the loss of insulin secreting capacity, and if untreated, may result in diabetic coma. Since numerous factors such as stress, hormones, growth, physical activity, medications, illness/infection, and fatigue effect insulin utilization, even a strictly monitored program of insulin administration does not mimic the endogenous functions of the pancreas, and as a result numerous complications develop.

Type 2 diabetes, also known as Non-Insulin Dependent Diabetes Mellitus (NIDDM), or adult-onset diabetes, is associated with impairment of peripheral tissue response to insulin. NIDDM is believed to afflict approximately 18.2 million people in the US and as a result of the obesity epidemic, substantially younger patients are beginning to be diagnosed with this condition. The economic burden of NIDDM is witnessed in statistics demonstrating that on average, the health care costs for NIDDM patients are approximately $13,243 for people with NIDDM, whereas age-matched controls is $2560 per year.

Insulin resistance is present in almost all obese individuals [782]. However, compensatory insulin production by beta-cells usually occurs, thus preventing hyperglycemia. In response to prolonged insulin resistance, as well as other factors, beta cell insulin production eventually lose ability to cope with the increasing insulin demands and postprandial hyperglycemia occurs, characterizing the transition between normal glucose tolerance and abnormal glucose tolerance. Subsequently, the liver starts secreting glucose through hepatic gluconeogenesis (generation of glucose from substrates that are not sugars, not from glycogen) and hyperglycemia is observed even in the fasting state. In contrast to IDDM, NIDDM presents only a small degree of ketonemia and acidosis although the insulin action is reduced from normal, and treatment with insulin is not always required.

The greatest clinical challenge in this disease is the prevention of the long-term complications, many of which involve vascular, ocular and renal systems. Although various agents are utilized to increase glucose sensitivity, insulin secretion, or exogenous insulin is used therapeutically, these do not exactly mimic the physiological control of post-prandial insulin secretion. Accordingly, the fluctuations of glucose, as well as downstream metabolic consequences end up causing macrovascular pathology such as coronary atherosclerosis, and increased risk of stroke, as well as microvascular pathology such as macular degeneration and renal failure. Additionally, neuropathies are often present associated with hyperglycemia.

### Current Treatments

There are numerous treatments available for NIDDM; these depend on patient-specific characteristics, as well as severity of disease. The treatment goal in diabetes treatment is to bring plasma glucose levels down to as near normal levels, for example 80-120 milligrams per deciliter (mg/dl) before meals and 100-140 mg/dl at night. Numerous medical tests are known in the art for monitoring glucose, as well as cholesterol and lipid levels. The goal of maintaining normal glucose levels is judged in some ways, by the ability to prevent secondary complications such as retinopathy, neuropathy, vascular disease, and strokes. In beginning phases of NIDDM patients may be treated with various oral drugs, as diabetes progresses, various forms of insulin may be administered. Although tight glucose control is known to decrease the rate of diabetic complications, such control is very difficult to achieve, and when achieved significant morbidity and mortality still occurs. Below are listed some of the non-insulin treatments for NIDDM. Mainstream oral treatments for diabetes can be separated by mechanism of action into two groups: hypoglycemics, such as sulfonylureas and meglitinides which induce beta cell insulin secretion and antihyperglycemics such as biguanides and alpha-glucosidase inhibitors which cause uptake of glucose.

Sulfonylureas are a type of drug that stimulate insulin release from beta cells. Essentially, these agents work by blocking ATP-sensitive potassium channels in the pancreatic beta-cell membrane. This effect is mediated by the binding of the drug to the sulfonylurea receptor (SUR) subunit of the channel. Inhibition of the potassium channel leads to depolarization of the cell membrane and insulin secretion, in a similar way as if glucose was added to the cell. Glyburide is a second generation sulfonylurea compound that is sold under the names Micronase, DiaBeta, or Glynase. Glipizide, sold under the names Glucotrol and Glucotrol XL, is also a second generation sulfonylurea drug. Third-generation sulfonylurea drugs include Glimepiride (Amaryl). This agent is believed to have greater safety in patients with ischemic heart disease as compared to other sulfonylurea drugs. Glimepiride is the only sulfonylurea based drug that is approved for use together with insulin or metformin. In general, sulfonylurea drugs suffer from the disadvantage that the amount of insulin secretion induced depends on the timing and dose of drug administration and not by the blood glucose levels. This causes not only various fluctuations in glucose level but also digestive symptoms such as anorexia in some patients.

Meglitinides (commonly called glinides) are a class of insulin secretagogues that have short-acting activity, given after meals. Similar to sulfonylurea drugs in that mechanistically they induce insulin secretion by closure of the ATP-dependent potassium channel, glinides appear to be more short-term in activity. Theoretically these drugs have less risk of inducing hypoclycemia and cause a physiological-like insulin release pattern. Repaglinide, sold under the name Prandin, and Nateglinide, sold under the name Starlix, are examples of two glinides. When compared with sulfonylurea drugs, glinides have been shown to provide a better control of postprandial hyperglycaemia, not to induce hypoglycaemia, and to generally have better safety profile, especially in patients with renal failure [783].

Biguanides are a class of drugs that decrease hepatic glucose production and increase insulin sensitivity. Metformin, sold under the names Glucophage, Glucophage XR, and Metformin XR is an example of a biguanide. It is also the most widely prescribed oral antidiabetic in the world and is in most circumstances the agent of choice for first line initial therapy of the typical obese patient with type 2 DM and mild to moderate hyperglycaemia [784]. Metformin administration is associated with weight loss and improvement in lipid profile. Metformin is effective as monotherapy and, in combination with both insulin secretagogues and thiazolidinediones (TZDs), may alleviate the need for insulin treatment [785]. It is known that metformin induces increased glucose utilization and reduction in leptin concentrations [786]. Additionally, metformin induces inhibition of dipeptidyl peptidase-IV activity, which allows for extended half-life of GLP-1 [787]. Classical mechanisms of action include increased glucose use by anaerobic glycolysis, inhibition of hepatic gluconeogenesis, and suppression of intestinal absorption of glucose. One adverse effect associated with various biguanides is lactic acidosis.

Thiazolidinediones (glitazones) are a family of drugs that decrease insulin resistance in both muscle and adipose tissue. They do not induce insulin secretion. Rosiglitazone, sold under the name Avandia, and Pioglitazone, sold under the name Actos are two thiazolidinediones. These agents induce insulin sensitivity through the activation of insulin receptor kinase, thereby promoting glucose uptake by peripheral tissues, and ameliorating increased liver glucose production. Known side effects include digestive symptoms and edema, and hematological alterations, and upregulation in plasma LDH. Glitazones are interesting not only from their ability to increase insulin signal transduction, but also due to anti-inflammatory effects. It is known, for example, that rosiglitazone inhibits ability of dendritic cells to secrete interleukin-12 after stimulation via CD40 [788]. This is believed to occur via activation of PPAR-gamma pathways. Additionally, treatment with rosiglitazone is able to inhibit onset of colitis in animal models through preferential induction of Th2 cytokine production [789]. Alpha-glucosidase inhibitors are used to delay rate of sugar absorption. Acarbose, sold under the name Precose, and Miglitol sold under the name Glyset are two examples of drugs in this family.

Incretin mimetics mirror glucose-dependent insulin secretion, cause inhibition of glucagon secretion, and delay gastric emptying. Exenatide, sold under the name Byetta, is a glucagon-like-peptide-1 (GLP-1) receptor agonist and stimulates insulin secretion from the beta cell. Controlled clinical trials provided evidence that glycaemic control under exenatide administered twice daily in a dose of 5 - 10 microg was not inferior to conventional insulin therapy.

### Clinical Trials of Mesenchymal Stem Cells in Type 2 Diabetes

Kong et al recruited 18 patients of T2DM who were intravenously transfused three times with umbilical cord mesenchymal stem cells.. All patients were followed up in the first, third, and sixth month. Age, gender, diabetes duration and medications as well as weight, height, and BMI were recorded. Fasting plasma glucose (FPG), postprandial blood glucose (PBG), HbA1c, C-peptide, and subsets of T cells were measured. All adverse reactions were carefully documented. UMSCs were successful obtained. Baseline clinical characteristics between the efficacy and control groups were not statistically different (p > 0.05). FBG and PBG of the patients in efficacy group were significantly reduced (p < 0.05) after UMSC transfusion. Plasma C-peptide levels and regulatory T (Treg) cell number in the efficacy group were numerically higher after UMSC transfusion; however, the difference of both parameters did not reach significance (p > 0.05). During the treatment course only 4 out of 18 patients (22.2%) had slight transient fever. Up to 6 months after UMSC transfusion, all patients continued to have a feeling of well-being and were physically more active [790]. A subsequent study used a similar cellular population, Wharton’s Jelly, of the umbilical cord to isolate mesenchymal stem cells. A total of 61 patients with T2DM were randomly divided into two groups on the basis of basal therapy; patients in group I were administered WJ-MSC intravenous infusion twice, with a four-week interval, and patients in group II were treated with normal saline as control. During the 36-month follow-up period, the occurrence of any adverse effects and the results of clinical and laboratory examinations were recorded and evaluated. The lack of acute or chronic adverse effects in group I was consistent with group II. Blood glucose, glycosylated hemoglobin, C-peptide, homeostasis model assessment of pancreatic islet β-cell function and incidence of diabetic complications in group I were significantly improved, as compared with group II during the 36-month follow-up. The results of the present study demonstrated that infusion of WJ-MSC improved the function of islet β-cells and reduced the incidence of diabetic complications [791].

Preservation of beta cell function was tested using administration of umbilical cord derived mesenchymal stem cells in type 2 diabetics in an independent study with a longer follow-up than previous ones. T he safety and efficacy of UCMSC application were evaluated in six patients with T2D during a minimum of a 24-month follow-up period. Following transplantation, the levels of fasting C-peptide, the peak value and the area under the C-peptide release curve increased significantly within one month and remained high during the follow-up period (P<0.05). Three of the six patients became insulin free for varying lengths of time between 25 and 43 months, while the additional three patients continued to require insulin injections, although with a reduced insulin requirement. Fasting plasma glucose and 2-h postprandial blood glucose levels were relatively stable in all the patients following transplantation. There was no immediate or delayed toxicity associated with the cell administration within the follow-up period. There were no safety issues observed during infusion and the long-term monitoring period [792].

Bone marrow derived mesenchymal stem cells termed “mesenchymal precursor cells”, developed by Mesoblast Inc where tested in a randomized placebo controlled trial in type 2 diabetics. Patients received one intravenous (IV) infusion of cells, at various concentrations depending on the dosing group they were randomized to: 0.3 × 10(6)/kg (n = 15), 1.0 × 10(6)/kg (n = 15), or 2.0 × 10(6)/kg (n = 15) or placebo (n = 16). Study duration was 12 weeks. Subjects (21 women, 40 men) with a mean ± SD baseline HbAlc 8.3 ± 1.0% (67 ± 10.9 mmol/mol), BMI 33.5 ± 5.5 kg/m(2), and diabetes duration 10.1 ± 6.0 years were enrolled at 18 U.S. sites. No acute adverse events (AEs) were associated with infusion. No serious AEs, serious hypoglycemia AEs, or discontinuations due to AEs over 12 weeks were found. No subjects developed donor-specific anti-HLA antibodies or became sensitized. The safety profile was comparable among treatment groups. Compared with placebo, a single IV infusion of cells reduced HbAlc at all time points after week 1. The adjusted least squares mean ± SE dose-related differences in HbAlc from placebo in the treated groups ranged from -0.1 ± 0.2% (-1.1 ± 2.2 mmol/mol) to -0.4 ± 0.2% (4.4 ± 2.2 mmol/mol) at 8 weeks and from 0.0 ± 0.25% to -0.3 ± 0.25% (-3.3 ± -2.7 mmol/mol) at 12 weeks (P < 0.05 for 2.0 × 10(6)/kg dose at 8 weeks). The clinical target HbAlc <7% (53 mmol/mol) was achieved by 33% (5 of 15) of the subjects who received the 2.0 × 10(6)/kg dose vs. 0% of those who received placebo (P < 0.05). The authors concluded that this short-term study demonstrates the safety and feasibility of up to 246 million mesenchymal precursor cells in subjects with type 2 diabetes [793].

### Treatment of Diabetic Complications by Mesenchymal Stem Cells

One of the important aspects of type 2 diabetes are the secondary complications. A case report described a patient who presented with severe post-traumatic infection and a non-healing skin defect in the hand, secondary to uncontrolled diabetes mellitus. An autologous bone marrow mesenchymal stem cell suspension was injected into the persistent skin defect after stabilizing the blood glucose level and appropriate infection control. During the course of a regular 18-month postoperative follow-up, the patient exhibited immediate recovery with no transplant-associated complications, as well as no evidence of tumorigenicity. Thus, transplantation of autologous MSCs may play a role in the clinical application of stem cells, particularly for treatment of skin defects following surgery in cases of diabetes and for those caused by various other traumas [794].

One of the complications of diabetes is peripheral artery disease. In one regenerative medicine study investigators used umbilical cord mesenchymal stem cells to treat 53 patients (72 limbs) with severe symptoms of Fontaine II-IV diabetic foot accompanied by varying degrees of lower extremity arterial disease. The patients were randomly apportioned to a control group (25 patients; 38 limbs) or an experimental group (28 patients; 34 limbs). Patients of both groups received interventional treatment with angioplasty; those in the experimental group also received umbilical cord mesenchymal stem cells by endovascular infusion and injection around the foot ulcer. Within the 3-month follow-up, relative to patients in the control group, those in the experimental group experienced significantly greater and more stable improvements in skin temperature, ankle-brachial pressure index, transcutaneous oxygen tension, and claudication distance. Notably, 3 months after treatment a significant increase in neovessels, accompanied by complete or gradual ulcer healing, was shown in the experimental group. In addition, no serious complications or adverse reactions were associated with the treatment [795].

Another study in diabetic foot disease described treatment of 15 diabetic patients with foot disease under insulin therapy received umbilical cord mesenchymal stem cell transplantation. The cells were directly injected into the quadriceps thigh muscles in patients with foot disease (cell quantity at 2 × 10⁶ per point). Physical attributes, blood cytokines, blood glucose and insulin dosage were evaluated before treatment and 1, 2, 4, 8, and 12 weeks thereafter. The ratios of Treg/Th17, Treg/Th1, and Th17/ Th1 cells were measured using flow cytometry and their correlation with various cytokines (FoxP3, IL-17, INF-y, C-RP, TNF-α, and VEGF) was scrutinized. Levels of blood glucose and insulin dosage were significantly reduced in all 15 patients following hUCB-MSC transplantation. The ratios of CD4⁺CD25(hi)FoxP3⁺ Treg/Th17 and CD4⁺CD25(hi)FoxP3⁺ Treg/Th1 cells were significantly increased 4 weeks after transplantation (p < 0.01), while the ratio of Th17/Th1 cells remained unchanged. Serum levels of VEGF peaked at 4 weeks following transplantation. Levels of C-RP and TNF-α were significantly reduced 4 weeks after transplantation. Intriguingly, the ratios of Treg/Th17 were positively correlated with VEGF levels, and were inversely correlated with plasma IL-6 levels. These data indicated that immune disorders are associated with the development of type 2 diabetes and its complications. Levels of blood glucose and required insulin dosage were reduced after stem cell transplantation accompanied with improved clinical profiles in diabetic patients. These data favor a role for Treg cells in the onset and progression of T2D [796].

Another embodiment of the current invention teaches the utilization of the novel cells for enhancing survival of organ transplants. Organ transplantation is the only option for patients with end-stage organ failure. Although the era of calcineurin inhibitors has more or less effectively solved the problem of acute rejection, chronic rejection, which is primarily an anti-body mediated process is still a significant problem. Chronic allograft rejection is attributed primarily to organ damage during the process of transplantation [606]. The advantage of using mesenchymal stem cells are part of transplantation conditioning or maintenance therapy is that the possibility exists of reducing damage to the organ through stimulating of regeneration, as well as activating tolerogenic signals that potentially could allow for reduced or no chronic immune suppressant therapy. The use of cell therapy as part of organ transplantation is not new in that early transplantation studies used donor lymphocyte infusions to attempt to prevent graft rejection, however with marginal success [797]. In vitro studies showed that admixing donor specific mesenchymal stem cells with recipient lymphocytes results in suppression of anti-donor antigen reactivity, providing support for utilizing these cells as immune modulators [798].

### Recipient Derived Mesenchymal Stem Cell Clinical Trials

Perico et al. reported a safety and clinical feasibility study infusing autologous (recipient) mesenchymal stem cells in 2 patients who received renal transplants from living-related donors. Patients were given T cell-depleting induction therapy and maintenance immunosuppression with cyclosporine and mycophenolate mofetil. On day 7 after the transplant, mesenchymal stem cells were administered intravenously. Serum creatinine levels increased 7 to 14 days after cell infusion in both MSC-treated patients. A graft biopsy in patient 2 excluded acute graft rejection, but showed a focal inflammatory infiltrate, mostly granulocytes. In patient 1 protocol biopsy at 1-year posttransplant showed a normal graft. Both MSC-treated patients are in good health with stable graft function. A progressive increase of the percentage of CD4+CD25highFoxP3+CD127- Treg and a marked inhibition of memory CD45RO+RA-CD8+ T cell expansion were observed posttransplant. Patient T cells showed a profound reduction of CD8+ T cell alloreactivity. This study suggested feasibility of immune modulation using recipient mesenchymal stem cells, however the reason of why an initial increase in creatinine in response to the autologous cells is unknown [799].

In a subsequent study, Mudrabettu et al performed autologous mesenchymal stem cell transplantation pre-transplant and one month post-transplant in 4 recipients of living donor renal transplants. None of the four patients developed any immediate or delayed adverse effects following MSC infusion. All had excellent graft function, and none developed graft dysfunction. Protocol biopsies at 1 and 3 months did not reveal any abnormality. Compared to baseline, there was an increase in the CD4 + CD25+FOXP3+ regulatory T cells and reduction in CD4 T cell proliferation [800] .

Reinders et al. performed a safety and feasibility study in kidney allograft recipients to whom two intravenous infusions (1 million cells per kilogram) of autologous bone marrow (BM) MSCs were given, when a protocol renal biopsy at 4 weeks or 6 months showed signs of rejection and/or an increase in interstitial fibrosis/tubular atrophy (IF/TA). Six patients received MSC infusions. Clinical and immune monitoring was performed up to 24 weeks after MSC infusions. MSCs fulfilled the release criteria, infusions were well-tolerated, and no treatment-related serious adverse events were reported. In two recipients with allograft rejection, we had a clinical indication to perform surveillance biopsies and are able to report on the potential effects of MSCs in rejection. Although maintenance immunosuppression remained unaltered, there was a resolution of tubulitis without IF/TA in both patients. Additionally, three patients developed an opportunistic viral infection, and five of the six patients displayed a donor-specific downregulation of the peripheral blood mononuclear cell proliferation assay, not reported in patients without MSC treatment. Autologous BM MSC treatment in transplant recipients with subclinical rejection and IF/TA is clinically feasible and safe, and the findings are suggestive of systemic immunosuppression [801].

Trivedi et al. reported a demographically balanced three-armed living donor renal transplant trial with 95 patients in each arm, group-1 received portal co-infusion of adipose derived mesenchymal stem cells and hematopoietic stem cells, group-2 received hematopoietic stem cells along and group-3 received no stem cells. Lymphoid irradiation and anti-thyroglobulin were used for conditioning. They demonstrated that cellular therapy was safe with no treatment associated serious adverse events. At 1 and 5 years post-transplant, patient survival was 100% and 94.7% in group-1, 100% and 95.7% in group-2, and 94.7% and 84% in group-3, death-censored graft survival was 100% and 94.6% in group-1, 100% and 91.3% in group-2, and 98.9% and 94.4% in group-3 with mean serum creatinine (mg/dL) of 1.38 and 1.39 in group-1, 1.48 and 1.51 in group-2, and 1.29 and 1.42 and in group-3. Rejection episodes and immunosuppression requirement were lesser in the stem cell treated groups versus controls with best results noted in group-1 [802]. Trivedi’s group reported a subsequent study in which 96 recipients of living donor renal transplants received a similar regiment of autologous adipose derived mesenchymal stem cells and donor hematopoietic stem cells. LDRT patients subjected to pretransplant SCT who had stable graft function for ≥2 years and serum creatinine (SCr) <2 mg/dL were recruited. Patients received non-myeloablative conditioning of total lymphoid irradiation (TLI)/bortezomib and cyclophosphamide, rabbit-antithymocyte globulin (r-ATG) and rituximab with cellular therapy. The maintenance immunosuppression consisted of calcineurin inhibitors (CNI) and/or antiproliferative agents and prednisone. Donor-specific antibodies (DSA) and peripheral T-regulatory cells (CD127(low/-)/4(+)/25(high)) (p-Tregs) were studied before and after withdrawal of major immunosuppressants; graft biopsy was taken after 100 days of withdrawal in willing patients. All immunosuppression but prednisone, 5-10 mg/day has been successfully withdrawn for a mean of 2.2 years in 76 patients with a mean age of 31.4 years and a mean donor-recipient HLA match of 2.9. The mean SCr of 1.4 mg/dL and p-Tregs of 3.5% was remained stable after withdrawal; DSA status was negative in 35.5% and positive in 47.4% patients. Protocol biopsies in all 10 patients who gave the consent were unremarkable. This clinical trial demonstrated feasibility of inducing a “tolerance-like” state in which Stable graft function was achieved without use of calcineurin inhibitors but onlylow-dose steroid monotherapy [803].

In a paper whose senior author was the renowned diabeteologist Camillo Ricordi, from Diabetes Research Institute, patients were administered with bone marrow-derived autologous mesenchymal stem cells (1-2 × 10(6)/kg) at kidney grafting and two weeks later. Fifty-three patients received standard-dose and 52 patients received low-dose CNIs (80% of standard); 51 patients in the control group received anti-IL-2 receptor antibody plus standard-dose CNIs. Patient and graft survival at 13 to 30 months was similar in all groups. After 6 months, 4 of 53 patients (7.5%) in the autologous MSC plus standard-dose CNI group (95% CI, 0.4%-14.7%; P = 0.04) and 4 of 52 patients (7.7%) in the low-dose group (95% CI, 0.5%-14.9%; P = 0.046) compared with 11 of 51 controls (21.6%; 95% CI, 10.5%-32.6%) had biopsy-confirmed acute rejection. None of the patients in either autologous MSC group had glucorticoid-resistant rejection, whereas 4 patients (7.8%) in the control group did (95% CI, 0.6%-15.1%; overall P = 0.02). Renal function recovered faster among both MSC groups showing increased eGFR levels during the first month after surgery than the control group. Patients receiving standard-dose CNI had a mean difference of 6.2 mL/min per 1.73 m(2) (95% CI, 0.4-11.9; P=0.04) and those in the low-dose CNI of 10.0 mL/min per 1.73 m(2) (95% CI, 3.8-16.2; P=0.002). Also, during the 1-year follow-up, combined analysis of MSC-treated groups revealed significantly decreased risk of opportunistic infections than the control group (hazard ratio, 0.42; 95% CI, 0.20-0.85, P=0.02). The authors concluded that the use of autologous MSCs compared with anti-IL-2 receptor antibody induction therapy resulted in lower incidence of acute rejection, decreased risk of opportunistic infection, and better estimated renal function at 1 year [804].

The majority of clinical explorations using mesenchymal stem cells have used living donor renal transplants because of the relative ability to manipulate the graft in a non-live threatening manner compared to other organ systems. An interesting study evaluated use of recipient bone marrow mesenchymal stem cell therapy on patients receiving small bowel transplants. Six isolated intestinal transplants have been performed with MSC therapy since 2009. The primary reasons for transplants were short gut syndrome caused by surgical intestine resection for superior mesenteric artery thrombosis (n = 4), Crohn’s disease (n = 1) and intestinal aganglionosis (n = 1). Two of the patients were children. At the time of reperfusion, the first dose of mesenchymal stem cells cultured from the patient’s bone marrow was passed into the transplanted intestinal artery at a dose of 1000000 cells/kg. The second and third doses of mesenchymal stem cells were given directly into the mesenteric artery through the arterial anastomosis using an angiography catheter on day 15 and 30 post-transplant. The median follow-up for these patients was 10.6 mo (min: 2 mo-max: 30 mo). Three of the patients developed severe acute rejection. One of these patients did not respond to bolus steroid therapy. Although the other two patients did respond to anti-rejection treatment, they developed severe fungal and bacterial infections. All of these patients died in the 2(nd) and 3(rd) months post-transplant due to sepsis. The remaining patients who did not have acute rejection had good quality of life with no complications observed during the follow-up period. In addition, their intestinal grafts were functioning properly in the 13(th), 25(th) and 30(th) month post-transplant. The patients who survived did not encounter any problems related to mesenchymal stem cells transplantation. Although it is impossible to judge efficacy due to the small number of patients in the study, the authors stated that in comparison to their experiences using standard immune suppression in small bowel transplants, the outcomes suggested value of continued exploration of mesenchymal stem cells as an adjuvant to inhibit rejection in small bowel transplants [805].

### Donor Derived Mesenchymal Stem Cell Clinical Trials

Peng et al. administered donor-derived bone marrow mesenchymal stem cells combined with a sparing dose of tacrolimus (50% of standard dose) to six de novo living-related kidney transplant recipients. Six other patients who received a standard dose of tacrolimus were enrolled as a control. The safety of mesenchymal stem cell infusion, acute rejection, graft function, and patient and graft survival within 12 months after kidney transplantation were observed. None of the mesenchymal stem cell recipients experienced immediate or long-term toxic side effects associated with MSC infusion. The tacrolimus dose (0.045±0.002 mg/kg) in the mesenchymal stem cell treated group was significantly reduced compared with the control group (0.077±0.005 mg/kg). One acute rejection occurred only in the control group. All patients survived with stable renal function at month 12 and no chimerism was detectable at month 3. Patients in the mesenchymal stem cell group showed significantly higher B-cell levels than the control group at month 3 [806].

In another embodiment, the invention teaches the utilization of novel stem cells for treatment of spinal cord injury. Spinal cord injury (SCI) is caused by direct mechanical damage to the spinal cord that usually results in complete or incomplete loss of neural functions such as mobility and sensory function. To date, no therapy exists to induce recovery in patients with SCI.

Although endogenous repair mechanisms are activated after SCI, such as proliferation of endogenous stem cells [807-809], their activity is in many times clinically insignificant. Administration of mesenchymal stem cells (MSC) in animal models of SCI induces a therapeutic benefit by: a) production of trophic factors that inhibit inflammation and induce endogenous neurogenesis [810]; b) differentiate into, or stimulating endogenous cells to differentiate into myelin-forming cells [811, 812]; and c) providing an environment suitable for axon regeneration through inhibition of fibrosis [813] and stimulation of angiogenesis [814, 815].

Nerve damage in SCI occurs in the majority of cases as a result of the combined effects of the initial physical injury, and subsequent inflammatory response caused in part by physical damage to the blood brain barrier, immune cell response to injury, and local ischemia. Typical causes of injury include contusive, compressive or stretch damage which is associated with severing of axons at the nodes of Ranvier, leading to axon retraction [816]. Furthermore, axons proximal to the area of injury that do not retract are known to develop abnormalities such as loss of myelination and swelling of the axonal body, resulting in loss of excitability [817]. Demyelination is in part believed to occur due to death of oligodendrocytes surrounding the axon, a process which occurs even at 3 weeks after the initial injury [818]. Importance of demyelination in this process is seen in experiments where remyelination induced by administration of Schwann cells has been demonstrated to elicit benefit in animal models of SCI [819].

Mechanistically, oligodendrocyte death appears to be related to the death receptor Fas based on: a) Pattern of expression is temporarily correlated with oligodendrocyte apoptosis in SCI models [820]; b) Genetic inactivation of Fas results in reduced oligodendrocyte death [821]; c) Administration of soluble Fas [822] has a protective effect on SCI associated demyelination. Interestingly, administration of human umbilical cord stem cells in a rat SCI model results in therapeutic benefit which seems to be mediated by reduction of Fas expression [823]. Death of neurons themselves subsequent to SCI is associated with release of glutamate and other excitotoxins such as free ATP [824-826]. Interestingly, excitotoxicity occurs not only as a result of initial injury, but has also been implicated in secondary, more long-term, neuronal damage [827].

Subsequent to spinal cord injuries, Schwann cells originating from the spinal root traffic to the area of injury and initiate a process of remyelinating injured axons [807]. An endogenous progenitor cell type, termed the ependymal cell, was observed in early studies to proliferate after spinal cord transaction in animal models [808, 809, 828]. It appears that ependymal cells purified from SCI rats proliferate in vitro almost 10-fold faster than ependymal cells from control animals, thus suggesting an injury-associated mitogenic event. Furthermore, in the same study it was demonstrated that transplantation of undifferentiated ependymal cells or differentiated oligodendrocyte precursor cells generated from ependymal cells, when administered to a rat model of severe spinal cord contusion induced recovery of motor activity 1 week after injury [829].

Using genetic cell fate mapping, it was demonstrated that primarily all neurogenic cells present post SCI are derived from ependymal cells, including glial cells associated with scar tissue, as well as a smaller number of oligodendrocytes [830]. Ependymal cells are known to react to exogenous growth factors, for example, intrathecal administration of EGF and FGF-2 was demonstrated to induce ependymal cell proliferation [831]. Thus one therapeutic approach may be administration of exogenous factors that stimulate/accelerate natural remyelination processed. Indeed administration of FGF-2 has been demonstrated to improve locomotor function in a rat SCI model [832]. Alternatively, administration of stem cells that naturally produce FGF-2 may be a more biological method of inducing accelerated recovery [833].

Angiogenesis is an integral part of numerous healing processes. In the context of spinal cord injury, hypoxia inducible factor (HIF)-1 alpha activates numerous downstream effectors such as BDNF, VEGF, SDF-1, TrkB, Nrp-1, CXCR4 and NO, that attempt to restore the “neurovascular niche” after damage has occurred [834]. These molecules act not only one creation of new vasculature but also are involved in neurogenesis. The critical link between neural recovery and angiogenesis may be seen in animal models of post-stroke regeneration where cord blood derived cells appear to elicit effects primarily by stimulating de novo vasculature which causes expansion of endogenous progenitors [835]. [835]. Transfection of neuronal progenitors with the angiogenic factor VEGF has been shown to increase angiogenesis and recovery [836]. Additionally, administration of human CD133 peripheral blood progenitor cells accelerates post-injury healing in part through secretion of VEGF [837]. There is some evidence that counter-angiogenic mechanisms are present in the late post-injury setting. For example Mueller et al showed that approximately 7 days post injury an accumulation of endostatin/collagen XVIII is observed in the areas associated with vascular remodeling [838]. Thus in any potential regenerative intervention for SCI, it is essential to ensure that the pro-angiogenic/anti-angiogenic balance is weighed on the side of pro-angiogenesis.

### Rationale for MSC Therapies in SCI

The known anti-inflammatory [839, 840], antifibrotic [841], angiogenic [842], and neurotrophic [843], activities of MSC have stimulated numerous preclinical studies in the area of SCI, based on the rationale provided above. Although 21 animal studies have demonstrated therapeutic effects of MSC in preclinical models were recently reviewed by Li et al [844], we provide some example studies below.

Kim et al used a chronic contusive SCI model in 36 Sprague-Dawley rats and randomly assigned to the intralesional (IL), intravenous (IV), or control saline injection. At 6 weeks post-injury, allogenic mesenchymal stem cells (MSCs, 1×10 cells) were transplanted either intralesionally or intravenously for the intervention groups. Therapeutic benefit in terms of improved mobility was assessed using the Basso, Beattie, and Bresnahan (BBB) score 6 weeks post stem cell administration. the mean BBB locomotor scales in the IL, IV and control group were 5.63±0.89, 5.63±1.03 and 2.88±0.44, respectively. This represents a significant clinical improvement. Additionally no ectopic tissue or adverse events were observed with either iv or intralesional injection methodology [845]. Boido et al utilize a compression injury model in the mouse spinal cord, MSC were acutely transplanted into the lesion cavity; injured mice without the graft served as controls. After 26 days, the survival of MSCs was investigated, and their effect on the formation of glial cyst and on injury-related inflammation was evaluated. The investigators found that the lesion volume was reduced by 31.6% compared with control mice despite the fact that astroglial and microglial activation was not altered by the graft. Sensory and motor tests showed that MSC cell therapy results in improvement on a battery of behavioral tests compared with control mice: MSC-treated mice versus control mice scored 0.00 versus 0.50 in the posture test, 0.00 versus 1.50 in the hindlimb flexion test, 3.00 versus 2.25 in the sensory test, and 7.50 mistakes versus 15.83 mistakes in the foot-fault test [846].

In order to assess whether exogenously administered MSC contribute to the activation of endogenous repair processes, Park et al. induced SCI by inducing a contusion using a weight-drop impactor in mice and human UC-MSC were transplanted into the boundary zone of the injured site. Animals received a daily injection of bromodeoxyuridine (BrdU) for 7 days after treatment to identity newly synthesized cells of ependymal and periependymal cells that immunohistochemically resembled stem/progenitor cells was evident. Behavior analysis revealed that locomotor functions of hUCB-MSCs group were restored significantly and the cavity volume was smaller in the MSCs-transplanted rats compared to the control group. In MSCs-transplanted group, TUNEL-positive cells were decreased and BrdU-positive cells were significantly increased rats compared with control group. In addition, more of BrdU-positive cells expressed neural stem/progenitor cell nestin and oligo-lineage cell such as NG2, CNPase, MBP and glial fibrillary acidic protein typical of astrocytes in the MSC-transplanted rats. Thus, endogenous cell proliferation and oligogenesis contribute to MSC-promoted functional recovery following SCI [810].

### Clinical Trials of Autologous BM MSC

One of the initial clinical trials using MSC in SCI was in 30 patients with clinically complete SCI at cervical or thoracic levels were recruited and divided into two groups based on the duration of injury. Patients with <6 months of post-SCI were recruited into group 1 and patients with >6 months of post-SCI were included into group 2. Autologous BM was harvested from the iliac crest of SCI patients under local anesthesia and BM MSC were isolated and expanded ex vivo. BM MSC were tested for quality control, characterized for cell surface markers and transplanted back to the patient via lumbar puncture at a dose of 1 × 10(6) cells/kg body weight. Rhree patients had completed 3 years of follow-up post-BM MSC administration, 10 patients 2 years follow-up and 10 patients 1 year follow-up. Five patients have been lost to follow-up. None of the patients have reported any adverse events associated with BM MSC transplantation [847].

A variation of the above study examined 64 patients, at a mean of 3.6 years after SCI. Forty-four subjects received monthly intrathecal autologous MSCs for 6 months and 20 subjects, who would not agree to the procedures, served as controls. All subjects received rehabilitation therapies 3 times weekly. Subjects were evaluated at entry and at 12 months after completing the 6-months intervention. By the ASIA Impairment Scale, ASIA grading of completeness of injury, Ashworth Spasticity Scale, Functional Ambulation Classification, and bladder and bowel control questionnaire. No differences were found in baseline measures and descriptors between the MSC group and control group. Although a higher percentage of the MSC group increased motor scores by 1-2 points and changed from ASIA A to B, no significant between-group improvements were found in clinical measures. Adverse effects of cells included spasticity and, in 24 out of the 43 patients developed neuropathic pain. One subject with a history of post-infectious myelitis developed encephalomyelitis after her third injection [848].

This nonrandomized clinical trial compared the results of autologous BMC transplantation into cerebrospinal fluid (CSF) via lumbar puncture (LP) in 11 patients having complete SCI, with 20 patients as control group who received conventional treatment without BMC transplantation. The patients underwent preoperative and follow-up neurological assessments using the American Spinal Injury Association (ASIA) impairment scale. Then, the participants were followed for 12-33 months. Eleven patients with the mean age of 33.2±8.9 years and 20 patients with the mean age of 33.5±7.2 years were enrolled in the study and in the control group, respectively. None of the patients in the study and control group experienced any adverse reaction and complications, neither after routine treatment nor after cell transplantation. Five patients out of 11 (45.5%) in the study group and three patients in the control group (15%) showed marked recovery, but the result was statistically borderline (P=0.095) [849].

From 2009 to 2010, a total of 20 SCI patients were enrolled in a clinical trial by Wuhan Hongqiao Brain Hospital; all patients completed and signed informed consent prior to autologous bone marrow-derived mesenchymal stem cell transplantation. Analysis of subsequent treatment results indicated significant improvements in sensory, motor and autonomic nerve function as assessed by the American Spinal Injury Association’s impairment scale. Thirty days after transplantation, a total of 15 patients (75%) demonstrated improvement, including four of the eight patients (50%) with grade A SCI, three of the four patients (75%) with grade B injury and all eight patients (100%) with grade C injury. The most common adverse events, fever and headache, disappeared within 24-48 h without treatment [850].

This study explored the efficacy of autologous bone marrow mesenchymal stem cells (BMMSCs) transplantation in the treatment of SCI. Forty patients with complete and chronic cervical SCI were selected and randomly assigned to one of the two experimental groups, treatment group and control group. The treatment group received BMMSCs transplantation to the area surrounding injury, while the control group was not treated with any cell transplantation. Both the transplant recipients and the control group were followed up to 6 months, postoperatively. Preoperative and postoperative neurological functions were evaluated with AIS grading, ASIA score, residual urine volume and neurophysiological examination. Results showed that in the treatment group 10 patients had a significant clinical improvement in terms of motor, light touch, pin prick sensory and residual urine volume, while nine patients showed changes in AIS grade. Neurophysiological examination was consistent with clinical observations. No sign of tumor was evident until 6 months postoperatively. In the control group, no improvement was observed in any of the neurological functions specified above. BMMSCs transplantation improves neurological function in patients with complete and chronic cervical SCI, providing valuable information on applications of BMMSCs for the treatment of SCI [851].

To further assess validity of this technique, in another study the authors conducted a phase I, non-controlled study in 14 subjects of both genders aging between 18 to 65 years, with chronic traumatic SCI (>6 months), at thoracic or lumbar levels, classified as American Spinal Injury Association (ASIA) A - complete injury. Baseline somatosensory evoked potentials (SSEP), spinal magnetic resonance imaging (MRI) and urodynamics were assessed before and after treatment. Pain rating was performed using the McGill Pain Questionnaire and a visual analogue score scale. Bone marrow-derived mesenchymal stem cells were cultured and characterized by flow cytometry, cell differentiation assays and G-band karyotyping. Mesenchymal stem cells were injected directly into the lesion following laminectomy and durotomy. Cell transplantation was an overall safe and well-tolerated procedure. All subjects displayed variable improvements in tactile sensitivity and eight subjects developed lower limbs motor functional gains, principally in the hip flexors. Seven subjects presented sacral sparing and improved American Spinal Injury Association impairment scale (AIS) grades to B or C - incomplete injury. Nine subjects had improvements in urologic function. One subject presented changes in SSEP 3 and 6 months after mesenchymal stem cells transplantation. Statistically significant correlations between the improvements in neurological function and both injury size and level were found. Intralesional transplantation of autologous mesenchymal stem cells in subjects with chronic, complete spinal cord injury is safe, feasible, and may promote neurological improvements [852].

A 33-year-old Thai man who sustained an incomplete spinal cord injury from the atlanto-axial subluxation was enrolled into a pilot study aiming to track bone marrow-derived mesenchymal stem cells, labeled with superparamagnetic iron oxide nanoparticles, from intrathecal transplantation in chronic cervical spinal cord injury. He had been dependent on respiratory support since 2005. There had been no improvement in his neurological function for the past 54 months. Bone marrow-derived mesenchymal stem cells were retrieved from his iliac crest and repopulated to the target number. One half of the total cells were labeled with superparamagnetic iron oxide nanoparticles before transplantation to the intrathecal space between L4 and L5. Magnetic resonance imaging studies were performed immediately after the transplantation and at 48 hours, two weeks, one month and seven months after the transplantation. His magnetic resonance imaging scan performed immediately after the transplantation showed hyposignal intensity of paramagnetic substance tagged stem cells in the subarachnoid space at the lumbar spine area. This phenomenon was observed at the surface around his cervical spinal cord at 48 hours. A focal hyposignal intensity of tagged bone marrow-derived stem cells was detected at his cervical spinal cord with magnetic resonance imaging at 48 hours, which faded after two weeks, and then disappeared after one month. No clinical improvement of the neurological function had occurred at the end of this study. However, at 48 hours after the transplantation, he presented with a fever, headache, myalgia and worsening of his motor function (by one grade of all key muscles by the American Spinal Injury Association impairment scale), which lasted for 48 hours. Intrathecal injection of bone marrow-derived stem cells at the lumbar spine level could deliver the cells to the injured cervical spinal cord. Transient complications should be observed closely in the first 48 hours after transplantation. Further study should be carried out to evaluate the result of the treatment [853].

Patients were selected based on the following criteria: chronic American Spinal Injury Association B status patients who had more than 12 months of cervical injury, and no neurological changes during the recent 3 months of vigorous rehabilitation. We injected 1.6 × 10 autologous MSCs into the intramedullary area at the injured level and 3.2 × 10 autologous MSCs into the subdural space. Outcome data were collected over 6 months regarding neurological examination, magnetic resonance imaging with diffusion tensor imaging, and electrophysiological analyses. Among the 16 patients, only 2 showed improvement in neurological status (unilateral right C8 segment from grade 1 to grade 3 in 1 patient and bilateral C6 from grade 3 to grade 4 and unilateral right C8 from grade 0 to grade 1 in 1 patient). Both patients with neurological improvement showed the appearance of continuity in the spinal cord tract by diffusion tensor imaging. There were no adverse effects associated with MSCs injection [854].

Patients with complete SCI at the thoracic level were divided into two groups: chronic (>6 months, group 1) and sub-acute SCI (<6 months, group 2), according to time elapsed since injury. MSCs were isolated by density gradient separation of autologous bone marrow harvested from the iliac crest. Cells were cultured in a Good Manufacturing Practice-compliant facility to produce clinical scale dose. After quality control testing, MSCs were injected back to patients by intrathecal injection. Safety was defined as absence of adverse event and side effects after 1 month after receiving the injection. Six patients had chronic SCI with a median duration of 33 months since date of injury (range: 10-55 months), and three patients were in sub-acute phase of disease. Each patient received two or three injections with a median of 1.2 × 10(6) MSCs/kg body weight. No treatment-related adverse event was observed during median follow-up of 720 days (range: 630-826 days) in group 1 and 366 days (range: 269-367 days) in group 2, respectively. It was shown that autologous MSCs can be safely administered through intrathecal injection in spinal cord injury patients [855].

### Autologous Adipose MSC

To assess efficacy of using adipose tissue as an alternative to BM, 10 patients with posttraumatic paraplegia of mean age 3.42 years were volunteered for SCT. Their mean age was 28 years, and they had variable associated complications. They were subjected to adipose tissue resection for in vitro generation of N-Ad-MSC and bone marrow aspiration for generation of HSC. Generated SCs were infused into the cerebrospinal fluid (CSF) below injury site in all patients. Total mean quantum of SC infused was 4.04 ml with a mean nucleated cell count of 4.5 × 10(4)/µL and mean CD34+ of 0.35%, CD45-/90+ and CD45-/73+ of 41.4%, and 10.04%, respectively. All of them expressed transcription factors beta-3 tubulin and glial fibrillary acid protein. No untoward effect of SCT was noted. Variable and sustained improvement in Hauser’s index and American Spinal Injury Association score was noted in all patients over a mean follow-up of 2.95 years. Mean injury duration was 3.42 years against the period of approximately 1-year required for natural recovery, suggesting a positive role of SCs [856].

Long-term results of 10 patients who underwent intramedullary direct MSCs transplantation into injured spinal cords. Autologous MSCs were harvested from the iliac bone of each patient and expanded by culturing for 4 weeks. MSCs (8 × 10) were directly injected into the spinal cord, and 4 × 10 cells were injected into the intradural space of 10 patients with American Spinal Injury Association class A or B injury caused by traumatic cervical SCI. After 4 and 8 weeks, an additional 5 × 10 MSCs were injected into each patient through lumbar tapping. Outcome assessments included changes in the motor power grade of the extremities, magnetic resonance imaging, and electrophysiological recordings. Although 6 of the 10 patients showed motor power improvement of the upper extremities at 6-month follow-up, 3 showed gradual improvement in activities of daily living, and changes on magnetic resonance imaging such as decreases in cavity size and the appearance of fiber-like low signal intensity streaks. They also showed electrophysiological improvement. All 10 patients did not experience any permanent complication associated with MSC transplantation [857].

### Combinations of Cells

In order to take advantage of potential trophic effects of MSC to support implanted progenitor cells a safety and feasibility of a bone marrow mesenchymal stromal cell and Schwann cell combination for the treatment of patients with chronic spinal cord injury was performed. Eight subjects who received a complete traumatic spinal cord injury (American Spinal Injury Association [ASIA] classification A) enrolled in this study. The patients received this autologous combination of cells directly into the injury site. The mean duration of follow-up was approximately 24 months. No magnetic resonance imaging evidence of neoplastic tissue overgrowth, syringomyelia or psuedomeningocele in any of the patients was seen during the study. There was no deterioration in sensory or motor function in any of the patients during the course of the study. Three patients had negligible improvement in ASIA sensory scale. No motor score improvement and no change in ASIA classification was seen. The patients had widely subjective changes in the course of the study such as urination and defecation sensation and more stability and trunk equilibrium in the sitting position. The authors concluded there were no adverse findings at least 2 years after autologous transplantation of Schwann cell and mesenchymal stromal cell combination into the injured spinal cord. It appears that the use of this combination of cells is safe for clinical application to spinal cord regeneration [858].

An unconventional combination of cells attempted in SCI was the use of immune cells together with MSC. BM MSC were cocultured with the patient’s autoimmune T (AT) cells to be transdifferentiated into NSC. Forty-eight hours prior to NSC implant, patients received an i.v. infusion of 5 x 10(8) to 1 x 10(9) AT cells. NSC were infused via a feeding artery of the lesion site. Safety evaluations were performed everyday, from the day of the first infusion until 96 h after the second infusion. After treatment, patients started a Vojta and Bobath neurorehabilitation program.b\Two patients have been treated. Patient 1 was a 19-year-old man who presented paraplegia at the eight thoracic vertebra (T8) with his sensitive level corresponding to his sixth thoracic metamere (T6). He received two AT-NSC treatments and neurorehabilitation for 6 months. At present his motor level corresponds to his first sacral metamere (S1) and his sensitive level to the fourth sacral metamere (S4). Patient 2 was a 21-year-old woman who had a lesion that extended from her third to her fifth cervical vertebrae (C3-C5). Prior to her first therapeutic cycle she had severe quadriplegia and her sensitive level corresponded to her second cervical metamere (C2). After 3 months of treatment her motor and sensitive levels reached her first and second thoracic metameres (T1-T2). No adverse events were detected in either patient [859].

While it is known that BM mononuclear cells can act as angiogenic cells, a study examined whether combination of these cells with MSC can achieve enhanced repair of SCI. A patient with total SC interruption at the Th2-3 level underwent experimental therapy with BMNC and MSC transplantations followed with intensive neurorehabilitation treatment. At admission, 6 h after SCI, the patient was scored ASIA A, had a Th1 sensation level, paraplegia with sphincter palsy, and was without the ability to control trunk movement. Neurophysiology examination showed bilateral axonal damage to the motor and sensory neural fibers with no motor unit potentials or peripheral motor nerve conduction in the lower extremities. The standard therapy had been applied and had not produced any positive results. The patient was treated with autologous BMNCs injected intravenously (3.2×10(9)) and intrathecally (0.5×10(9)) 10 weeks after the SCI and with five rounds of MSCs every 3-4 months (1.3-3.65×10(7)) administered via lumbar puncture. Total number of transplanted MSC cells during the course of treatment was 1.54×10(8). There were no complications related to transplantations and no side effects related to the therapy during 2 years of treatment. The ASIA score improved from A to C/D (from 112 to 231 points). The sensation level expanded from Th1 to L3-4, and the patient’s ability to control the body trunk was fully restored. Bladder filling sensation, bladder control, and anal sensation were also restored. Muscle strength in the left lower extremities improved from plegia to deep paresis (1 on the Lovett scale). The patient’s ability to move lower extremities against gravity supported by the movements in quadriceps was restored. The patient gained the ability to stand in a standing frame and was able to walk with the support of hip and knee ortheses. Magnetic resonance imaging (MRI) revealed that at the Th2/Th3 level, where the hemorrhagic necrosis was initially observed, small tissue structures appeared. The results suggest that repeated intrathecal infusions of MSCs might have the potential to produce clinically meaningful improvements for SCI patients [860].

Another combination study involved administration to 6 patients a combination of autologous co-transplantation of MSC and Scwhann Cells (SC) through lumbar puncture. Neurological status of the patients was determined by ISNCSCI, as well as by assessment of functional status by Spinal Cord Independent Measure. Before and after cell transplantation, magnetic resonance imaging (MRI) was performed for all the patients. Before the procedure, all the patients underwent electromyography, urodynamic study (UDS) and MRI tractograghy. After transplantation, these assessments were performed in special cases when the patients reported any changes in motor function or any changes in urinary sensation. Over the mean 30 months of follow-up, the radiological findings were unchanged without any evidence of neoplastic tissue overgrowth. American Spinal Injury Association class in one patient was changed from A to B, in addition to the improvement in indexes of UDS, especially bladder compliance, which was congruous with axonal regeneration detected in MRI tractography. No motor score improvement was observed among the patients [861].

A case of acute penetrative SCI (gunshot wound), 40 years old, was treated with intrathecal bone marrow derived stem cells and parenteral Granulocyte-Colony Stimulating Factor (G-CSF) along with rehabilitation program. The neurological outcomes as well as safety issues have been reported. Assessment with American Spinal Injury Association (ASIA), showed neurological improvement, meanwhile he reported neuropathic pain, which was amenable to oral medication. In the acute setting, combination therapy of G-CSF and intrathecal Mesenchymal Stem Cells (MSCs) was safe in our case as an adjunct to conventional rehabilitation programs. Further controlled studies are needed to find possible side effects, and establish net efficacy [862].

### EXAMPLES

### Example 1 Generation of Umbilical Cord Potentiated MSC

Umbilical cord tissue was obtained from healthy pregnancy and washed twice with phosphate buffered saline (PBS). The Wharton’s jelly portion of the cord was extracted. Upon extraction, mechanical mincing was performed utilizing sterile forceps and a scalpel. Minced pieces were cultivated in a cell culture dish with Dulbecco’s modified Eagle’s medium F12 (DMEM)-low glucose no L-Glutamine with 10% human AB serum, 1% 10.000 U/mL penicillin and 10.000 µg/mL streptomycin. Culture was performed at initial 12 hours at 2% oxygen, 24 hours at 5% oxygen, subsequent to which CD73 positive cells were selected using magnetic activated cell sorting and expanded. The culture-expanded cells were cryopreserved at P3 using standard cryopreservation protocols until their use in the following experiment. The cells were characterized at the time of cryopreservation with flow cytometric analysis to determine the expression of positive surface markers CD90, CD105, CD73, CD44, CD29, CD56 and negative for CD34, CD45 and CD14. Additionally, quality control analyses like mycoplasma analysis (using PCR), endotoxin analysis (using the LAL test and sterility analysis) were also completed. Cells were solubilized from cryopreservation before being made ready for injection. Average cell viability for each treatment was over 95%.

### Example 2: Suppression of TNF-Alpha Production From Activated Macrophages

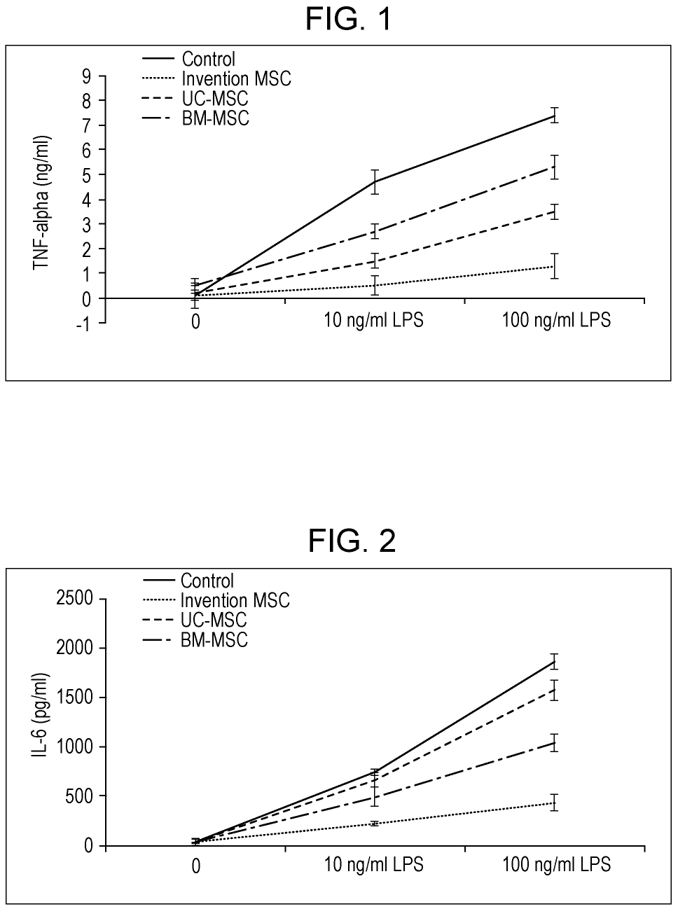

Human peripheral blood mononuclear cells (PBMC) were obtained from healthy volunteers through the use of ficoll density gradient. A total of 5 million cells per ml where plated in DMEM complete media on T75 plates for a period of 6 hours in order to allow monocytes to adhere to the plates. Non-adherent cells were washed off with PBS with 3% serum albumin. Subsequently, adherent monocytes where extracted by trypsinization and plated at a one to one ratio of cells extracted from Example 1 (Invention MSC), as well as with conventional umbilical cord MSC (UC-MSC) which were not selected for CD73 expression or bone marrow MSC (BM-MSC). Cells received stimulation with the indicated amount of endotoxin for 48 hours and quantification of TNF-alpha was performed by ELISA. Results are shown in FIG. 1.

### Example 3: Suppression of IL-6 Production From Activated Macrophages

Human peripheral blood mononuclear cells (PBMC) were obtained from healthy volunteers through the use of ficoll density gradient. A total of 5 million cells per ml where plated in DMEM complete media on T75 plates for a period of 6 hours in order to allow monocytes to adhere to the plates. Non-adherent cells were washed off with PBS with 3% serum albumin. Subsequently, adherent monocytes where extracted by trypsinization and plated at a one to one ratio of cells extracted from Example 1 (Invention MSC), as well as with conventional umbilical cord MSC (UC-MSC) which were not selected for CD73 expression or bone marrow MSC (BM-MSC). Cells received stimulation with the indicated amount of endotoxin for 48 hours and quantification of interleukin-6 was performed by ELISA. Results are shown in FIG. 2.

### Example 4: Suppression of IL-8 Production From Activated Macrophages

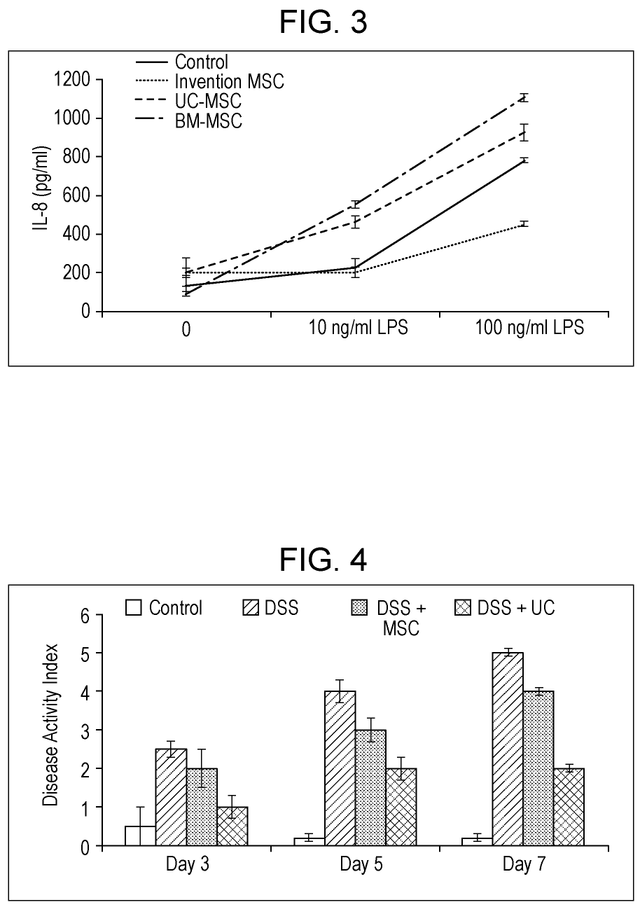

Human peripheral blood mononuclear cells (PBMC) were obtained from healthy volunteers through the use of ficoll density gradient. A total of 5 million cells per ml where plated in DMEM complete media on T75 plates for a period of 6 hours in order to allow monocytes to adhere to the plates. Non-adherent cells were washed off with PBS with 3% serum albumin. Subsequently, adherent monocytes where extracted by trypsinization and plated at a one to one ratio of cells extracted from Example 1 (Invention MSC), as well as with conventional umbilical cord MSC (UC-MSC) which were not selected for CD73 expression or bone marrow MSC (BM-MSC). Cells received stimulation with the indicated amount of endotoxin for 48 hours and quantification of interleukin-8 was performed by ELISA. Results are shown in FIG. 3.

### Example 4: Suppression of Colitis by Administration of Umbilical Cord Cells of the Invention

Female BALB/c mice at eight weeks of age were treated for 7 days with dextran sodium sulfate (DSS) at 5% in their drinking water in order to induce a murine version of inflammatory bowel disease (IBD).

Mice were divided into groups of 10 mice per group.

Group 1 saline;

Group 2 DSS;

Group 3 DSS and mesenchymal stem cells (500,000 intravenously days 1 and 3)

Group 4 DSS and umbilical cord cells of example 1 (500,000 intravenously days 1 and 3);

Disease activity index (DAI) scores were calculated according to the Hamamoto et al. Clin Exp Immunol. 1999 Sep; 117(3):462-8.This is seen below

| Score | Weight loss (%) | Stool consistency | Visible blood in feces |
|---|---|---|---|
| 0 | None | Normal | None |
| 1 | 1-5 | | |
| 2 | 6-10 | Loose | Slight bleeding |
| 3 | 11-20 | | |
| 4 | >20 | Diarrhea | Gross bleeding |

Results are shown in FIG. 4.

### Example 5: Production of Cytokines by Cells of the Invention Compared to Bone Marrow MSC and Adipose Tissue MSC

Cells (Invention cells or BM-MSC or Adipose-MSC) were utilized at passage 5. Cells were cultured in standard in fully humidified environment with 5% carbon dioxide. Cultured in 96 well plates for 48 hours, 50,000 cells per well. Unstimulated or stimulated with 50 or 100 ng/ml LPS, 10 or 20 ng/ml TNF, 10 or 20 ng/ml IL-1 beta. Cytokines assessed by EL

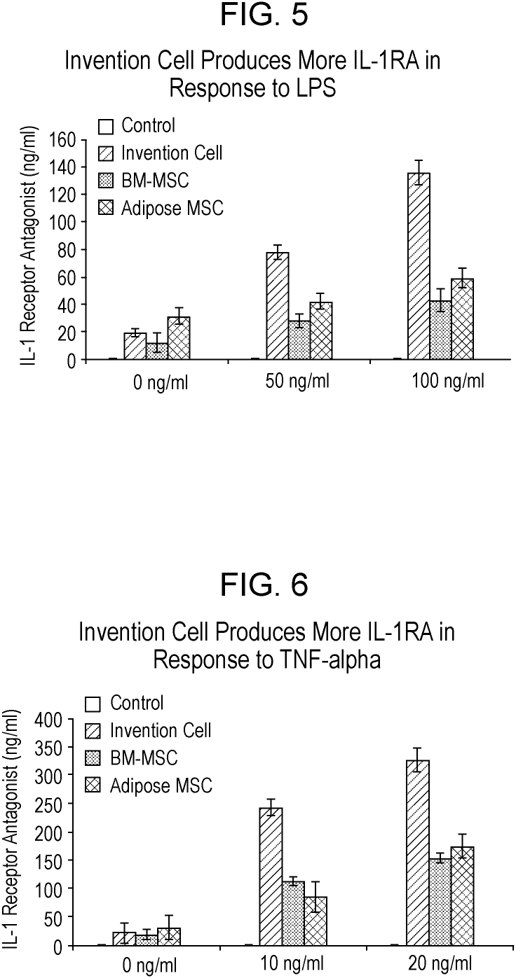

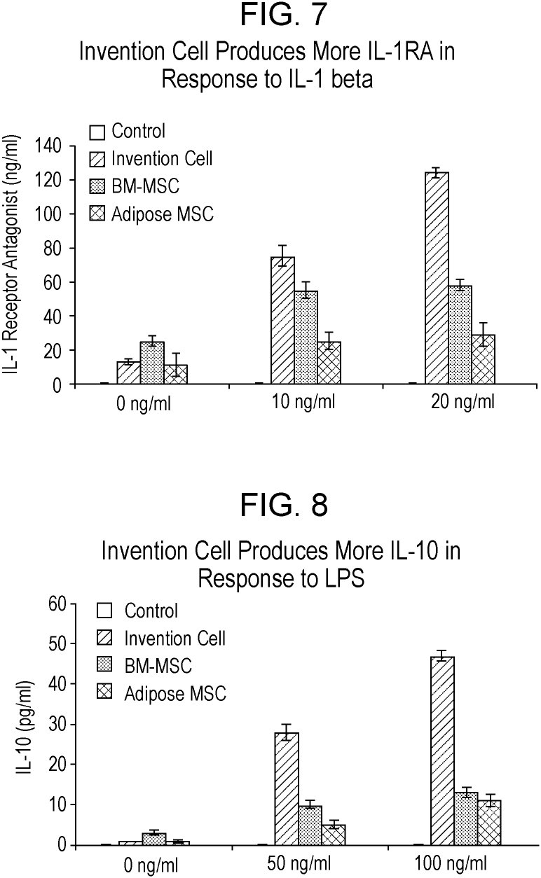

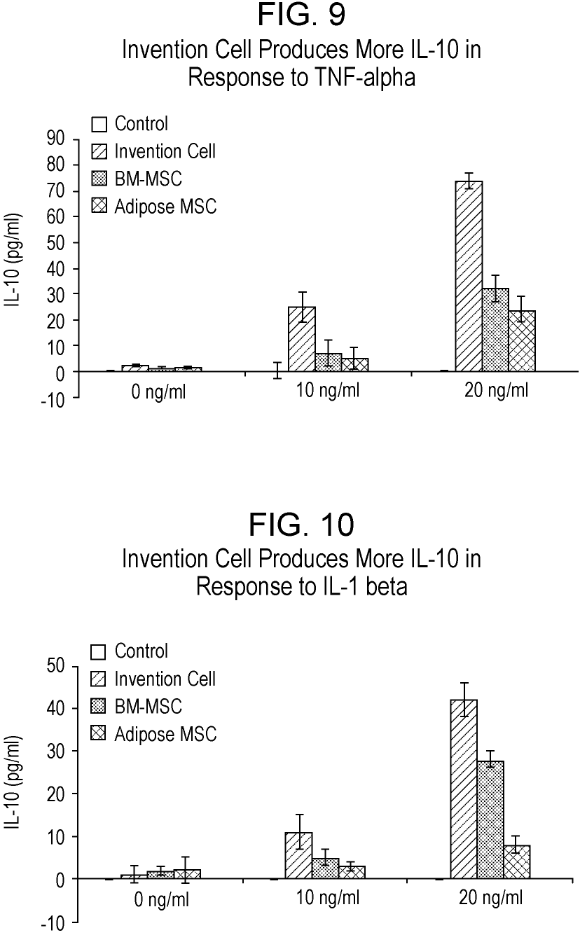

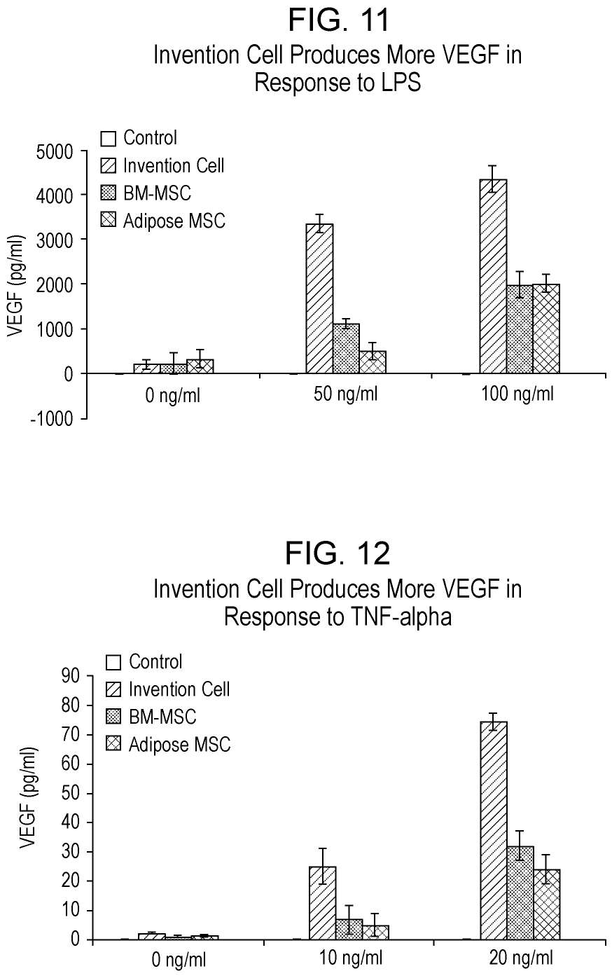

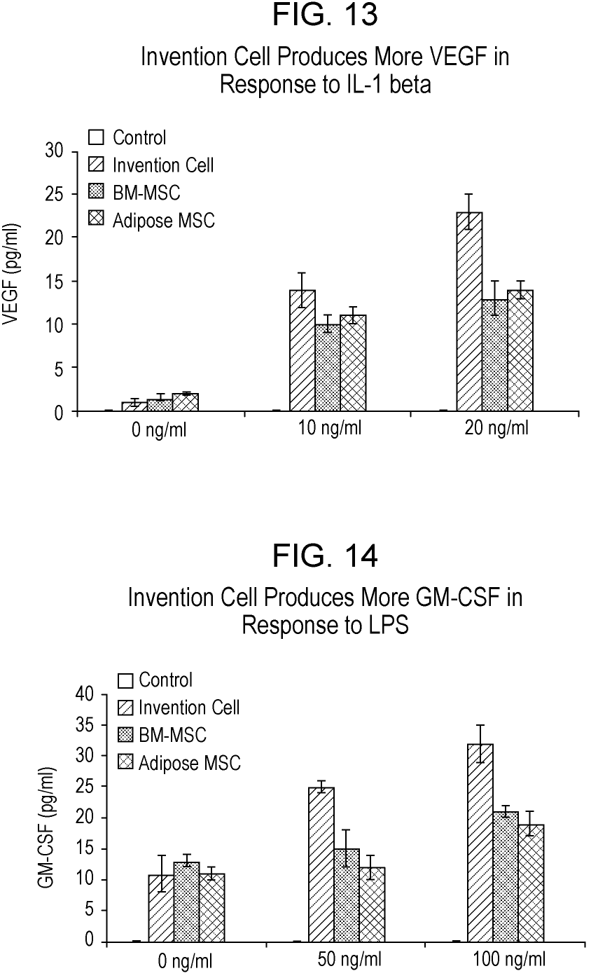

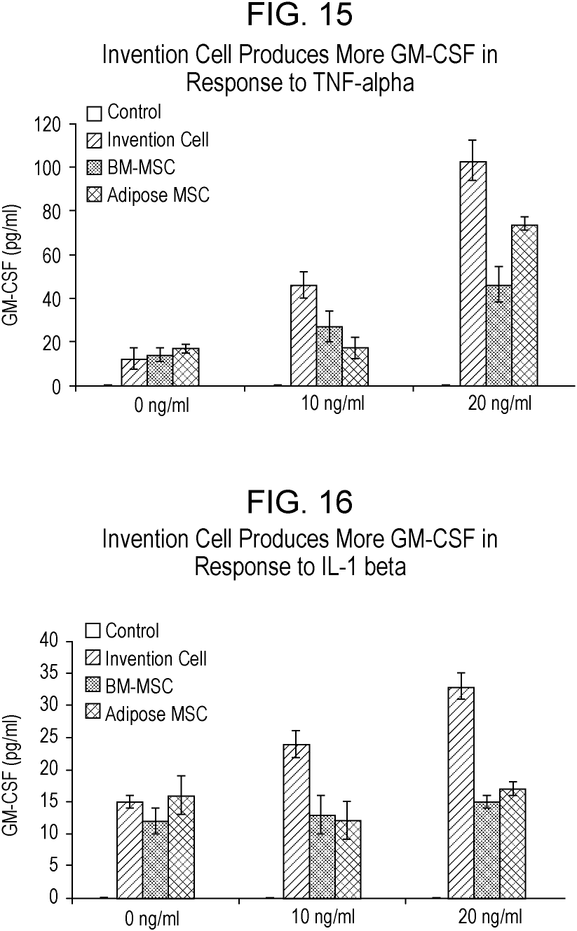

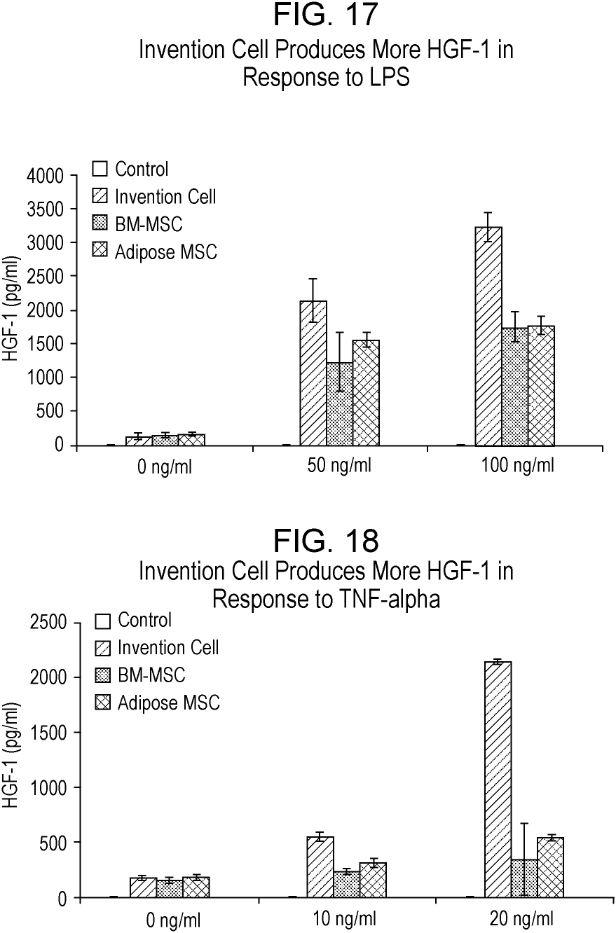

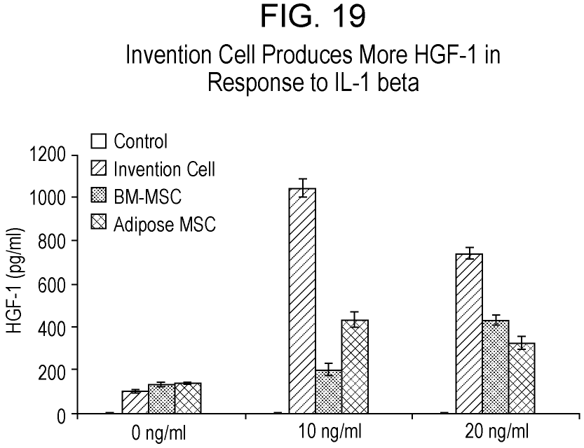

Results are shown in FIGS. 5-19.

### REFERENCES

1. Peters OM, Ghasemi M, Brown RH, Jr.: Emerging mechanisms of molecular pathology in ALS. The Journal of clinical investigation 2015, 125(5): 1767-1779.

2. Ling SC, Polymenidou M, Cleveland DW: Converging mechanisms in ALS and FTD: disrupted RNA and protein homeostasis. Neuron 2013, 79(3):416-438.

3. O′Toole O, Traynor BJ, Brennan P, Sheehan C, Frost E, Corr B, Hardiman O: Epidemiology and clinical features of amyotrophic lateral sclerosis in Ireland between 1995 and 2004. Journal of neurology, neurosurgery, and psychiatry 2008, 79(1):30-32

4. Armon C: Sports and trauma in amyotrophic lateral sclerosis revisited. Journal of the neurological sciences 2007, 262(1-2):45-53.

5. Alonso A, Logroscino G, Jick SS, Hernan MA: Incidence and lifetime risk of motor neuron disease in the United Kingdom: a population-based study. European journal of neurology 2009, 16(6):745-751.

6. Sreedharan J, Brown RH, Jr.: Amyotrophic lateral sclerosis: Problems and prospects. Annals of neurology 2013, 74(3):309-316.

7. Mazzini L, Fagioli F, Boccaletti R, Mareschi K, Oliveri G, Olivieri C, Pastore I, Marasso R, Madon E: Stem cell therapy in amyotrophic lateral sclerosis: a methodological approach in humans. Amyotrophic lateral sclerosis and other motor neuron disorders : official publication of the World Federation of Neurology, Research Group on Motor Neuron Diseases 2003, 4(3):158-161.

8. Pittenger MF, Mackay AM, Beck SC, Jaiswal RK, Douglas R, Mosca JD, Moorman MA, Simonetti DW, Craig S, Marshak DR: Multilineage potential of adult human mesenchymal stem cells. Science 1999, 284(5411):143-147.

9. Mazzini L, Mareschi K, Ferrero I, Vassallo E, Oliveri G, Boccaletti R, Testa L, Livigni S, Fagioli F: Autologous mesenchymal stem cells: clinical applications in amyotrophic lateral sclerosis. Neurological research 2006, 28(5):523-526.

10. Mazzini L, Ferrero I, Luparello V, Rustichelli D, Gunetti M, Mareschi K, Testa L, Stecco A, Tarletti R, Miglioretti M et al: Mesenchymal stem cell transplantation in amyotrophic lateral sclerosis: A Phase I clinical trial. Experimental neurology 2010, 223(1):229-237.

11. Mazzini L, Mareschi K, Ferrero I, Miglioretti M, Stecco A, Servo S, Carriero A, Monaco F, Fagioli F: Mesenchymal stromal cell transplantation in amyotrophic lateral sclerosis: a long-term safety study. Cytotherapy 2012, 14(1):56-60.

12. Oh KW, Moon C, Kim HY, Oh SI, Park J, Lee JH, Chang IY, Kim KS, Kim SH: Phase I trial of repeated intrathecal autologous bone marrow-derived mesenchymal stromal cells in amyotrophic lateral sclerosis. Stem cells translational medicine 2015, 4(6):590-597.

13. Rushkevich YN, Kosmacheva SM, Zabrodets GV, Ignatenko SI, Goncharova NV, Severin IN, Likhachev SA, Potapnev MP: The Use of Autologous Mesenchymal Stem Cells for Cell Therapy of Patients with Amyotrophic Lateral Sclerosis in Belarus. Bulletin of experimental biology and medicine 2015, 159(4):576-581.

14. Baek W, Kim YS, Koh SH, Lim SW, Kim HY, Yi HJ, Kim H: Stem cell transplantation into the intraventricular space via an Ommaya reservoir in a patient with amyotrophic lateral sclerosis. Journal of neurosurgical sciences 2012, 56(3):261-263.

15. Sundaresan N, Suite ND: Optimal use of the Ommaya reservoir in clinical oncology. Oncology (Williston Park) 1989, 3(12):15-22; discussion 23.

16. Quattrocchi KB, Miller CH, Cush S, Bernard SA, Dull ST, Smith M, Gudeman S, Varia MA: Pilot study of local autologous tumor infiltrating lymphocytes for the treatment of recurrent malignant gliomas. Journal of neuro-oncology 1999, 45(2):141-157.

17. Jung G, Brandl M, Eisner W, Fraunberger P, Reifenberger G, Schlegel U, Wiestler OD, Reulen HJ, Wilmanns W: Local immunotherapy of glioma patients with a combination of 2 bispecific antibody fragments and resting autologous lymphocytes: evidence for in situ t-cell activation and therapeutic efficacy. International journal of cancer Journal international du cancer 2001, 91(2):225-230.

18. Clemons-Miller AR, Chatta GS, Hutchins L, Angtuaco EJ, Ravaggi A, Santin AD, Cannon MJ: Intrathecal cytotoxic T-cell immunotherapy for metastatic leptomeningeal melanoma. Clinical cancer research : an official journal of the American Association for Cancer Research 2001, 7(3 Suppl):917s-924s.

19. Yamanaka R, Abe T, Yajima N, Tsuchiya N, Homma J, Kobayashi T, Narita M, Takahashi M, Tanaka R: Vaccination of recurrent glioma patients with tumour lysate-pulsed dendritic cells elicits immune responses: results of a clinical phase I/II trial. British journal of cancer 2003, 89(7):1172-1179.

20. Berry JD, Cudkowicz ME, Windebank AJ, Staff NP, Owegi M, Nicholson K, McKenna-Yasek D, Levy YS, Abramov N, Kaspi H et al: NurOwn, phase 2, randomized, clinical trial in patients with ALS: Safety, clinical, and biomarker results. Neurology 2019, 93(24):e2294-e2305.

21. Ferensztajn-Rochowiak E, Rybakowski JK: The effect of lithium on hematopoietic, mesenchymal and neural stem cells. Pharmacological reports : PR 2016, 68(2):224-230.

22. Yoneyama M, Shiba T, Hasebe S, Umeda K, Yamaguchi T, Ogita K: Lithium promotes neuronal repair and ameliorates depression-like behavior following trimethyltin-induced neuronal loss in the dentate gyrus. PloS one 2014, 9(2):e87953.

23. Boku S, Nakagawa S, Masuda T, Nishikawa H, Kato A, Takamura N, Omiya Y, Kitaichi Y, Inoue T, Kusumi I: Valproate recovers the inhibitory effect of dexamethasone on the proliferation of the adult dentate gyrus-derived neural precursor cells via GSK-3beta and beta-catenin pathway. European journal of pharmacology 2014, 723:425-430.

24. Belayev L, Khoutorova L, Zhao KL, Davidoff AW, Moore AF, Cramer SC: A novel neurotrophic therapeutic strategy for experimental stroke. Brain research 2009, 1280:117-123.

25. Boll MC, Bayliss L, Vargas-Canas S, Burgos J, Montes S, Penaloza-Solano G, Rios C, Alcaraz-Zubeldia M: Clinical and biological changes under treatment with lithium carbonate and valproic acid in sporadic amyotrophic lateral sclerosis. Journal of the neurological sciences 2014, 340(1-2):103-108.

26. Kordasti S, Marsh J, Al-Khan S, Jiang J, Smith A, Mohamedali A, Abellan PP, Veen C, Costantini B, Kulasekararaj AG et al: Functional characterization of CD4+ T cells in aplastic anemia. Blood 2012, 119(9):2033-2043.

27. de Latour RP, Visconte V, Takaku T, Wu C, Erie AJ, Sarcon AK, Desierto MJ, Scheinberg P, Keyvanfar K, Nunez O et al: Th17 immune responses contribute to the pathophysiology of aplastic anemia. Blood 2010, 116(20):4175-4184.

28. Sloand E, Kim S, Maciejewski JP, Tisdale J, Follmann D, Young NS: Intracellular interferon-gamma in circulating and marrow T cells detected by flow cytometry and the response to immunosuppressive therapy in patients with aplastic anemia. Blood 2002, 100(4):1185-1191.

29. Atta EH, Dias DS, Marra VL, de Azevedo AM: Comparison between horse and rabbit antithymocyte globulin as first-line treatment for patients with severe aplastic anemia: a single-center retrospective study.Annals of hematology 2010, 89(9):851-859.

30. Fuhrer M, Rampf U, Baumann I, Faldum A, Niemeyer C, Janka-Schaub G, Friedrich W, Ebell W, Borkhardt A, Bender-Goetze C: Immunosuppressive therapy for aplastic anemia in children: a more severe disease predicts better survival. Blood 2005, 106(6):2102-2104.

31. Lee JH, Choi SJ, Lee JH, Lee YS, Seol M, Ryu SG, Lee JS, Kim WK, Lee KH: Non-total body irradiation containing preparative regimen in alternative donor bone marrow transplantation for severe aplastic anemia. Bone marrow transplantation 2005, 35(8):755-761.

32. Gupta V, Gordon-Smith EC, Cook G, Parker A, Duguid JK, Wilson KM, Yi QL, Marsh JC: A third course of anti-thymocyte globulin in aplastic anaemia is only beneficial in previous responders. British journal of haematology 2005, 129(1):110-117.

33. Keohane EM: Acquired aplastic anemia. Clinical laboratory science : journal of the American Society for Medical Technology 2004, 17(3):165-171.

34. Bagby GC, Lipton JM, Sloand EM, Schiffer CA: Marrow failure. Hematology / the Education Program of the American Society of Hematology American Society of Hematology Education Program 2004:318-336.

35. Gerds AT, Scott BL: Last Marrow Standing: Bone Marrow Transplantation for Acquired Bone Marrow Failure Conditions. Current hematologic malignancy reports 2012.

36. Lin F: Adipose tissue-derived mesenchymal stem cells: a fat chance of curing kidney disease? Kidney international 2012, 82(7):731-733.

37. Bassi EJ, Moraes-Vieira PM, Moreira-Sa CS, Almeida DC, Vieira LM, Cunha CS, Hiyane MI, Basso AS, Pacheco-Silva A, Camara NO: Immune regulatory properties of allogeneic adipose-derived mesenchymal stem cells in the treatment of experimental autoimmune diabetes. Diabetes 2012, 61(10):2534-2545.

38. De Miguel MP, Fuentes-Julian S, Blazquez-Martinez A, Pascual CY, Aller MA, Arias J, Arnalich-Montiel F: Immunosuppressive properties of mesenchymal stem cells: advances and applications. Current molecular medicine 2012, 12(5):574-591.

39. Xiao J, Zhang C, Zhang Y, Zhang X, Zhao J, Liang J, Zhong X, Chen Y: Transplantation of adipose-derived mesenchymal stem cells into a murine model of passive chronic immune thrombocytopenia. Transfusion 2012.

40. Zhou B, Yuan J, Zhou Y, Ghawji M, Jr., Deng YP, Lee AJ, Lee AJ, Nair U, Kang AH, Brand DD et al: Administering human adipose-derived mesenchymal stem cells to prevent and treat experimental arthritis. Clinical immunology 2011, 141(3):328-337.

41. Solomou EE, Rezvani K, Mielke S, Malide D, Keyvanfar K, Visconte V, Kajigaya S, Barrett AJ, Young NS: Deficient CD4+ CD25+ FOXP3+ T regulatory cells in acquired aplastic anemia. Blood 2007, 110(5):1603-1606.

42. Yin XX, Liu CF, Li LZ, Chen WZ: [Significance of CD4+ CD25+ CD127(low) regulatory T cells and notch1 pathway in the pathogenesis of aplastic anemia]. Zhonghua xue ye xue za zhi = Zhonghua xueyexue zazhi 2008, 29(5):308-311.

43. Kelley TW, Parker CJ: CD4 (+)CD25 (+)Foxp3 (+) regulatory T cells and hematologic malignancies. Frontiers in bioscience 2010, 2:980-992.

44. Gonzalez-Rey E, Gonzalez MA, Varela N, O′Valle F, Hernandez-Cortes P, Rico L, Buscher D, Delgado M: Human adipose-derived mesenchymal stem cells reduce inflammatory and T cell responses and induce regulatory T cells in vitro in rheumatoid arthritis. Ann Rheum Dis 2010, 69(1):241-248.

45. Madec AM, Mallone R, Afonso G, Abou Mrad E, Mesnier A, Eljaafari A, Thivolet C: Mesenchymal stem cells protect NOD mice from diabetes by inducing regulatory T cells. Diabetologia 2009, 52(7):1391-1399.

46. Casiraghi F, Azzollini N, Cassis P, Imberti B, Morigi M, Cugini D, Cavinato RA, Todeschini M, Solini S, Sonzogni A et al: Pretransplant infusion of mesenchymal stem cells prolongs the survival of a semiallogeneic heart transplant through the generation of regulatory T cells. J Immunol 2008, 181(6):3933-3946.

47. Selmani Z, Naji A, Zidi I, Favier B, Gaiffe E, Obert L, Borg C, Saas P, Tiberghien P, Rouas-Freiss N et al: Human leukocyte antigen-G5 secretion by human mesenchymal stem cells is required to suppress T lymphocyte and natural killer function and to induce CD4+CD25highFOXP3+ regulatory T cells. Stem Cells 2008, 26(1):212-222.

48. Prevosto C, Zancolli M, Canevali P, Zocchi MR, Poggi A: Generation of CD4+ or CD8+ regulatory T cells upon mesenchymal stem cell-lymphocyte interaction. Haematologica 2007, 92(7):881-888.

49. Jui HY, Lin CH, Hsu WT, Liu YR, Hsu RB, Chiang BL, Tseng WY, Chen MF, Wu KK, Lee CM: Autologous mesenchymal stem cells prevent transplant arteriosclerosis by enhancing local expression of interleukin-10, interferon-gamma, and indoleamine 2,3-dioxygenase. Cell transplantation 2012, 21(5):971-984.

50. Carrancio S, Romo C, Ramos T, Lopez-Holgado N, Muntion S, Prins HJ, Martens AC, Brinon JG, Miguel JF, Canizo MC et al: Effects of MSC-co-administration and route of delivery on cord blood hematopoietic stem cell engraftment. Cell transplantation 2012.

51. Patel SA, Rameshwar P: Stem Cell Transplantation for Hematological Malignancies: Prospects for Personalized Medicine and Co-therapy with Mesenchymal Stem Cells. Current pharmacogenomics and personalized medicine 2011, 9(3):229-239.

52. Wang H, Wang Z, Xue M, Liu J, Yan H, Guo Z: Co-transfusion of haplo-identical hematopoietic and mesenchymal stromal cells to treat a patient with severe aplastic. Cytotherapy 2010, 12(4):563-565.

53. Wang H, Yan H, Wang Z, Zhu L, Liu J, Guo Z: Cotransplantation of allogeneic mesenchymal and hematopoietic stem cells in children with aplastic anemia. Pediatrics 2012, 129(6):e1612-1615.

54. Luan C, Chen R, Chen B, Ding J, Ni M: Umbilical cord blood transplantation supplemented with the infusion of mesenchymal stem cell for an adolescent patient with severe aplastic anemia: a case report and review of literature. Patient preference and adherence 2015, 9:759-765.

55. Ozdogu H, Yeral M, Boga C, Kozanoglu I: Use of mesenchymal cells to modulate immune suppression and immune reconstruction in a patient with aplastic anemia complicated by invasive sino-orbital aspergillosis. Turkish journal of haematology : official journal of Turkish Society of Haematology 2014, 31(2):181-183.

56. Fu Y, Wang Q, Zhou J, Liu S, Fang B, Wei X, Song Y: Reduced intensity conditioning and co-transplantation of unrelated peripheral stem cells combined with umbilical cord mesenchymal stem/stroma cells for young patients with refractory severe aplastic anemia. International journal of hematology 2013, 98(6):658-663.

57. Cle DV, Santana-Lemos B, Tellechea MF, Prata KL, Orellana MD, Covas DT, Calado RT: Intravenous infusion of allogeneic mesenchymal stromal cells in refractory or relapsed aplastic anemia. Cytotherapy 2015, 17(12):1696-1705.

58. Gartner S, Kaplan HS: Long-term culture of human bone marrow cells. Proc Natl Acad Sci USA 1980, 77(8):4756-4759.

59. Al-Lebban ZS, Lange RD, Jones JB, Lothrop CD: Long-term bone marrow culture systems: normal and cyclic hematopoietic dogs. Can J Vet Res 1987, 51(2):162-168.

60. Bagby GC, Jr., McCall E, Bergstrom KA, Burger D: A monokine regulates colony-stimulating activity production by vascular endothelial cells. Blood 1983, 62(3):663-668.

61. Quesenberry PJ, Gimbrone MA, Jr.: Vascular endothelium as a regulator of granulopoiesis: production of colony-stimulating activity by cultured human endothelial cells. Blood 1980, 56(6):1060-1067.

62. Gerson SL, Friedman HM, Cines DB: Viral infection of vascular endothelial cells alters production of colony-stimulating activity. J Clin Invest 1985, 76(4):1382-1390.

63. Tavassoli M: Structure and function of sinusoidal endothelium of bone marrow. Prog Clin Biol Res 1981, 59B:249-256.

64. Soda R, Tavassoli M: Mapping of the bone marrow sinus endothelium with lectins and glycosylated ferritins: identification of differentiated microdomains and their functional significance. J Ultrastruct Res 1983, 84(3):299-310.

65. Soda R, Tavassoli M: Modulation of WGA binding sites on marrow sinus endothelium in state of stimulated erythropoiesis: a possible mechanism regulating the rate of cell egress. J Ultrastruct Res 1984, 87(3):242-251.

66. Irie S, Tavassoli M: Structural features of isolated, fractionated bone marrow endothelium compared to sinus endothelium in situ. Scan Electron Microsc 1986(Pt 2):615-619.

67. Knudtzon S, Mortensen BT: Growth stimulation of human bone marrow cells in agar culture by vascular cells. Blood 1975, 46(6):937-943.

68. Munker R, Gasson J, Ogawa M, Koeffler HP: Recombinant human TNF induces production of granulocyte-monocyte colony-stimulating factor. Nature 1986, 323(6083):79-82.

69. Sieff CA, Tsai S, Faller DV: Interleukin 1 induces cultured human endothelial cell production of granulocyte-macrophage colony-stimulating factor. J Clin Invest 1987, 79(1):48-51.

70. Ascensao JL, Vercellotti GM, Jacob HS, Zanjani ED: Role of endothelial cells in human hematopoiesis: modulation of mixed colony growth in vitro. Blood 1984, 63(3):553-558.

71. Sieff CA, Niemeyer CM, Faller DV: The production of hematopoietic growth factors by endothelial accessory cells. Blood Cells 1987, 13(1-2):65-74.

72. Malone DG, Pierce JH, Falko JP, Metcalfe DD: Production of granulocyte-macrophage colony-stimulating factor by primary cultures of unstimulated rat microvascular endothelial cells. Blood 1988, 71(3):684-689.

73. Surugiu R, Olaru A, Hermann DM, Glavan D, Catalin B, Popa-Wagner A: Recent Advances in Mono- and Combined Stem Cell Therapies of Stroke in Animal Models and Humans. Int J Mol Sci 2019, 20(23).

74. Yvernogeau L, Gautier R, Petit L, Khoury H, Relaix F, Ribes V, Sang H, Charbord P, Souyri M, Robin C et al: In vivo generation of haematopoietic stem/progenitor cells from bone marrow-derived haemogenic endothelium. Nat Cell Biol 2019, 21(11):1334-1345.

75. Molbay M, Kipmen-Korgun D, Korkmaz G, Ozekinci M, Turkay Korgun E: Human Trophoblast Progenitor Cells Express and Release Angiogenic Factors. Int J Mol Cell Med 2018, 7(4):203-211.

76. Clark DA: Shall we properly re-examine the status of allogeneic lymphocyte therapy for recurrent early pregnancy failure? Am J Reprod Immunol 2004, 51(1):7-15.

77. Wang Y, Underwood J, Vaughan R, Harmer A, Doyle C, Lehner T: Allo-immunization elicits CCR5 antibodies, SDF-1 chemokines, and CD8-suppressor factors that inhibit transmission of R5 and X4 HIV-1 in women. Clin Exp Immunol 2002, 129(3):493-501.

78. Okaji Y, Tsuno NH, Tanaka M, Yoneyama S, Matsuhashi M, Kitayama J, Saito S, Nagura Y, Tsuchiya T, Yamada J et al: Pilot study of anti-angiogenic vaccine using fixed whole endothelium in patients with progressive malignancy after failure of conventional therapy.Eur J Cancer 2008, 44(3):383-390.

79. Zhuo Y, Fang F, Lu L, Li T, Lian J, Xiong Y, Kong D, Li K: White matter impairment in type 2 diabetes mellitus with and without microvascular disease. Neuroimage Clin2019, 24:101945.

80. Mendez MF: The neuropsychiatric aspects of boxing. Int J Psychiatry Med 1995, 25(3):249-262.

81. Jordan BD: Neurologic aspects of boxing. Arch Neurol 1987, 44(4):453-459.

82. McKee AC, Cantu RC, Nowinski CJ, Hedley-Whyte ET, Gavett BE, Budson AE, Santini VE, Lee HS, Kubilus CA, Stern RA: Chronic traumatic encephalopathy in athletes: progressive tauopathy after repetitive head injury. J Neuropathol Exp Neurol 2009, 68(7):709-735.

83. Gavett BE, Cantu RC, Shenton M, Lin AP, Nowinski CJ, McKee AC, Stern RA: Clinical appraisal of chronic traumatic encephalopathy: current perspectives and future directions. Curr Opin Neurol 2011, 24(6):525-531.

84. Stem RA, Riley DO, Daneshvar DH, Nowinski CJ, Cantu RC, McKee AC: Long-term consequences of repetitive brain trauma: chronic traumatic encephalopathy. PM R 2011, 3(10 Suppl 2):S460-467.

85. McKee AC, Stein TD, Kiernan PT, Alvarez VE: The neuropathology of chronic traumatic encephalopathy. Brain Pathol 2015, 25(3):350-364.

86. McKee AC, Cairns NJ, Dickson DW, Folkerth RD, Keene CD, Litvan I, Perl DP, Stein TD, Vonsattel JP, Stewart W et al: The first NINDS/NIBIB consensus meeting to define neuropathological criteria for the diagnosis of chronic traumatic encephalopathy. Acta Neuropathol 2016, 131(1):75-86.

87. 91:1103-07. MHPdJ: Punch Drunk. JAMA 1928, 91:1103.

88. Adams CW, Bruton CJ: The cerebral vasculature in dementia pugilistica. J Neurol Neurosurg Psychiatry 1989, 52(5):600-604.

89. Critchley M: Medical aspects of boxing, particularly from a neurological standpoint. Br Med J 1957, 1(5015):357-362.

90. Fesharaki-Zadeh A: Chronic Traumatic Encephalopathy: A Brief Overview. Front Neurol 2019, 10:713.

91. Omalu BI, DeKosky ST, Minster RL, Kamboh MI, Hamilton RL, Wecht CH: Chronic traumatic encephalopathy in a National Football League player. Neurosurgery 2005, 57(1):128-134; discussion 128-134.

92. Gandy S, Ikonomovic MD, Mitsis E, Elder G, Ahlers ST, Barth J, Stone JR, DeKosky ST: Chronic traumatic encephalopathy: clinical-biomarker correlations and current concepts in pathogenesis. Mol Neurodegener 2014, 9:37.

93. Blennow K, Hardy J, Zetterberg H: The neuropathology and neurobiology of traumatic brain injury. Neuron 2012, 76(5):886-899.

94. Mullally WJ: Concussion. Am J Med 2017, 130(8):885-892.

95. Luerssen TG, Klauber MR, Marshall LF: Outcome from head injury related to patient’s age. A longitudinal prospective study of adult and pediatric head injury. J Neurosurg 1988, 68(3):409-416.

96. Crisco JJ, Fiore R, Beckwith JG, Chu JJ, Brolinson PG, Duma S, McAllister TW, Duhaime AC, Greenwald RM: Frequency and location of head impact exposures in individual collegiate football players. J Athl Train 2010, 45(6):549-559.

97. Hoge CW, McGurk D, Thomas JL, Cox AL, Engel CC, Castro CA: Mild traumatic brain injury in U.S. Soldiers returning from Iraq. N Engl J Med 2008, 358(5):453-463.

98. Theeler BJ, Flynn FG, Erickson JC: Headaches after concussion in US soldiers returning from Iraq or Afghanistan. Headache 2010, 50(8):1262-1272.

99. MacGregor AJ, Dougherty AL, Morrison RH, Quinn KH, Galarneau MR: Repeated concussion among U.S. military personnel during Operation Iraqi Freedom. J Rehabil Res Dev 2011, 48(10):1269-1278.

100. McCrory P, Zazryn T, Cameron P: The evidence for chronic traumatic encephalopathy in boxing. Sports Med 2007, 37(6):467-476.

101. Bouziane C, Filatova OG, Schrantee A, Caan MWA, Vos FM, Reneman L: White Matter by Diffusion MRI Following Methylphenidate Treatment: A Randomized Control Trial in Males with Attention-Deficit/Hyperactivity Disorder. Radiology 2019:182528.

102. Kochunov P, Huang J, Chen S, Li Y, Tan S, Fan F, Feng W, Wang Y, Rowland LM, Savransky A et al: White Matter in Schizophrenia Treatment Resistance. Am J Psychiatry 2019:appiajp201918101212.

103. Herweh C, Hess K, Meyding-Lamade U, Bartsch AJ, Stippich C, Jost J, Friedmann-Bette B, Heiland S, Bendszus M, Hahnel S: Reduced white matter integrity in amateur boxers. Neuroradiology 2016, 58(9):911-920.

104. Foster JB, Leiguarda R, Tilley PJ: Brain damage in National Hunt jockeys. Lancet 1976, 1(7967):981-983.

105. McCrory P, Turner M, Murray J: A punch drunk jockey? Br J Sports Med 2004, 38(3):e3.

106. Berstad JR, Baerum B, Lochen EA, Mogstad TE, Sjaastad O: Whiplash: chronic organic brain syndrome without hydrocephalus ex vacuo. Acta Neurol Scand 1975, 51(4):268-284.

107. Squier W: Shaken baby syndrome: the quest for evidence. Dev Med Child Neurol 2008, 50(1):10-14.

108. Omalu BI, Fitzsimmons RP, Hammers J, Bailes J: Chronic traumatic encephalopathy in a professional American wrestler. J Forensic Nurs 2010, 6(3):130-136.

109. Omalu B, Hammers JL, Bailes J, Hamilton RL, Kamboh MI, Webster G, Fitzsimmons RP: Chronic traumatic encephalopathy in an Iraqi war veteran with posttraumatic stress disorder who committed suicide. Neurosurg Focus 2011, 31(5):E3.

110. Hasoon J: Blast-associated traumatic brain injury in the military as a potential trigger for dementia and chronic traumatic encephalopathy. US Army Med Dep J 2017(1-17): 102-105.

111. Omalu BI, DeKosky ST, Hamilton RL, Minster RL, Kamboh MI, Shakir AM, Wecht CH: Chronic traumatic encephalopathy in a national football league player: part II. Neurosurgery 2006, 59(5):1086-1092; discussion 1092-1083.

112. Breedlove EL, Robinson M, Talavage TM, Morigaki KE, Yoruk U, O′Keefe K, King J, Leverenz LJ, Gilger JW, Nauman EA: Biomechanical correlates of symptomatic and asymptomatic neurophysiological impairment in high school football. J Biomech 2012, 45(7): 1265-1272.

113. Maroon JC, Winkelman R, Bost J, Amos A, Mathyssek C, Miele V: Chronic traumatic encephalopathy in contact sports: a systematic review of all reported pathological cases. PLoS One 2015, 10(2):e0117338.

114. Bieniek KF, Blessing MM, Heckman MG, Diehl NN, Serie AM, Paolini MA, 2nd, Boeve BF, Savica R, Reichard RR, Dickson DW: Association between contact sports participation and chronic traumatic encephalopathy: a retrospective cohort study. Brain Pathol 2019.

115. McMillan TM, McSkimming P, Wainman-Lefley J, Maclean LM, Hay J, McConnachie A, Stewart W: Long-term health outcomes after exposure to repeated concussion in elite level: rugby union players. J Neurol Neurosurg Psychiatry 2017, 88(6):505-511.

116. Ling H, Morris HR, Neal JW, Lees AJ, Hardy J, Holton JL, Revesz T, Williams DD: Mixed pathologies including chronic traumatic encephalopathy account for dementia in retired association football (soccer) players. Acta Neuropathol 2017, 133(3):337-352.

117. Nitrini R: Soccer (Football Association) and chronic traumatic encephalopathy: A short review and recommendation. Dement Neuropsychol 2017, 11(3):218-220.

118. Siegler A, Rosner Z, MacDonald R, Ford E, Venters H: Head Trauma in Jail and Implications for Chronic Traumatic Encephalopathy in the United States: Case Report and Results of Injury Surveillance in NYC Jails. J Health Care Poor Underserved 2017, 28(3):1042-1049.

119. Tribett T, Erskine B, Bailey K, Brown T, Castellani RJ: Chronic Traumatic Encephalopathy Pathology After Shotgun Injury to the Brain. J Forensic Sci 2019, 64(4): 1248-1252.

120. Lim LJH, Ho RCM, Ho CSH: Dangers of Mixed Martial Arts in the Development of Chronic Traumatic Encephalopathy. Int J Environ Res Public Health 2019, 16(2).

121. Mez J, Daneshvar DH, Kiernan PT, Abdolmohammadi B, Alvarez VE, Huber BR, Alosco ML, Solomon TM, Nowinski CJ, McHale L et al: Clinicopathological Evaluation of Chronic Traumatic Encephalopathy in Players of American Football. JAMA 2017, 318(4):360-370.

122. Alosco ML, Mez J, Tripodis Y, Kiernan PT, Abdolmohammadi B, Murphy L, Kowall NW, Stein TD, Huber BR, Goldstein LE et al: Age of first exposure to tackle football and chronic traumatic encephalopathy. Ann Neurol 2018, 83(5):886-901.

123. Liu MC, Kobeissy F, Zheng W, Zhang Z, Hayes RL, Wang KK: Dual vulnerability of tau to calpains and caspase-3 proteolysis under neurotoxic and neurodegenerative conditions. ASN Neuro 2011, 3(1):e00051.

124. Johnson VE, Stewart W, Smith DH: Widespread tau and amyloid-beta pathology many years after a single traumatic brain injury in humans. Brain Pathol 2012, 22(2):142-149.

125. McKee AC, Stem RA, Nowinski CJ, Stein TD, Alvarez VE, Daneshvar DH, Lee HS, Wojtowicz SM, Hall G, Baugh CM et al: The spectrum of disease in chronic traumatic encephalopathy. Brain 2013, 136(Pt 1):43-64.

126. Yi J, Padalino DJ, Chin LS, Montenegro P, Cantu RC: Chronic traumatic encephalopathy. Curr Sports Med Rep 2013, 12(1):28-32.

127. Olivera A, Lejbman N, Jeromin A, French LM, Kim HS, Cashion A, Mysliwiec V, Diaz-Arrastia R, Gill J: Peripheral Total Tau in Military Personnel Who Sustain Traumatic Brain Injuries During Deployment. JAMA Neurol 2015, 72(10):1109-1116.

128. Simic G, Babic Leko M, Wray S, Harrington C, Delalle I, Jovanov-Milosevic N, Bazadona D, Buee L, de Silva R, Di Giovanni G et al: Tau Protein Hyperphosphorylation and Aggregation in Alzheimer’s Disease and Other Tauopathies, and Possible Neuroprotective Strategies. Biomolecules 2016, 6(1):6.

129. Grundke-Iqbal I, Iqbal K, Tung YC, Quinlan M, Wisniewski HM, Binder LI: Abnormal phosphorylation of the microtubule-associated protein tau (tau) in Alzheimer cytoskeletal pathology. Proc Natl Acad Sci U S A 1986, 83(13):4913-4917.

130. Binder LI, Frankfurter A, Rebhun LI: The distribution of tau in the mammalian central nervous system. J Cell Biol 1985, 101(4):1371-1378.

131. Hirokawa N, Funakoshi T, Sato-Harada R, Kanai Y: Selective stabilization of tau in axons and microtubule-associated protein 2C in cell bodies and dendrites contributes to polarized localization of cytoskeletal proteins in mature neurons. J Cell Biol1996, 132(4):667-679.

132. Ittner LM, Ke YD, Delerue F, Bi M, Gladbach A, van Eersel J, Wolfing H, Chieng BC, Christie MJ, Napier IA et al: Dendritic function of tau mediates amyloid-beta toxicity in Alzheimer’s disease mouse models. Cell 2010, 142(3):387-397.

133. Jameson L, Frey T, Zeeberg B, Dalldorf F, Caplow M: Inhibition of microtubule assembly by phosphorylation of microtubule-associated proteins. Biochemistry 1980, 19(11):2472-2479.

134. Rodriguez-Martin T, Cuchillo-Ibanez I, Noble W, Nyenya F, Anderton BH, Hanger DP: Tau phosphorylation affects its axonal transport and degradation. Neurobiol Aging 2013, 34(9):2146-2157.

135. Glushakova OY, Glushakov AO, Borlongan CV, Valadka AB, Hayes RL, Glushakov AV: Role of Caspase-3-Mediated Apoptosis in Chronic Caspase-3-Cleaved Tau Accumulation and Blood-Brain Barrier Damage in the Corpus Callosum after Traumatic Brain Injury in Rats. J Neurotrauma 2018, 35(1):157-173.

136. Schneider L, Reichert E, Faulkner J, Reichert B, Sonnen J, Hawryluk GWJ: CNS inflammation and neurodegeneration: sequelae of peripheral inoculation with spinal cord tissue in rat. J Neurosurg 2019:1-12.

137. Arun P, Oguntayo S, Albert SV, Gist I, Wang Y, Nambiar MP, Long JB: Acute decrease in alkaline phosphatase after brain injury: A potential mechanism for tauopathy. Neurosci Lett 2015, 609:152-158.

138. Woerman AL, Aoyagi A, Patel S, Kazmi SA, Lobach I, Grinberg LT, McKee AC, Seeley WW, Olson SH, Prusiner SB: Tau prions from Alzheimer’s disease and chronic traumatic encephalopathy patients propagate in cultured cells. Proc Natl Acad Sci U S A 2016, 113(50):E8187-E8196.

139. Edwards G, 3rd, Zhao J, Dash PK, Soto C, Moreno-Gonzalez I: Traumatic Brain Injury Induces Tau Aggregation and Spreading. J Neurotrauma 2019.

140. Zanier ER, Bertani I, Sammali E, Pischiutta F, Chiaravalloti MA, Vegliante G, Masone A, Corbelli A, Smith DH, Menon DK et al: Induction of a transmissible tau pathology by traumatic brain injury. Brain 2018, 141(9):2685-2699.

141. Kriegel J, Papadopoulos Z, McKee AC: Chronic Traumatic Encephalopathy: Is Latency in Symptom Onset Explained by Tau Propagation? Cold Spring Harb Perspect Med 2018, 8(2).

142. Kondo A, Shahpasand K, Mannix R, Qiu J, Moncaster J, Chen CH, Yao Y, Lin YM, Driver JA, Sun Y et al: Antibody against early driver of neurodegeneration cis P-tau blocks brain injury and tauopathy.Nature 2015, 523(7561):431-436.

143. Smith C, Gentleman SM, Leclercq PD, Murray LS, Griffin WS, Graham DI, Nicoll JA: The neuroinflammatory response in humans after traumatic brain injury. Neuropathol Appl Neurobiol 2013, 39(6):654-666.

144. Cherry JD, Tripodis Y, Alvarez VE, Huber B, Kiernan PT, Daneshvar DH, Mez J, Montenigro PH, Solomon TM, Alosco ML et al: Microglial neuroinflammation contributes to tau accumulation in chronic traumatic encephalopathy. Acta Neuropathol Commun 2016, 4(1):112.

145. Alberati-Giani D, Ricciardi-Castagnoli P, Kohler C, Cesura AM: Regulation of the kynurenine metabolic pathway by interferon-gamma in murine cloned macrophages and microglial cells. J Neurochem 1996, 66(3):996-1004.

146. Alberati-Giani D, Cesura AM: Expression of the kynurenine enzymes in macrophages and microglial cells: regulation by immune modulators. Amino Acids 1998, 14(1-3):251-255.

147. Guillemin GJ, Brew BJ, Noonan CE, Takikawa O, Cullen KM: Indoleamine 2,3 dioxygenase and quinolinic acid immunoreactivity in Alzheimer’s disease hippocampus. Neuropathol Appl Neurobiol 2005, 31(4):395-404.

148. O′Farrell K, Fagan E, Connor TJ, Harkin A: Inhibition of the kynurenine pathway protects against reactive microglial-associated reductions in the complexity of primary cortical neurons. Eur J Pharmacol 2017, 810:163-173.

149. Garrison AM, Parrott JM, Tunon A, Delgado J, Redus L, O′Connor JC: Kynurenine pathway metabolic balance influences microglia activity: Targeting kynurenine monooxygenase to dampen neuroinflammation. Psychoneuroendocrinology 2018, 94:1-10.

150. Singh R, Savitz J, Teague TK, Polanski DW, Mayer AR, Bellgowan PS, Meier TB: Mood symptoms correlate with kynurenine pathway metabolites following sports-related concussion. J Neurol Neurosurg Psychiatry 2016, 87(6):670-675.

151. Coughlin JM, Wang Y, Munro CA, Ma S, Yue C, Chen S, Airan R, Kim PK, Adams AV, Garcia C et al: Neuroinflammation and brain atrophy in former NFL players: An in vivo multimodal imaging pilot study. Neurobiol Dis 2015, 74:58-65.

152. Wang S, Cheng H, Dai G, Wang X, Hua R, Liu X, Wang P, Chen G, Yue W, An Y: Umbilical cord mesenchymal stem cell transplantation significantly improves neurological function in patients with sequelae of traumatic brain injury. Brain research 2013, 1532:76-84.

153. Tian C, Wang X, Wang X, Wang L, Wang X, Wu S, Wan Z: Autologous bone marrow mesenchymal stem cell therapy in the subacute stage of traumatic brain injury by lumbar puncture. Experimental and clinical transplantation : official journal of the Middle East Society for Organ Transplantation 2013, 11(2):176-181.

154. Thaisetthawatkul P: Pure Autonomic Failure. Current neurology and neuroscience reports 2016, 16(8):74.

155. Ozawa T, Healy DG, Abou-Sleiman PM, Ahmadi KR, Quinn N, Lees AJ, Shaw K, Wullner U, Berciano J, Moller JC et al: The alpha-synuclein gene in multiple system atrophy. Journal of neurology, neurosurgery, and psychiatry 2006, 77(4):464-467.

156. Yoshida M: Multiple system atrophy: alpha-synuclein and neuronal degeneration. Neuropathology : official journal of the Japanese Society of Neuropathology 2007, 27(5):484-493.

157. Jellinger KA, Seppi K, Wenning GK: Grading of neuropathology in multiple system atrophy: proposal for a novel scale. Movement disorders : official journal of the Movement Disorder Society 2005, 20 Suppl 12:S29-36.

158. Jellinger KA: Neuropathology of multiple system atrophy: new thoughts about pathogenesis. Movement disorders : official journal of the Movement Disorder Society 2014, 29(14):1720-1741.

159. Lee PH, Lee JE, Kim HS, Song SK, Lee HS, Nam HS, Cheong JW, Jeong Y, Park HJ, Kim DJ et al: A randomized trial of mesenchymal stem cells in multiple system atrophy. Annals of neurology 2012, 72(1):32-40.

160. Lee PH, Kim JW, Bang OY, Ahn YH, Joo IS, Huh K: Autologous mesenchymal stem cell therapy delays the progression of neurological deficits in patients with multiple system atrophy. Clinical pharmacology and therapeutics 2008, 83(5):723-730.

161. Dongmei H, Jing L, Mei X, Ling Z, Hongmin Y, Zhidong W, Li D, Zikuan G, Hengxiang W: Clinical analysis of the treatment of spinocerebellar ataxia and multiple system atrophy-cerebellar type with umbilical cord mesenchymal stromal cells. Cytotherapy 2011, 13(8):913-917.

162. Xi H, Chen L, Huang H, Zhang F, Liu Y, Chen D, Xiao J: Preliminary report of multiple cell therapy for patients with multiple system atrophy. Cell transplantation 2013, 22 Suppl 1:S93-99.

163. Canesi M, Giordano R, Lazzari L, Isalberti M, Isaias IU, Benti R, Rampini P, Marotta G, Colombo A, Cereda E et al: Finding a new therapeutic approach for no-option Parkinsonisms: mesenchymal stromal cells for progressive supranuclear palsy. Journal of translational medicine 2016, 14(1):127.

164. Venkataramana NK, Kumar SK, Balaraju S, Radhakrishnan RC, Bansal A, Dixit A, Rao DK, Das M, Jan M, Gupta PK et al: Open-labeled study of unilateral autologous bone-marrow-derived mesenchymal stem cell transplantation in Parkinson’s disease. Translational research : the journal of laboratory and clinical medicine 2010, 155(2):62-70.

165. Duijvestein M, Vos AC, Roelofs H, Wildenberg ME, Wendrich BB, Verspaget HW, Kooy-Winkelaar EM, Koning F, Zwaginga JJ, Fidder HH et al: Autologous bone marrow-derived mesenchymal stromal cell treatment for refractory luminal Crohn’s disease: results of a phase I study. Gut 2010, 59(12):1662-1669.

166. Taddio A, Tommasini A, Valencic E, Biagi E, Decorti G, De Iudicibus S, Cuzzoni E, Gaipa G, Badolato R, Prandini A et al: Failure of interferon-gamma pre-treated mesenchymal stem cell treatment in a patient with Crohn’s disease. World journal of gastroenterology 2015, 21(14):4379-4384.

167. Dhere T, Copland I, Garcia M, Chiang KY, Chinnadurai R, Prasad M, Galipeau J, Kugathasan S: The safety of autologous and metabolically fit bone marrow mesenchymal stromal cells in medically refractory Crohn’s disease - a phase 1 trial with three doses. Alimentary pharmacology & therapeutics 2016, 44(5):471-481.

168. Garcia-Olmo D, Garcia-Arranz M, Herreros D, Pascual I, Peiro C, Rodriguez-Montes JA: A phase I clinical trial of the treatment of Crohn’s fistula by adipose mesenchymal stem cell transplantation. Diseases of the colon and rectum 2005, 48(7):1416-1423.

169. Herreros MD, Garcia-Arranz M, Guadalajara H, De-La-Quintana P, Garcia-Olmo D, Group FC: Autologous expanded adipose-derived stem cells for the treatment of complex cryptoglandular perianal fistulas: a phase III randomized clinical trial (FATT 1: fistula Advanced Therapy Trial 1) and long-term evaluation. Diseases of the colon and rectum 2012, 55(7):762-772.

170. Ciccocioppo R, Bernardo ME, Sgarella A, Maccario R, Avanzini MA, Ubezio C, Minelli A, Alvisi C, Vanoli A, Calliada F et al: Autologous bone marrow-derived mesenchymal stromal cells in the treatment of fistulising Crohn’s disease. Gut 2011, 60(6):788-798.

171. Ciccocioppo R, Gallia A, Sgarella A, Kruzliak P, Gobbi PG, Corazza GR: Long-Term Follow-Up of Crohn Disease Fistulas After Local Injections of Bone Marrow-Derived Mesenchymal Stem Cells. Mayo Clinic proceedings 2015, 90(6):747-755.

172. de la Portilla F, Alba F, Garcia-Olmo D, Herrerias JM, Gonzalez FX, Galindo A: Expanded allogeneic adipose-derived stem cells (eASCs) for the treatment of complex perianal fistula in Crohn’s disease: results from a multicenter phase I/IIa clinical trial. International journal of colorectal disease 2013, 28(3):313-323.

173. Panes J, Garcia-Olmo D, Van Assche G, Colombel JF, Reinisch W, Baumgart DC, Dignass A, Nachury M, Ferrante M, Kazemi-Shirazi L et al: Expanded allogeneic adipose-derived mesenchymal stem cells (Cx601) for complex perianal fistulas in Crohn’s disease: a phase 3 randomised, double-blind controlled trial. Lancet 2016, 388(10051):1281-1290.

174. Molendijk I, Bonsing BA, Roelofs H, Peeters KC, Wasser MN, Dijkstra G, van der Woude CJ, Duijvestein M, Veenendaal RA, Zwaginga JJ et al: Allogeneic Bone Marrow-Derived Mesenchymal Stromal Cells Promote Healing of Refractory Perianal Fistulas in Patients With Crohn’s Disease. Gastroenterology 2015, 149(4):918-927 e916.

175. Le Blanc K, Rasmusson I, Sundberg B, Gotherstrom C, Hassan M, Uzunel M, Ringden O: Treatment of severe acute graft-versus-host disease with third party haploidentical mesenchymal stem cells. Lancet 2004, 363(9419): 1439-1441.

176. Ringden O, Uzunel M, Rasmusson I, Remberger M, Sundberg B, Lonnies H, Marschall HU, Dlugosz A, Szakos A, Hassan Z et al: Mesenchymal stem cells for treatment of therapy-resistant graft-versus-host disease. Transplantation 2006, 81(10):1390-1397.

177. Le Blanc K, Frassoni F, Ball L, Locatelli F, Roelofs H, Lewis I, Lanino E, Sundberg B, Bernardo ME, Remberger M et al: Mesenchymal stem cells for treatment of steroid-resistant, severe, acute graft-versus-host disease: a phase II study. Lancet 2008, 371(9624):1579-1586.

178. Lim JH, Lee MH, Yi HG, Kim CS, Kim JH, Song SU: Mesenchymal stromal cells for steroid-refractory acute graft-versus-host disease: a report of two cases. International journal of hematology 2010, 92(1):204-207.

179. Herrmann R, Sturm M, Shaw K, Purtill D, Cooney J, Wright M, Phillips M, Cannell P: Mesenchymal stromal cell therapy for steroid-refractory acute and chronic graft versus host disease: a phase 1 study. International journal of hematology 2012, 95(2): 182-188.

180. Muroi K, Miyamura K, Okada M, Yamashita T, Murata M, Ishikawa T, Uike N, Hidaka M, Kobayashi R, Imamura M et al: Bone marrow-derived mesenchymal stem cells (JR-031) for steroid-refractory grade III or IV acute graft-versus-host disease: a phase II/III study. International journal of hematology 2016, 103(2):243-250.

181. Kuci Z, Bonig H, Kreyenberg H, Bunos M, Jauch A, Janssen JW, Skific M, Michel K, Eising B, Lucchini G et al: Mesenchymal stromal cells from pooled mononuclear cells of multiple bone marrow donors as rescue therapy in pediatric severe steroid-refractory graft-versus-host disease: a multicenter survey. Haematologica 2016, 101(8):985-994.

182. von Bonin M, Stolzel F, Goedecke A, Richter K, Wuschek N, Holig K, Platzbecker U, Illmer T, Schaich M, Schetelig J et al: Treatment of refractory acute GVHD with third-party MSC expanded in platelet lysate-containing medium. Bone marrow transplantation 2009, 43(3):245-251.

183. Lucchini G, Introna M, Dander E, Rovelli A, Balduzzi A, Bonanomi S, Salvade A, Capelli C, Belotti D, Gaipa G et al: Platelet-lysate-expanded mesenchymal stromal cells as a salvage therapy for severe resistant graft-versus-host disease in a pediatric population. Biology of blood and marrow transplantation : journal of the American Society for Blood and Marrow Transplantation 2010, 16(9):1293-1301.

184. von Dalowski F, Kramer M, Wermke M, Wehner R, Rollig C, Alakel N, Stolzel F, Parmentier S, Sockel K, Krech M et al: Mesenchymal Stromal Cells for Treatment of Acute Steroid-Refractory Graft Versus Host Disease: Clinical Responses and Long-Term Outcome. Stem cells 2016, 34(2):357-366.

185. Muroi K, Miyamura K, Ohashi K, Murata M, Eto T, Kobayashi N, Taniguchi S, Imamura M, Ando K, Kato S et al: Unrelated allogeneic bone marrow-derived mesenchymal stem cells for steroid-refractory acute graft-versus-host disease: a phase I/II study. International journal of hematology 2013, 98(2):206-213.

186. Bernardo ME, Ball LM, Cometa AM, Roelofs H, Zecca M, Avanzini MA, Bertaina A, Vinti L, Lankester A, Maccario R et al: Co-infusion of ex vivo-expanded, parental MSCs prevents life-threatening acute GVHD, but does not reduce the risk of graft failure in pediatric patients undergoing allogeneic umbilical cord blood transplantation. Bone marrow transplantation 2011, 46(2):200-207.

187. Kuzmina LA, Petinati NA, Parovichnikova EN, Lubimova LS, Gribanova EO, Gaponova TV, Shipounova IN, Zhironkina OA, Bigildeev AE, Svinareva DA et al: Multipotent Mesenchymal Stromal Cells for the Prophylaxis of Acute Graft-versus-Host Disease-A Phase II Study. Stem cells international 2012, 2012:968213.

188. Prasad VK, Lucas KG, Kleiner GI, Talano JA, Jacobsohn D, Broadwater G, Monroy R, Kurtzberg J: Efficacy and safety of ex vivo cultured adult human mesenchymal stem cells (Prochymal) in pediatric patients with severe refractory acute graft-versus-host disease in a compassionate use study. Biology of blood and marrow transplantation : journal of the American Society for Blood and Marrow Transplantation 2011, 17(4):534-541.

189. Ball LM, Bernardo ME, Roelofs H, van Tol MJ, Contoli B, Zwaginga JJ, Avanzini MA, Conforti A, Bertaina A, Giorgiani G et al: Multiple infusions of mesenchymal stromal cells induce sustained remission in children with steroid-refractory, grade III-IV acute graft-versus-host disease. British journal of haematology 2013, 163(4):501-509.

190. Kurtzberg J, Prockop S, Teira P, Bittencourt H, Lewis V, Chan KW, Horn B, Yu L, Talano JA, Nemecek E et al: Allogeneic human mesenchymal stem cell therapy (remestemcel-L, Prochymal) as a rescue agent for severe refractory acute graft-versus-host disease in pediatric patients. Biology of blood and marrow transplantation : journal of the American Society for Blood and Marrow Transplantation 2014, 20(2):229-235.

191. Erbey F, Atay D, Akcay A, Ovali E, Ozturk G: Mesenchymal Stem Cell Treatment for Steroid Refractory Graft-versus-Host Disease in Children: A Pilot and First Study from Turkey. Stem cells international 2016, 2016:1641402.

192. Introna M, Lucchini G, Dander E, Galimberti S, Rovelli A, Balduzzi A, Longoni D, Pavan F, Masciocchi F, Algarotti A et al: Treatment of graft versus host disease with mesenchymal stromal cells: a phase I study on 40 adult and pediatric patients. Biology of blood and marrow transplantation : journal of the American Society for Blood and Marrow Transplantation 2014, 20(3):375-381.

193. Kebriaei P, Isola L, Bahceci E, Holland K, Rowley S, McGuirk J, Devetten M, Jansen J, Herzig R, Schuster M et al: Adult human mesenchymal stem cells added to corticosteroid therapy for the treatment of acute graft-versus-host disease. Biology of blood and marrow transplantation : journal of the American Society for Blood and Marrow Transplantation 2009, 15(7):804-811.

194. Zhou H, Guo M, Bian C, Sun Z, Yang Z, Zeng Y, Ai H, Zhao RC: Efficacy of bone marrow-derived mesenchymal stem cells in the treatment of sclerodermatous chronic graft-versus-host disease: clinical report. Biology of blood and marrow transplantation : journal of the American Society for Blood and Marrow Transplantation 2010, 16(3):403-412.

195. Ringden O, Uzunel M, Sundberg B, Lonnies L, Nava S, Gustafsson J, Henningsohn L, Le Blanc K: Tissue repair using allogeneic mesenchymal stem cells for hemorrhagic cystitis, pneumomediastinum and perforated colon. Leukemia2007, 21(11):2271-2276.

196. Weng J, He C, Lai P, Luo C, Guo R, Wu S, Geng S, Xiangpeng A, Liu X, Du X: Mesenchymal stromal cells treatment attenuates dry eye in patients with chronic graft-versus-host disease. Molecular therapy : the journal of the American Society of Gene Therapy 2012, 20(12):2347-2354.

197. Fang B, Song Y, Zhao RC, Han Q, Lin Q: Using human adipose tissue-derived mesenchymal stem cells as salvage therapy for hepatic graft-versus-host disease resembling acute hepatitis. Transplantation proceedings 2007, 39(5):1710-1713.

198. Fang B, Song Y, Lin Q, Zhang Y, Cao Y, Zhao RC, Ma Y: Human adipose tissue-derived mesenchymal stromal cells as salvage therapy for treatment of severe refractory acute graft-vs.-host disease in two children. Pediatric transplantation 2007, 11(7):814-817.

199. Wang Y, Chen F, Gu B, Chen G, Chang H, Wu D: Mesenchymal stromal cells as an adjuvant treatment for severe late-onset hemorrhagic cystitis after allogeneic hematopoietic stem cell transplantation. Acta haematologica 2015, 133(1):72-77.

200. Wu Y, Wang Z, Cao Y, Xu L, Li X, Liu P, Yan P, Liu Z, Zhao D, Wang J et al: Cotransplantation of haploidentical hematopoietic and umbilical cord mesenchymal stem cells with a myeloablative regimen for refractory/relapsed hematologic malignancy. Annals of hematology 2013, 92(12):1675-1684.

201. Wu QL, Liu XY, Nie DM, Zhu XX, Fang J, You Y, Zhong ZD, Xia LH, Hong M: Umbilical cord blood-derived mesenchymal stem cells ameliorate graft-versus-host disease following allogeneic hematopoietic stem cell transplantation through multiple immunoregulations. Journal of Huazhong University of Science and Technology Medical sciences = Hua zhong ke ji da xue xue bao Yi xue Ying De wen ban = Huazhong keji daxue xuebao Yixue Yingdewen ban 2015, 35(4):477-484.

202. Gao L, Zhang Y, Hu B, Liu J, Kong P, Lou S, Su Y, Yang T, Li H, Liu Y et al: Phase II Multicenter, Randomized, Double-Blind Controlled Study of Efficacy and Safety of Umbilical Cord-Derived Mesenchymal Stromal Cells in the Prophylaxis of Chronic Graft-Versus-Host Disease After HLA-Haploidentical Stem-Cell Transplantation. Journal of clinical oncology : official journal of the American Society of Clinical Oncology 2016, 34(24):2843-2850.

203. Yin F, Battiwalla M, Ito S, Feng X, Chinian F, Melenhorst JJ, Koklanaris E, Sabatino M, Stroncek D, Samsel L et al: Bone marrow mesenchymal stromal cells to treat tissue damage in allogeneic stem cell transplant recipients: correlation of biological markers with clinical responses. Stem cells 2014, 32(5):1278-1288.

204. Peng Y, Chen X, Liu Q, Xu D, Zheng H, Liu L, Liu Q, Liu M, Fan Z, Sun J et al: Alteration of naive and memory B-cell subset in chronic graft-versus-host disease patients after treatment with mesenchymal stromal cells. Stem cells translational medicine 2014, 3(9):1023-1031.

205. Peng Y, Chen X, Liu Q, Zhang X, Huang K, Liu L, Li H, Zhou M, Huang F, Fan Z et al: Mesenchymal stromal cells infusions improve refractory chronic graft versus host disease through an increase of CD5+ regulatory B cells producing interleukin 10. Leukemia 2015, 29(3):636-646.

206. Zhao K, Lou R, Huang F, Peng Y, Jiang Z, Huang K, Wu X, Zhang Y, Fan Z, Zhou H et al: Immunomodulation effects of mesenchymal stromal cells on acute graft-versus-host disease after hematopoietic stem cell transplantation. Biology of blood and marrow transplantation : journal of the American Society for Blood and Marrow Transplantation 2015, 21(1):97-104.

207. Shipounova IN, Petinati NA, Bigildeev AE, Zezina EA, Drize NI, Kuzmina LA, Parovichnikova EN, Savchenko VG: Analysis of results of acute graft-versus-host disease prophylaxis with donor multipotent mesenchymal stromal cells in patients with hemoblastoses after allogeneic bone marrow transplantation. Biochemistry Biokhimiia 2014, 79(12):1363-1370.

208. Kuzmina LA, Petinati NA, Shipounova IN, Sats NV, Bigildeev AE, Zezina EA, Popova MD, Drize NJ, Parovichnikova EN, Savchenko VG: Analysis of multipotent mesenchymal stromal cells used for acute graft-versus-host disease prophylaxis. European journal of haematology 2016, 96(4):425-434.

209. Ning H, Yang F, Jiang M, Hu L, Feng K, Zhang J, Yu Z, Li B, Xu C, Li Y et al: The correlation between cotransplantation of mesenchymal stem cells and higher recurrence rate in hematologic malignancy patients: outcome of a pilot clinical study. Leukemia 2008, 22(3):593-599.

210. von Bahr L, Sundberg B, Lonnies L, Sander B, Karbach H, Hagglund H, Ljungman P, Gustafsson B, Karlsson H, Le Blanc K et al: Long-term complications, immunologic effects, and role of passage for outcome in mesenchymal stromal cell therapy. Biology of blood and marrow transplantation : journal of the American Society for Blood and Marrow Transplantation 2012, 18(4):557-564.

211. Baron F, Lechanteur C, Willems E, Bruck F, Baudoux E, Seidel L, Vanbellinghen JF, Hafraoui K, Lejeune M, Gothot A et al: Cotransplantation of mesenchymal stem cells might prevent death from graft-versus-host disease (GVHD) without abrogating graft-versus-tumor effects after HLA-mismatched allogeneic transplantation following nonmyeloablative conditioning. Biology of blood and marrow transplantation : journal of the American Society for Blood and Marrow Transplantation 2010, 16(6):838-847.

212. Sanchez-Lazaro IJ, Almenar L, Reganon E, Vila V, Martinez-Dolz L, Martinez-Sales V, Moro J, Aguero J, Ortiz-Martinez V, Salvador A: Inflammatory markers in stable heart failure and their relationship with functional class. Int J Cardiol 2007.

213. Alonso-Martinez JL, Llorente-Diez B, Echegaray-Agara M, Olaz-Preciado F, Urbieta-Echezarreta M, Gonzalez-Arencibia C: C-reactive protein as a predictor of improvement and readmission in heart failure. Eur J Heart Fail 2002, 4(3):331-336.

214. Nakou ES, Liberopoulos EN, Milionis HJ, Elisaf MS: The role of C-reactive protein in atherosclerotic cardiovascular disease: an overview. Curr Vasc Pharmacol 2008, 6(4):258-270.

215. Galarraga B, Khan F, Kumar P, Pullar T, Belch JJ: C-reactive protein: the underlying cause of microvascular dysfunction in rheumatoid arthritis. Rheumatology (Oxford) 2008.

216. Nabata A, Kuroki M, Ueba H, Hashimoto S, Umemoto T, Wada H, Yasu T, Saito M, Momomura S, Kawakami M: C-reactive protein induces endothelial cell apoptosis and matrix metalloproteinase-9 production in human mononuclear cells: Implications for the destabilization of atherosclerotic plaque. Atherosclerosis 2008, 196(1):129-135.

217. Griselli M, Herbert J, Hutchinson WL, Taylor KM, Sohail M, Krausz T, Pepys MB: C-reactive protein and complement are important mediators of tissue damage in acute myocardial infarction. J Exp Med 1999, 190(12):1733-1740.

218. Pepys MB, Hirschfield GM, Tennent GA, Gallimore JR, Kahan MC, Bellotti V, Hawkins PN, Myers RM, Smith MD, Polara A et al: Targeting C-reactive protein for the treatment of cardiovascular disease. Nature 2006, 440(7088):1217-1221.

219. Satoh M, Minami Y, Takahashi Y, Nakamura M: Immune modulation: role of the inflammatory cytokine cascade in the failing human heart. Curr Heart Fail Rep 2008, 5(2):69-74.

220. Yokoyama T, Sekiguchi K, Tanaka T, Tomaru K, Arai M, Suzuki T, Nagai R: Angiotensin II and mechanical stretch induce production of tumor necrosis factor in cardiac fibroblasts. Am J Physiol 1999, 276(6 Pt 2):H1968-1976.

221. Wang BW, Hung HF, Chang H, Kuan P, Shyu KG: Mechanical stretch enhances the expression of resistin gene in cultured cardiomyocytes via tumor necrosis factor-alpha. Am J Physiol Heart Circ Physiol 2007, 293(4):H2305-2312.

222. Satoh S, Oyama J, Suematsu N, Kadokami T, Shimoyama N, Okutsu M, Inoue T, Sugano M, Makino N: Increased productivity of tumor necrosis factor-alpha in helper T cells in patients with systolic heart failure. Int J Cardiol 2006, 111(3):405-412.

223. Conraads VM, Bosmans JM, Schuerwegh AJ, Goovaerts I, De Clerck LS, Stevens WJ, Bridts CH, Vrints CJ: Intracellular monocyte cytokine production and CD 14 expression are up-regulated in severe vs mild chronic heart failure. J Heart Lung Transplant 2005, 24(7):854-859.

224. Haudek SB, Taffet GE, Schneider MD, Mann DL: TNF provokes cardiomyocyte apoptosis and cardiac remodeling through activation of multiple cell death pathways. J Clin Invest 2007, 117(9):2692-2701.

225. Kubota T, Bounoutas GS, Miyagishima M, Kadokami T, Sanders VJ, Bruton C, Robbins PD, McTiernan CF, Feldman AM: Soluble tumor necrosis factor receptor abrogates myocardial inflammation but not hypertrophy in cytokine-induced cardiomyopathy. Circulation 2000, 101(21):2518-2525.

226. Scheibner KA, Lutz MA, Boodoo S, Fenton MJ, Powell JD, Horton MR: Hyaluronan fragments act as an endogenous danger signal by engaging TLR2. J Immunol 2006, 177(2):1272-1281.

227. Termeer C, Benedix F, Sleeman J, Fieber C, Voith U, Ahrens T, Miyake K, Freudenberg M, Galanos C, Simon JC: Oligosaccharides of Hyaluronan activate dendritic cells via toll-like receptor 4. J Exp Med 2002, 195(1):99-111.

228. Asea A: Heat shock proteins and toll-like receptors. Handb Exp Pharmacol 2008(183): 111-127.

229. Nozaki N, Shishido T, Takeishi Y, Kubota I: Modulation of doxorubicin-induced cardiac dysfunction in toll-like receptor-2-knockout mice. Circulation 2004, 110(18):2869-2874.

230. Riad A, Bien S, Gratz M, Escher F, Westermann D, Heimesaat MM, Bereswill S, Krieg T, Felix SB, Schultheiss HP et al: Toll-like receptor-4 deficiency attenuates doxorubicin-induced cardiomyopathy in mice. Eur J Heart Fail 2008, 10(3):233-243.

231. Shishido T, Nozaki N, Yamaguchi S, Shibata Y, Nitobe J, Miyamoto T, Takahashi H, Arimoto T, Maeda K, Yamakawa M et al: Toll-like receptor-2 modulates ventricular remodeling after myocardial infarction. Circulation 2003, 108(23):2905-2910.

232. Sheu JJ, Chang LT, Chiang CH, Youssef AA, Wu CJ, Lee FY, Yip HK: Prognostic value of activated toll-like receptor-4 in monocytes following acute myocardial infarction. Int Heart J 2008, 49(1):1-11.

233. Zannettino AC, Paton S, Arthur A, Khor F, Itescu S, Gimble JM, Gronthos S: Multipotential human adipose-derived stromal stem cells exhibit a perivascular phenotype in vitro and in vivo. J Cell Physiol 2008, 214(2):413-421.

234. Hoogduijn MJ, Crop MJ, Peeters AM, Van Osch GJ, Balk AH, Ijzermans JN, Weimar W, Baan CC: Human heart, spleen, and perirenal fat-derived mesenchymal stem cells have immunomodulatory capacities. Stem Cells Dev 2007, 16(4):597-604.

235. Chao KC, Chao KF, Fu YS, Liu SH: Islet-like clusters derived from mesenchymal stem cells in Wharton’s Jelly of the human umbilical cord for transplantation to control type 1 diabetes. PLoS ONE 2008, 3(1):e1451.

236. Jo YY, Lee HJ, Kook SY, Choung HW, Park JY, Chung JH, Choung YH, Kim ES, Yang HC, Choung PH: Isolation and characterization of postnatal stem cells from human dental tissues. Tissue Eng 2007, 13(4):767-773.

237. He Q, Wan C, Li G: Concise review: multipotent mesenchymal stromal cells in blood. Stem Cells 2007, 25(1):69-77.

238. Oh W, Kim DS, Yang YS, Lee JK: Immunological properties of umbilical cord blood-derived mesenchymal stromal cells. Cell Immunol 2008.

239. Meng X, Ichim TE, Zhong J, Rogers A, Yin Z, Jackson J, Wang H, Ge W, Bogin V, Chan KW et al: Endometrial regenerative cells: a novel stem cell population. J Transl Med 2007, 5:57.

240. Hida N, Nishiyama N, Miyoshi S, Kira S, Segawa K, Uyama T, Mori T, Miyado K, Ikegami Y, Cui C et al: Novel Cardiac Precursor-Like Cells from Human Menstrual Blood-Derived Mesenchymal Cells. Stem Cells 2008.

241. Patel AN, Park E, Kuzman M, Benetti F, Silva FJ, Allickson JG: Multipotent menstrual blood stromal stem cells: isolation, characterization, and differentiation. Cell Transplant 2008, 17(3):303-311.

242. Le Blanc K, Ringden O: Immunomodulation by mesenchymal stem cells and clinical experience. J Intern Med 2007, 262(5):509-525.

243. Keyser KA, Beagles KE, Kiem HP: Comparison of mesenchymal stem cells from different tissues to suppress T-cell activation. Cell Transplant 2007, 16(5):555-562.

244. Nasef A, Chapel A, Mazurier C, Bouchet S, Lopez M, Mathieu N, Sensebe L, Zhang Y, Gorin NC, Thierry D et al: Identification of IL-10 and TGF-beta transcripts involved in the inhibition of T-lymphocyte proliferation during cell contact with human mesenchymal stem cells. Gene Expr 2007, 13(4-5):217-226.

245. Ryan JM, Barry F, Murphy JM, Mahon BP: Interferon-gamma does not break, but promotes the immunosuppressive capacity of adult human mesenchymal stem cells. Clin Exp Immunol 2007, 149(2):353-363.

246. Nasef A, Mazurier C, Bouchet S, Francois S, Chapel A, Thierry D, Gorin NC, Fouillard L: Leukemia inhibitory factor: Role in human mesenchymal stem cells mediated immunosuppression. Cell Immunol 2008, 253(1-2):16-22.

247. Ortiz LA, Dutreil M, Fattman C, Pandey AC, Torres G, Go K, Phinney DG: Interleukin 1 receptor antagonist mediates the antiinflammatory and antifibrotic effect of mesenchymal stem cells during lung injury. Proc Natl Acad Sci U S A 2007, 104(26): 11002-11007.

248. English K, Barry FP, Field-Corbett CP, Mahon BP: IFN-gamma and TNF-alpha differentially regulate immunomodulation by murine mesenchymal stem cells. Immunol Lett 2007, 110(2):91-100.

249. Jones BJ, Brooke G, Atkinson K, McTaggart SJ: Immunosuppression by placental indoleamine 2,3-dioxygenase: a role for mesenchymal stem cells. Placenta 2007, 28(11-12):1174-1181.

250. Kassis I, Grigoriadis N, Gowda-Kurkalli B, Mizrachi-Kol R, Ben-Hur T, Slavin S, Abramsky O, Karussis D: Neuroprotection and immunomodulation with mesenchymal stem cells in chronic experimental autoimmune encephalomyelitis. Arch Neurol 2008, 65(6):753-761.

251. Parekkadan B, Tilles AW, Yarmush ML: Bone marrow-derived mesenchymal stem cells ameliorate autoimmune enteropathy independently of regulatory T cells. Stem Cells 2008, 26(7): 1913-1919.

252. Li H, Guo Z, Jiang X, Zhu H, Li X, Mao N: Mesenchymal Stem Cells Alter Migratory Property of T and Dendritic Cells to Delay the Development of Murine Lethal Acute Graft-Versus-Host Disease. Stem Cells 2008.

253. Augello A, Tasso R, Negrini SM, Cancedda R, Pennesi G: Cell therapy using allogeneic bone marrow mesenchymal stem cells prevents tissue damage in collagen-induced arthritis. Arthritis Rheum2007, 56(4):1175-1186.

254. Semedo P, Wang PM, Andreucci TH, Cenedeze MA, Teixeira VP, Reis MA, Pacheco-Silva A, Camara NO: Mesenchymal stem cells ameliorate tissue damages triggered by renal ischemia and reperfusion injury. Transplant Proc 2007, 39(2):421-423.

255. Du YY, Zhou SH, Zhou T, Su H, Pan HW, Du WH, Liu B, Liu QM: Immuno-inflammatory regulation effect of mesenchymal stem cell transplantation in a rat model of myocardial infarction. Cytotherapy 2008, 10(5):469-478.

256. Ball L, Bredius R, Lankester A, Schweizer J, van den Heuvel-Eibrink M, Escher H, Fibbe W, Egeler M: Third party mesenchymal stromal cell infusions fail to induce tissue repair despite successful control of severe grade IV acute graft-versus-host disease in a child with juvenile myelo-monocytic leukemia. Leukemia 2008, 22(6):1256-1257.

257. Muller I, Kordowich S, Holzwarth C, Isensee G, Lang P, Neunhoeffer F, Dominici M, Greil J, Handgretinger R: Application of multipotent mesenchymal stromal cells in pediatric patients following allogeneic stem cell transplantation. Blood Cells Mol Dis 2008, 40(1):25-32.

258. Narula J, Haider N, Virmani R, DiSalvo TG, Kolodgie FD, Hajjar RJ, Schmidt U, Semigran MJ, Dec GW, Khaw BA: Apoptosis in myocytes in end-stage heart failure. N Engl J Med 1996, 335(16):1182-1189.

259. Dorn GW, 2nd: Apoptotic and non-apoptotic programmed cardiomyocyte death in ventricular remodelling. Cardiovasc Res 2008.

260. Rodriguez M, Lucchesi BR, Schaper J: Apoptosis in myocardial infarction. Ann Med 2002, 34(6):470-479.

261. Odashima M, Usui S, Takagi H, Hong C, Liu J, Yokota M, Sadoshima J: Inhibition of endogenous Mst1 prevents apoptosis and cardiac dysfunction without affecting cardiac hypertrophy after myocardial infarction. Circ Res 2007, 100(9):1344-1352.

262. Chua CC, Gao J, Ho YS, Xiong Y, Xu X, Chen Z, Hamdy RC, Chua BH: Overexpression of IAP-2 attenuates apoptosis and protects against myocardial ischemia/reperfusion injury in transgenic mice. Biochim Biophys Acta 2007, 1773(4):577-583.

263. Jayasankar V, Woo YJ, Bish LT, Pirolli TJ, Chatterjee S, Berry MF, Burdick J, Gardner TJ, Sweeney HL: Gene transfer of hepatocyte growth factor attenuates postinfarction heart failure. Circulation 2003, 108 Suppl 1:II230-236.

264. Filippatos G, Uhal BD: Blockade of apoptosis by ACE inhibitors and angiotensin receptor antagonists. Curr Pharm Des 2003, 9(9):707-714.

265. Fransioli J, Bailey B, Gude NA, Cottage CT, Muraski JA, Emmanuel G, Wu W, Alvarez R, Rubio M, Ottolenghi S et al: Evolution of the c-kit-positive cell response to pathological challenge in the myocardium. Stem Cells 2008, 26(5):1315-1324.

266. Urbanek K, Torella D, Sheikh F, De Angelis A, Nurzynska D, Silvestri F, Beltrami CA, Bussani R, Beltrami AP, Quaini F et al: Myocardial regeneration by activation of multipotent cardiac stem cells in ischemic heart failure. Proceedings of the National Academy of Sciences 2005, 102(24):8692-8697.

267. Dawn B, Stein AB, Urbanek K, Rota M, Whang B, Rastaldo R, Torella D, Tang XL, Rezazadeh A, Kajstura J et al: Cardiac stem cells delivered intravascularly traverse the vessel barrier, regenerate infarcted myocardium, and improve cardiac function. Proc Natl Acad Sci U S A 2005, 102(10):3766-3771.

268. Raffaghello L, Bianchi G, Bertolotto M, Montecucco F, Busca A, Dallegri F, Ottonello L, Pistoia V: Human mesenchymal stem cells inhibit neutrophil apoptosis: a model for neutrophil preservation in the bone marrow niche. Stem Cells 2008, 26(1):151-162.

269. Mirotsou M, Zhang Z, Deb A, Zhang L, Gnecchi M, Noiseux N, Mu H, Pachori A, Dzau V: Secreted frizzled related protein 2 (Sfrp2) is the key Akt-mesenchymal stem cell-released paracrine factor mediating myocardial survival and repair. Proc Natl Acad SciU S A 2007, 104(5):1643-1648.

270. Wang M, Crisostomo PR, Herring C, Meldrum KK, Meldrum DR: Human progenitor cells from bone marrow or adipose tissue produce VEGF, HGF, and IGF-I in response to TNF by a p38 MAPK-dependent mechanism. Am J Physiol Regul Integr Comp Physiol 2006, 291(4):R880-884.

271. Li TS, Takahashi M, Ohshima M, Qin SL, Kubo M, Muramatsu K, Hamano K: Myocardial repair achieved by the intramyocardial implantation of adult cardiomyocytes in combination with bone marrow cells. Cell Transplant 2008, 17(6):695-703.

272. Crisostomo PR, Wang Y, Markel TA, Wang M, Lahm T, Meldrum DR: Human mesenchymal stem cells stimulated by TNF-alpha, LPS, or hypoxia produce growth factors by an NF kappa B- but not JNK-dependent mechanism. Am J Physiol Cell Physiol 2008, 294(3):C675-682.

273. Fu X, He Y, Xie C, Liu W: Bone marrow mesenchymal stem cell transplantation improves ovarian function and structure in rats with chemotherapy-induced ovarian damage. Cytotherapy 2008, 10(4):353-363.

274. Urbanek K, Rota M, Cascapera S, Bearzi C, Nascimbene A, De Angelis A, Hosoda T, Chimenti S, Baker M, Limana F et al: Cardiac stem cells possess growth factor-receptor systems that after activation regenerate the infarcted myocardium, improving ventricular function and long-term survival. Circ Res 2005, 97(7):663-673.

275. Zeng F, Chen MJ, Baldwin DA, Gong ZJ, Yan JB, Qian H, Wang J, Jiang X, Ren ZR, Sun D et al: Multiorgan engraftment and differentiation of human cord blood CD34+ Lin- cells in goats assessed by gene expression profiling. Proc Natl Acad Sci U S A 2006, 103(20):7801-7806.

276. Nishiyama N, Miyoshi S, Hida N, Uyama T, Okamoto K, Ikegami Y, Miyado K, Segawa K, Terai M, Sakamoto M et al: The significant cardiomyogenic potential of human umbilical cord blood-derived mesenchymal stem cells in vitro. Stem Cells 2007, 25(8):2017-2024.

277. Kawada H, Fujita J, Kinjo K, Matsuzaki Y, Tsuma M, Miyatake H, Muguruma Y, Tsuboi K, Itabashi Y, Ikeda Y et al: Nonhematopoietic mesenchymal stem cells can be mobilized and differentiate into cardiomyocytes after myocardial infarction. Blood 2004, 104(12):3581-3587.

278. Vieyra DS, Jackson KA, Goodell MA: Plasticity and tissue regenerative potential of bone marrow-derived cells. Stem Cell Rev 2005, 1(1):65-69.

279. Fazel S, Cimini M, Chen L, Li S, Angoulvant D, Fedak P, Verma S, Weisel RD, Keating A, Li RK: Cardioprotective c-kit+ cells are from the bone marrow and regulate the myocardial balance of angiogenic cytokines. J Clin Invest 2006, 116(7):1865-1877.

280. Kocher AA, Schuster MD, Szabolcs MJ, Takuma S, Burkhoff D, Wang J, Homma S, Edwards NM, Itescu S: Neovascularization of ischemic myocardium by human bone-marrow-derived angioblasts prevents cardiomyocyte apoptosis, reduces remodeling and improves cardiac function. Nat Med 2001, 7(4):430-436.

281. Li RK JZ, Weisel RD, et al.: Cardiomyocyte transplantation improves heart function. Ann Thorac Surg 1996, 62:654-661.

282. El Oakley RM OO, Bongso A.: Myocyte transplantation for myocardial repair: a few good cells can mend a broken heart. Ann Thorac Surg 2001, 71:1724-1733.

283. Klug MG SM, Koh GY.: Genetically selected cardiomyocytes from differentiating embryonic stem cells form stable intracardiac grafts. J Clin Invest 1996, 98:216-224.

284. Tomita S, Li RK, Weisel RD, Mickle DA, Kim EJ, Sakai T, Jia ZQ: Autologous transplantation of bone marrow cells improves damaged heart function. Circulation 1999, 100(19 Suppl):II247-256.

285. Taylor DA AB, Hungspreugs P.: Regenerating functional myocardium: improved performance after skeletal myoblast transplantation. Nat Med 1998, 4:929-933.

286. Dib N MP, Campbell A, Yeager M, Pagani FD, Wright S, et al.: Feasibility and safety of autologous myoblast transplantation in patients with ischemic cardiomyopathy. Cell Transplant 2005, 14(1):1-9.

287. Dib N MR, Pagani FD, Wright S, Kereiakes DJ, Lengerich R, et al.: Safety and feasibility of autologous myoblast transplantation in patients with ischemic cardiomyopathy: four-year follow-up. Circulation 2005, 112(12):1748-1755.

288. Dimarakis I HN, Gordon MY.: Adult bone marrow-derived stem cells and the injured heart: just the beginning? . Eur J Cardiothorac Surg 2005, 28(5):665-676.

289. GW. D: Management of heart failure: crossing boundary over to the surgical country. Surg Clin N Am 2004, 84:1-25.

290. Erbs S LA, Adams V, Lenk K, Thiele H, Diederich KW, et al.: Transplantation of blood-derived progenitor cells after recanalization of chronic coronary artery occlusion: first randomized and placebo-controlled study. Cir Res 2005, 97(8):756-762.

291. Evers BM WI, Flake AW, Tabar V, Weisel RD.: Stem cells in clinical practice. J Am Coll Surg 2003, 197(3):458–478.

292. Forrester JS PM, Makkar RR.: Stem cell repair of infarcted myocardium: an overview for clinicians.Circulation 2003, 108(9):1139-1145.

293. Li RK MD, Weisel RD.: In vivo survival and function of transplanted rat cardiomyocytes. Cir Res 1996, 78:283-288.

294. Scorsin M MF, Sabri A, Le Dref O, Demirag M, Samuel J-L, Rappaport L, Menasche P. : Can grafted cardiomyocytes colonize periinfarction myocardial areas ? Circulation 1996, 94(ii):337-340.

295. Chiu RC-J ZA, Kao RL.. : Cellular cardiomyoplasty : myocardial regeneration with satellite cell implantation. Ann Thorac Surg1995, 60:12-18.

296. Murry C WR, Schwartz S, Hauschka S.: Skeletal myoblast transplantation for repair of myocardial necrosis. J Clin Invest 1996, 98:2512-2523.

297. Scorsin M HA, Vilguin J-T: Comparison of the effects of fetal cardiomyocyte and skeletal myoblast transplantation on postinfarction left ventricular function.. J Thorac Cardiovasc Surg 2000, 119:1169-1175.

298. Taylor DA, Atkins BZ, Hungspreugs P, Jones TR, Reedy MC, Hutcheson KA, Glower DD, Kraus WE: Regenerating functional myocardium: improved performance after skeletal myoblast transplantation. Nat Med 1998, 4(8):929-933.

299. Atkins BZ LC, Kraus WE.: Intracardiac transplantation of skeletal myoblasts yields two populations of striated cells in situ. Ann Thorac Surg 1999, 67:124-129.

300. Menasché P HA, Scorsin M.: Myoblast transplantation for heart failure. Lancet 2001, 357:279-280.

301. Siminiak T KR, Kurpisz M.: Myoblast transplantation in the treatment of postinfarction myocardial contractility impairment. Kardiol Pol 2002, 56:131-137.

302. Herreros J, et al.: Autologous intramyocardial injection of cultured skeletal muscle-derived stem cells in patients with non-acute myocardial infarction. Eur Heart J 2003, 24(22):2012.

303. Bittner RE, Schofer, C., Weipoltshammer, K., Ivanova, S., Streubel, B., Hauser, E., Freilinger, M., Hoger, H., Elbe-Burger, A., and Wachtler, F. 199:391-396, 1999: Recruitment of bone-marrow-derived cells by skeletal and cardiac muscle in adult dystrophic mdx mice. Anat Embryol (Berl) 1999, 199:391-396.

304. Goodell MA, Brose, K., Paradis, G., Conner, A. S., and Mulligan, R. C.: Isolation and functional properties of murine hematopoietic stem cells that are replicating in vivo. J Exp Med 1996, 183:1797-1806.

305. Zhou S, Schuetz, J. D., Bunting, K. D., Colapietro, A. M., Sampath, J., Morris, J. J., Lagutina, I., Grosveld, G. C., Osawa, M., Nakauchi, H., and Sorrentino, B. P.: The ABC transporter Bcrp1/ABCG2 is expressed in a wide variety of stem cells and is a molecular determinant of the side-population phenotype.Nat Med 2001, 7:1028-1304.

306. Jackson KA, Majka, S. M., Wang, H., Pocius, J., Hartley, C. J., Majesky, M. W., Entman, M. L., Michael, L. H., Hirschi, K. K., and Goodell, M. A. : Regeneration of ischemic cardiac muscle and vascular endothelium by adult stem cells. J Clin Invest 2001, 107:1395-1402.

307. Toma C, Pittenger MF, Cahill KS, Byrne BJ, Kessler PD: Human mesenchymal stem cells differentiate to a cardiomyocyte phenotype in the adult murine heart. Circulation 2002, 105(1):93-98.

308. Nishikawa S: A complex linkage in the developmental pathway of endothelial and hematopoietic. Curr Opin Cell 2001, 13:673-678.

309. Reyes M, Dudek, A, Jahagirdar, B, Koodie, L, Marker, PH, Verfaillie, CM Origin of endothelial progenitors in human postnatal bone marrow.. J Clin Invest 2002, 109:337-346.

310. Majka SM, Jackson, K. A., Kienstra, K. A., Majesky, M. W., Goodell, M. A., and Hirschi, K. K.: Distinct progenitor populations in skeletal muscle are bone marrow derived and exhibit different cell fates during vascular regeneration. J Clin Invest 2003, 111:71-79.

311. Yoon YS PJ, Tkebuchava T, Luedeman C, Losordo DW.: Unexpected severe calcification after transplantation of bone marrow cells in acute myocardial infarction. Circulation 2004, 109:3154-3157.

312. Strauer BE, Brehm M, Zeus T, Gattermann N, Hernandez A, Sorg RV, Kogler G, Wernet P: [Intracoronary, human autologous stem cell transplantation for myocardial regeneration following myocardial infarction]. Dtsch Med Wochenschr 2001, 126(34-35):932-938.

313. Hamano K, Nishida M, Hirata K, Mikamo A, Li TS, Harada M, Miura T, Matsuzaki M, Esato K: Local implantation of autologous bone marrow cells for therapeutic angiogenesis in patients with ischemic heart disease: clinical trial and preliminary results. Jpn Circ J 2001, 65(9):845-847.

314. Abdel-Latif A, Bolli R, Tleyjeh IM, Montori VM, Perin EC, Hornung CA, Zuba-Surma EK, Al-Mallah M, Dawn B: Adult bone marrow-derived cells for cardiac repair: a systematic review and meta-analysis. Arch Intern Med 2007, 167(10):989-997.

315. Martin-Rendon E, Brunskill S, Doree C, Hyde C, Watt S, Mathur A, Stanworth S: Stem cell treatment for acute myocardial infarction. Cochrane Database Syst Rev 2008(4):CD006536.

316. Kang S, Yang YJ, Li CJ, Gao RL: Effects of intracoronary autologous bone marrow cells on left ventricular function in acute myocardial infarction: a systematic review and meta-analysis for randomized controlled trials. Coron Artery Dis 2008, 19(5):327-335.

317. Cheng Z, Ou L, Zhou X, Li F, Jia X, Zhang Y, Liu X, Li Y, Ward CA, Melo LG et al: Targeted migration of mesenchymal stem cells modified with CXCR4 gene to infarcted myocardium improves cardiac performance. Mol Ther 2008, 16(3):571-579.

318. Fisher SA, Doree C, Mathur A, Martin-Rendon E: Meta-Analysis of Cell Therapy Trials for Patients with Heart Failure - An Update. Circulation research 2015.

319. Murry CE ea: Haematopoietic stem cells do not transdifferentiate into cardiac myocytes in myocardial infarcts.Nature 2004, 429:664-668

320. Balsam LB ea: Haematopoietic stem cells adopt mature haematopoietic fates in ischaemic myocardium.Nature 2004, 428:668-673

321. Assmus B SV, Teupe C, Britten M, Lehmann R, Dobert N, et al. : Transplantation of cells and regeneration enhancement in acute myocardial infarction (TOPCARE-AMI). Circulation 2002, 106:3009-3017.

322. Strauer BE BM, Zeus T, Kostering M, Hernandez A, Sorg RV: Repair of infarcted myocardium by autologous intracoronary mononuclear bone marrow cell transplantation in humans. Circulation 2002, 106:1913-1918.

323. Stamm C, B. Westphal, H.D. Kleine: Autologous bone-marrow stem-cell transplantation for myocardial regeneration.Lancet 2003, 361:45-46.

324. Gehling UM ES, Schumacher U, Wagener C, Pantel K, Otte M, Schuch G, Schafhausen P, Mende T, Kilic N, Kluge K, Schafer B, Hossfeld DK, Fiedler W.: In vitro differentiation of endothelial cells from AC133-positive progenitor cells. Blood 2000, 95(10):3106-3112.

325. Stamm C, et al.,: CABG and bone marrow stem cell transplantation after myocardial infarction. Thorac Cardiovasc Surg 2004, 52(3):152-158.

326. Bartunek J, Vanderheyden, M, Vandekerckhove, B, Mansour, S, De Bruyne, B, De Bondt, P, Van Haute, I, Lootens, N, Heyndrickx, G, Wijns, W.,: Intracoronary injection of CD133-positive enriched bone marrow progenitor cells promotes cardiac recovery after recent myocardial infarction: feasibility and safety. Circulation 2005, 112(9):178-183.

327. Pierelli L BG, Rutella S, Marone M, Scambia G, Leone G.: CD105 (endoglin) expression on hematopoietic stem/progenitor cells. Leuk Lymphoma 2001, 42(6):1195-1206.

328. Barry FP BR, Haynesworth S, Murphy JM, Zaia J.: The monoclonal antibody SH-2, raised against human mesenchymal stem cells, recognizes an epitope on endoglin (CD105). Biochem Biophys Res Commun 1999, 265(1):134-139.

329. Cheng T SD: Cell cycle entry of hematopoietic stem and progenitor cells controlled by distinct cyclin-dependent kinase inhibitors. Int J Hematol 2002, 75(5):460-465.

330. Wang Z MN, Bonelli A, Mole P, Carlesso N, Olson DP, Scadden DT.: Receptor tyrosine kinase, EphB4 (HTK), accelerates differentiation of select human hematopoietic cells. Blood 2002, 99(8):2740-2747.

331. Herrera MB, Bussolati B, Bruno S, Morando L, Mauriello-Romanazzi G, Sanavio F, Stamenkovic I, Biancone L, Camussi G: Exogenous mesenchymal stem cells localize to the kidney by means of CD44 following acute tubular injury. Kidney Int 2007, 72(4):430-441.

332. Sackstein R, Merzaban JS, Cain DW, Dagia NM, Spencer JA, Lin CP, Wohlgemuth R: Ex vivo glycan engineering of CD44 programs human multipotent mesenchymal stromal cell trafficking to bone. Nat Med 2008, 14(2):181-187.

333. Zhu H, Mitsuhashi N, Klein A, Barsky LW, Weinberg K, Barr ML, Demetriou A, Wu GD: The role of the hyaluronan receptor CD44 in mesenchymal stem cell migration in the extracellular matrix. Stem Cells 2006, 24(4):928-935.

334. Wang Y, Deng Y, Zhou GQ: SDF-1alpha/CXCR4-mediated migration of systemically transplanted bone marrow stromal cells towards ischemic brain lesion in a rat model. Brain Res 2008, 1195:104-112.

335. Neschadim A, McCart JA, Keating A, Medin JA: A roadmap to safe, efficient, and stable lentivirus-mediated gene therapy with hematopoietic cell transplantation. Biol Blood Marrow Transplant 2007, 13(12):1407-1416.

336. Hung SC, Pochampally RR, Hsu SC, Sanchez C, Chen SC, Spees J, Prockop DJ: Short-term exposure of multipotent stromal cells to low oxygen increases their expression of CX3CR1 and CXCR4 and their engraftment in vivo. PLoS One 2007, 2(5):e416.

337. Shi M, Li J, Liao L, Chen B, Li B, Chen L, Jia H, Zhao RC: Regulation of CXCR4 expression in human mesenchymal stem cells by cytokine treatment: role in homing efficiency in NOD/SCID mice. Haematologica 2007, 92(7):897-904.

338. Tang YL, Qian K, Zhang YC, Shen L, Phillips MI: Mobilizing of haematopoietic stem cells to ischemic myocardium by plasmid mediated stromal-cell-derived factor-1alpha (SDF-1alpha) treatment. Regul Pept 2005, 125(1-3):1-8.

339. Gibble JW, Ness PM: Fibrin glue: the perfect operative sealant? Transfusion 1990, 30(8):741-747.

340. Zhang G, Nakamura Y, Wang X, Hu Q, Suggs LJ, Zhang J: Controlled release of stromal cell-derived factor-1 alpha in situ increases c-kit+ cell homing to the infarcted heart. Tissue Eng 2007, 13(8):2063-2071.

341. Barile L, Messina E, Giacomello A, Marban E: Endogenous cardiac stem cells. Prog Cardiovasc Dis 2007, 50(1):31-48.

342. Tan Y, Shao H, Eton D, Yang Z, Alonso-Diaz L, Zhang H, Schulick A, Livingstone AS, Yu H: Stromal cell-derived factor-1 enhances pro-angiogenic effect of granulocyte-colony stimulating factor. Cardiovasc Res 2007, 73(4):823-832.

343. Latini R, Brines M, Fiordaliso F: Do non-hemopoietic effects of erythropoietin play a beneficial role in heart failure? Heart Fail Rev 2008, 13(4):415-423.

344. Brunner S, Winogradow J, Huber BC, Zaruba MM, Fischer R, David R, Assmann G, Herbach N, Wanke R, Mueller-Hoecker J et al: Erythropoietin administration after myocardial infarction in mice attenuates ischemic cardiomyopathy associated with enhanced homing of bone marrow-derived progenitor cells via the CXCR-4/SDF-1 axis. Faseb J 2008.

345. Chambers SM, Shaw CA, Gatza C, Fisk CJ, Donehower LA, Goodell MA: Aging hematopoietic stem cells decline in function and exhibit epigenetic dysregulation. PLoS Biol 2007, 5(8):e201.

346. Schmidt-Lucke C, Rossig, L, Fichtlscherer, S, Vasa, M, Britten, M, Kamper, U, Dimmeler, S, Zeiher, AM.,: Reduced number of circulating endothelial progenitor cells predicts future cardiovascular events: proof of concept for the clinical importance of endogenous vascular repair. Circulation 2005, 111(22):2981-2987.

347. Wagers AJ, Sherwood RI, Christensen JL, Weissman IL: Little evidence for developmental plasticity of adult hematopoietic stem cells. Science 2002, 297(5590):2256-2259.

348. Rose RA, Jiang H, Wang X, Helke S, Tsoporis JN, Gong N, Keating SC, Parker TG, Backx PH, Keating A: Bone Marrow-Derived Mesenchymal Stromal Cells Express Cardiac-Specific Markers, Retain the Stromal Phenotype and do not Become Functional Cardiomyocytes In Vitro. Stem Cells 2008.

349. Makino S, Fukuda K, Miyoshi S, Konishi F, Kodama H, Pan J, Sano M, Takahashi T, Hori S, Abe H et al: Cardiomyocytes can be generated from marrow stromal cells in vitro. J Clin Invest 1999, 103(5):697-705.

350. De Felice L, Tatarelli C, Mascolo MG, Gregorj C, Agostini F, Fiorini R, Gelmetti V, Pascale S, Padula F, Petrucci MT et al: Histone deacetylase inhibitor valproic acid enhances the cytokine-induced expansion of human hematopoietic stem cells. Cancer research 2005, 65(4):1505-1513.

351. Bug G, Gul H, Schwarz K, Pfeifer H, Kampfmann M, Zheng X, Beissert T, Boehrer S, Hoelzer D, Ottmann OG et al: Valproic acid stimulates proliferation and self-renewal of hematopoietic stem cells. Cancer research 2005, 65(7):2537-2541.

352. Lee TM, Lin MS, Chang NC: Inhibition of histone deacetylase on ventricular remodeling in infarcted rats. Am J Physiol Heart Circ Physiol 2007, 293(2):H968-977.

353. Hattori N, Imao Y, Nishino K, Ohgane J, Yagi S, Tanaka S, Shiota K: Epigenetic regulation of Nanog gene in embryonic stem and trophoblast stem cells. Genes Cells 2007, 12(3):387-396.

354. Go MJ, Takenaka C, Ohgushi H: Forced expression of Sox2 or Nanog in human bone marrow derived mesenchymal stem cells maintains their expansion and differentiation capabilities. Exp Cell Res 2008, 314(5):1147-1154.

355. Takahashi K, Yamanaka S: Induction of pluripotent stem cells from mouse embryonic and adult fibroblast cultures by defined factors. cell 2006, 126(4):663-676.

356. Takahashi K, Tanabe K, Ohnuki M, Narita M, Ichisaka T, Tomoda K, Yamanaka S: Induction of pluripotent stem cells from adult human fibroblasts by defined factors. cell 2007, 131(5):861-872.

357. Hanna J, Wernig M, Markoulaki S, Sun CW, Meissner A, Cassady JP, Beard C, Brambrink T, Wu LC, Townes TM et al: Treatment of sickle cell anemia mouse model with iPS cells generated from autologous skin. Science 2007, 318(5858):1920-1923.

358. Kim JB, Zaehres H, Wu G, Gentile L, Ko K, Sebastiano V, Arauzo-Bravo MJ, Ruau D, Han DW, Zenke M et al: Pluripotent stem cells induced from adult neural stem cells by reprogramming with two factors. Nature 2008, 454(7204):646-650.

359. Okita K, Nakagawa M, Hyenjong H, Ichisaka T, Yamanaka S: Generation of Mouse Induced Pluripotent Stem Cells Without Viral Vectors. Science 2008.

360. Yang S, Lin G, Tan YQ, Zhou D, Deng LY, Cheng DH, Luo SW, Liu TC, Zhou XY, Sun Z et al: Tumor progression of culture-adapted human embryonic stem cells during long-term culture. Genes Chromosomes Cancer 2008, 47(8):665-679.

361. Ben-Porath I, Thomson MW, Carey VJ, Ge R, Bell GW, Regev A, Weinberg RA: An embryonic stem cell-like gene expression signature in poorly differentiated aggressive human tumors. Nat Genet 2008, 40(5):499-507.

362. Li L, Baroja ML, Majumdar A, Chadwick K, Rouleau A, Gallacher L, Ferber I, Lebkowski J, Martin T, Madrenas J et al: Human embryonic stem cells possess immune-privileged properties. Stem Cells 2004, 22(4):448-456.

363. Slavin S, Kurkalli BG, Karussis D: The potential use of adult stem cells for the treatment of multiple sclerosis and other neurodegenerative disorders. Clin Neurol Neurosurg 2008.

364. von Bonin M, Stolzel F, Goedecke A, Richter K, Wuschek N, Holig K, Platzbecker U, Illmer T, Schaich M, Schetelig J et al: Treatment of refractory acute GVHD with third-party MSC expanded in platelet lysate-containing medium. Bone Marrow Transplant 2008.

365. Dunac A, Frelin C, Popolo-Blondeau M, Chatel M, Mahagne MH, Philip PJ: Neurological and functional recovery in human stroke are associated with peripheral blood CD34+ cell mobilization. J Neurol 2007, 254(3):327-332.

366. Taguchi A, Nakagomi N, Matsuyama T, Kikuchi-Taura A, Yoshikawa H, Kasahara Y, Hirose H, Moriwaki H, Nakagomi T, Soma T et al: Circulating CD34-positive cells have prognostic value for neurologic function in patients with past cerebral infarction. J Cereb Blood Flow Metab 2008.

367. Grundmann F, Scheid C, Braun D, Zobel C, Reuter H, Schwinger RH, Muller-Ehmsen J: Differential increase of CD34, KDR/CD34, CD133/CD34 and CD117/CD34 positive cells in peripheral blood of patients with acute myocardial infarction. Clin Res Cardiol 2007, 96(9):621-627.

368. Hill JM, Zalos G, Halcox JP, Schenke WH, Waclawiw MA, Quyyumi AA, Finkel T: Circulating endothelial progenitor cells, vascular function, and cardiovascular risk. N Engl J Med 2003, 348(7):593-600.

369. Van Zant G, Liang Y: The role of stem cells in aging. Exp Hematol 2003, 31(8):659-672.

370. Wojakowski W, Tendera, M, Michalowska, A, Majka, M, Kucia, M, Maslankiewicz, K, Wyderka, R, Ochala, A, Ratajczak, MZ,: Mobilization of CD34/CXCR4+, CD34/CD117+, c-met+ stem cells, and mononuclear cells expressing early cardiac, muscle, and endothelial markers into peripheral blood in patients with acute myocardial infarction. Circulation 2004, 110(20):3213-3220.

371. Locatelli F, Rocha V, Reed W, Bernaudin F, Ertem M, Grafakos S, Brichard B, Li X, Nagler A, Giorgiani G et al: Related umbilical cord blood transplantation in patients with thalassemia and sickle cell disease. Blood 2003, 101(6):2137-2143.

372. Cornetta K, Laughlin M, Carter S, Wall D, Weinthal J, Delaney C, Wagner J, Sweetman R, McCarthy P, Chao N: Umbilical cord blood transplantation in adults: results of the prospective Cord Blood Transplantation (COBLT). Biol Blood Marrow Transplant 2005, 11(2):149-160.

373. Lubin BHaG, M.F.: Collection and storage of umbilical cord blood for hematopoietic cell transplantation. In.; 2007.

374. Gluckman E, Rocha V, Boyer-Chammard A, Locatelli F, Arcese W, Pasquini R, Ortega J, Souillet G, Ferreira E, Laporte JP et al: Outcome of cord-blood transplantation from related and unrelated donors. Eurocord Transplant Group and the European Blood and Marrow Transplantation Group. N Engl J Med 1997, 337(6):373-381.

375. Laughlin MJ, Barker, J., Bambach, B., Koc O., Rizzieri D., Wagner J., Gerson S.L., Lazarus H.M., Cairo M., Stevens C.E., Rubinstein P., Kurtzberg J.: Hematopoietic engraftment and survival in adult recipients of umbilical-cord blood from unrelated donors. N Engl J Med, 2001, 344(24):1815-1822.

376. Matsumura T, Narimatsu H, Kami M, Yuji K, Kusumi E, Hori A, Murashige N, Tanaka Y, Masuoka K, Wake A et al: Cytomegalovirus infections following umbilical cord blood transplantation using reduced intensity conditioning regimens for adult patients. Biol Blood Marrow Transplant 2007, 13(5):577-583.

377. Sasazuki T, Juji T, Morishima Y, Kinukawa N, Kashiwabara H, Inoko H, Yoshida T, Kimura A, Akaza T, Kamikawaji N et al: Effect of matching of class I HLA alleles on clinical outcome after transplantation of hematopoietic stem cells from an unrelated donor. Japan Marrow Donor Program. N Engl J Med 1998, 339(17):1177-1185.

378. Barker JN, Krepski TP, DeFor TE, Davies SM, Wagner JE, Weisdorf DJ: Searching for unrelated donor hematopoietic stem cells: availability and speed of umbilical cord blood versus bone marrow. Biol Blood Marrow Transplant 2002, 8(5):257-260.

379. Limbourg F, Ringes-Lichtenberg, S, Schaefer, A, Jacoby, C, Mehraein, Y, Jager, MD, Limbourg, A, Fuchs, M, Klein, G, Ballmaier, M, Schlitt, HJ, Schrader, J, Hilfiker-Kleiner, D, Drexler, H,: Haematopoietic stem cells improve cardiac function after infarction without permanent cardiac engraftment. Eur J Heart Fail 2005, 7(5):722-729.

380. Asahara T: Stem cell biology for vascular regeneration. Ernst Schering Res Found Workshop 2005, 54:111-129.

381. Distler JH, Hirth A, Kurowska-Stolarska M, Gay RE, Gay S, Distler O: Angiogenic and angiostatic factors in the molecular control of angiogenesis. Q J Nucl Med 2003, 47(3):149-161.

382. Brogi E, Wu T, Namiki A, Isner JM: Indirect angiogenic cytokines upregulate VEGF and bFGF gene expression in vascular smooth muscle cells, whereas hypoxia upregulates VEGF expression only. Circulation 1994, 90(2):649-652.

383. Nicosia RF, Nicosia SV, Smith M: Vascular endothelial growth factor, platelet-derived growth factor, and insulin-like growth factor-1 promote rat aortic angiogenesis in vitro. Am J Pathol 1994, 145(5):1023-1029.

384. Bos R, van Diest PJ, de Jong JS, van der Groep P, van der Valk P, van der Wall E: Hypoxia-inducible factor-1alpha is associated with angiogenesis, and expression of bFGF, PDGF-BB, and EGFR in invasive breast cancer. Histopathology 2005, 46(1):31-36.

385. Schlingemann RO: Role of growth factors and the wound healing response in age-related macular degeneration. Graefes Arch Clin Exp Ophthalmol 2004, 242(1):91-101.

386. Mentlein R, Held-Feindt J: Angiogenesis factors in gliomas: a new key to tumour therapy? Naturwissenschaften 2003, 90(9):385-394.

387. Zhao L, Eghbali-Webb M: Release of pro- and anti-angiogenic factors by human cardiac fibroblasts: effects on DNA synthesis and protection under hypoxia in human endothelial cells. Biochim Biophys Acta 2001, 1538(2-3):273-282.

388. Dunn IF, Heese O, Black PM: Growth factors in glioma angiogenesis: FGFs, PDGF, EGF, and TGFs. J Neurooncol 2000, 50(1-2):121-137.

389. Kano MR, Morishita Y, Iwata C, Iwasaka S, Watabe T, Ouchi Y, Miyazono K, Miyazawa K: VEGF-A and FGF-2 synergistically promote neoangiogenesis through enhancement of endogenous PDGF-B-PDGFRbeta signaling. J Cell Sci 2005, 118(Pt 16):3759-3768.

390. Laschke MW, Elitzsch A, Vollmar B, Vajkoczy P, Menger MD: Combined inhibition of vascular endothelial growth factor (VEGF), fibroblast growth factor and platelet-derived growth factor, but not inhibition of VEGF alone, effectively suppresses angiogenesis and vessel maturation in endometriotic lesions. Hum Reprod 2006, 21(1):262-268.

391. Kelly BD, Hackett SF, Hirota K, Oshima Y, Cai Z, Berg-Dixon S, Rowan A, Yan Z, Campochiaro PA, Semenza GL: Cell type-specific regulation of angiogenic growth factor gene expression and induction of angiogenesis in nonischemic tissue by a constitutively active form of hypoxia-inducible factor 1. Circ Res 2003, 93(11): 1074-1081.

392. Chauhan A MR, Mullins PA, Taylor G, Petch C, Schofield PM.: Aging-associated endothelial dysfunction in humans is reversed by L-arginine. J Am Coll Cardiol 1996, 28(7):1796-1804.

393. Tschudi MR BM, Bersinger NA, Moreau P, Cosentino F, Noll G, Malinski T, Luscher TF.: Effect of age on kinetics of nitric oxide release in rat aorta and pulmonary artery. J Clin Invest 1996, 98(4):899-905.

394. Hill JM, Syed MA, Arai AE, Powell TM, Paul JD, Zalos G, Read EJ, Khuu HM, Leitman SF, Horne M et al: Outcomes and risks of granulocyte colony-stimulating factor in patients with coronary artery disease. J Am Coll Cardiol 2005, 46(9):1643-1648.

395. 2008. NIoHSCIPAJ.

396. Bonanno G MA, Procoli A: Bonanno G, Mariotti A, Procoli A, et al. Human cord blood CD133+ cells immunoselected by a clinical-grade apparatus differentiate in vitro into endothelial- and cardiomyocyte-like cells. Transfusion. 2007;47(2):280-9.. Transfusion 2007, 47(2):280-289.

397. Cheng F ZP, Handong Y: Induced differentiation of human cord blood mesenchymal stem/progenitor cells into cardiomyocyte-like cells in vitro. J Huazong Univ Sci and Tech 2003, 23(2): 154-157.

398. Yamada Y YS, Fukuda N, : A novel approach for myocardial regeneration with educated cord blood cells cocultured with cells from brown adipose tissue.. Biochem Biophys Res Commun 2007, 353(1):182-188.

399. Nartprayut K, Y UP, Kheolamai P, Manochantr S, Chayosumrit M, Issaragrisil S, Supokawej A: Cardiomyocyte differentiation of perinatallyderived mesenchymal stem cells. Molecular medicine reports 2013, 7(5):1465-1469.

400. Yannarelli G, Dayan V, Pacienza N, Lee CJ, Medin J, Keating A: Human Umbilical Cord Perivascular Cells Exhibit Enhanced Cardiomyocyte Reprogramming and Cardiac Function after Experimental Acute Myocardial Infarction. Cell transplantation 2012.

401. Liang J, Zhang H, Hua B, Wang H, Wang J, Han Z, Sun L: Allogeneic mesenchymal stem cells transplantation in treatment of multiple sclerosis. Multiple sclerosis 2009, 15(5):644-646.

402. Wu KH, Tsai C, Wu HP, Sieber M, Peng CT, Chao YH: Human Application of Ex-Vivo Expanded Umbilical Cord-Derived Mesenchymal Stem Cells: Enhance Hematopoiesis after Cord Blood Transplantation. Cell transplantation 2013.

403. Wu KH, Sheu JN, Wu HP, Tsai C, Sieber M, Peng CT, Chao YH: Cotransplantation of umbilical cord-derived mesenchymal stem cells promote hematopoietic engraftment in cord blood transplantation: a pilot study. Transplantation 2013, 95(5):773-777.

404. Hu J, Yu X, Wang Z, Wang F, Wang L, Gao H, Chen Y, Zhao W, Jia Z, Yan S et al: Long term effects of the implantation of Wharton’s jelly-derived mesenchymal stem cells from the umbilical cord for newly-onset type 1 diabetes mellitus. Endocrine journal 2013, 60(3):347-357.

405. Ma L, Zhou Z, Zhang D, Yang S, Wang J, Xue F, Yang Y, Yang R: Immunosuppressive function of mesenchymal stem cells from human umbilical cord matrix in immune thrombocytopenia patients. Thrombosis and haemostasis 2012, 107(5):937-950.

406. Shi M, Zhang Z, Xu R, Lin H, Fu J, Zou Z, Zhang A, Shi J, Chen L, Lv S et al: Human mesenchymal stem cell transfusion is safe and improves liver function in acute-on-chronic liver failure patients. Stem cells translational medicine 2012, 1(10):725-731.

407. Zhang Z, Lin H, Shi M, Xu R, Fu J, Lv J, Chen L, Lv S, Li Y, Yu S et al: Human umbilical cord mesenchymal stem cells improve liver function and ascites in decompensated liver cirrhosis patients. Journal of gastroenterology and hepatology 2012, 27 Suppl 2:112-120.

408. Lopez Y, Lutjemeier B, Seshareddy K, Trevino EM, Hageman KS, Musch TI, Borgarelli M, Weiss ML: Wharton’s jelly or bone marrow mesenchymal stromal cells improve cardiac function following myocardial infarction for more than 32 weeks in a rat model: a preliminary report. Current stem cell research & therapy 2013, 8(1):46-59.

409. Latifpour M, Nematollahi-Mahani SN, Deilamy M, Azimzadeh BS, Eftekhar-Vaghefi SH, Nabipour F, Najafipour H, Nakhaee N, Yaghoubi M, Eftekhar-Vaghefi R et al: Improvement in cardiac function following transplantation of human umbilical cord matrix-derived mesenchymal cells. Cardiology 2011, 120(1):9-18.

410. Lee EJ, Choi EK, Kang SK, Kim GH, Park JY, Kang HJ, Lee SW, Kim KH, Kwon JS, Lee KH et al: N-cadherin determines individual variations in the therapeutic efficacy of human umbilical cord blood-derived mesenchymal stem cells in a rat model of myocardial infarction. Molecular therapy : the journal of the American Society of Gene Therapy 2012, 20(1):155-167.

411. Liao W, Xie J, Zhong J, Liu Y, Du L, Zhou B, Xu J, Liu P, Yang S, Wang J et al: Therapeutic effect of human umbilical cord multipotent mesenchymal stromal cells in a rat model of stroke. Transplantation 2009, 87(3):350-359.

412. Dayan V, Yannarelli G, Billia F, Filomeno P, Wang XH, Davies JE, Keating A: Mesenchymal stromal cells mediate a switch to alternatively activated monocytes/macrophages after acute myocardial infarction. Basic research in cardiology 2011, 106(6):1299-1310.

413. Corrao S, La Rocca G, Lo Iacono M, Zummo G, Gerbino A, Farina F, Anzalone R: New frontiers in regenerative medicine in cardiology: the potential of Wharton’s jelly mesenchymal stem cells. Current stem cell research & therapy 2013, 8(1):39-45.

414. Prianishnikov VA: On the concept of stem cell and a model of functional-morphological structure of the endometrium. Contraception 1978, 18(3):213-223.

415. Kearns M, Lala PK: Bone marrow origin of decidual cell precursors in the pseudopregnant mouse uterus. J Exp Med 1982, 155(5):1537-1554.

416. Kyo S, Takakura M, Kohama T, Inoue M: Telomerase activity in human endometrium. Cancer Res3 1997, 57(4):610-614.

417. Williams CD, Boggess JF, LaMarque LR, Meyer WR, Murray MJ, Fritz MA, Lessey BA: A prospective, randomized study of endometrial telomerase during the menstrual cycle. J Clin Endocrinol Metab 2001, 86(8):3912-3917.

418. Meng X, Ichim TE, Zhong J, Rogers A, Yin Z, Jackson J, Wang H, Ge W, Bogin V, Chan KW et al: Endometrial regenerative cells: a novel stem cell population. J Transl Med 2007, 5:57.

419. Patel AN, Park E, Kuzman M, Benetti F, Silva FJ, Allickson JG: Multipotent menstrual blood stromal stem cells: isolation, characterization, and differentiation. Cell Transplant 2008, 17(3):303-311.

420. Park JH, Daheron L, Kantarci S, Lee BS, Teixeira JM: Human endometrial cells express elevated levels of pluripotent factors and are more amenable to reprogramming into induced pluripotent stem cells. Endocrinology 2011, 152(3):1080-1089.

421. Cervello I, Gil-Sanchis C, Mas A, Delgado-Rosas F, Martinez-Conejero JA, Galan A, Martinez-Romero A, Martinez S, Navarro I, Ferro J et al: Human endometrial side population cells exhibit genotypic, phenotypic and functional features of somatic stem cells. PLoS One 2010, 5(6):e10964.

422. Niklaus AL, Aberdeen GW, Babischkin JS, Pepe GJ, Albrecht ED: Effect of estrogen on vascular endothelial growth/permeability factor expression by glandular epithelial and stromal cells in the baboon endometrium. Biol Reprod 2003, 68(6):1997-2004.

423. Niklaus AL, Babischkin JS, Aberdeen GW, Pepe GJ, Albrecht ED: Expression of vascular endothelial growth/permeability factor by endometrial glandular epithelial and stromal cells in baboons during the menstrual cycle and after ovariectomy. Endocrinology 2002, 143(10):4007-4017.

424. Albrecht ED, Aberdeen GW, Niklaus AL, Babischkin JS, Suresch DL, Pepe GJ: Acute temporal regulation of vascular endothelial growth/permeability factor expression and endothelial morphology in the baboon endometrium by ovarian steroids. J Clin Endocrinol Metab 2003, 88(6):2844-2852.

425. Heryanto B, Girling JE, Rogers PA: Intravascular neutrophils partially mediate the endometrial endothelial cell proliferative response to oestrogen in ovariectomised mice. Reproduction 2004, 127(5):613-620.

426. Gargett CE, Rogers PA: Human endometrial angiogenesis. Reproduction 2001, 121(2):181-186.

427. Zhang J, Chen Z, Smith GN, Croy BA: Natural killer cell-triggered vascular transformation: maternal care before birth? Cell Mol Immunol 2011, 8(1):1-11.

428. Elsheikh E, Sylven C, Ericzon BG, Palmblad J, Mints M: Cyclic variability of stromal cell-derived factor-1 and endothelial progenitor cells during the menstrual cycle. Int J Mol Med 2011, 27(2):221-226.

429. Robb AO, Mills NL, Smith IB, Short A, Tura-Ceide O, Barclay GR, Blomberg A, Critchley HO, Newby DE, Denison FC: Influence of menstrual cycle on circulating endothelial progenitor cells. Hum Reprod 2009, 24(3):619-625.

430. Lemieux C, Cloutier I, Tanguay JF: Menstrual cycle influences endothelial progenitor cell regulation: a link to gender differences in vascular protection? Int J Cardiol 2009, 136(2):200-210.

431. Taylor HS: Endometrial cells derived from donor stem cells in bone marrow transplant recipients. JAMA 2004, 292(1):81-85.

432. Myers TJ, Granero-Molto F, Longobardi L, Li T, Yan Y, Spagnoli A: Mesenchymal stem cells at the intersection of cell and gene therapy. Expert Opin Biol Ther 2010, 10(12):1663-1679.

433. Mints M, Jansson M, Sadeghi B, Westgren M, Uzunel M, Hassan M, Palmblad J: Endometrial endothelial cells are derived from donor stem cells in a bone marrow transplant recipient. Hum Reprod 2008, 23(1):139-143.

434. Dincer S: Collection of hemopoetic stem cells in allogeneic female donors during menstrual bleeding. Transfus Apher Sci 2004, 30(2):175-176.

435. Murphy MP, Wang H, Patel AN, Kambhampati S, Angle N, Chan K, Marleau AM, Pyszniak A, Carrier E, Ichim TE et al: Allogeneic endometrial regenerative cells: an “Off the shelf solution” for critical limb ischemia? J Transl Med 2008, 6:45.

436. Cui CH, Uyama T, Miyado K, Terai M, Kyo S, Kiyono T, Umezawa A: Menstrual blood-derived cells confer human dystrophin expression in the murine model of Duchenne muscular dystrophy via cell fusion and myogenic transdifferentiation. Mol Biol Cell 2007, 18(5):1586-1594.

437. Hida N, Nishiyama N, Miyoshi S, Kira S, Segawa K, Uyama T, Mori T, Miyado K, Ikegami Y, Cui C et al: Novel cardiac precursor-like cells from human menstrual blood-derived mesenchymal cells. Stem Cells 2008, 26(7):1695-1704.

438. Taguchi A, Soma T, Tanaka H, Kanda T, Nishimura H, Yoshikawa H, Tsukamoto Y, Iso H, Fujimori Y, Stern DM et al: Administration of CD34+ cells after stroke enhances neurogenesis via angiogenesis in a mouse model. J Clin Invest 2004, 114(3):330-338.

439. Borlongan CV, Kaneko Y, Maki M, Yu SJ, Ali M, Allickson JG, Sanberg CD, Kuzmin-Nichols N, Sanberg PR: Menstrual blood cells display stem cell-like phenotypic markers and exert neuroprotection following transplantation in experimental stroke. Stem Cells Dev 2010, 19(4):439-452.

440. Wolff EF, Gao XB, Yao KV, Andrews ZB, Du H, Elsworth JD, Taylor HS: Endometrial stem cell transplantation restores dopamine production in a Parkinson’s disease model. J Cell Mol Med 2011, 15(4):747-755.

441. Li HY, Chen YJ, Chen SJ, Kao CL, Tseng LM, Lo WL, Chang CM, Yang DM, Ku HH, Twu NF et al: Induction of insulin-producing cells derived from endometrial mesenchymal stem-like cells. J Pharmacol Exp Ther 2010, 335(3):817-829.

442. Han X, Meng X, Yin Z, Rogers A, Zhong J, Rillema P, Jackson JA, Ichim TE, Minev B, Carrier E et al: Inhibition of intracranial glioma growth by endometrial regenerative cells. Cell Cycle 2009, 8(4):606-610.

443. Bockeria L, Bogin V, Bockeria O, Le T, Alekyan B, Woods EJ, Brown AA, Ichim TE, Patel AN: Endometrial regenerative cells for treatment of heart failure: a new stem cell enters the clinic. Journal of translational medicine 2013, 11:56.

444. Herrera MB, Bussolati B, Bruno S, Fonsato V, Romanazzi GM, Camussi G: Mesenchymal stem cells contribute to the renal repair of acute tubular epithelial injury. Int J Mol Med 2004, 14(6):1035-1041.

445. Seebach C, Henrich D, Tewksbury R, Wilhelm K, Marzi I: Number and proliferative capacity of human mesenchymal stem cells are modulated positively in multiple trauma patients and negatively in atrophic nonunions. Calcif Tissue Int 2007, 80(4):294-300.

446. Ciulla MM, Ferrero S, Gianelli U, Paliotti R, Magrini F, Braidotti P: Direct visualization of neo-vessel formation following peripheral injection of bone marrow derived CD34+ cells in experimental myocardial damage. Micron 2007, 38(3):321-322.

447. Wojakowski W, Tendera M: Mobilization of bone marrow-derived progenitor cells in acute coronary syndromes. Folia Histochem Cytobiol 2005, 43(4):229-232.

448. Paczkowska E, Larysz B, Rzeuski R, Karbicka A, Jalowinski R, Kornacewicz-Jach Z, Ratajczak MZ, Machalinski B: Human hematopoietic stem/progenitor-enriched CD34(+) cells are mobilized into peripheral blood during stress related to ischemic stroke or acute myocardial infarction. Eur J Haematol 2005, 75(6):461-467.

449. Wang XY, Lan Y, He WY, Zhang L, Yao HY, Hou CM, Tong Y, Liu YL, Yang G, Liu XD et al: Identification of mesenchymal stem cells in aorta-gonad-mesonephros and yolk sac of human embryos. Blood 2008, 111(4):2436-2443.

450. Dexter TM: Stromal cell associated haemopoiesis. J Cell Physiol Suppl 1982, 1:87-94.

451. Bakhshi T, Zabriskie RC, Bodie S, Kidd S, Ramin S, Paganessi LA, Gregory SA, Fung HC, Christopherson KW, 2nd: Mesenchymal stem cells from the Wharton’s jelly of umbilical cord segments provide stromal support for the maintenance of cord blood hematopoietic stem cells during long-term ex vivo culture. Transfusion 2008.

452. Huang GP, Pan ZJ, Jia BB, Zheng Q, Xie CG, Gu JH, McNiece IK, Wang JF: Ex vivo expansion and transplantation of hematopoietic stem/progenitor cells supported by mesenchymal stem cells from human umbilical cord blood. Cell Transplant 2007, 16(6):579-585.

453. Urban VS, Kiss J, Kovacs J, Gocza E, Vas V, Monostori E, Uher F: Mesenchymal stem cells cooperate with bone marrow cells in therapy of diabetes. Stem Cells 2008, 26(1):244-253.

454. Ichim TE, Solano F, Brenes R, Glenn E, Chang J, Chan K, Riordan NH: Placental mesenchymal and cord blood stem cell therapy for dilated cardiomyopathy. Reproductive biomedicine online 2008, 16(6):898-905.

455. Guiducci S, Porta F, Saccardi R, Guidi S, Ibba-Manneschi L, Manetti M, Mazzanti B, Dal Pozzo S, Milia AF, Bellando-Randone S et al: Autologous mesenchymal stem cells foster revascularization of ischemic limbs in systemic sclerosis: a case report. Annals of internal medicine 2010, 153(10):650-654.

456. Lu D, Chen B, Liang Z, Deng W, Jiang Y, Li S, Xu J, Wu Q, Zhang Z, Xie B et al: Comparison of bone marrow mesenchymal stem cells with bone marrow-derived mononuclear cells for treatment of diabetic critical limb ischemia and foot ulcer: a double-blind, randomized, controlled trial. Diabetes research and clinical practice 2011, 92(1):26-36.

457. Gupta PK, Chullikana A, Parakh R, Desai S, Das A, Gottipamula S, Krishnamurthy S, Anthony N, Pherwani A, Majumdar AS: A double blind randomized placebo controlled phase I/II study assessing the safety and efficacy of allogeneic bone marrow derived mesenchymal stem cell in critical limb ischemia. Journal of translational medicine 2013, 11:143.

458. Bura A, Planat-Benard V, Bourin P, Silvestre JS, Gross F, Grolleau JL, Saint-Lebese B, Peyrafitte JA, Fleury S, Gadelorge M et al: Phase I trial: the use of autologous cultured adipose-derived stroma/stem cells to treat patients with non-revascularizable critical limb ischemia. Cytotherapy 2014, 16(2):245-257.

459. Lee HC, An SG, Lee HW, Park JS, Cha KS, Hong TJ, Park JH, Lee SY, Kim SP, Kim YD et al: Safety and effect of adipose tissue-derived stem cell implantation in patients with critical limb ischemia: a pilot study. Circulation journal : official journal of the Japanese Circulation Society 2012, 76(7):1750-1760.

460. Das AK, Bin Abdullah BJ, Dhillon SS, Vijanari A, Anoop CH, Gupta PK: Intra-arterial allogeneic mesenchymal stem cells for critical limb ischemia are safe and efficacious: report of a phase I study. World journal of surgery 2013, 37(4):915-922.

461. Yang SS, Kim NR, Park KB, Do YS, Roh K, Kang KS, Kim DI: A phase I study of human cord blood-derived mesenchymal stem cell therapy in patients with peripheral arterial occlusive disease. International journal of stem cells 2013, 6(1):37-44.

462. Kharaziha P, Hellstrom PM, Noorinayer B, Farzaneh F, Aghajani K, Jafari F, Telkabadi M, Atashi A, Honardoost M, Zali MR et al: Improvement of liver function in liver cirrhosis patients after autologous mesenchymal stem cell injection: a phase I-II clinical trial. European journal of gastroenterology & hepatology 2009, 21(10):1199-1205.

463. Peng L, Xie DY, Lin BL, Liu J, Zhu HP, Xie C, Zheng YB, Gao ZL: Autologous bone marrow mesenchymal stem cell transplantation in liver failure patients caused by hepatitis B: short-term and long-term outcomes. Hepatology 2011, 54(3):820-828.

464. El-Ansary M, Abdel-Aziz I, Mogawer S, Abdel-Hamid S, Hammam O, Teaema S, Wahdan M: Phase II trial: undifferentiated versus differentiated autologous mesenchymal stem cells transplantation in Egyptian patients with HCV induced liver cirrhosis. Stem cell reviews 2012, 8(3):972-981.

465. Kantarcioglu M, Demirci H, Avcu F, Karslioglu Y, Babayigit MA, Karaman B, Ozturk K, Gurel H, Akdogan Kayhan M, Kacar S et al: Efficacy of autologous mesenchymal stem cell transplantation in patients with liver cirrhosis. The Turkish journal of gastroenterology : the official journal of Turkish Society of Gastroenterology 2015, 26(3):244-250.

466. Salama H, Zekri AR, Medhat E, Al Alim SA, Ahmed OS, Bahnassy AA, Lotfy MM, Ahmed R, Musa S: Peripheral vein infusion of autologous mesenchymal stem cells in Egyptian HCV-positive patients with end-stage liver disease. Stem cell research & therapy 2014, 5(3):70.

467. Mohamadnejad M, Alimoghaddam K, Bagheri M, Ashrafi M, Abdollahzadeh L, Akhlaghpoor S, Bashtar M, Ghavamzadeh A, Malekzadeh R: Randomized placebo-controlled trial of mesenchymal stem cell transplantation in decompensated cirrhosis. Liver international : official journal of the International Association for the Study of the Liver 2013, 33(10):1490-1496.

468. Suk KT, Yoon JH, Kim MY, Kim CW, Kim JK, Park H, Hwang SG, Kim DJ, Lee BS, Lee SH et al: Transplantation with autologous bone marrow-derived mesenchymal stem cells for alcoholic cirrhosis: Phase 2 trial. Hepatology 2016.

469. Jang YO, Kim YJ, Baik SK, Kim MY, Eom YW, Cho MY, Park HJ, Park SY, Kim BR, Kim JW et al: Histological improvement following administration of autologous bone marrow-derived mesenchymal stem cells for alcoholic cirrhosis: a pilot study. Liver international : official journal of the International Association for the Study of the Liver 2014, 34(1):33-41.

470. Vosough M, Moossavi S, Mardpour S, Akhlaghpoor S, Azimian V, Jarughi N, Hosseini SE, Ashrafi M, Nikfam S, Aghdami N et al: Repeated Intraportal Injection of Mesenchymal Stem Cells in Combination with Pioglitazone in Patients with Compensated Cirrhosis: A Clinical Report of Two Cases. Archives of Iranian medicine 2016, 19(2):131-136.

471. Wang L, Li J, Liu H, Li Y, Fu J, Sun Y, Xu R, Lin H, Wang S, Lv S et al: Pilot study of umbilical cord-derived mesenchymal stem cell transfusion in patients with primary biliary cirrhosis. Journal of gastroenterology and hepatology 2013, 28 Suppl 1:85-92.

472. Wang L, Han Q, Chen H, Wang K, Shan GL, Kong F, Yang YJ, Li YZ, Zhang X, Dong F et al: Allogeneic bone marrow mesenchymal stem cell transplantation in patients with UDCA-resistant primary biliary cirrhosis. Stem cells and development 2014, 23(20):2482-2489.

473. Guo CH, Han LX, Wan MR, Deng GJ, Gan JH: Immunomodulatory effect of bone marrow mesenchymal stem cells on T lymphocytes in patients with decompensated liver cirrhosis. Genetics and molecular research : GMR 2015, 14(2):7039-7046.

474. Xu L, Gong Y, Wang B, Shi K, Hou Y, Wang L, Lin Z, Han Y, Lu L, Chen D et al: Randomized trial of autologous bone marrow mesenchymal stem cells transplantation for hepatitis B virus cirrhosis: regulation of Treg/Th17 cells. Journal of gastroenterology and hepatology 2014, 29(8):1620-1628.

475. Yoshikawa T, Ueda Y, Miyazaki K, Koizumi M, Takakura Y: Disc regeneration therapy using marrow mesenchymal cell transplantation: a report of two case studies. Spine 2010, 35(11):E475-480.

476. Orozco L, Soler R, Morera C, Alberca M, Sanchez A, Garcia-Sancho J: Intervertebral disc repair by autologous mesenchymal bone marrow cells: a pilot study. Transplantation 2011, 92(7):822-828.

477. Piccirilli M, Delfinis CP, Santoro A, Salvati M: Mesenchymal Stem cells (MSCs) in lumbar spine surgery: a single institution experience about red bone marrow and fat tissue derived MSCs. Clinico radiological remarks on a consecutive series of 22 patients. Journal of neurosurgical sciences 2015.

478. Elabd C, Centeno CJ, Schultz JR, Lutz G, Ichim T, Silva FJ: Intra-discal injection of autologous, hypoxic cultured bone marrow-derived mesenchymal stem cells in five patients with chronic lower back pain: a long-term safety and feasibility study. Journal of translational medicine 2016, 14:253.

479. Noriega DC, Ardura F, Hemandez-Ramajo R, Martin-Ferrero MA, Sanchez-Lite I, Toribio B, Alberca M, Garcia V, Moraleda JM, Sanchez A et al: Intervertebral disc repair by allogeneic mesenchymal bone marrow cells: a randomized controlled trial.T Transplantation 2016.

480. Pang X, Yang H, Peng B: Human umbilical cord mesenchymal stem cell transplantation for the treatment of chronic discogenic low back pain. Pain physician 2014, 17(4):E525-530.

481. McAnany SJ, Ahn J, Elboghdady IM, Marquez-Lara A, Ashraf N, Svovrlj B, Overley SC, Singh K, Qureshi SA: Mesenchymal stem cell allograft as a fusion adjunct in one- and two-level anterior cervical discectomy and fusion: a matched cohort analysis. The spine journal : official journal of the North American Spine Society 2016, 16(2): 163-167.

482. Mapel DW, Dutro MP, Marton JP, Woodruff K, Make B: Identifying and characterizing COPD patients in US managed care. A retrospective, cross-sectional analysis of administrative claims data. BMC health services research 2011, 11:43.

483. Ejiofor S, Turner AM: Pharmacotherapies for COPD. Clinical medicine insights Circulatory, respiratory and pulmonary medicine 2013, 7:17-34.

484. Miravitlles M, Sicras A, Crespo C, Cuesta M, Brosa M, Galera J, Lahoz R, Lleonart M, Riera MI: Costs of chronic obstructive pulmonary disease in relation to compliance with guidelines: a study in the primary care setting. Therapeutic advances in respiratory disease 2013, 7(3):139-150.

485. Martin-Rendon E, Brunskill SJ, Hyde CJ, Stanworth SJ, Mathur A, Watt SM: Autologous bone marrow stem cells to treat acute myocardial infarction: a systematic review. Eur Heart J 2008, 29(15):1807-1818.

486. www.vet-stem.com.

487. Riordan NH, Ichim TE, Min WP, Wang H, Solano F, Lara F, Alfaro M, Rodriguez JP, Harman RJ, Patel AN et al: Non-expanded adipose stromal vascular fraction cell therapy for multiple sclerosis. J Transl Med 2009, 7:29.

488. Holloway RA, Donnelly LE: Immunopathogenesis of chronic obstructive pulmonary disease. Current opinion in pulmonary medicine 2013, 19(2):95-102.

489. Motz GT, Eppert BL, Sun G, Wesselkamper SC, Linke MJ, Deka R, Borchers MT: Persistence of lung CD8 T cell oligoclonal expansions upon smoking cessation in a mouse model of cigarette smoke-induced emphysema. J Immunol 2008, 181(11):8036-8043.

490. Maeno T, Houghton AM, Quintero PA, Grumelli S, Owen CA, Shapiro SD: CD8+ T Cells are required for inflammation and destruction in cigarette smoke-induced emphysema in mice. J Immunol 2007, 178(12):8090-8096.

491. Woodruff PG, Koth LL, Yang YH, Rodriguez MW, Favoreto S, Dolganov GM, Paquet AC, Erle DJ: A distinctive alveolar macrophage activation state induced by cigarette smoking. Am JRespir Crit Care Med 2005, 172(11):1383-1392.

492. Stefanska AM, Walsh PT: Chronic obstructive pulmonary disease: evidence for an autoimmune component. Cell Mol Immunol 2009, 6(2):81-86.

493. Gonzalez-Rey E, Gonzalez MA, Varela N, O′Valle F, Hernandez-Cortes P, Rico L, Buscher D, Delgado M: Human adipose-derived mesenchymal stem cells reduce inflammatory and T cell responses and induce regulatory T cells in vitro in rheumatoid arthritis. Ann Rheum Dis, 69(1):241-248.

494. Lepelletier Y, Lecourt S, Arnulf B, Vanneaux V, Fermand JP, Menasche P, Domet T, Marolleau JP, Hermine O, Larghero J: Galectin-1 and Semaphorin-3A are two soluble factors conferring T cell immunosuppression to bone marrow mesenchymal stem cell. Stem Cells Dev 2009.

495. Tsyb AF, Petrov VN, Konoplyannikov AG, Saypina EV, Lepechina LA, Kalsina S, Semenkova IV, Agaeva EV: In vitro inhibitory effect of mesenchymal stem cells on zymosan-induced production of reactive oxygen species. Bulletin of experimental biology and medicine 2008, 146(1):158-164.

496. Sun L, Akiyama K, Zhang H, Yamaza T, Hou Y, Zhao S, Xu T, Le A, Shi S: Mesenchymal stem cell transplantation reverses multiorgan dysfunction in systemic lupus erythematosus mice and humans. Stem cells 2009, 27(6): 1421-1432.

497. Feuerer M, Herrero L, Cipolletta D, Naaz A, Wong J, Nayer A, Lee J, Goldfine AB, Benoist C, Shoelson S et al: Lean, but not obese, fat is enriched for a unique population of regulatory T cells that affect metabolic parameters. Nat Med 2009, 15(8):930-939.

498. Ogawa Y, Duru EA, Ameredes BT: Role of IL-10 in the resolution of airway inflammation. Curr Mol Med 2008, 8(5):437-445.

499. Serrano-Mollar A, Nacher M, Gay-Jordi G, Closa D, Xaubet A, Bulbena O: Intratracheal transplantation of alveolar type II cells reverses bleomycin-induced lung fibrosis. Am J Respir Crit Care Med 2007, 176(12):1261-1268.

500. Aslam M, Baveja R, Liang OD, Fernandez-Gonzalez A, Lee C, Mitsialis SA, Kourembanas S: Bone marrow stromal cells attenuate lung injury in a murine model of neonatal chronic lung disease. Am J Respir Crit Care Med 2009, 180(11):1122-1130.

501. van Haaften T, Byrne R, Bonnet S, Rochefort GY, Akabutu J, Bouchentouf M, Rey-Parra GJ, Galipeau J, Haromy A, Eaton F et al: Airway delivery of mesenchymal stem cells prevents arrested alveolar growth in neonatal lung injury in rats. Am J Respir Crit Care Med 2009, 180(11): 1131-1142.

502. Seimetz M, Parajuli N, Pichl A, Veit F, Kwapiszewska G, Weisel FC, Milger K, Egemnazarov B, Turowska A, Fuchs B et al: Inducible NOS inhibition reverses tobacco-smoke-induced emphysema and pulmonary hypertension in mice. Cell 2011, 147(2):293-305.

503. Iho S, Tanaka Y, Takauji R, Kobayashi C, Muramatsu I, Iwasaki H, Nakamura K, Sasaki Y, Nakao K, Takahashi T: Nicotine induces human neutrophils to produce IL-8 through the generation of peroxynitrite and subsequent activation of NF-kappaB. Journal of leukocyte biology 2003, 74(5):942-951.

504. Tanni SE, Pelegrino NR, Angeleli AY, Correa C, Godoy I: Smoking status and tumor necrosis factor-alpha mediated systemic inflammation in COPD patients. Journal of inflammation 2010, 7:29.

505. Weathington NM, van Houwelingen AH, Noerager BD, Jackson PL, Kraneveld AD, Galin FS, Folkerts G, Nijkamp FP, Blalock JE: A novel peptide CXCR ligand derived from extracellular matrix degradation during airway inflammation. Nature medicine 2006, 12(3):317-323.

506. Xu X, Jackson PL, Tanner S, Hardison MT, Abdul Roda M, Blalock JE, Gaggar A: A self-propagating matrix metalloprotease-9 (MMP-9) dependent cycle of chronic neutrophilic inflammation. PloS one 2011, 6(1):e15781.

507. Shoshani T, Faerman A, Mett I, Zelin E, Tenne T, Gorodin S, Moshel Y, Elbaz S, Budanov A, Chajut A et al: Identification of a novel hypoxia-inducible factor 1-responsive gene, RTP801, involved in apoptosis. Molecular and cellular biology 2002, 22(7):2283-2293.

508. Yoshida T, Mett I, Bhunia AK, Bowman J, Perez M, Zhang L, Gandjeva A, Zhen L, Chukwueke U, Mao T et al: Rtp801, a suppressor of mTOR signaling, is an essential mediator of cigarette smoke-induced pulmonary injury and emphysema. Nature medicine 2010, 16(7):767-773.

509. Kamocki K, Van Demark M, Fisher A, Rush NI, Presson RG, Jr., Hubbard W, Berdyshev EV, Adamsky S, Feinstein E, Gandjeva A et al: RTP801 is required for ceramide-induced cell-specific death in the murine lung. American journal of respiratory cell and molecular biology 2013, 48(1):87-93.

510. Buckley S, Shi W, Carraro G, Sedrakyan S, Da Sacco S, Driscoll BA, Perin L, De Filippo RE, Warburton D: The milieu of damaged alveolar epithelial type 2 cells stimulates alveolar wound repair by endogenous and exogenous progenitors. American journal of respiratory cell and molecular biology 2011, 45(6):1212-1221.

511. Medler TR, Petrusca DN, Lee PJ, Hubbard WC, Berdyshev EV, Skirball J, Kamocki K, Schuchman E, Tuder RM, Petrache I: Apoptotic sphingolipid signaling by ceramides in lung endothelial cells. American journal of respiratory cell and molecular biology 2008, 38(6):639-646.

512. Thon L, Mohlig H, Mathieu S, Lange A, Bulanova E, Winoto-Morbach S, Schutze S, Bulfone-Paus S, Adam D: Ceramide mediates caspase-independent programmed cell death. FASEB journal : official publication of the Federation of American Societies for Experimental Biology 2005, 19(14):1945-1956.

513. Chung KF: Cytokines as targets in chronic obstructive pulmonary disease. Current drug targets 2006, 7(6):675-681.

514. Tuder RM, Yoshida T: Stress responses affecting homeostasis of the alveolar capillary unit. Proceedings of the American Thoracic Society 2011, 8(6):485-491.

515. Sukkar MB, Xie S, Khorasani NM, Kon OM, Stanbridge R, Issa R, Chung KF: Toll-like receptor 2, 3, and 4 expression and function in human airway smooth muscle. The Journal of allergy and clinical immunology 2006, 118(3):641-648.

516. Pace E, Giarratano A, Ferraro M, Bruno A, Siena L, Mangione S, Johnson M, Gjomarkaj M: TLR4 upregulation underpins airway neutrophilia in smokers with chronic obstructive pulmonary disease and acute respiratory failure. Human immunology 2011, 72(1):54-62.

517. Speletas M, Merentiti V, Kostikas K, Liadaki K, Minas M, Gourgoulianis K, Germenis AE: Association of TLR4-T399I polymorphism with chronic obstructive pulmonary disease in smokers. Clinical & developmental immunology 2009, 2009:260286.

518. Wang J, Urbanowicz RA, Tighe PJ, Todd I, Corne JM, Fairclough LC: Differential activation of killer cells in the circulation and the lung: a study of current smoking status and chronic obstructive pulmonary disease (COPD). PloS one 2013, 8(3):e58556.

519. Tassi I, Klesney-Tait J, Colonna M: Dissecting natural killer cell activation pathways through analysis of genetic mutations in human and mouse. Immunological reviews 2006, 214:92-105.

520. Wortham BW, Eppert BL, Motz GT, Flury JL, Orozco-Levi M, Hoebe K, Panos RJ, Maxfield M, Glasser SW, Senft AP et al: NKG2D mediates NK cell hyperresponsiveness and influenza-induced pathologies in a mouse model of chronic obstructive pulmonary disease. Journal of immunology 2012, 188(9):4468-4475.

521. Borchers MT, Harris NL, Wesselkamper SC, Zhang S, Chen Y, Young L, Lau GW: The NKG2D-activating receptor mediates pulmonary clearance of Pseudomonas aeruginosa. Infection and immunity 2006, 74(5):2578-2586.

522. Borchers MT, Wesselkamper SC, Curull V, Ramirez-Sarmiento A, Sanchez-Font A, Garcia-Aymerich J, Coronell C, Lloreta J, Agusti AG, Gea J et al: Sustained CTL activation by murine pulmonary epithelial cells promotes the development of COPD-like disease. The Journal of clinical investigation 2009, 119(3):636-649.

523. Lie ML, White LE, Santora RJ, Park JM, Rabb H, Hassoun HT: Lung T lymphocyte trafficking and activation during ischemic acute kidney injury. Journal of immunology 2012, 189(6):2843-2851.

524. Shen X, Wang Y, Gao F, Ren F, Busuttil RW, Kupiec-Weglinski JW, Zhai Y: CD4 T cells promote tissue inflammation via CD40 signaling without de novo activation in a murine model of liver ischemia/reperfusion injury. Hepatology 2009, 50(5): 1537-1546.

525. Ichim CV: Revisiting immunosurveillance and immunostimulation: Implications for cancer immunotherapy. Journal of translational medicine 2005, 3(1):8.

526. Fraschini F, Scaglione F, Coppi G, Piazza G, Marchi E, Montoli CC, Scarpazza G: Some aspects of “deep lung” cellular immunity in chronic bronchitis before and after therapy with tiopronin. International journal of clinical pharmacology research 1987, 7(2): 129-133.

527. Mattoli S, Kleimberg J, Stacey MA, Bellini A, Sun G, Marini M: The role of CD8+ Th2 lymphocytes in the development of smoking-related lung damage. Biochemical and biophysical research communications 1997, 239(1):146-149.

528. Cosio MG, Guerassimov A: Chronic obstructive pulmonary disease. Inflammation of small airways and lung parenchyma. American journal of respiratory and critical care medicine 1999, 160(5 Pt 2):S21-25.

529. Stankiewicz W, Dabrowski MP, Chcialowski A, Plusa T: Cellular and cytokine immunoregulation in patients with chronic obstructive pulmonary disease and bronchial asthma. Mediators of inflammation 2002, 11(5):307-312.

530. Agostini C, Trentin L, Adami F: Chronic obstructive pulmonary disease (COPD): new insights on the events leading to pulmonary inflammation. Sarcoidosis, vasculitis, and diffuse lung diseases : official journal of WASOG / World Association of Sarcoidosis and Other Granulomatous Disorders 2003, 20(1):3-7.

531. Glader P, von Wachenfeldt K, Lofdahl CG: Systemic CD4+ T-cell activation is correlated with FEV1 in smokers. Respiratory medicine 2006, 100(6):1088-1093.

532. Di Stefano A, Caramori G, Capelli A, Gnemmi I, Ricciardolo FL, Oates T, Donner CF, Chung KF, Barnes PJ, Adcock IM: STAT4 activation in smokers and patients with chronic obstructive pulmonary disease. The European respiratory journal : official journal of the European Society for Clinical Respiratory Physiology 2004, 24(1):78-85.

533. Roos-Engstrand E, Pourazar J, Behndig AF, Blomberg A, Bucht A: Cytotoxic T cells expressing the co-stimulatory receptor NKG2 D are increased in cigarette smoking and COPD. Respiratory research 2010, 11:128.

534. Hou J, Sun Y, Hao Y, Zhuo J, Liu X, Bai P, Han J, Zheng X, Zeng H: Imbalance between subpopulations of regulatory T cells in COPD. Thorax 2013.

535. Wang H, Peng W, Weng Y, Ying H, Li H, Xia D, Yu W: Imbalance of Th17/Treg cells in mice with chronic cigarette smoke exposure. International immunopharmacology 2012, 14(4):504-512.

536. Eppert BL, Wortham BW, Flury JL, Borchers MT: Functional characterization of T cell populations in a mouse model of chronic obstructive pulmonary disease. Journal of immunology 2013, 190(3):1331-1340.

537. Weiss DJ, Casaburi R, Flannery R, LeRoux-Williams M, Tashkin DP: A placebo-controlled, randomized trial of mesenchymal stem cells in COPD. Chest 2013, 143(6): 1590-1598.

538. Stolk J, Broekman W, Mauad T, Zwaginga JJ, Roelofs H, Fibbe WE, Oostendorp J, Bajema I, Versteegh MI, Taube C et al: A phase I study for intravenous autologous mesenchymal stromal cell administration to patients with severe emphysema. QJM : monthly journal of the Association of Physicians 2016, 109(5):331-336.

539. Chang YS, Ahn SY, Yoo HS, Sung SI, Choi SJ, Oh WI, Park WS: Mesenchymal stem cells for bronchopulmonary dysplasia: phase 1 dose-escalation clinical trial. The Journal of pediatrics 2014, 164(5):966-972 e966.

540. Chang Y, Park SH, Huh JW, Lim CM, Koh Y, Hong SB: Intratracheal administration of umbilical cord blood-derived mesenchymal stem cells in a patient with acute respiratory distress syndrome. J Korean Med Sci 2014, 29(3):438-440.

541. Zheng G, Huang L, Tong H, Shu Q, Hu Y, Ge M, Deng K, Zhang L, Zou B, Cheng B et al: Treatment of acute respiratory distress syndrome with allogeneic adipose-derived mesenchymal stem cells: a randomized, placebo-controlled pilot study. Respir Res 2014, 15:39.

542. Wilson JG, Liu KD, Zhuo H, Caballero L, McMillan M, Fang X, Cosgrove K, Vojnik R, Calfee CS, Lee JW et al: Mesenchymal stem (stromal) cells for treatment of ARDS: a phase 1 clinical trial. The Lancet Respiratory medicine 2015, 3(1):24-32.

543. Simonson OE, Mougiakakos D, Heldring N, Bassi G, Johansson HJ, Dalen M, Jitschin R, Rodin S, Corbascio M, El Andaloussi S et al: In Vivo Effects of Mesenchymal Stromal Cells in Two Patients With Severe Acute Respiratory Distress Syndrome. Stem cells translational medicine 2015, 4(10): 1199-1213.

544. Thangakunam B, Christopher DJ, Mathews V, Srivastava A: Mesenchymal stromal stem cell therapy in advanced interstitial lung disease - Anaphylaxis and short-term follow-up. Lung India : official organ of Indian Chest Society 2015, 32(5):486-488.

545. Chambers DC, Enever D, Ilic N, Sparks L, Whitelaw K, Ayres J, Yerkovich ST, Khalil D, Atkinson KM, Hopkins PM: A phase 1b study of placenta-derived mesenchymal stromal cells in patients with idiopathic pulmonary fibrosis. Respirology 2014, 19(7):1013-1018.

546. Baughman RP, Culver DA, Jankovi V, Fischkoff S, Brockway G, Lower EE: Placenta-derived mesenchymal-like cells (PDA-001) as therapy for chronic pulmonary sarcoidosis: a phase 1 study. Sarcoidosis, vasculitis, and diffuse lung diseases : official journal of WASOG / World Association of Sarcoidosis and Other Granulomatous Disorders 2015, 32(2): 106-114.

547. Skrahin A, Jenkins HE, Hurevich H, Solodovnikova V, Isaikina Y, Klimuk D, Rohava Z, Skrahina A: Effectiveness of a novel cellular therapy to treat multidrug-resistant tuberculosis. Journal of clinical tuberculosis and other mycobacterial diseases 2016, 4:21-27.

548. Skrahin A, Ahmed RK, Ferrara G, Rane L, Poiret T, Isaikina Y, Skrahina A, Zumla A, Maeurer MJ: Autologous mesenchymal stromal cell infusion as adjunct treatment in patients with multidrug and extensively drug-resistant tuberculosis: an open-label phase 1 safety trial. The Lancet Respiratory medicine 2014, 2(2): 108-122.

549. Carrion F, Nova E, Ruiz C, Diaz F, Inostroza C, Rojo D, Monckeberg G, Figueroa FE: Autologous mesenchymal stem cell treatment increased T regulatory cells with no effect on disease activity in two systemic lupus erythematosus patients. Lupus 2010, 19(3):317-322.

550. Sun L, Wang D, Liang J, Zhang H, Feng X, Wang H, Hua B, Liu B, Ye S, Hu X et al: Umbilical cord mesenchymal stem cell transplantation in severe and refractory systemic lupus erythematosus. Arthritis and rheumatism 2010, 62(8):2467-2475.

551. Shi D, Wang D, Li X, Zhang H, Che N, Lu Z, Sun L: Allogeneic transplantation of umbilical cord-derived mesenchymal stem cells for diffuse alveolar hemorrhage in systemic lupus erythematosus. Clinical rheumatology 2012, 31(5):841-846.

552. Wang D, Akiyama K, Zhang H, Yamaza T, Li X, Feng X, Wang H, Hua B, Liu B, Xu H et al: Double allogenic mesenchymal stem cells transplantations could not enhance therapeutic effect compared with single transplantation in systemic lupus erythematosus. Clinical & developmental immunology 2012, 2012:273291.

553. Wang D, Zhang H, Liang J, Li X, Feng X, Wang H, Hua B, Liu B, Lu L, Gilkeson GS et al: Allogeneic mesenchymal stem cell transplantation in severe and refractory systemic lupus erythematosus: 4 years of experience. Cell transplantation 2013, 22(12):2267-2277.

554. Wang D, Li J, Zhang Y, Zhang M, Chen J, Li X, Hu X, Jiang S, Shi S, Sun L: Umbilical cord mesenchymal stem cell transplantation in active and refractory systemic lupus erythematosus: a multicenter clinical study. Arthritis research & therapy 2014, 16(2):R79.

555. Gu F, Wang D, Zhang H, Feng X, Gilkeson GS, Shi S, Sun L: Allogeneic mesenchymal stem cell transplantation for lupus nephritis patients refractory to conventional therapy. Clinical rheumatology 2014, 33(11):1611-1619.

556. Wang D, Niu L, Feng X, Yuan X, Zhao S, Zhang H, Liang J, Zhao C, Wang H, Hua B et al: Long-term safety of umbilical cord mesenchymal stem cells transplantation for systemic lupus erythematosus: a 6-year follow-up study. Clinical and experimental medicine 2016.

557. Wang D, Huang S, Yuan X, Liang J, Xu R, Yao G, Feng X, Sun L: The regulation of the Treg/Th17 balance by mesenchymal stem cells in human systemic lupus erythematosus. Cellular & molecular immunology 2015.

558. Rosati G: The prevalence of multiple sclerosis in the world: an update. Neurol Sci 2001, 22(2): 117-139.

559. Pittock SJ, Lucchinetti CF: The pathology of MS: new insights and potential clinical applications. Neurologist 2007, 13(2):45-56.

560. Saresella M, Marventano I, Longhi R, Lissoni F, Trabattoni D, Mendozzi L, Caputo D, Clerici M: CD4+CD25+FoxP3+PD1- regulatory T cells in acute and stable relapsing-remitting multiple sclerosis and their modulation by therapy. FASEB J 2008, 22(10):3500-3508.

561. Korporal M, Haas J, Balint B, Fritzsching B, Schwarz A, Moeller S, Fritz B, Suri-Payer E, Wildemann B: Interferon beta-induced restoration of regulatory T-cell function in multiple sclerosis is prompted by an increase in newly generated naive regulatory T cells. Arch Neurol 2008, 65(11):1434-1439.

562. Akirav EM, Bergman CM, Hill M, Ruddle NH: Depletion of CD4(+)CD25(+) T cells exacerbates experimental autoimmune encephalomyelitis induced by mouse, but not rat, antigens. J Neurosci Res 2009.

563. Reddy J, Illes Z, Zhang X, Encinas J, Pyrdol J, Nicholson L, Sobel RA, Wucherpfennig KW, Kuchroo VK: Myelin proteolipid protein-specific CD4+CD25+ regulatory cells mediate genetic resistance to experimental autoimmune encephalomyelitis. Proc Natl Acad Sci U S A 2004, 101(43):15434-15439.

564. Gregg C, Shikar V, Larsen P, Mak G, Chojnacki A, Yong VW, Weiss S: White matter plasticity and enhanced remyelination in the maternal CNS. J Neurosci 2007, 27(8): 1812-1823.

565. Penner IK, Kappos L, Rausch M, Opwis K, Radu EW: Therapy-induced plasticity of cognitive functions in MS patients: insights from fMRI. J Physiol Paris 2006, 99(4-6):455-462.

566. Nait-Oumesmar B, Picard-Riera N, Kerninon C, Decker L, Seilhean D, Hoglinger GU, Hirsch EC, Reynolds R, Baron-Van Evercooren A: Activation of the subventricular zone in multiple sclerosis: evidence for early glial progenitors. Proc Natl Acad Sci U S A 2007, 104(11):4694-4699.

567. Zappia E, Casazza S, Pedemonte E, Benvenuto F, Bonanni I, Gerdoni E, Giunti D, Ceravolo A, Cazzanti F, Frassoni F et al: Mesenchymal stem cells ameliorate experimental autoimmune encephalomyelitis inducing T-cell anergy. Blood 2005, 106(5):1755-1761.

568. Bai L, Lennon DP, Eaton V, Maier K, Caplan AI, Miller SD, Miller RH: Human bone marrow-derived mesenchymal stem cells induce Th2-polarized immune response and promote endogenous repair in animal models of multiple sclerosis. Glia 2009.

569. Mohyeddin Bonab M, Yazdanbakhsh S, Lotfi J, Alimoghaddom K, Talebian F, Hooshmand F, Ghavamzadeh A, Nikbin B: Does mesenchymal stem cell therapy help multiple sclerosis patients? Report of a pilot study. Iranian journal of immunology : IJI 2007, 4(1):50-57.

570. Yamout B, Hourani R, Salti H, Barada W, El-Hajj T, Al-Kutoubi A, Herlopian A, Baz EK, Mahfouz R, Khalil-Hamdan R et al: Bone marrow mesenchymal stem cell transplantation in patients with multiple sclerosis: a pilot study. Journal of neuroimmunology 2010, 227(1-2): 185-189.

571. Karussis D, Karageorgiou C, Vaknin-Dembinsky A, Gowda-Kurkalli B, Gomori JM, Kassis I, Bulte JW, Petrou P, Ben-Hur T, Abramsky O et al: Safety and immunological effects of mesenchymal stem cell transplantation in patients with multiple sclerosis and amyotrophic lateral sclerosis. Archives of neurology 2010, 67(10):1187-1194.

572. Connick P, Kolappan M, Crawley C, Webber DJ, Patani R, Michell AW, Du MQ, Luan SL, Altmann DR, Thompson AJ et al: Autologous mesenchymal stem cells for the treatment of secondary progressive multiple sclerosis: an open-label phase 2a proof-of-concept study. The Lancet Neurology 2012, 11(2): 150-156.

573. Bonab MM, Sahraian MA, Aghsaie A, Karvigh SA, Hosseinian SM, Nikbin B, Lotfi J, Khorramnia S, Motamed MR, Togha M et al: Autologous mesenchymal stem cell therapy in progressive multiple sclerosis: an open label study. Current stem cell research & therapy 2012, 7(6):407-414.

574. Llufriu S, Sepulveda M, Blanco Y, Marin P, Moreno B, Berenguer J, Gabilondo I, Martinez-Heras E, Sola-Valls N, Arnaiz JA et al: Randomized placebo-controlled phase II trial of autologous mesenchymal stem cells in multiple sclerosis. PloS one 2014, 9(12):e113936.

575. Fu Y, Yan Y, Qi Y, Yang L, Li T, Zhang N, Yu C, Su L, Zhang R, Shen Y et al: Impact of Autologous Mesenchymal Stem Cell Infusion on Neuromyelitis Optica Spectrum Disorder: A Pilot, 2-Year Observational Study. CNS neuroscience & therapeutics 2016, 22(8):677-685.

576. Dulamea AO, Sirbu-Boeti MP, Bleotu C, Dragu D, Moldovan L, Lupescu I, Comi G: Autologous mesenchymal stem cells applied on the pressure ulcers had produced a surprising outcome in a severe case of neuromyelitis optica. Neural regeneration research 2015, 10(11): 1841-1845.

577. Hou ZL, Liu Y, Mao XH, Wei CY, Meng MY, Liu YH, Zhuyun Yang Z, Zhu H, Short M, Bernard C et al: Transplantation of umbilical cord and bone marrow-derived mesenchymal stem cells in a patient with relapsing-remitting multiple sclerosis. Cell adhesion & migration 2013, 7(5):404-407.

578. Li JF, Zhang DJ, Geng T, Chen L, Huang H, Yin HL, Zhang YZ, Lou JY, Cao B, Wang YL: The potential of human umbilical cord-derived mesenchymal stem cells as a novel cellular therapy for multiple sclerosis. Cell transplantation 2014, 23 Suppl 1:S113-122.

579. Lublin FD, Bowen JD, Huddlestone J, Kremenchutzky M, Carpenter A, Corboy JR, Freedman MS, Krupp L, Paulo C, Hariri RJ et al: Human placenta-derived cells (PDA-001) for the treatment of adults with multiple sclerosis: a randomized, placebo-controlled, multiple-dose study. Multiple sclerosis and related disorders 2014, 3(6):696-704.

580. FIRESTEIN GEaPoRAIHR, Edward D.;, Genovese MCF, Gary S.; Sargent, John S.; Sledge, Clement B., editors. Kelley’s, Textbook of Rheumatology. Vol. 7. Philadelphia P, USA: Elsevier Saunders; 2005. p. 996-1042.

581. Sun S, Rao NL, Venable J, Thurmond R, Karlsson L: TLR7/9 antagonists as therapeutics for immune-mediated inflammatory disorders. Inflamm Allergy Drug Targets 2007, 6(4):223-235.

582. Chong AS, Huang W, Liu W, Luo J, Shen J, Xu W, Ma L, Blinder L, Xiao F, Xu X et al: In vivo activity of leflunomide: pharmacokinetic analyses and mechanism of immunosuppression. Transplantation 1999, 68(1): 100-109.

583. Dimitrova P, Skapenko A, Herrmann ML, Schleyerbach R, Kalden JR, Schulze-Koops H: Restriction of de novo pyrimidine biosynthesis inhibits Th1 cell activation and promotes Th2 cell differentiation. J Immunol 2002, 169(6):3392-3399.

584. Kirsch BM, Zeyda M, Stuhlmeier K, Grisar J, Smolen JS, Watschinger B, Stulnig TM, Horl WH, Zlabinger GJ, Saemann MD: The active metabolite of leflunomide, A77 1726, interferes with dendritic cell function. Arthritis Res Ther 2005, 7(3):R694-703.

585. Tepperman K, Roy PW, Moloney BF, Elder RC: Dicyanogold effects on lymphokine production. Met Based Drugs 1999, 6(4-5):301-309.

586. Han S, Kim K, Song Y, Kim H, Kwon J, Lee YH, Lee CK, Lee SJ, Ha N: Auranofin, an immunosuppressive drug, inhibits MHC class I and MHC class II pathways of antigen presentation in dendritic cells. Arch Pharm Res 2008, 31(3):370-376.

587. Kim TS, Kang BY, Lee MH, Choe YK, Hwang SY: Inhibition of interleukin-12 production by auranofin, an anti-rheumatic gold compound, deviates CD4(+) T cells from the Th1 to the Th2 pathway. Br J Pharmacol 2001, 134(3):571-578.

588. Taggart AJ: Sulphasalazine in arthritis--an old drug rediscovered. Clin Rheumatol 1987, 6(3):378-383.

589. Bansard C, Lequerre T, Daveau M, Boyer O, Tron F, Salier JP, Vittecoq O, Le-Loet X: Can rheumatoid arthritis responsiveness to methotrexate and biologics be predicted? Rheumatology (Oxford) 2009, 48(9):1021-1028.

590. Bijlsma JW, Hoes JN, Van Everdingen AA, Verstappen SM, Jacobs JW: Are glucocorticoids DMARDs? Ann N Y Acad Sci 2006, 1069:268-274.

591. Nanda S, Bathon JM: Etanercept: a clinical review of current and emerging indications. Expert Opin Pharmacother 2004, 5(5): 1175-1186.

592. Shakoor N, Michalska M, Harris CA, Block JA: Drug-induced systemic lupus erythematosus associated with etanercept therapy. Lancet 2002, 359(9306):579-580.

593. Dinarello CA: Anti-cytokine therapeutics and infections. Vaccine 2003, 21 Suppl 2:S24-34.

594. Trentham DE, Townes AS, Kang AH: Autoimmunity to type II collagen an experimental model of arthritis. J Exp Med 1977, 146(3):857-868.

595. Trentham DE, Dynesius RA, David JR: Passive transfer by cells of type II collagen-induced arthritis in rats. J Clin Invest 1978, 62(2):359-366.

596. Londei M, Savill CM, Verhoef A, Brennan F, Leech ZA, Duance V, Maini RN, Feldmann M: Persistence of collagen type II-specific T-cell clones in the synovial membrane of a patient with rheumatoid arthritis. Proc Natl Acad Sci U S A 1989, 86(2):636-640.

597. Sekine T, Kato T, Masuko-Hongo K, Nakamura H, Yoshino S, Nishioka K, Yamamoto K: Type II collagen is a target antigen of clonally expanded T cells in the synovium of patients with rheumatoid arthritis. Ann Rheum Dis 1999, 58(7):446-450.

598. Trentham DE, Dynesius-Trentham RA, Orav EJ, Combitchi D, Lorenzo C, Sewell KL, Hafler DA, Weiner HL: Effects of oral administration of type II collagen on rheumatoid arthritis. Science 1993, 261(5129):1727-1730.

599. http://www.autoimmuneinc.com/clinic/coll.html.

600. Min WP, Zhou D, Ichim TE, Xia X, Zhang X, Yang J, Huang X, Garcia B, Dutartre P, Jevnikar AM et al: Synergistic tolerance induced by LF15-0195 and anti-CD45RB monoclonal antibody through suppressive dendritic cells. Transplantation 2003, 75(8): 1160-1165.

601. Min WP, Zhou D, T.E. I, Strejan GH, X. X, Yang J, X H, Garcia B, White DJ, Dutartre P et al: Inhibitory feedback loop between tolerogenic dendritic cells and regularoty T cells in transplant tolerance. J Immunol 2003, 170:1304-1312.

602. Yang J, Bernier SM, Ichim TE, Li M, Xia X, Zhou D, Huang X, Strejan GH, White DJ, Zhong R et al: LF15-0195 generates tolerogenic dendritic cells by suppression of NF-kappaB signaling through inhibition of IKK activity. J Leukoc Biol 2003, 74(3):438-447.

603. Min WP, Gorczynski R, Huang XY, Kushida M, Kim P, Obataki M, Lei J, Suri RM, Cattral MS: Dendritic cells genetically engineered to express Fas ligand induce donor-specific hyporesponsiveness and prolong allograft survival. J Immunol 2000, 164(1):161-167.

604. Gainer AL, Suarez-Pinzon WL, Min WP, Swiston JR, Hancock-Friesen C, Korbutt GS, Rajotte RV, Warnock GL, Elliott JF: Improved survival of biolistically transfected mouse islet allografts expressing CTLA4-Ig or soluble Fas ligand. Transplantation 1998, 66(2): 194-199.

605. Ichim TE, Li M, Qian H, Popov IA, Rycerz K, Zheng X, White D, Zhong R, Min WP: RNA interference: a potent tool for gene-specific therapeutics. Am J Transplant 2004, 4(8): 1227-1236.

606. Ichim TE, Zhong R, Min WP: Prevention of allograft rejection by in vitro generated tolerogenic dendritic cells. Transplant immunology 2003, 11(3-4):295-306.

607. Hill JA, Ichim TE, Kusznieruk KP, Li M, Huang X, Yan X, Zhong R, Cairns E, Bell DA, Min WP: Immune modulation by silencing IL-12 production in dendritic cells using small interfering RNA. J Immunol 2003, 171(2):691-696.

608. Li M, Qian H, Ichim TE, Ge WW, Popov IA, Rycerz K, Neu J, White D, Zhong R, Min WP: Induction of RNA interference in dendritic cells. Immunol Res 2004, 30(2):215-230.

609. Nasef A, Zhang YZ, Mazurier C, Bouchet S, Bensidhoum M, Francois S, Gorin NC, Lopez M, Thierry D, Fouillard L et al: Selected Stro-1-enriched bone marrow stromal cells display a major suppressive effect on lymphocyte proliferation. Int J Lab Hematol 2009, 31(1):9-19.

610. Renner P, Eggenhofer E, Rosenauer A, Popp FC, Steinmann JF, Slowik P, Geissler EK, Piso P, Schlitt HJ, Dahlke MH: Mesenchymal stem cells require a sufficient, ongoing immune response to exert their immunosuppressive function. Transplant Proc 2009, 41(6):2607-2611.

611. Lisheng W, Meng E, Guo Z: High mobility group box 1 protein inhibits the proliferation of human mesenchymal stem cells and promotes their migration and differentiation along osteoblastic pathway. Stem Cells Dev 2008.

612. Kitaori T, Ito H, Schwarz EM, Tsutsumi R, Yoshitomi H, Oishi S, Nakano M, Fujii N, Nagasawa T, Nakamura T: Stromal cell-derived factor ⅟CXCR4 signaling is critical for the recruitment of mesenchymal stem cells to the fracture site during skeletal repair in a mouse model. Arthritis Rheum 2009, 60(3):813-823.

613. Gunzberg WH, Salmons B: Stem cell therapies: on track but suffer setback. Curr Opin Mol Ther 2009, 11(4):360-363.

614. Dryden GW: Overview of stem cell therapy for Crohn’s disease. Expert Opin Biol Ther 2009, 9(7):841-847.

615. Richardson SM, Hoyland JA, Mobasheri R, Csaki C, Shakibaei M, Mobasheri A: Mesenchymal stem cells in regenerative medicine: opportunities and challenges for articular cartilage and intervertebral disc tissue engineering. J Cell Physiol, 222(1):23-32.

616. Bouffi C, Djouad F, Mathieu M, Noel D, Jorgensen C: Multipotent mesenchymal stromal cells and rheumatoid arthritis: risk or benefit? Rheumatology (Oxford) 2009, 48(10):1185-1189.

617. Nakagawa S, Toritsuka Y, Wakitani S, Denno K, Tomita T, Owaki H, Kimura T, Shino K, Ochi T: Bone marrow stromal cells contribute to synovial cell proliferation in rats with collagen induced arthritis. J Rheumatol 1996, 23(12):2098-2103.

618. Ochi T, Yoshikawa H, Toyosaki-Maeda T, Lipsky PE: Mesenchymal stromal cells. Nurse-like cells reside in the synovial tissue and bone marrow in rheumatoid arthritis. Arthritis Res Ther 2007, 9(1):201.

619. Kastrinaki MC, Sidiropoulos P, Roche S, Ringe J, Lehmann S, Kritikos H, Vlahava VM, Delorme B, Eliopoulos GD, Jorgensen C et al: Functional, molecular and proteomic characterisation of bone marrow mesenchymal stem cells in rheumatoid arthritis. Ann Rheum Dis 2008, 67(6):741-749.

620. Papadaki HA, Kritikos HD, Gemetzi C, Koutala H, Marsh JC, Boumpas DT, Eliopoulos GD: Bone marrow progenitor cell reserve and function and stromal cell function are defective in rheumatoid arthritis: evidence for a tumor necrosis factor alpha-mediated effect. Blood 2002, 99(5): 1610-1619.

621. Djouad F, Fritz V, Apparailly F, Louis-Plence P, Bony C, Sany J, Jorgensen C, Noel D: Reversal of the immunosuppressive properties of mesenchymal stem cells by tumor necrosis factor alpha in collagen-induced arthritis. Arthritis Rheum 2005, 52(5): 1595-1603.

622. Mao F, Xu WR, Qian H, Zhu W, Yan YM, Shao QX, Xu HX: Immunosuppressive effects of mesenchymal stem cells in collagen-induced mouse arthritis. Inflamm Res 2009.

623. Gonzalez MA, Gonzalez-Rey E, Rico L, Buscher D, Delgado M: Treatment of experimental arthritis by inducing immune tolerance with human adipose-derived mesenchymal stem cells. Arthritis Rheum 2009, 60(4): 1006-1019.

624. Zheng ZH, Li XY, Ding J, Jia JF, Zhu P: Allogeneic mesenchymal stem cell and mesenchymal stem cell-differentiated chondrocyte suppress the responses of type II collagen-reactive T cells in rheumatoid arthritis. Rheumatology (Oxford) 2008, 47(1):22-30.

625. Karussis D, Kassis I: The potential use of stem cells in multiple sclerosis: an overview of the preclinical experience. Clin Neurol Neurosurg 2008, 110(9):889-896.

626. Boumaza I, Srinivasan S, Witt WT, Feghali-Bostwick C, Dai Y, Garcia-Ocana A, Feili-Hariri M: Autologous bone marrow-derived rat mesenchymal stem cells promote PDX-1 and insulin expression in the islets, alter T cell cytokine pattern and preserve regulatory T cells in the periphery and induce sustained normoglycemia. J Autoimmun 2008.

627. Zhou K, Zhang H, Jin O, Feng X, Yao G, Hou Y, Sun L: Transplantation of human bone marrow mesenchymal stem cell ameliorates the autoimmune pathogenesis in MRL/lpr mice. Cell Mol Immunol 2008, 5(6):417-424.

628. Liang J, Li X, Zhang H, Wang D, Feng X, Wang H, Hua B, Liu B, Sun L: Allogeneic mesenchymal stem cells transplantation in patients with refractory RA. Clinical rheumatology 2012, 31(1):157-161.

629. Wang L, Wang L, Cong X, Liu G, Zhou J, Bai B, Li Y, Bai W, Li M, Ji H et al: Human umbilical cord mesenchymal stem cell therapy for patients with active rheumatoid arthritis: safety and efficacy. Stem cells and development 2013, 22(24):3192-3202.

630. Wang L, Zhang Y, Li H, Hong J, Chen X, Li M, Bai W, Wang J, Liu Y, Wu M: Clinical Observation of Employment of Umbilical Cord Derived Mesenchymal Stem Cell for Juvenile Idiopathic Arthritis Therapy. Stem cells international 2016, 2016:9165267.

631. Alvaro-Gracia JM, Jover JA, Garcia-Vicuna R, Carreno L, Alonso A, Marsal S, Blanco F, Martinez-Taboada VM, Taylor P, Martin-Martin C et al: Intravenous administration of expanded allogeneic adipose-derived mesenchymal stem cells in refractory rheumatoid arthritis (Cx611): results of a multicentre, dose escalation, randomised, single-blind, placebo-controlled phase Ib/IIa clinical trial. Annals of the rheumatic diseases 2016.

632. Bang OY, Lee JS, Lee PH, Lee G: Autologous mesenchymal stem cell transplantation in stroke patients. Annals of neurology 2005, 57(6):874-882.

633. Lee JS, Hong JM, Moon GJ, Lee PH, Ahn YH, Bang OY, collaborators S: A long-term follow-up study of intravenous autologous mesenchymal stem cell transplantation in patients with ischemic stroke. Stem cells 2010, 28(6): 1099-1106.

634. Bhasin A, Srivastava MV, Kumaran SS, Mohanty S, Bhatia R, Bose S, Gaikwad S, Garg A, Airan B: Autologous mesenchymal stem cells in chronic stroke. Cerebrovascular diseases extra 2011, 1(1):93-104.

635. Honmou O, Houkin K, Matsunaga T, Niitsu Y, Ishiai S, Onodera R, Waxman SG, Kocsis JD: Intravenous administration of auto serum-expanded autologous mesenchymal stem cells in stroke. Brain : a journal of neurology 2011, 134(Pt 6): 1790-1807.

636. Han H, Chang SK, Chang JJ, Hwang SH, Han SH, Chun BH: Intrathecal injection of human umbilical cord blood-derived mesenchymal stem cells for the treatment of basilar artery dissection: a case report. Journal of medical case reports 2011, 5:562.

637. Qiao LY, Huang FJ, Zhao M, Xie JH, Shi J, Wang J, Lin XZ, Zuo H, Wang YL, Geng TC: A two-year follow-up study of cotransplantation with neural stem/progenitor cells and mesenchymal stromal cells in ischemic stroke patients. Cell transplantation 2014, 23 Suppl 1:S65-72.

638. Steinberg GK, Kondziolka D, Wechsler LR, Lunsford LD, Coburn ML, Billigen JB, Kim AS, Johnson JN, Bates D, King B et al: Clinical Outcomes of Transplanted Modified Bone Marrow-Derived Mesenchymal Stem Cells in Stroke: A Phase ½ a Study. Stroke; a journal of cerebral circulation 2016, 47(7):1817-1824.

639. Hogan P, Dall T, Nikolov P, American Diabetes A: Economic costs of diabetes in the US in 2002. Diabetes care 2003, 26(3):917-932.

640. Barker JM, Goehrig SH, Barriga K, Hoffman M, Slover R, Eisenbarth GS, Norris JM, Klingensmith GJ, Rewers M, study D: Clinical characteristics of children diagnosed with type 1 diabetes through intensive screening and follow-up. Diabetes care 2004, 27(6): 1399-1404.

641. Pitkaniemi J, Onkamo P, Tuomilehto J, Arjas E: Increasing incidence of Type 1 diabetes--role for genes? BMC genetics 2004, 5:5.

642. van Belle TL, Coppieters KT, von Herrath MG: Type 1 diabetes: etiology, immunology, and therapeutic strategies. Physiological reviews 2011, 91(1):79-118.

643. Eisenbarth GS: Update in type 1 diabetes. The Journal of clinical endocrinology and metabolism 2007, 92(7):2403-2407.

644. Meloche RM: Transplantation for the treatment of type 1 diabetes. World journal of gastroenterology : WJG 2007, 13(47):6347-6355.

645. Couri CE, Oliveira MC, Stracieri AB, Moraes DA, Pieroni F, Barros GM, Madeira MI, Malmegrim KC, Foss-Freitas MC, Simoes BP et al: C-peptide levels and insulin independence following autologous nonmyeloablative hematopoietic stem cell transplantation in newly diagnosed type 1 diabetes mellitus. JAMA : the journal of the American Medical Association 2009, 301(15):1573-1579.

646. Friedenstein AJ, Chailakhyan RK, Latsinik NV, Panasyuk AF, Keiliss-Borok IV: Stromal cells responsible for transferring the microenvironment of the hemopoietic tissues. Cloning in vitro and retransplantation in vivo. Transplantation 1974, 17(4):331-340.

647. Sugiyama T, Nagasawa T: Bone marrow niches for hematopoietic stem cells and immune cells. Inflammation & allergy drug targets 2012, 11(3):201-206.

648. Lalu MM, McIntyre L, Pugliese C, Fergusson D, Winston BW, Marshall JC, Granton J, Stewart DJ, Canadian Critical Care Trials G: Safety of cell therapy with mesenchymal stromal cells (SafeCell): a systematic review and meta-analysis of clinical trials. PloS one 2012, 7(10):e47559.

649. Djouad F, Charbonnier LM, Bouffi C, Louis-Plence P, Bony C, Apparailly F, Cantos C, Jorgensen C, Noel D: Mesenchymal stem cells inhibit the differentiation of dendritic cells through an interleukin-6-dependent mechanism. Stem cells 2007, 25(8):2025-2032.

650. English K, Barry FP, Mahon BP: Murine mesenchymal stem cells suppress dendritic cell migration, maturation and antigen presentation. Immunology letters 2008, 115(1):50-58.

651. Nemeth K, Leelahavanichkul A, Yuen PS, Mayer B, Parmelee A, Doi K, Robey PG, Leelahavanichkul K, Koller BH, Brown JM et al: Bone marrow stromal cells attenuate sepsis via prostaglandin E(2)-dependent reprogramming of host macrophages to increase their interleukin-10 production. Nature medicine 2009, 15(1):42-49.

652. Liang J, Zhang H, Hua B, Wang H, Lu L, Shi S, Hou Y, Zeng X, Gilkeson GS, Sun L: Allogenic mesenchymal stem cells transplantation in refractory systemic lupus erythematosus: a pilot clinical study. Annals of the rheumatic diseases 2010, 69(8):1423-1429.

653. Hu J, Yu X, Wang Z, Wang F, Wang L, Gao H, Chen Y, Zhao W, Jia Z, Yan S et al: Long term effects of the implantation of Wharton’s jelly-derived mesenchymal stem cells from the umbilical cord for newly-onset type 1 diabetes mellitus. Endocrine journal 2012.

654. Xu J, Wang D, Liu D, Fan Z, Zhang H, Liu O, Ding G, Gao R, Zhang C, Ding Y et al: Allogeneic mesenchymal stem cell treatment alleviates experimental and clinical Sjogren syndrome. Blood 2012, 120(15):3142-3151.

655. Bodansky HJ, Staines A, Stephenson C, Haigh D, Cartwright R: Evidence for an environmental effect in the aetiology of insulin dependent diabetes in a transmigratory population. Bmj 1992, 304(6833):1020-1022.

656. Gillespie KM, Bain SC, Barnett AH, Bingley PJ, Christie MR, Gill GV, Gale EA: The rising incidence of childhood type 1 diabetes and reduced contribution of high-risk HLA haplotypes. Lancet 2004, 364(9446):1699-1700.

657. Wenzlau JM, Juhl K, Yu L, Moua O, Sarkar SA, Gottlieb P, Rewers M, Eisenbarth GS, Jensen J, Davidson HW et al: The cation efflux transporter ZnT8 (Slc30A8) is a major autoantigen in human type 1 diabetes. Proceedings of the National Academy of Sciences of the United States of America 2007, 104(43): 17040-17045.

658. Bottazzo GF, Dean BM, McNally JM, MacKay EH, Swift PG, Gamble DR: In situ characterization of autoimmune phenomena and expression of HLA molecules in the pancreas in diabetic insulitis. The New England journal of medicine 1985, 313(6):353-360.

659. Hu CY, Rodriguez-Pinto D, Du W, Ahuja A, Henegariu O, Wong FS, Shlomchik MJ, Wen L: Treatment with CD20-specific antibody prevents and reverses autoimmune diabetes in mice. The Journal of clinical investigation 2007, 117(12):3857-3867.

660. Xiu Y, Wong CP, Bouaziz JD, Hamaguchi Y, Wang Y, Pop SM, Tisch RM, Tedder TF: B lymphocyte depletion by CD20 monoclonal antibody prevents diabetes in nonobese diabetic mice despite isotype-specific differences in Fc gamma R effector functions. Journal of immunology 2008, 180(5):2863-2875.

661. Pescovitz MD, Greenbaum CJ, Krause-Steinrauf H, Becker DJ, Gitelman SE, Goland R, Gottlieb PA, Marks JB, McGee PF, Moran AM et al: Rituximab, B-lymphocyte depletion, and preservation of beta-cell function. The New England journal of medicine 2009, 361(22):2143-2152.

662. Ziegler AG, Erhard J, Lampeter EF, Nagelkerken LM, Standl E: Involvement of dendritic cells in early insulitis of BB rats. Journal of autoimmunity 1992, 5(5):571-579.

663. Jansen A, Homo-Delarche F, Hooijkaas H, Leenen PJ, Dardenne M, Drexhage HA: Immunohistochemical characterization of monocytes-macrophages and dendritic cells involved in the initiation of the insulitis and beta-cell destruction in NOD mice. Diabetes 1994, 43(5):667-675.

664. Turley S, Poirot L, Hattori M, Benoist C, Mathis D: Physiological beta cell death triggers priming of self-reactive T cells by dendritic cells in a type-1 diabetes model. The Journal of experimental medicine 2003, 198(10):1527-1537.

665. Hamilton-Williams EE, Palmer SE, Charlton B, Slattery RM: Beta cell MHC class I is a late requirement for diabetes. Proceedings of the National Academy of Sciences of the United States of America 2003, 100(11):6688-6693.

666. Serreze DV, Leiter EH, Christianson GJ, Greiner D, Roopenian DC: Major histocompatibility complex class I-deficient NOD-B2mnull mice are diabetes and insulitis resistant. Diabetes 1994, 43(3):505-509.

667. Di Lorenzo TP, Peakman M, Roep BO: Translational mini-review series on type 1 diabetes: Systematic analysis of T cell epitopes in autoimmune diabetes. Clinical and experimental immunology 2007, 148(1): 1-16.

668. Skowera A, Ellis RJ, Varela-Calvino R, Arif S, Huang GC, Van-Krinks C, Zaremba A, Rackham C, Allen JS, Tree TI et al: CTLs are targeted to kill beta cells in patients with type 1 diabetes through recognition of a glucose-regulated preproinsulin epitope. The Journal of clinical investigation 2008, 118(10):3390-3402.

669. Panagiotopoulos C, Qin H, Tan R, Verchere CB: Identification of a beta-cell-specific HLA class I restricted epitope in type 1 diabetes. Diabetes 2003, 52(11):2647-2651.

670. Panina-Bordignon P, Lang R, van Endert PM, Benazzi E, Felix AM, Pastore RM, Spinas GA, Sinigaglia F: Cytotoxic T cells specific for glutamic acid decarboxylase in autoimmune diabetes. The Journal of experimental medicine 1995, 181(5):1923-1927.

671. Stiller CR, Dupre J, Gent M, Jenner MR, Keown PA, Laupacis A, Martell R, Rodger NW, von Graffenried B, Wolfe BM: Effects of cyclosporine immunosuppression in insulin-dependent diabetes mellitus of recent onset. Science 1984, 223(4643): 1362-1367.

672. Feutren G, Papoz L, Assan R, Vialettes B, Karsenty G, Vexiau P, Du Rostu H, Rodier M, Sirmai J, Lallemand A et al: Cyclosporin increases the rate and length of remissions in insulin-dependent diabetes of recent onset. Results of a multicentre double-blind trial. Lancet 1986, 2(8499):119-124.

673. Martin S, Schernthaner G, Nerup J, Gries FA, Koivisto VA, Dupre J, Standl E, Hamet P, McArthur R, Tan MH et al: Follow-up of cyclosporin A treatment in type 1 (insulin-dependent) diabetes mellitus: lack of long-term effects. Diabetologia 1991, 34(6):429-434.

674. Orban T, Bundy B, Becker DJ, DiMeglio LA, Gitelman SE, Goland R, Gottlieb PA, Greenbaum CJ, Marks JB, Monzavi R et al: Co-stimulation modulation with abatacept in patients with recent-onset type 1 diabetes: a randomised, double-blind, placebo-controlled trial. Lancet 2011, 378(9789):412-419.

675. Herold KC, Gitelman SE, Masharani U, Hagopian W, Bisikirska B, Donaldson D, Rother K, Diamond B, Harlan DM, Bluestone JA: A single course of anti-CD3 monoclonal antibody hOKT3gamma1(Ala-Ala) results in improvement in C-peptide responses and clinical parameters for at least 2 years after onset of type 1 diabetes. Diabetes 2005, 54(6): 1763-1769.

676. Keymeulen B, Vandemeulebroucke E, Ziegler AG, Mathieu C, Kaufman L, Hale G, Gorus F, Goldman M, Walter M, Candon S et al: Insulin needs after CD3-antibody therapy in new-onset type 1 diabetes. The New England journal of medicine 2005, 352(25):2598-2608.

677. Sherry N, Hagopian W, Ludvigsson J, Jain SM, Wahlen J, Ferry RJ, Jr., Bode B, Aronoff S, Holland C, Carlin D et al: Teplizumab for treatment of type 1 diabetes (Protege study): 1-year results from a randomised, placebo-controlled trial. Lancet 2011, 378(9790):487-497.

678. Staeva TP, Chatenoud L, Insel R, Atkinson MA: Recent lessons learned from prevention and recent-onset type 1 diabetes immunotherapy trials. Diabetes 2013, 62(1): 9-17.

679. Keymeulen B, Candon S, Fafi-Kremer S, Ziegler A, Leruez-Ville M, Mathieu C, Vandemeulebroucke E, Walter M, Crenier L, Thervet E et al: Transient Epstein-Barr virus reactivation in CD3 monoclonal antibody-treated patients. Blood 2010, 115(6): 1145-1155.

680. Moran A, Bundy B, Becker DJ, DiMeglio LA, Gitelman SE, Goland R, Greenbaum CJ, Herold KC, Marks JB, Raskin P et al: Interleukin-1 antagonism in type 1 diabetes of recent onset: two multicentre, randomised, double-blind, placebo-controlled trials. Lancet 2013, 381(9881):1905-1915.

681. Park KS, Park MJ, Cho ML, Kwok SK, Ju JH, Ko HJ, Park SH, Kim HY: Type II collagen oral tolerance; mechanism and role in collagen-induced arthritis and rheumatoid arthritis. Mod Rheumatol 2009.

682. Womer KL, Magee CC, Najafian N, Vella JP, Milford EL, Sayegh MH, Carpenter CB: A pilot study on the immunological effects of oral administration of donor major histocompatibility complex class II peptides in renal transplant recipients. Clin Transplant 2008, 22(6):754-759.

683. Faria AM, Weiner HL: Oral tolerance: therapeutic implications for autoimmune diseases. Clin Dev Immunol 2006, 13(2-4): 143-157.

684. Thompson HS, Harper N, Bevan DJ, Staines NA: Suppression of collagen induced arthritis by oral administration of type II collagen: changes in immune and arthritic responses mediated by active peripheral suppression. Autoimmunity 1993, 16(3): 189-199.

685. Song F, Gienapp IE, Shawler T, Guan Z, Whitacre CC: The thymus plays a role in oral tolerance induction in experimental autoimmune encephalomyelitis. Ann N Y Acad Sci 2004, 1029:402-404.

686. Hanninen A, Harrison LC: Mucosal tolerance to prevent type 1 diabetes: can the outcome be improved in humans? Rev Diabet Stud 2004, 1(3):113-121.

687. Streilein JW, Niederkorn JY: Characterization of the suppressor cell(s) responsible for anterior chamber-associated immune deviation (ACAID) induced in BALB/c mice by P815 cells. J Immunol 1985, 134(3): 1381-1387.

688. Katagiri K, Zhang-Hoover J, Mo JS, Stein-Streilein J, Streilein JW: Using tolerance induced via the anterior chamber of the eye to inhibit Th2-dependent pulmonary pathology. J Immunol 2002, 169(1):84-89.

689. Miller A, Lider O, Weiner HL: Antigen-driven bystander suppression after oral administration of antigens. The Journal of experimental medicine 1991, 174(4):791-798.

690. Tian J, Lehmann PV, Kaufman DL: Determinant spreading of T helper cell 2 (Th2) responses to pancreatic islet autoantigens. The Journal of experimental medicine 1997, 186(12):2039-2043.

691. Ludvigsson J, Krisky D, Casas R, Battelino T, Castano L, Greening J, Kordonouri O, Otonkoski T, Pozzilli P, Robert JJ et al: GAD65 antigen therapy in recently diagnosed type 1 diabetes mellitus. The New England journal of medicine 2012, 366(5):433-442.

692. Wherrett DK, Bundy B, Becker DJ, DiMeglio LA, Gitelman SE, Goland R, Gottlieb PA, Greenbaum CJ, Herold KC, Marks JB et al: Antigen-based therapy with glutamic acid decarboxylase (GAD) vaccine in patients with recent-onset type 1 diabetes: a randomised double-blind trial. Lancet 2011, 378(9788):319-327.

693. Prockop DJ: Marrow stromal cells as stem cells for nonhematopoietic tissues. Science 1997, 276(5309):71-74.

694. Friedenstein AJ, Petrakova KV, Kurolesova AI, Frolova GP: Heterotopic of bone marrow.Analysis of precursor cells for osteogenic and hematopoietic tissues. Transplantation 1968, 6(2):230-247.

695. Horwitz EM, Gordon PL, Koo WK, Marx JC, Neel MD, McNall RY, Muul L, Hofmann T: Isolated allogeneic bone marrow-derived mesenchymal cells engraft and stimulate growth in children with osteogenesis imperfecta: Implications for cell therapy of bone. Proc Natl Acad Sci USA 2002, 99(13):8932-8937.

696. Koc ON, Day J, Nieder M, Gerson SL, Lazarus HM, Krivit W: Allogeneic mesenchymal stem cell infusion for treatment of metachromatic leukodystrophy (MLD) and Hurler syndrome (MPS-IH). Bone Marrow Transplant 2002, 30(4):215-222.

697. Le Blanc K, Samuelsson H, Gustafsson B, Remberger M, Sundberg B, Arvidson J, Ljungman P, Lonnies H, Nava S, Ringden O: Transplantation of mesenchymal stem cells to enhance engraftment of hematopoietic stem cells. Leukemia 2007, 21(8):1733-1738.

698. Lazarus HM, Koc ON, Devine SM, Curtin P, Maziarz RT, Holland HK, Shpall EJ, McCarthy P, Atkinson K, Cooper BW et al: Cotransplantation of HLA-identical sibling culture-expanded mesenchymal stem cells and hematopoietic stem cells in hematologic malignancy patients. Biol Blood Marrow Transplant 2005, 11(5):389-398.

699. Ball LM, Bernardo ME, Roelofs H, Lankester A, Cometa A, Egeler RM, Locatelli F, Fibbe WE: Cotransplantation of ex vivo expanded mesenchymal stem cells accelerates lymphocyte recovery and may reduce the risk of graft failure in haploidentical hematopoietic stem-cell transplantation. Blood 2007, 110(7):2764-2767.

700. Steinman RM, Hawiger D, Nussenzweig MC: Tolerogenic dendritic cells. Annual review of immunology 2003, 21:685-711.

701. Adema GJ: Dendritic cells from bench to bedside and back. Immunology letters 2009, 122(2): 128-130.

702. Steinman RM, Inaba K: Myeloid dendritic cells. Journal of leukocyte biology 1999, 66(2):205-208.

703. Steinman RM: Dendritic cells and the control of immunity: enhancing the efficiency of antigen presentation. The Mount Sinai journal of medicine, New York 2001, 68(3):160-166.

704. Bonifaz L, Bonnyay D, Mahnke K, Rivera M, Nussenzweig MC, Steinman RM: Efficient targeting of protein antigen to the dendritic cell receptor DEC-205 in the steady state leads to antigen presentation on major histocompatibility complex class I products and peripheral CD8+ T cell tolerance. The Journal of experimental medicine 2002, 196(12):1627-1638.

705. Mukhopadhaya A, Hanafusa T, Jarchum I, Chen YG, Iwai Y, Serreze DV, Steinman RM, Tarbell KV, DiLorenzo TP: Selective delivery of beta cell antigen to dendritic cells in vivo leads to deletion and tolerance of autoreactive CD8+ T cells in NOD mice. Proceedings of the National Academy of Sciences of the United States of America 2008, 105(17):6374-6379.

706. Giannoukakis N, Phillips B, Finegold D, Hamaha J, Trucco M: Phase I (safety) study of autologous tolerogenic dendritic cells in type 1 diabetic patients. Diabetes care 2011, 34(9):2026-2032.

707. Krampera M, Glennie S, Dyson J, Scott D, Laylor R, Simpson E, Dazzi F: Bone marrow mesenchymal stem cells inhibit the response of naive and memory antigen-specific T cells to their cognate peptide. Blood 2003, 101(9):3722-3729.

708. Zhang W, Ge W, Li C, You S, Liao L, Han Q, Deng W, Zhao RC: Effects of mesenchymal stem cells on differentiation, maturation, and function of human monocyte-derived dendritic cells. Stem cells and development 2004, 13(3):263-271.

709. Beyth S, Borovsky Z, Mevorach D, Liebergall M, Gazit Z, Aslan H, Galun E, Rachmilewitz J: Human mesenchymal stem cells alter antigen-presenting cell maturation and induce T-cell unresponsiveness. Blood 2005, 105(5):2214-2219.

710. Jiang XX, Zhang Y, Liu B, Zhang SX, Wu Y, Yu XD, Mao N: Human mesenchymal stem cells inhibit differentiation and function of monocyte-derived dendritic cells. Blood 2005, 105(10):4120-4126.

711. Fibbe WE, Nauta AJ, Roelofs H: Modulation of immune responses by mesenchymal stem cells. Annals of the New York Academy of Sciences 2007, 1106:272-278.

712. Jung YJ, Ju SY, Yoo ES, Cho S, Cho KA, Woo SY, Seoh JY, Park JW, Han HS, Ryu KH: MSC-DC interactions: MSC inhibit maturation and migration of BM-derived DC. Cytotherapy 2007, 9(5):451-458.

713. Chen L, Zhang W, Yue H, Han Q, Chen B, Shi M, Li J, Li B, You S, Shi Y et al: Effects of human mesenchymal stem cells on the differentiation of dendritic cells from CD34+ cells. Stem cells and development 2007, 16(5):719-731.

714. Wang Q, Sun B, Wang D, Ji Y, Kong Q, Wang G, Wang J, Zhao W, Jin L, Li H: Murine bone marrow mesenchymal stem cells cause mature dendritic cells to promote T-cell tolerance. Scandinavian journal of immunology 2008, 68(6):607-615.

715. Oh JY, Lee RH, Yu JM, Ko JH, Lee HJ, Ko AY, Roddy GW, Prockop DJ: Intravenous mesenchymal stem cells prevented rejection of allogeneic corneal transplants by aborting the early inflammatory response. Molecular therapy : the journal of the American Society of Gene Therapy 2012, 20(11):2143-2152.

716. Li YP, Paczesny S, Lauret E, Poirault S, Bordigoni P, Mekhloufi F, Hequet O, Bertrand Y, Ou-Yang JP, Stoltz JF et al: Human mesenchymal stem cells license adult CD34+ hemopoietic progenitor cells to differentiate into regulatory dendritic cells through activation of the Notch pathway. Journal of immunology 2008, 180(3): 1598-1608.

717. Chiesa S, Morbelli S, Morando S, Massollo M, Marini C, Bertoni A, Frassoni F, Bartolome ST, Sambuceti G, Traggiai E et al: Mesenchymal stem cells impair in vivo T-cell priming by dendritic cells. Proceedings of the National Academy of Sciences of the United States of America 2011, 108(42):17384-17389.

718. Li H, Guo Z, Jiang X, Zhu H, Li X, Mao N: Mesenchymal stem cells alter migratory property of T and dendritic cells to delay the development of murine lethal acute graft-versus-host disease. Stem cells 2008, 26(10):2531-2541.

719. Opitz CA, Litzenburger UM, Lutz C, Lanz TV, Tritschler I, Koppel A, Tolosa E, Hoberg M, Anderl J, Aicher WK et al: Toll-like receptor engagement enhances the immunosuppressive properties of human bone marrow-derived mesenchymal stem cells by inducing indoleamine-2,3-dioxygenase-1 via interferon-beta and protein kinase R. Stem cells 2009, 27(4):909-919.

720. Fan H, Zhao G, Liu L, Liu F, Gong W, Liu X, Yang L, Wang J, Hou Y: Pre-treatment with IL-1beta enhances the efficacy of MSC transplantation in DSS-induced colitis. Cellular & molecular immunology 2012, 9(6):473-481.

721. Duijvestein M, Wildenberg ME, Welling MM, Hennink S, Molendijk I, van Zuylen VL, Bosse T, Vos AC, de Jonge-Muller ES, Roelofs H et al: Pretreatment with interferon-gamma enhances the therapeutic activity of mesenchymal stromal cells in animal models of colitis. Stem cells 2011, 29(10):1549-1558.

722. Krampera M, Cosmi L, Angeli R, Pasini A, Liotta F, Andreini A, Santarlasci V, Mazzinghi B, Pizzolo G, Vinante F et al: Role for interferon-gamma in the immunomodulatory activity of human bone marrow mesenchymal stem cells. Stem cells 2006, 24(2):386-398.

723. Polchert D, Sobinsky J, Douglas G, Kidd M, Moadsiri A, Reina E, Genrich K, Mehrotra S, Setty S, Smith B et al: IFN-gamma activation of mesenchymal stem cells for treatment and prevention of graft versus host disease. European journal of immunology 2008, 38(6):1745-1755.

724. Groh ME, Maitra B, Szekely E, Koc ON: Human mesenchymal stem cells require monocyte-mediated activation to suppress alloreactive T cells. Experimental hematology 2005, 33(8):928-934.

725. Bartholomew A, Sturgeon C, Siatskas M, Ferrer K, McIntosh K, Patil S, Hardy W, Devine S, Ucker D, Deans R et al: Mesenchymal stem cells suppress lymphocyte proliferation in vitro and prolong skin graft survival in vivo. Experimental hematology 2002, 30(1):42-48.

726. Beggs KJ, Lyubimov A, Borneman JN, Bartholomew A, Moseley A, Dodds R, Archambault MP, Smith AK, McIntosh KR: Immunologic consequences of multiple, high-dose administration of allogeneic mesenchymal stem cells to baboons. Cell transplantation 2006, 15(8-9):711-721.

727. Tse WT, Pendleton JD, Beyer WM, Egalka MC, Guinan EC: Suppression of allogeneic T-cell proliferation by human marrow stromal cells: implications in transplantation. Transplantation 2003, 75(3):389-397.

728. Maitra B, Szekely E, Gjini K, Laughlin MJ, Dennis J, Haynesworth SE, Koc ON: Human mesenchymal stem cells support unrelated donor hematopoietic stem cells and suppress T-cell activation. Bone marrow transplantation 2004, 33(6):597-604.

729. Rasmusson I, Ringden O, Sundberg B, Le Blanc K: Mesenchymal stem cells inhibit the formation of cytotoxic T lymphocytes, but not activated cytotoxic T lymphocytes or natural killer cells. Transplantation 2003, 76(8): 1208-1213.

730. Angoulvant D, Clerc A, Benchalal S, Galambrun C, Farre A, Bertrand Y, Eljaafari A: Human mesenchymal stem cells suppress induction of cytotoxic response to alloantigens. Biorheology 2004, 41(3-4):469-476.

731. Le Blanc K, Rasmusson I, Gotherstrom C, Seidel C, Sundberg B, Sundin M, Rosendahl K, Tammik C, Ringden O: Mesenchymal stem cells inhibit the expression of CD25 (interleukin-2 receptor) and CD38 on phytohaemagglutinin-activated lymphocytes. Scandinavian journal of immunology 2004, 60(3):307-315.

732. Glennie S, Soeiro I, Dyson PJ, Lam EW, Dazzi F: Bone marrow mesenchymal stem cells induce division arrest anergy of activated T cells. Blood 2005, 105(7):2821-2827.

733. Kim JA, Hong S, Lee B, Hong JW, Kwak JY, Cho S, Kim CC: The inhibition of T-cells proliferation by mouse mesenchymal stem cells through the induction of p16INK4A-cyclin D1/cdk4 and p21waf1, p27kip1-cyclin E/cdk2 pathways. Cellular immunology 2007, 245(1):16-23.

734. Plumas J, Chaperot L, Richard MJ, Molens JP, Bensa JC, Favrot MC: Mesenchymal stem cells induce apoptosis of activated T cells. Leukemia 2005, 19(9): 1597-1604.

735. Lim JH, Kim JS, Yoon IH, Shin JS, Nam HY, Yang SH, Kim SJ, Park CG: Immunomodulation of delayed-type hypersensitivity responses by mesenchymal stem cells is associated with bystander T cell apoptosis in the draining lymph node. Journal of immunology 2010, 185(7):4022-4029.

736. Rasmusson I, Ringden O, Sundberg B, Le Blanc K: Mesenchymal stem cells inhibit lymphocyte proliferation by mitogens and alloantigens by different mechanisms. Experimental cell research 2005, 305(1):33-41.

737. Xu G, Zhang L, Ren G, Yuan Z, Zhang Y, Zhao RC, Shi Y: Immunosuppressive properties of cloned bone marrow mesenchymal stem cells. Cell research 2007, 17(3):240-248.

738. English K, Ryan JM, Tobin L, Murphy MJ, Barry FP, Mahon BP: Cell contact, prostaglandin E(2) and transforming growth factor beta 1 play non-redundant roles in human mesenchymal stem cell induction of CD4+CD25(High) forkhead box P3+ regulatory T cells. Clinical and experimental immunology 2009, 156(1): 149-160.

739. Spaggiari GM, Abdelrazik H, Becchetti F, Moretta L: MSCs inhibit monocyte-derived DC maturation and function by selectively interfering with the generation of immature DCs: central role of MSC-derived prostaglandin E2. Blood 2009, 113(26):6576-6583.

740. Yanez R, Oviedo A, Aldea M, Bueren JA, Lamana ML: Prostaglandin E2 plays a key role in the immunosuppressive properties of adipose and bone marrow tissue-derived mesenchymal stromal cells. Experimental cell research 2010, 316(19):3109-3123.

741. Zafranskaya M, Nizheharodava D, Yurkevich M, Ivanchik G, Demidchik Y, Kozhukh H, Fedulov A: PGE2 Contributes to In vitro MSC-Mediated Inhibition of Non-Specific and Antigen-Specific T Cell Proliferation in MS Patients. Scandinavian journal of immunology 2013, 78(5):455-462.

742. Magatti M, De Munari S, Vertua E, Gibelli L, Wengler GS, Parolini O: Human amnion mesenchyme harbors cells with allogeneic T-cell suppression and stimulation capabilities. Stem cells 2008, 26(1): 182-192.

743. El Haddad N, Heathcote D, Moore R, Yang S, Azzi J, Mfarrej B, Atkinson M, Sayegh MH, Lee JS, Ashton-Rickardt PG et al: Mesenchymal stem cells express serine protease inhibitor to evade the host immune response. Blood 2011, 117(4): 1176-1183.

744. Sato K, Ozaki K, Oh I, Meguro A, Hatanaka K, Nagai T, Muroi K, Ozawa K: Nitric oxide plays a critical role in suppression of T-cell proliferation by mesenchymal stem cells. Blood 2007, 109(1):228-234.

745. Oh I, Ozaki K, Sato K, Meguro A, Tatara R, Hatanaka K, Nagai T, Muroi K, Ozawa K: Interferon-gamma and NF-kappaB mediate nitric oxide production by mesenchymal stromal cells. Biochemical and biophysical research communications 2007, 355(4):956-962.

746. Ren G, Zhang L, Zhao X, Xu G, Zhang Y, Roberts AI, Zhao RC, Shi Y: Mesenchymal stem cell-mediated immunosuppression occurs via concerted action of chemokines and nitric oxide. Cell stem cell 2008, 2(2): 141-150.

747. DelaRosa O, Lombardo E, Beraza A, Mancheno-Corvo P, Ramirez C, Menta R, Rico L, Camarillo E, Garcia L, Abad JL et al: Requirement of IFN-gamma-mediated indoleamine 2,3-dioxygenase expression in the modulation of lymphocyte proliferation by human adipose-derived stem cells. Tissue engineering Part A 2009, 15(10):2795-2806.

748. Tipnis S, Viswanathan C, Majumdar AS: Immunosuppressive properties of human umbilical cord-derived mesenchymal stem cells: role of B7-H1 and IDO. Immunology and cell biology 2010, 88(8):795-806.

749. Ge W, Jiang J, Arp J, Liu W, Garcia B, Wang H: Regulatory T-cell generation and kidney allograft tolerance induced by mesenchymal stem cells associated with indoleamine 2,3-dioxygenase expression. Transplantation 2010, 90(12):1312-1320.

750. Francois M, Romieu-Mourez R, Li M, Galipeau J: Human MSC suppression correlates with cytokine induction of indoleamine 2,3-dioxygenase and bystander M2 macrophage differentiation. Molecular therapy : the journal of the American Society of Gene Therapy 2012, 20(1): 187-195.

751. Sattler C, Steinsdoerfer M, Offers M, Fischer E, Schierl R, Heseler K, Daubener W, Seissler J: Inhibition of T-cell proliferation by murine multipotent mesenchymal stromal cells is mediated by CD39 expression and adenosine generation. Cell transplantation 2011, 20(8):1221-1230.

752. Saldanha-Araujo F, Ferreira FI, Palma PV, Araujo AG, Queiroz RH, Covas DT, Zago MA, Panepucci RA: Mesenchymal stromal cells up-regulate CD39 and increase adenosine production to suppress activated T-lymphocytes. Stem cell research 2011, 7(1):66-74.

753. Xue Q, Luan XY, Gu YZ, Wu HY, Zhang GB, Yu GH, Zhu HT, Wang M, Dong W, Geng YJ et al: The negative co-signaling molecule b7-h4 is expressed by human bone marrow-derived mesenchymal stem cells and mediates its T-cell modulatory activity. Stem cells and development 2010, 19(1):27-38.

754. Gieseke F, Bohringer J, Bussolari R, Dominici M, Handgretinger R, Muller I: Human multipotent mesenchymal stromal cells use galectin-1 to inhibit immune effector cells. Blood 2010, 116(19):3770-3779.

755. Chabannes D, Hill M, Merieau E, Rossignol J, Brion R, Soulillou JP, Anegon I, Cuturi MC: A role for heme oxygenase-1 in the immunosuppressive effect of adult rat and human mesenchymal stem cells. Blood 2007, 110(10):3691-3694.

756. Mougiakakos D, Jitschin R, Johansson CC, Okita R, Kiessling R, Le Blanc K: The impact of inflammatory licensing on heme oxygenase-1-mediated induction of regulatory T cells by human mesenchymal stem cells. Blood 2011, 117(18):4826-4835.

757. Augello A, Tasso R, Negrini SM, Amateis A, Indiveri F, Cancedda R, Pennesi G: Bone marrow mesenchymal progenitor cells inhibit lymphocyte proliferation by activation of the programmed death 1 pathway. European journal of immunology 2005, 35(5):1482-1490.

758. Sheng H, Wang Y, Jin Y, Zhang Q, Zhang Y, Wang L, Shen B, Yin S, Liu W, Cui L et al: A critical role of IFNgamma in priming MSC-mediated suppression of T cell proliferation through up-regulation of B7-H1. Cell research 2008, 18(8):846-857.

759. Luz-Crawford P, Noel D, Fernandez X, Khoury M, Figueroa F, Carrion F, Jorgensen C, Djouad F: Mesenchymal stem cells repress Th17 molecular program through the PD-1 pathway. PloS one 2012, 7(9):e45272.

760. Akiyama K, Chen C, Wang D, Xu X, Qu C, Yamaza T, Cai T, Chen W, Sun L, Shi S: Mesenchymal-stem-cell-induced immunoregulation involves FAS-ligand-/FAS-mediated T cell apoptosis. Cell stem cell 2012, 10(5):544-555.

761. Gu YZ, Xue Q, Chen YJ, Yu GH, Qing MD, Shen Y, Wang MY, Shi Q, Zhang XG: Different roles of PD-L1 and FasL in immunomodulation mediated by human placenta-derived mesenchymal stem cells. Human immunology 2013, 74(3):267-276.

762. Najar M, Raicevic G, Jebbawi F, De Bruyn C, Meuleman N, Bron D, Toungouz M, Lagneaux L: Characterization and functionality of the CD200-CD200R system during mesenchymal stromal cell interactions with T-lymphocytes. Immunology letters 2012, 146(1-2):50-56.

763. Batten P, Sarathchandra P, Antoniw JW, Tay SS, Lowdell MW, Taylor PM, Yacoub MH: Human mesenchymal stem cells induce T cell anergy and downregulate T cell allo-responses via the TH2 pathway: relevance to tissue engineering human heart valves. Tissue engineering 2006, 12(8):2263-2273.

764. Lu X, Liu T, Gu L, Huang C, Zhu H, Meng W, Xi Y, Li S, Liu Y: Immunomodulatory effects of mesenchymal stem cells involved in favoring type 2 T cell subsets. Transplant immunology 2009, 22(1-2):55-61.

765. Zanone MM, Favaro E, Miceli I, Grassi G, Camussi E, Caorsi C, Amoroso A, Giovarelli M, Perin PC, Camussi G: Human mesenchymal stem cells modulate cellular immune response to islet antigen glutamic acid decarboxylase in type 1 diabetes. The Journal of clinical endocrinology and metabolism 2010, 95(8):3788-3797.

766. Ko E, Cho KA, Ju SY, Woo SY: Mesenchymal stem cells inhibit the differentiation of CD4+ T cells into interleukin-17-secreting T cells. Acta haematologica 2008, 120(3): 165-167.

767. Rafei M, Campeau PM, Aguilar-Mahecha A, Buchanan M, Williams P, Birman E, Yuan S, Young YK, Boivin MN, Forner K et al: Mesenchymal stromal cells ameliorate experimental autoimmune encephalomyelitis by inhibiting CD4 Th17 T cells in a CC chemokine ligand 2-dependent manner. Journal of immunology 2009, 182(10):5994-6002.

768. Tatara R, Ozaki K, Kikuchi Y, Hatanaka K, Oh I, Meguro A, Matsu H, Sato K, Ozawa K: Mesenchymal stromal cells inhibit Th17 but not regulatory T-cell differentiation. Cytotherapy 2011, 13(6):686-694.

769. Duffy MM, Pindjakova J, Hanley SA, McCarthy C, Weidhofer GA, Sweeney EM, English K, Shaw G, Murphy JM, Barry FP et al: Mesenchymal stem cell inhibition of T-helper 17 cell- differentiation is triggered by cell-cell contact and mediated by prostaglandin E2 via the EP4 receptor. European journal of immunology 2011, 41(10):2840-2851.

770. Luz-Crawford P, Kurte M, Bravo-Alegria J, Contreras R, Nova-Lamperti E, Tejedor G, Noel D, Jorgensen C, Figueroa F, Djouad F et al: Mesenchymal stem cells generate a CD4+CD25+Foxp3+ regulatory T cell population during the differentiation process of Th1 and Th17 cells. Stem Cell Res Ther 2013, 4(3):65.

771. Kota DJ, Wiggins LL, Yoon N, Lee RH: TSG-6 produced by hMSCs delays the onset of autoimmune diabetes by suppressing Th1 development and enhancing tolerogenicity. Diabetes 2013, 62(6):2048-2058.

772. Del Papa B, Sportoletti P, Cecchini D, Rosati E, Balucani C, Baldoni S, Fettucciari K, Marconi P, Martelli MF, Falzetti F et al: Notch1 modulates mesenchymal stem cells mediated regulatory T-cell induction. European journal of immunology 2013, 43(1):182-187.

773. Maccario R, Podesta M, Moretta A, Cometa A, Comoli P, Montagna D, Daudt L, Ibatici A, Piaggio G, Pozzi S et al: Interaction of human mesenchymal stem cells with cells involved in alloantigen-specific immune response favors the differentiation of CD4+ T-cell subsets expressing a regulatory/suppressive phenotype. Haematologica 2005, 90(4):516-525.

774. Di Ianni M, Del Papa B, De Ioanni M, Moretti L, Bonifacio E, Cecchini D, Sportoletti P, Falzetti F, Tabilio A: Mesenchymal cells recruit and regulate T regulatory cells. Experimental hematology 2008, 36(3):309-318.

775. Boumaza I, Srinivasan S, Witt WT, Feghali-Bostwick C, Dai Y, Garcia-Ocana A, Feili-Hariri M: Autologous bone marrow-derived rat mesenchymal stem cells promote PDX-1 and insulin expression in the islets, alter T cell cytokine pattern and preserve regulatory T cells in the periphery and induce sustained normoglycemia. Journal of autoimmunity 2009, 32(1):33-42.

776. Ye Z, Wang Y, Xie HY, Zheng SS: Immunosuppressive effects of rat mesenchymal stem cells: involvement of CD4+CD25+ regulatory T cells. Hepatobiliary & pancreatic diseases international: HBPD INT 2008, 7(6):608-614.

777. Melief SM, Schrama E, Brugman MH, Tiemessen MM, Hoogduijn MJ, Fibbe WE, Roelofs H: Multipotent stromal cells induce human regulatory T cells through a novel pathway involving skewing of monocytes toward anti-inflammatory macrophages. Stem Cells 2013, 31(9): 1980-1991.

778. Cai J, Wu Z, Xu X, Liao L, Chen J, Huang L, Wu W, Luo F, Wu C, Pugliese A et al: Umbilical Cord Mesenchymal Stromal Cell With Autologous Bone Marrow Cell Transplantation in Established Type 1 Diabetes: A Pilot Randomized Controlled Open-Label Clinical Study to Assess Safety and Impact on Insulin Secretion. Diabetes Care 2016, 39(1): 149-157.

779. Dave SD, Trivedi HL, Gopal SC, Chandra T: Combined therapy of insulin-producing cells and haematopoietic stem cells offers better diabetic control than only haematopoietic stem cells’ infusion for patients with insulin-dependent diabetes. BMJ case reports 2014, 2014.

780. Dave SD, Vanikar AV, Trivedi HL: Co-infusion of adipose tissue derived mesenchymal stem cell-differentiated insulin-making cells and haematopoietic cells with renal transplantation: a novel therapy for type 1 diabetes mellitus with end-stage renal disease. BMJ case reports 2013, 2013.

781. Carlsson PO, Schwarcz E, Korsgren O, Le Blanc K: Preserved beta-cell function in type 1 diabetes by mesenchymal stromal cells. Diabetes 2015, 64(2):587-592.

782. Paoletti R, Bolego C, Poli A, Cignarella A: Metabolic syndrome, inflammation and atherosclerosis. Vasc Health Risk Manag 2006, 2(2): 145-152.

783. Scheen AJ: Drug-drug and food-drug pharmacokinetic interactions with new insulinotropic agents repaglinide and nateglinide. Clin Pharmacokinet 2007, 46(2):93-108.

784. Joshi SR: Metformin: old wine in new bottle--evolving technology and therapy in diabetes. J Assoc Physicians India 2005, 53:963-972.

785. Goodarzi MO, Bryer-Ash M: Metformin revisited: re-evaluation of its properties and role in the pharmacopoeia of modern antidiabetic agents. Diabetes Obes Metab 2005, 7(6):654-665.

786. Eriksson A, Attvall S, Bonnier M, Eriksson JW, Rosander B, Karlsson FA: Short-term effects of metformin in type 2 diabetes. Diabetes Obes Metab 2007, 9(3):330-336.

787. Green BD, Irwin N, Duffy NA, Gault VA, O′Harte F P, Flatt PR: Inhibition of dipeptidyl peptidase-IV activity by metformin enhances the antidiabetic effects of glucagon-like peptide-1. Eur J Pharmacol 2006, 547(1-3): 192-199.

788. Faveeuw C, Fougeray S, Angeli V, Fontaine J, Chinetti G, Gosset P, Delerive P, Maliszewski C, Capron M, Staels B et al: Peroxisome proliferator-activated receptor gamma activators inhibit interleukin-12 production in murine dendritic cells. FEBS Lett 2000, 486(3):261-266.

789. Saubermann LJ, Nakajima A, Wada K, Zhao S, Terauchi Y, Kadowaki T, Aburatani H, Matsuhashi N, Nagai R, Blumberg RS: Peroxisome proliferator-activated receptor gamma agonist ligands stimulate a Th2 cytokine response and prevent acute colitis. Inflamm Bowel Dis 2002, 8(5):330-339.

790. Kong D, Zhuang X, Wang D, Qu H, Jiang Y, Li X, Wu W, Xiao J, Liu X, Liu J et al: Umbilical cord mesenchymal stem cell transfusion ameliorated hyperglycemia in patients with type 2 diabetes mellitus. Clinical laboratory 2014, 60(12):1969-1976.

791. Hu J, Wang Y, Gong H, Yu C, Guo C, Wang F, Yan S, Xu H: Long term effect and safety of Wharton’s jelly-derived mesenchymal stem cells on type 2 diabetes. Experimental and therapeutic medicine 2016, 12(3): 1857-1866.

792. Guan LX, Guan H, Li HB, Ren CA, Liu L, Chu JJ, Dai LJ: Therapeutic efficacy of umbilical cord-derived mesenchymal stem cells in patients with type 2 diabetes. Experimental and therapeutic medicine 2015, 9(5): 1623-1630.

793. Skyler JS, Fonseca VA, Segal KR, Rosenstock J, Investigators M-D: Allogeneic Mesenchymal Precursor Cells in Type 2 Diabetes: A Randomized, Placebo-Controlled, Dose-Escalation Safety and Tolerability Pilot Study. Diabetes care 2015, 38(9): 1742-1749.

794. Liu Y, Liu Y, Wang P, Tian H, Ai J, Liu Y, Zhou Y, Liu Z, Guo W, Yang S: Autologous bone marrow stem cell transplantation for the treatment of postoperative hand infection with a skin defect in diabetes mellitus: A case report. Oncology letters 2014, 7(6): 1857-1862.

795. Qin HL, Zhu XH, Zhang B, Zhou L, Wang WY: Clinical Evaluation of Human Umbilical Cord Mesenchymal Stem Cell Transplantation After Angioplasty for Diabetic Foot. Experimental and clinical endocrinology & diabetes : official journal, German Society of Endocrinology [and] German Diabetes Association2016.

796. Li XY, Zheng ZH, Li XY, Guo J, Zhang Y, Li H, Wang YW, Ren J, Wu ZB: Treatment of foot disease in patients with type 2 diabetes mellitus using human umbilical cord blood mesenchymal stem cells: response and correction of immunological anomalies. Current pharmaceutical design 2013, 19(27):4893-4899.

797. Macleod AM, Hillis AN, Mather A, Bone JM, Catto GR: Effect of cyclosporin, previous third-party transfusion, and pregnancy on antibody development after donor-specific transfusion before renal transplantation. Lancet 1987, 1(8530):416-418.

798. Crop MJ, Baan CC, Korevaar SS, Ijzermans JN, Alwayn IP, Weimar W, Hoogduijn MJ: Donor-derived mesenchymal stem cells suppress alloreactivity of kidney transplant patients. Transplantation 2009, 87(6):896-906.

799. Perico N, Casiraghi F, Introna M, Gotti E, Todeschini M, Cavinato RA, Capelli C, Rambaldi A, Cassis P, Rizzo P et al: Autologous mesenchymal stromal cells and kidney transplantation: a pilot study of safety and clinical feasibility. Clinical journal of the American Society of Nephrology : CJASN 2011, 6(2):412-422.

800. Mudrabettu C, Kumar V, Rakha A, Yadav AK, Ramachandran R, Kanwar DB, Nada R, Minz M, Sakhuja V, Marwaha N et al: Safety and efficacy of autologous mesenchymal stromal cells transplantation in patients undergoing living donor kidney transplantation: a pilot study. Nephrology 2015, 20(1):25-33.

801. Reinders ME, de Fijter JW, Roelofs H, Bajema IM, de Vries DK, Schaapherder AF, Claas FH, van Miert PP, Roelen DL, van Kooten C et al: Autologous bone marrow-derived mesenchymal stromal cells for the treatment of allograft rejection after renal transplantation: results of a phase I study. Stem cells translational medicine 2013, 2(2): 107-111.

802. Vanikar AV, Trivedi HL, Kumar A, Gopal SC, Patel HV, Gumber MR, Kute VB, Shah PR, Dave SD: Co-infusion of donor adipose tissue-derived mesenchymal and hematopoietic stem cells helps safe minimization of immunosuppression in renal transplantation - single center experience. Renal failure 2014, 36(9): 1376-1384.

803. Trivedi HL, Vanikar AV, Kute VB, Patel HV, Gumber MR, Shah PR, Dave SD, Trivedi VB: The effect of stem cell transplantation on immunosuppression in living donor renal transplantation: a clinical trial. International journal of organ transplantation medicine 2013, 4(4):155-162.

804. Tan J, Wu W, Xu X, Liao L, Zheng F, Messinger S, Sun X, Chen J, Yang S, Cai J et al: Induction therapy with autologous mesenchymal stem cells in living-related kidney transplants: a randomized controlled trial. Jama 2012, 307(11):1169-1177.

805. Dogan SM, Kilinc S, Kebapci E, Tugmen C, Gurkan A, Baran M, Kurtulmus Y, Olmez M, Karaca C: Mesenchymal stem cell therapy in patients with small bowel transplantation: single center experience. World journal of gastroenterology 2014, 20(25):8215-8220.

806. Peng Y, Ke M, Xu L, Liu L, Chen X, Xia W, Li X, Chen Z, Ma J, Liao D et al: Donor-derived mesenchymal stem cells combined with low-dose tacrolimus prevent acute rejection after renal transplantation: a clinical pilot study. Transplantation 2013, 95(1):161-168.

807. Hagg T, Oudega M: Degenerative and spontaneous regenerative processes after spinal cord injury. J Neurotrauma 2006, 23(3-4):264-280.

808. Matthews MA, St Onge MF, Faciane CL: An electron microscopic analysis of abnormal ependymal cell proliferation and envelopment of sprouting axons following spinal cord transection in the rat. Acta Neuropathol 1979, 45(1):27-36.

809. Bruni JE: Ependymal development, proliferation, and functions: a review. Microsc Res Tech 1998, 41(1):2-13.

810. Park SI, Lim JY, Jeong CH, Kim SM, Jun JA, Jeun SS, Oh WI: Human umbilical cord blood-derived mesenchymal stem cell therapy promotes functional recovery of contused rat spinal cord through enhancement of endogenous cell proliferation and oligogenesis. Journal of biomedicine & biotechnology 2012, 2012:362473.

811. Steffenhagen C, Dechant FX, Oberbauer E, Furtner T, Weidner N, Kury P, Aigner L, Rivera FJ: Mesenchymal stem cells prime proliferating adult neural progenitors toward an oligodendrocyte fate. Stem cells and development 2012, 21(11):1838-1851.

812. Keilhoff G, Stang F, Goihl A, Wolf G, Fansa H: Transdifferentiated mesenchymal stem cells as alternative therapy in supporting nerve regeneration and myelination. Cellular and molecular neurobiology 2006, 26(7-8):1235-1252.

813. Veeravalli KK, Dasari VR, Tsung AJ, Dinh DH, Gujrati M, Fassett D, Rao JS: Human umbilical cord blood stem cells upregulate matrix metalloproteinase-2 in rats after spinal cord injury. Neurobiology of disease 2009, 36(1):200-212.

814. Quertainmont R, Cantinieaux D, Botman O, Sid S, Schoenen J, Franzen R: Mesenchymal stem cell graft improves recovery after spinal cord injury in adult rats through neurotrophic and pro-angiogenic actions. PloS one 2012, 7(6):e39500.

815. Zeng X, Zeng YS, Ma YH, Lu LY, Du BL, Zhang W, Li Y, Chan WY: Bone marrow mesenchymal stem cells in a three-dimensional gelatin sponge scaffold attenuate inflammation, promote angiogenesis, and reduce cavity formation in experimental spinal cord injury. Cell transplantation 2011, 20(11-12):1881-1899.

816. Blight AR, Decrescito V: Morphometric analysis of experimental spinal cord injury in the cat: the relation of injury intensity to survival of myelinated axons. Neuroscience 1986, 19(1):321-341.

817. Nashmi R, Fehlings MG: Changes in axonal physiology and morphology after chronic compressive injury of the rat thoracic spinal cord. Neuroscience 2001, 104(1):235-251.

818. Crowe MJ, Bresnahan JC, Shuman SL, Masters JN, Beattie MS: Apoptosis and delayed degeneration after spinal cord injury in rats and monkeys. Nat Med 1997, 3(1):73-76.

819. Levi AD, Bunge RP: Studies of myelin formation after transplantation of human Schwann cells into the severe combined immunodeficient mouse. Exp Neurol 1994, 130(1):41-52.

820. Casha S, Yu WR, Fehlings MG: Oligodendroglial apoptosis occurs along degenerating axons and is associated with FAS and p75 expression following spinal cord injury in the rat. Neuroscience 2001, 103(1):203-218.

821. Casha S, Yu WR, Fehlings MG: FAS deficiency reduces apoptosis, spares axons and improves function after spinal cord injury. Exp Neurol 2005, 196(2):390-400.

822. Ackery A, Robins S, Fehlings MG: Inhibition of Fas-mediated apoptosis through administration of soluble Fas receptor improves functional outcome and reduces posttraumatic axonal degeneration after acute spinal cord injury. J Neurotrauma 2006, 23(5):604-616.

823. Dasari VR, Spomar DG, Li L, Gujrati M, Rao JS, Dinh DH: Umbilical cord blood stem cell mediated downregulation of fas improves functional recovery of rats after spinal cord injury. Neurochem Res 2008, 33(1):134-149.

824. Lipton SA, Rosenberg PA: Excitatory amino acids as a final common pathway for neurologic disorders. N Engl J Med 1994, 330(9):613-622.

825. Peng W, Cotrina ML, Han X, Yu H, Bekar L, Blum L, Takano T, Tian GF, Goldman SA, Nedergaard M: Systemic administration of an antagonist of the ATP-sensitive receptor P2X7 improves recovery after spinal cord injury. Proc Natl Acad Sci U S A 2009, 106(30): 12489-12493.

826. Wang X, Arcuino G, Takano T, Lin J, Peng WG, Wan P, Li P, Xu Q, Liu QS, Goldman SA et al: P2X7 receptor inhibition improves recovery after spinal cord injury. Nat Med 2004, 10(8):821-827.

827. Park E, Velumian AA, Fehlings MG: The role of excitotoxicity in secondary mechanisms of spinal cord injury: a review with an emphasis on the implications for white matter degeneration. J Neurotrauma 2004, 21(6):754-774.

828. Cizkova D, Nagyova M, Slovinska L, Novotna I, Radonak J, Cizek M, Mechirova E, Tomori Z, Hlucilova J, Motlik J et al: Response of ependymal progenitors to spinal cord injury or enhanced physical activity in adult rat. Cell Mol Neurobiol 2009, 29(6-7):999-1013.

829. Moreno-Manzano V, Rodriguez-Jimenez FJ, Garcia-Rosello M, Lainez S, Erceg S, Calvo MT, Ronaghi M, Lloret M, Planells-Cases R, Sanchez-Puelles JM et al: Activated spinal cord ependymal stem cells rescue neurological function. Stem Cells 2009, 27(3):733-743.

830. Meletis K, Bamabe-Heider F, Carlen M, Evergren E, Tomilin N, Shupliakov O, Frisen J: Spinal cord injury reveals multilineage differentiation of ependymal cells. PLoS Biol 2008, 6(7):e182.

831. Jimenez Hamann MC, Tator CH, Shoichet MS: Injectable intrathecal delivery system for localized administration of EGF and FGF-2 to the injured rat spinal cord. Exp Neurol 2005, 194(1): 106-119.

832. Furukawa S, Furukawa Y: [FGF-2-treatment improves locomotor function via axonal regeneration in the transected rat spinal cord]. Brain Nerve 2007, 59(12):1333-1339.

833. Ribeiro CA, Fraga JS, Graos M, Neves NM, Reis RL, Gimble JM, Sousa N, Salgado AJ: The secretome of stem cells isolated from the adipose tissue and Wharton jelly acts differently on central nervous system derived cell populations. Stem cell research & therapy 2012, 3(3): 18.

834. Madri JA: Modeling the neurovascular niche: implications for recovery from CNS injury. J Physiol Pharmacol 2009, 60 Suppl 4:95-104.

835. Taguchi A, Soma T, Tanaka H, Kanda T, Nishimura H, Yoshikawa H, Tsukamoto Y, Iso H, Fujimori Y, Stern DM et al: Administration of CD34+ cells after stroke enhances neurogenesis via angiogenesis in a mouse model. J Clin Invest 2004, 114(3):330-338.

836. Kim HM, Hwang DH, Lee JE, Kim SU, Kim BG: Ex vivo VEGF delivery by neural stem cells enhances proliferation of glial progenitors, angiogenesis, and tissue sparing after spinal cord injury. PLoS One 2009, 4(3):e4987.

837. Sasaki H, Ishikawa M, Tanaka N, Nakanishi K, Kamei N, Asahara T, Ochi M: Administration of human peripheral blood-derived CD133+ cells accelerates functional recovery in a rat spinal cord injury model. Spine (Phila Pa 1976) 2009, 34(3):249-254.

838. Mueller CA, Conrad S, Schluesener HJ, Pietsch T, Schwab JM: Spinal cord injury-induced expression of the antiangiogenic endostatin/collagen XVIII in areas of vascular remodelling. J Neurosurg Spine 2007, 7(2):205-214.

839. Nair M, Saxena P: Recent patents on mesenchymal stem cell mediated therapy in inflammatory diseases. Recent patents on inflammation & allergy drug discovery 2013, 7(2):105-113.

840. Dazzi F, Lopes L, Weng L: Mesenchymal stromal cells: a key player in ‘innate tolerance’? Immunology 2012, 137(3):206-213.

841. Jackson WM, Nesti LJ, Tuan RS: Mesenchymal stem cell therapy for attenuation of scar formation during wound healing. Stem cell research & therapy 2012, 3(3):20.

842. Meirelles Lda S, Fontes AM, Covas DT, Caplan AI: Mechanisms involved in the therapeutic properties of mesenchymal stem cells. Cytokine & growth factor reviews 2009, 20(5-6):419-427.

843. Seo JH, Cho SR: Neurorestoration induced by mesenchymal stem cells: potential therapeutic mechanisms for clinical trials. Yonsei medical journal 2012, 53(6): 1059-1067.

844. Li J, Lepski G: Cell transplantation for spinal cord injury: a systematic review. BioMed research international 2013, 2013:786475.

845. Kim JW, Ha KY, Molon JN, Kim YH: Bone Marrow Derived Mesenchymal Stem Cell Transplantation for Chronic Spinal Cord Injury in Rats: Comparative Study Between Intralesional and Intravenous Transplantation. Spine 2013.

846. Boido M, Garbossa D, Fontanella M, Ducati A, Vercelli A: Mesenchymal Stem Cell Transplantation Reduces Glial Cyst and Improves Functional Outcome After Spinal Cord Compression. World neurosurgery 2012.

847. Pal R, Venkataramana NK, Bansal A, Balaraju S, Jan M, Chandra R, Dixit A, Rauthan A, Murgod U, Totey S: Ex vivo-expanded autologous bone marrow-derived mesenchymal stromal cells in human spinal cord injury/paraplegia: a pilot clinical study. Cytotherapy 2009, 11(7):897-911.

848. Kishk NA, Gabr H, Hamdy S, Afifi L, Abokresha N, Mahmoud H, Wafaie A, Bilal D: Case control series of intrathecal autologous bone marrow mesenchymal stem cell therapy for chronic spinal cord injury. Neurorehabilitation and neural repair 2010, 24(8):702-708.

849. Karamouzian S, Nematollahi-Mahani SN, Nakhaee N, Eskandary H: Clinical safety and primary efficacy of bone marrow mesenchymal cell transplantation in subacute spinal cord injured patients. Clinical neurology and neurosurgery 2012, 114(7):935-939.

850. Jiang PC, Xiong WP, Wang G, Ma C, Yao WQ, Kendell SF, Mehling BM, Yuan XH, Wu DC: A clinical trial report of autologous bone marrow-derived mesenchymal stem cell transplantation in patients with spinal cord injury. Experimental and therapeutic medicine 2013, 6(1): 140-146.

851. Dai G, Liu X, Zhang Z, Yang Z, Dai Y, Xu R: Transplantation of autologous bone marrow mesenchymal stem cells in the treatment of complete and chronic cervical spinal cord injury. Brain research 2013, 1533:73-79.

852. Mendonca MV, Larocca TF, de Freitas Souza BS, Villarreal CF, Silva LF, Matos AC, Novaes MA, Bahia CM, de Oliveira Melo Martinez AC, Kaneto CM et al: Safety and neurological assessments after autologous transplantation of bone marrow mesenchymal stem cells in subjects with chronic spinal cord injury. Stem cell research & therapy 2014, 5(6): 126.

853. Chotivichit A, Ruangchainikom M, Chiewvit P, Wongkajomsilp A, Sujirattanawimol K: Chronic spinal cord injury treated with transplanted autologous bone marrow-derived mesenchymal stem cells tracked by magnetic resonance imaging: a case report. Journal of medical case reports 2015, 9:79.

854. Oh SK, Choi KH, Yoo JY, Kim DY, Kim SJ, Jeon SR: A Phase III Clinical Trial Showing Limited Efficacy of Autologous Mesenchymal Stem Cell Therapy for Spinal Cord Injury. Neurosurgery2016, 78(3):436-447; discussion 447.

855. Satti HS, Waheed A, Ahmed P, Ahmed K, Akram Z, Aziz T, Satti TM, Shahbaz N, Khan MA, Malik SA: Autologous mesenchymal stromal cell transplantation for spinal cord injury: A Phase I pilot study. Cytotherapy 2016, 18(4):518-522.

856. Thakkar UG, Vanikar AV, Trivedi HL, Shah VR, Dave SD, Dixit SB, Tiwari BB, Shah HH: Infusion of autologous adipose tissue derived neuronal differentiated mesenchymal stem cells and hematopoietic stem cells in post-traumatic paraplegia offers a viable therapeutic approach. Advanced biomedical research 2016, 5:51.

857. Park JH, Kim DY, Sung IY, Choi GH, Jeon MH, Kim KK, Jeon SR: Long-term results of spinal cord injury therapy using mesenchymal stem cells derived from bone marrow in humans. Neurosurgery 2012, 70(5): 1238-1247; discussion 1247.

858. Yazdani SO, Hafizi M, Zali AR, Atashi A, Ashrafi F, Seddighi AS, Soleimani M: Safety and possible outcome assessment of autologous Schwann cell and bone marrow mesenchymal stromal cell co-transplantation for treatment of patients with chronic spinal cord injury. Cytotherapy 2013, 15(7):782-791.

859. Moviglia GA, Fernandez Vina R, Brizuela JA, Saslavsky J, Vrsalovic F, Varela G, Bastos F, Farina P, Etchegaray G, Barbieri M et al: Combined protocol of cell therapy for chronic spinal cord injury. Report on the electrical and functional recovery of two patients. Cytotherapy 2006, 8(3):202-209.

860. Jarocha D, Milczarek O, Wedrychowicz A, Kwiatkowski S, Majka M: Continuous improvement after multiple mesenchymal stem cell transplantations in a patient with complete spinal cord injury. Cell transplantation 2015, 24(4):661-672.

861. Oraee-Yazdani S, Hafizi M, Atashi A, Ashrafi F, Seddighi AS, Hashemi SM, Seddighi A, Soleimani M, Zali A: Co-transplantation of autologous bone marrow mesenchymal stem cells and Schwann cells through cerebral spinal fluid for the treatment of patients with chronic spinal cord injury: safety and possible outcome. Spinal cord 2016, 54(2): 102-109.

862. Derakhshanrad N, Saberi H, Tayebi Meybodi K, Taghvaei M, Arjmand B, Aghayan HR, Kohan AH, Haghpanahi M, Rahmani S: Case Report: Combination Therapy with Mesenchymal Stem Cells and Granulocyte-Colony Stimulating Factor in a Case of Spinal Cord Injury. Basic and clinical neuroscience 2015, 6(4):299-305.

1. A regenerative Wharton Jelly derived cellular composition possessing immune modulatory, neurogenic, anti-inflammatory, angiogenic and regenerative activity.

2. A method of creating a regenerative Wharton Jelly derived cellular composition possessing immune modulatory, neurogenic, anti-inflammatory, angiogenic and regenerative activity, comprising the following steps: a) obtaining an isolated umbilical cord; b) dissociating Wharton’s Jelly to obtain mononuclear cells; c) optionally purifying subsets of said mononuclear cells; d) exposing said mononuclear cells to a regenerative adjuvant; e) culturing said mononuclear cells in absence of, or in the presence of said regenerative adjuvant and/or adding a secondary regenerative adjuvant; and f) selecting said cultured cells for further use, optionally carrying out another purification step.

3. The method of claim 2, wherein said cell isolated from said Wharton’s Jelly express a marker selected from a group of markers comprising: CD144, CD105, and CD31.

4. The method of claim 2, wherein said cells are obtained from placenta perivascular tissue as a substitute for Wharton’s Jelly.

5. The method of claim 4, wherein said cells are s isolated from fetal vascular lobules of a hemochorial placenta.

6. The method of claim 4, wherein said cells are isolated by: dissociating fetal vascular lobules from a hemochorial placenta; digesting the dissociated fetal vascular lobes with an enzymatic mixture or by mechanical means; applying a filtration means to said dissociated lobes in order to remove particulates; obtaining mononuclear cells; plating said mononuclear cells in a substrate allowing for growth of said mononuclear cells to confluency; detaching the confluent cells from the plate; and isolating for expression of CD144 and substantially lack of expression of CD45, optionally one or more steps are performed in the presence of hypoxia, wherein hypoxia is sufficient to induce translocation of HIF-1 alpha.

7. The method of claim 6, wherein dissociation of fetal vascular lobes is accomplishing by incubation with a mixture of about 2% collagenase, about 0.25% trypsin and about 0.1% DNAse in tissue culture medium.

8. The method of claim 2, wherein dissociation of fetal vascular lobes is accomplishing by incubation with a mixture of about 2% collagenase, about 0.25% trypsin and about 0.1% DNAse in tissue culture medium.

9. The method of claim 2, wherein cells isolated are comprised of adherent cells expressing the marker CD73 but substantially lacking CD105.

10. The method of claim 2, wherein cells isolated are comprised of adherent cells expressing the marker CD73 and CD105 but lacking in CD90.

11. The method of claim 2, wherein said regenerative adjuvant is an anti-inflammatory cytokine.

12. The method of claim 11, wherein said anti-inflammatory cytokine is selected from the group consisting of IL-4, IL-10, IL-13, IL-20, IL-22 and IL-35.

13. The method of claim 11, wherein said anti-inflammatory cytokine is TGF-beta.

14. The method of claim 11, wherein said anti-inflammatory cytokine is PGE-2.

15. The method of claim 11, wherein said anti-inflammatory cytokine is VEGF.

16. The method of claim 2, wherein said method is composed of mesenchymal stem cells, and wherein said first regenerative adjuvant is hypoxia.

17. The method of claim 2, wherein said mesenchymal stem cells possess one or more markers selected from a group consisting of: a) CD11b; b) CD11c; c) CD20; d) CD56; e) CD57 f) CD73; g) CD90; h) CD105; i) membrane bound TGF-beta; and j) neuropilin.

18. The method of claim 2, wherein said method is capable of inhibiting T cell mediated immune responses.

19. The method of claim 18, wherein said T cell mediated immune responses comprise of Th1 cell production of cytokines.

20. The method of claim 19, wherein said cytokine is selected from the group consisting of: a) IL-2; b) IL-6; c) IL-8; d) IL-12; e) IL-15; f) IL-18; g) interferon gamma; h) TNF-alpha; and i) interleukin-33.
